(19) United States
(12) Patent Application Publication
(10) Pub. No.: US 2007/0014776 A1
(43) Pub. Date: Jan. 18, 2007

(54) Identification of adiponutrin-related proteins as esterases and methods of use for the same

(76) Inventors: Ruth E. Gimeno, Wellesley, MA (US); Janet E. Paulsen, Londonderry, NH (US); Jian-Liang Li, Medford, MA (US); Andrew C. Lake, Newton, MA (US); Wei Liu, Lexington, MA (US); Jae Eun Kim, Waltham, MA (US)

Correspondence Address: FITZPATRICK CELLA (WYETH), 30 ROCKEFELLER PLAZA, NEW YORK, NY, 10112-3800, US

(21) Appl. No.: 11/450,935

(22) Filed: Jun. 9, 2006

Related U.S. Application Data
(60) Provisional application No. 60/689,408, filed on Jun. 9, 2005.

(51) Int. Cl. A61K 38/54; C12Q 1/34; C07H 21/04; C12P 21/06; C12N 9/99

(52) U.S. Cl. 424/94.2; 435/18; 435/69.1; 435/184; 435/320.1; 435/325; 536/23.2

(57) ABSTRACT

The present invention provides methods of identifying polypeptides that have enzymatic activity associated with nutrient and/or energy homeostasis, and thus, are involved in the development of one or more cardiovascular and metabolic disorders, e.g., cardiovascular disease, obesity, insulin resistance, type 2 diabetes, dyslipidemia, nonalcoholic fatty liver disease, and metabolic syndrome. One such method comprises identifying a polypeptide as a member of the adiponutrin family of proteins. As such, the invention is related to the polynucleotides and polypeptides belonging to the adiponutrin family, and provides novel isolated and purified polynucleotides and polypeptides of a novel member of the adiponutrin family, patatin-like phospholipase domain 1 (PNPLA1). Also provided are methods of using the polynucleotides and polypeptides related to or provided by the invention for screening a test compound, e.g., a small molecule, antibody, etc., for the ability of the test compound to detect and/or modulate the activity of one or more members of the adiponutrin family of proteins. The present invention also is directed to novel methods for diagnosing, prognosing, and monitoring the progress of at least one cardiovascular and metabolic disorder using polynucleotides or polypeptides belonging to the adiponutrin family, and/or modulators of one or more members of the adiponutrin family. The present invention is further directed to novel therapeutics and therapeutic targets for the intervention (treatment) and prevention of cardiovascular and metabolic disorders arising from dysregulated energy homeostasis, as related to one or more members of the adiponutrin family of proteins.

This application claims the benefit of priority from U.S. Provisional Patent Application No. 60/689,408, filed Jun. 9, 2005, the content of which is hereby incorporated by reference herein in its entirety.

### BACKGROUND OF THE INVENTION

1. Field of the Invention

The present invention is directed to novel methods for identifying polypeptides that have enzymatic activity related to nutrient and/or energy homeostasis, e.g., polypeptides belonging to the adiponutrin family of proteins, and thus are involved in the development of cardiovascular and metabolic diseases. The present invention is further directed to novel therapeutics and therapeutic targets, and to methods of screening and assessing test compounds for the intervention (treatment) and prevention of disorders arising from dysregulation of nutrient and/or energy homeostasis, as related to one or more members of the adiponutrin family of proteins. The invention also provides methods of diagnosing, prognosing, monitoring the progress of, and treating disorders arising from dysregulation of nutrient and/or energy homeostasis (e.g., cardiovascular and metabolic disorders including, but not limited to, cardiovascular disease, obesity, insulin resistance, type 2 diabetes, dyslipidemia, nonalcoholic fatty liver disease, metabolic syndrome, etc.) related to one or more members of the adiponutrin family and modulators related thereto.

2. Related Background Art

Adipose tissue is a key regulator of energy balance, not only as a storage depot for fat, but also as an important source of paracrine and endocrine factors (Kershaw and Flier (2004) J. Clin. Endocrinol. Metab. 89:2548-56; Klaus (2004) Curr. Drug Targets 5:241-50; Guerre-Millo (2004) Diabetes Metab. 30:13-19; Fruhbeck (2004) Curr. Med. Chem. Cardiovasc. Hematol. Agents 2:197-208). Fatty acid storage (lipogenesis) and release (lipolysis) from adipose tissue is tightly regulated (Haemmerle et al. (2003) Curr. Opin. Lipidol. 14:289-97), and dysregulation of these processes has been implicated in the pathophysiology of one or more cardiovascular and metabolic disorders (e.g., cardiovascular disease (i.e., any disease that affects the heart or blood vessels, e.g., by restricting the flow of blood), obesity, insulin resistance, type 2 diabetes, dyslipidemia, nonalcoholic fatty liver disease, metabolic syndrome (i.e., a syndrome involving the simultaneous occurrence of a group of health conditions, which may include insulin resistance, abdominal obesity, dyslipidemia, hypertension, chronic inflammation, a prothrombotic state, etc. that places a person at high risk for type 2 diabetes and/or heart disease), etc. (see, e.g., Bergman et al. (2001) J. Investig. Med. 49:119-26; Blaak (2003) Proc. Nutr. Soc. 62:753-60; Arner (2002) Diabetes Metab. Res. Rev. 18 (Suppl. 2):S5-9; Semenkovich (2004) Trends Cardiovasc. Med. 14:72-76)).

Lipolysis is mediated by intracellular lipases that act sequentially to remove fatty acid groups from the glycerol backbone of triglycerides to ultimately form glycerol and free fatty acids. Until recently, the major triglyceride lipase was thought to be hormone-sensitive lipase (HSL), a lipid-droplet associated protein whose activity and subcellular localization is regulated by lipogenic and lipolytic stimuli (Haemmerle et al. (2003) supra; Large et al. (2004) Diabetes Metab. 30:294-309). The presence of significant residual lipolysis in adipose tissue of HSL knockout mice suggested the existence of an additional triglyceride lipase. Recently, a candidate for this activity, desnutrin/adipocyte triglyceride lipase (desnutrin/ATGL), was identified and shown to be responsible for most, if not all, lipolysis remaining in an HSL-deletion mouse (Villena et al (2004) J. Biol. Chem. 279(45):47066-75; Zimmermann et al. (2004) Science 306:1383-86).

Interestingly, desnutrin/ATGL belongs to a family of proteins defined by adiponutrin, an adipocyte-specific protein of unknown function, which recently has been shown to have lipid hydrolysis activity; two additional adiponutrin-related proteins have been identified recently in the literature (Jenkins et al. (2004) J. Biol. Chem. 279:48968-75). In other words, the adiponutrin amino acid sequence shows homology to the amino acid sequences of desnutrin/ATGL, GS2, and GS2-like proteins.

Adiponutrin is a gene that encodes a 413-amino acid membrane-bound protein that is expressed primarily in adipose tissues and is induced early in the differentiation of 3T3-L1 cells to adipocytes (Baulande et al. (2001) J. Biol. Chem. 276:33336-44; Polson and Thompson (2003) Biochem. Biophys. Res. Commun. 301:261-66). In contrast to desnutrin/ATGL, the expression of which is upregulated under conditions of increased lipolysis (i.e., fasting), adiponutrin mRNA is dramatically decreased during fasting (Polson and Thompson (2003) supra; Liu et al. (2004) J. Clin. Endocrinol. Metab. 89:2684-89; Polson and Thompson (2004) J. Nutr. Biochem. 15:242-46; Bertile and Raclot (2004) Biochim. Biophys. Acta 1683:101-09). Since the expression levels of adiponutrin are regulated by different metabolic paradigms, e.g., its expression is downregulated after animals are subject to starvation and is increased in genetically obese fa/fa Zucker rats, it is hypothesized that adiponutrin may be important in the regulation of nutrient and/or energy homeostasis by adipose tissue (Baulande, supra; Polson and Thompson (2003) supra). However, because the function of adiponutrin is currently unknown, the hypothesis remains unproven.

Adiponutrin, desnutrin/ATGL, GS2, and GS2-like proteins all contain a patatin-like domain at their N-terminus (Villena et al. (2004) supra). Patatin is a member of a family of proteins found in potato and other solanaceous plants (Rydel et al. (2003) Biochemistry 42:6696-708). The proteins in the patatin family have been shown to have lipid acyl hydrolase activity that catalyzes the nonspecific hydrolysis of phospholipids, glycolipids, sulfolipids, and mono- and diacylglycerols (Rydel, supra). Comparison of patatin with other lipases indicates that it has two conserved amino acid motifs characteristic of esterases: a Gly-X-Ser-X-Gly motif and an Asp-X-Gly/Ala motif (Mignery et al. (1984) Nucleic Acids Res. 12:7987-8000; Mignery et al. (1988) Gene 62:27-44; Stiekema et al. (1988) Plant Mol. Biol. 11:255-69; Rosahl et al. (1986) Mol. Gen. Genet. 203:214-20; Rydel, supra). The enzymatic activity of patatin has been localized to a catalytic dyad consisting of the Ser residue in the Gly-X-Ser-X-Gly motif and a conserved aspartate residue in an Asp-X-Gly/Ala motif (Rydel, supra).

The presence of a patatin-like domain (i.e., the Gly-X-Ser-X-Gly and Asp-X-Gly/Ala motifs) in adiponutrin, desnutrin/ATGL, GS2, and GS2-like proteins has led to the hypothesis that these proteins have enzymatic activity similar to patatin, and further supports the hypothesis that these proteins are involved in a metabolic paradigm (Villena, supra). Only recently have adiponutrin, desnutrin/ATGL and GS2 been shown to have triacylglycerol lipase activity (Jenkins, supra). Studies involving desnutrin/ATGL have demonstrated that ectopic expression of desnutrin/ATGL in COS-7 cells resulted in significantly decreased triglyceride levels in the cells, and suggests that desnutrin/ATGL may be implicated in cardiovascular and metabolic disorders associated with altered adipocyte function or lipid metabolism, such as cardiovascular disease, obesity, insulin resistance, type 2 diabetes, dyslipidemia, nonalcoholic fatty liver disease, metabolic syndrome (i.e., a syndrome involving the simultaneous occurrence of a group of health conditions, which may include insulin resistance, abdominal obesity, dyslipidemia, hypertension, chronic inflammation, a prothrombotic state, etc. that places a person at high risk for type 2 diabetes and/or heart disease), etc. Unlike adiponutrin, GS2 is expressed in several tissues, such as muscle and liver tissue; its expression in adipose tissue and its ability to fimction as a lipase in a cell-based system has been unclear. Furthermore, the expression patterns of GS2-like in adipose tissue and its enzymatic activity and function in cells have not yet been examined. Additionally, the existence of other adiponutrin family members has been unclear. Consequently, the existence of other adiponutrin related proteins needs to be determined and assays that measure the function of adiponutrin-related proteins need to be developed for targeted examination of the role of these proteins in energy homeostasis, and for development of methods of diagnosing, prognosing, monitoring the progress of, and treating disorders arising from, dysregulation of nutrient and/or energy homeostasis.

### SUMMARY OF THE INVENTION

The present invention provides methods that determine the existence of other adiponutrin related proteins, these methods comprising a comprehensive bioinformatic analysis of the adiponutrin family. Use of such methods herein identified five family members, i.e., adiponutrin, desnutrin/ATGL, GS2, GS2-like and novel PNPLA1. Additionally, the present invention provides evidence that adiponutrin, desnutrin/ATGL, GS2, and GS2-like are biologically active esterases, e.g., lipases. First, the present invention demonstrates that adiponutrin family members are spatially and temporally expressed in a manner consistent with playing an important role in nutrient and/or energy homeostasis, e.g., that GS2-like protein is predominantly expressed in adipose tissue and is regulated by different metabolic paradigms. Second, provided herein is a demonstration of lipid hydrolase activity and the effects of overexpression of adiponutrin, desnutrin/ATGL, GS2 and GS2-like on fatty acid metabolism, e.g., that GS2-like protein is regulated in metabolic paradigms both in vitro and in vivo, suggesting that it may play a role in the pathogenesis of metabolic disorders and/or that modulating its function might be beneficial. The present invention supports desnutrin/ATGL as the major adiponutrin family lipase in mouse adipocytes, and raises the possibility that an adiponutrin homolog, GS2, may contribute to lipolysis in human adipocytes. In addition, the present invention supports a possible role for adiponutrin and GS2-like in lipid metabolism in the liver.

Thus, in one embodiment, the present invention provides a method for identifying a polypeptide associated with at least one of the group of cardiovascular and metabolic disorders including, but not limited to, cardiovascular disease (i.e., any disease that affects the heart or blood vessels, e.g., by restricting the flow of blood), obesity, insulin resistance, type 2 diabetes, dyslipidemia, nonalcoholic fatty liver disease, metabolic syndrome (i.e., a syndrome involving the simultaneous occurrence of a group of health conditions, which may include insulin resistance, abdominal obesity, dyslipidemia, hypertension, chronic inflammation, a prothrombotic state, etc. that places a person at high risk for type 2 diabetes and/or heart disease), etc., the method comprising the steps of determining that the expression of the polypeptide is regulated by one or more metabolic paradigms, and determining that the polypeptide has enzymatic activity related to nutrient and/or energy homeostasis. In another embodiment, one of the one or more metabolic paradigms is determining that expression of the polypeptide is enriched in or specific to adipose tissue. In a further embodiment, the adipose tissue is white adipose tissue. In another further embodiment, the adipose tissue is brown adipose tissue. In another embodiment, the enzymatic activity is esterase activity. In a further embodiment, the esterase activity is lipid acyl hydrolase activity. In another further embodiment, the esterase activity is lipase activity. In another embodiment, the invention provides an isolated polypeptide identified by the aforementioned method, or an active fragment thereof.

In another embodiment, the invention provides a method for identifying a polypeptide as a member of the adiponutrin family of proteins, the method comprising the steps of identifying a patatin-like domain at the N-terminus of the polypeptide, determining that the polypeptide has enzymatic activity related to nutrient and/or energy homeostasis, and demonstrating a close evolutionary relationship between the polypeptide and other members of the adiponutrin family of proteins. In another embodiment, the enzymatic activity is esterase activity. In a further embodiment, the esterase activity is lipid acyl hydrolase activity. In another further embodiment, the esterase activity is lipase activity. In another embodiment, the invention provides an isolated polypeptide identified by the aforementioned method, or an active fragment thereof.

In another embodiment, the invention provides a novel member of the adiponutrin protein family, PNPLA1. In particular, the invention provides an isolated nucleic acid molecule having the nucleotide sequence of SEQ ID NO:22 or SEQ ID NO:25. In another embodiment, the nucleic acid molecule is operably linked to at least one expression control sequence. In another embodiment, the invention provides a host cell transformed or transfected with the nucleic acid molecule. In another embodiment, the invention provides an isolated nucleic acid molecule that specifically hybridizes under highly stringent conditions to the aforementioned nucleotide sequence, or to its complement.

In another embodiment, the invention provides an isolated nucleic acid molecule that encodes a protein having the amino acid sequence of SEQ ID NO:23 or SEQ ID NO:26.

In another embodiment, the invention provides an antisense oligonucleotide complementary to an MRNA corresponding to a nucleic acid molecule having the nucleotide sequence of SEQ ID NO:22 or SEQ ID NO:25, wherein the antisense oligonucleotide inhibits production of PNPLA1.

In another embodiment, the invention provides an siRNA molecule targeted to an mRNA corresponding to a nucleic acid molecule having the nucleotide sequence of SEQ ID NO:22 or SEQ ID NO:25, wherein the siRNA molecule inhibits production of PNPLA1.

In another embodiment, the invention provides an inhibitory polynucleotide targeted to a regulatory region of a gene corresponding to SEQ ID NO:24 or SEQ ID NO:27, wherein the inhibitory polynucleotide inhibits production of PNPLA1.

In another embodiment, the invention provides an isolated gene having the nucleotide sequence of SEQ ID NO:24 or SEQ ID NO:27. In another embodiment, the invention provides an isolated allele of a nucleic acid molecule having the nucleotide sequence of SEQ ID NO:22, SEQ ID NO:24, SEQ ID NO:25, or SEQ ID NO:27.

In another embodiment, the invention provides an isolated protein having the amino acid sequence of SEQ ID NO:23 or SEQ ID NO:26, or an active fragment thereof.

In another embodiment, the invention provides a nonhuman knockout animal in which the somatic and germ cells exhibit inhibited PNPLA1 production. In another embodiment, the invention provides a nonhuman transgenic animal in which the somatic and germ cells contain DNA comprising a nucleic acid molecule having the nucleotide sequence of SEQ ID NO:22 or SEQ ID NO:24.

The present invention provides expression vectors that may be used to modulate (e.g., inhibit, enhance) esterase activity. In one embodiment, the invention provides an expression vector for inhibiting esterase activity, wherein the expression vector comprises a nucleic acid sequence selected from the group consisting of SEQ ID NOs:1, 4, 7, 10, 13, 16, 19, 22, 25, and portions thereof. In another embodiment, the invention provides a cell exhibiting an inhibited esterase activity, wherein the cell comprises the aforementioned expression vector. In another embodiment, the invention provides a nonhuman knockout animal exhibiting an inhibited esterase activity, wherein the animal comprises the aforementioned expression vector. In a further embodiment, the invention provides the knockout animal wherein the inhibited esterase activity is lipid acyl hydrolase activity. In another further embodiment, the invention provides the knockout animal wherein the inhibited esterase activity is lipase activity.

In another embodiment, the invention provides an expression vector for enhancing esterase activity, wherein the expression vector comprises a nucleic acid sequence selected from the group consisting of SEQ ID NOs:1, 4, 7, 10, 13, 16, 19, 22, 25, and portions thereof encoding active fragments of the polypeptides encoded by said SEQ ID NOs. In another embodiment, the invention provides a cell exhibiting an enhanced esterase activity, wherein the cell comprises the aforementioned expression vector. In another embodiment, the invention provides a nonhuman transgenic animal with an enhanced esterase activity, wherein the animal comprises the aforementioned expression vector. In a further embodiment, the invention provides the transgenic animal wherein the enhanced esterase activity is lipid acyl hydrolase activity. In another further embodiment, the invention provides the transgenic animal wherein the enhanced esterase activity is lipase activity.

In another embodiment, the invention provides a method for identifying a modulator of the esterase activity of a member of the adiponutrin family of proteins, the method comprising the steps of contacting the member of the adiponutrin family with a candidate modulator and determining whether the candidate modulator modulates the esterase activity of the member of the adiponutrin family of proteins. In another embodiment, the esterase activity is lipid acyl hydrolase activity. In another embodiment, the esterase activity is lipase activity. In another embodiment, the step of contacting the member of the adiponutrin family of proteins with the candidate modulator comprises contacting a cell with the candidate modulator, wherein the cell comprises an expression vector comprising a nucleic acid sequence selected from the group consisting of SEQ ID NOs:1, 4, 7, 10, 13, 16, 19, 22, 25, 52, and 55, and portions thereof encoding active fragments of the polypeptides encoded by the same SEQ ID NOs. In one embodiment, the cell is an adipocyte. In another embodiment, the cell is a preadipocyte. In one embodiment, the adipocyte or preadipocyte is a 3T3-L1 cell. In another embodiment, the step of contacting the cell (e.g., adipocyte, preadipocyte, 3T3-L1 cell, etc.) with the candidate modulator comprises contacting the cell with isoproterenol. In another embodiment, the step of contacting the cell with the candidate modulator comprises contacting the cell with insulin, e.g., before contacting the cell with isoproterenol.

In another embodiment, the invention provides a method for identifying a modulator of the lipid acyl hydrolase activity of a member of the adiponutrin family of proteins, the method comprising the steps of contacting the member of the adiponutrin family with a candidate modulator and determining whether the candidate modulator modulates the lipid acyl hydrolase activity of the member of the adiponutrin family. In one embodiment, the modulator is selected from the group consisting of small molecules and antibodies. In another embodiment, the invention provides such a modulator of one or more members of the adiponutrin family. In another embodiment, the step of contacting the member of the adiponutrin family of proteins with the candidate modulator comprises contacting a cell with the candidate modulator, wherein the cell comprises an expression vector comprising a nucleic acid sequence selected from the group consisting of SEQ ID NOs:1, 4, 7, 10, 13, 16, 19, 22, 25, 52, and 55, and portions thereof encoding active fragments of the polypeptides encoded by the same SEQ ID NOs. In one embodiment, the cell is an adipocyte. In another embodiment, the cell is a preadipocyte. In one embodiment, the adipocyte or preadipocyte is a 3T3-L1 cell. In another embodiment, the step of contacting the cell (e.g., adipocyte, preadipocyte, 3T3-L1 cell, etc.) with the candidate modulator comprises contacting the cell with isoproterenol. In another embodiment, the step of contacting the cell with the candidate modulator comprises contacting the cell with insulin, e.g., before contacting the cell with isoproterenol.

In another embodiment, the invention provides a method for identifying a modulator of the lipase activity of a member of the adiponutrin family of proteins, the method comprising the steps of contacting the member of the adiponutrin family with a candidate modulator and determining whether the candidate modulator modulates the lipase activity of the member of the adiponutrin family. In one embodiment, the modulator is selected from the group consisting of small molecules and antibodies. In another embodiment, the invention provides such a modulator of one or more members of the adiponutrin family. In another embodiment, the step of contacting the member of the adiponutrin family of proteins with the candidate modulator comprises contacting a cell with the candidate modulator, wherein the cell comprises an expression vector comprising a nucleic acid sequence selected from the group consisting of SEQ ID NOs:1, 4, 7, 10, 13, 16, 19, 22, 25, 52, and 55, and portions thereof encoding active fragments of the polypeptides encoded by the same SEQ ID NOs. In one embodiment, the cell is an adipocyte. In another embodiment, the cell is a preadipocyte. In one embodiment, the adipocyte or preadipocyte is a 3T3-L1 cell. In another embodiment, the step of contacting the cell (e.g., adipocyte, preadipocyte, 3T3-L1 cell, etc.) with the candidate modulator comprises contacting the cell with isoproterenol. In another embodiment, the step of contacting the cell with the candidate modulator comprises contacting the cell with insulin, e.g., before contacting the cell with isoproterenol.

The invention also provides a method for modulating esterase activity in a subject, the method comprising administering to the subject a modulator of the esterase activity of one or more members of the adiponutrin family of proteins. In one embodiment, the modulator is identified by a method of the invention. In another embodiment, the esterase activity that is modulated is lipid acyl hydrolase activity. In another embodiment, the esterase activity that is modulated is lipase activity.

In another embodiment, the invention provides a method for modulating lipid acyl hydrolase activity in a subject comprising administering to the subject a modulator of the lipid acyl hydrolase activity of one or more members of the adiponutrin family of proteins. In one embodiment, the modulator is identified by a method of the invention. The invention also provides a method for modulating lipase activity in a subject comprising administering to the subject a modulator of the lipase activity of one or more members of the adiponutrin family of proteins.

The invention also provides a method for identifying a modulator for the treatment of a cardiovascular or metabolic disease selected from the group consisting of cardiovascular disease, obesity, insulin resistance, type 2 diabetes, dyslipidemia, nonalcoholic fatty liver disease, and metabolic syndrome, the method comprising determining whether a candidate modulator modulates the enzymatic activity of one or more members of the adiponutrin family of proteins. In one embodiment, the enzymatic activity is esterase activity. In another embodiment, the esterase activity is lipid acyl hydrolase activity. In another embodiment, the esterase activity is lipase activity.

In one embodiment, the invention provides a method for treating a cardiovascular or metabolic disease selected from the group consisting of cardiovascular disease, obesity, insulin resistance type 2 diabetes, dyslipidemia, nonalcoholic fatty liver disease, and metabolic syndrome, the method comprising administering to a subject a modulator of the enzymatic activity of one or more members of the adiponutrin family of proteins. In one embodiment, the administered modulator is identified by a method of the invention. In another embodiment, the enzymatic activity is esterase activity. In another embodiment, the enzymatic activity is lipid acyl hydrolase activity. In another embodiment, the enzymatic activity is lipase activity.

The invention also provides a pharmaceutical composition comprising a modulator of the activity of one or more members of the adiponutrin family of proteins. In one embodiment, the pharmaceutical composition comprises a modulator identified by a method of the invention. In another embodiment, the pharmaceutical composition comprises a modulator of the activity of one or more members of the adiponutrin family of proteins and a pharmaceutically acceptable carrier.

The invention also provides a kit comprising a modulator of the activity of one or more members of the adiponutrin family of proteins. In one embodiment, the kit comprises an antibody to one or more members of the adiponutrin family of proteins. The invention also provides a kit comprising a detecting antibody to one or more members of the adiponutrin family.

The invention also provides an expression vector for inhibiting lipase activity, wherein the expression vector comprises a nucleic acid sequence selected from the group consisting of SEQ ID NOs: 1, 13, and 52, and portions thereof encoding active fragments of the polypeptides encoded by the same SEQ ID NOs. The invention also provides a cell exhibiting inhibited lipase activity, wherein the cell comprises an expression vector comprising a nucleic acid sequence selected from the group consisting of SEQ ID NOs:1, 13, and 52, and portions thereof encoding active fragments of the polypeptides encoded by the same SEQ ID NOs. Additionally, in another embodiment, the invention provides a nonhuman transgenic animal, wherein the animal comprises an expression vector comprising a nucleic acid sequence selected from the group consisting of SEQ ID NOs:1, 13, and 52, and portions thereof encoding active fragments of the polypeptides encoded by the same SEQ ID NOs.

The method also provides a method for identifying a modulator of a member of the adiponutrin family of proteins, the method comprising the step of contacting a cell with a candidate modulator, wherein the cell comprises an expression vector comprising a nucleic acid sequence selected from the group consisting of SEQ ID NOs:1, 4, 7, 10, 13, 16, 19, 22, 25, 52, and 55, and portions thereof encoding active fragments of the polypeptides encoded by the same SEQ ID NOs. In one embodiment, the expression vector comprises the nucleic acid sequence of SEQ ID NO:1. In another embodiment, the cell is an adipocyte. In another embodiment, the cell is a preadipocyte. In one embodiment, the adipocyte or preadipocyte is a 3T3-L1 cell. In another embodiment, the step of contacting the cell (e.g., adipocyte, preadipocyte, 3T3-L1 cell, etc.) with the candidate modulator comprises contacting the cell with isoproterenol. In another embodiment, the step of contacting the cell with the candidate modulator comprises contacting the cell with insulin, e.g., before contacting the cell with isoproterenol.

### BRIEF DESCRIPTION OF THE DRAWINGS

Shown in FIG. 1 is a Venn diagram of datasets of genes that demonstrate one or more of the following characteristics: 1) the presence of a signal peptide, 2) enriched or specific expression in adipose tissue, and 3) the presence of one or more transmembrane domains.

FIG. 2A is a bootstrap neighbor-joining phylogenetic tree showing the evolutionary relationship between the amino acid sequence of the patatin-like domains of potato patatin (*) and the amino acid sequences of the patatin-like domains of human (h), mouse (m), and/or rat (r) NTE, NTE-like, PLA2G6, IPLA2, IPLA2-like, GS2, adiponutrin, desnutrin/ATGL, PNPLA1, and GS2-like proteins. The box indicates the close evolutionary relationship between the predicted amino acid sequences of the patatin-like domains of human and rat GS2; murine, human, and putative rat adiponutrin; murine, rat and human desnutrin/ATGL; putative rat, putative murine, and putative human PNPLA1; and rat, murine and human GS2-like proteins. PF01734 is the consensus amino acid sequence that was generated from Pfam model alignment. Shown in FIG. 2B are the amino acid alignments of the patatin-like domains of human (h) adiponutrin, hDesnutrin/ATGL, hGS2-like, predicted hPNPLA1, and hGS2 proteins and the conserved hydrolase motifs (the Gly-X-Ser-X-Gly (GXSXG) motif and the conserved Asp (D)) that are predicted to form the active site. FIG. 2C depicts the patatin-like domain at the N-terminus of each member of the adiponutrin family and the size of each protein.

Shown in FIG. 3A are Northern blots of human poly(A) (3^{rd} panel) or total RNA (1^{st}, 2^{nd} and 4^{th} panels) derived from the tissues indicated and probed with probes specific for human (h) desnutrin/ATGL or hGS2. The lower panels are the corresponding blots probed with β-actin. FIG. 3B demonstrates Q-PCR analysis of mouse (m) adiponutrin, mDesnutrin/ATGL, or mGS2-like RNA in multiple mouse tissues: spleen (Spl), epididymal fat (Efat), small intestine (S In), liver (Liv), kidney (Kid), brown adipose (BAT), lung (Lun), heart (H), colon (Col), stomach (Sto), gastrocnemius muscle (Ske) and brain (Br). Inset graphs are standard curves generated from plasmid DNA for each gene that were used to determine copy number/ng RNA (y-axes). Error bars depict standard deviation of the mean copy number.

FIG. 4 shows the RNA quantities (fold change; y-axes) of mouse adiponutrin, mouse desnutrin/ATGL, and mouse GS2-like in the following samples: stromal fraction (Stroma) or primary adipocytes (Fat) isolated from epididymal adipose tissue of C57BL/6 mice (column 1); undifferentiated 3T3-L1 cells (preadipocytes; PreAdipo) and differentiated 3T3-L1 adipocytes (Adipo; column 2); epididymal white adipose tissue (WAT) or liver tissue (Liv) isolated from either wild-type control mice (WT) or genetically obese ob/ob mice (columns 3-4); and epididymal WAT or Liv tissue isolated from C57BL/6 mice that were either fed ad libitum (Fed), or fasted for 24 hours (Fast; columns 5-6). Error bars represent the fold change range for the standard error of the mean and an asterisk (*) denotes a p-value of less than 0.05 relative to control.

The graphs of FIG. 5 demonstrate lipase activity (OD581/second; y-axes) of protein A beads immunoprecipitated from cells transfected with expression vectors comprising untagged (Un) adiponutrin, desnutrin/ATGL, GS2, or GS2-like; V5-tagged (V5) adiponutrin, desnutrin/ATGL, GS2, or GS2-like, or green fluorescence protein (GFP; upper left graph only). An asterisk (*) denotes a p-value of less than 0.05 relative to control (GFP and/or Un). The insets of FIG. 5 are Western blots probed with anti-V5 antibody showing V5-tagged protein expression in cell lysates from transfected cells (Lysate), lysates after immunoprecipitation (Post), the first wash (Wash) and Protein A beads after immunoprecipitation (Bead).

FIG. 6 demonstrates lipase activity (OD581/second; y-axes) of protein A beads immunoprecipitated from cells transfected with expression constructs comprising V5-tagged wild type (WT) or V5-tagged patatin-like domain mutant (MUT) human adiponutrin (FIG. 6A), human desnutrin/ATGL (FIG. 6B), human GS2 (FIG. 6C), or human GS2-like (FIG. 6D) expression constructs. An asterisk (*) denotes a p-value of less than 0.05 relative to control. The insets of FIG. 6 are Western blots probed with anti-V5 antibody. The lanes in each were loaded with either cell lysates (L) or immunoprecipitated beads (B) from cells transfected with V5-tagged WT adiponutrin family members (WT) or transfected with V5-tagged mutant adiponutrin family members (MUT).

FIG. 7 demonstrates [1-⁴C]-oleic acid incorporation into triglycerides by HEK293 cells transfected with an empty expression construct (Vector) or expression constructs comprising human adiponutrin, human desnutrin/ATGL, human GS2, or human GS2-like. Error bars represent standard error of the mean. An asterisk (*) denotes a p-value of less than 0.05 relative to control (Vector).

FIG. 8 shows the lipase activity of 3T3-L1 cells transfected with either green fluorescence protein (GFP; ♦) or human adiponutrin (ADPN; ▪) after isoproterenol treatment (hrs; x-axes) as a measure of (A) free fatty acid release (OD550; y-axis) and (B) glycerol release (OD540; y-axis).

FIG. 9 demonstrates glycerol release (OD540; y-axis) by green fluorescence protein (GFP; □) or human adiponutrin (ADPN; ▪)-transfected 3T3-L1 cells that were untreated (−) or pretreated (+) with insulin and subsequently incubated in the absence (−) or presence (+) of isoproterenol.

### DETAILED DESCRIPTION OF THE INVENTION

The present invention is based the finding that, unlike other proteins containing a patatin-like domain, the patatin-like domains of adiponutrin, desnutrin/ATGL, GS2, and GS2-like proteins have a close evolutionary relationship and can thus be classified as members of a protein family (hereinafter “the adiponutrin family” or “the adiponutrin family of proteins”). Additionally, the present invention is based on the findings that members of the adiponutrin family are regulated with several metabolic paradigms and have enzymatic activity. These findings strongly suggest that the members of the adiponutrin family are contributors to the regulatory role played by adipose tissue in nutrient and/or energy homeostasis, and as such, may be involved in at least one cardiovascular and metabolic disorder (e.g., cardiovascular disease (i.e., any disease that affects the heart or blood vessels, e.g., by restricting the flow of blood (as a nonlimiting set of examples: hypertension, heart failure, stroke)), obesity, insulin resistance, type 2 diabetes, dyslipidemia, nonalcoholic fatty liver disease, metabolic syndrome (i.e., a syndrome involving the simultaneous occurrence of a group of health conditions, which may include insulin resistance, abdominal obesity, dyslipidemia, hypertension, chronic inflammation, a prothrombotic state, etc. that places a person at high risk for type 2 diabetes and/or heart disease), etc.). Other disorders or diseases for which the members of the adiponutrin family may be contributors to the regulatory role played by adipose tissue in nutrient and/or energy homeostasis include, but are not limited to: wasting disorders/cachexia; arrhythmias, and cancer. The findings of the present invention thus allow for methods of identifying other proteins that may be involved in nutrient and/or energy homeostasis and/or the development of cardiovascular and metabolic disorders. The methods of identifying other proteins involved in nutrient and/or energy homeostasis and/or the development of at least one cardiovascular and metabolic disorder may be based on the relationship of the protein to the adiponutrin family, or based on the regulated expression of the protein by different metabolic paradigms and demonstration of enzymatic activity of the protein. Additionally, the findings of the present invention allow methods for measuring the function of members of the adiponutrin family, methods for examining the roles played by members of the adiponutrin family in nutrient and/or energy homeostasis, and methods for screening test compounds (e.g., small molecules, antibodies, etc.) for the ability to modulate the activity of one or more members of the adiponutrin family. Since members of the adiponutrin family are likely involved in nutrient and/or energy homeostasis, and therefore, the development of cardiovascular and metabolic disorders, the invention also provides test compounds capable of modulating the activity of one or more members of the adiponutrin family that may be used to diagnose, prognose, monitor and/or treat such cardiovascular and metabolic disorders.

Thus the invention provides methods of identifying other proteins that may be involved in nutrient and/or energy homeostasis and/or the development of cardiovascular and metabolic disorders. The methods of identifying other proteins involved in nutrient and/or energy homeostasis and/or the development of cardiovascular and metabolic disorders may be based on the regulated expression of the protein by different metabolic paradigms and demonstration of enzymatic activity of the protein, or based on the relationship of the protein to the adiponutrin family. As such, the invention is related to polynucleotides and polypeptides belonging to the adiponutrin family, and provides the novel polynucleotides and polypeptides of PNPLA1. The invention also provides methods to use these polynucleotides and polypeptides for the direct examination of the function of these polynucleotides and polypeptides. Additionally, the invention provides methods for identifying modulators (e.g., small molecules, antibodies, etc.) capable of detecting, and/or enhancing or inhibiting the activity of, one or more members of the adiponutrin family. Also, the present invention provides methods of using an identified modulator of the activity of one or more members of the adiponutrin family (including those identified by a method of the invention) to diagnose, prognose, monitor and/or treat at least one cardiovascular and metabolic disorder. Finally, the invention provides pharmaceutical compositions comprising a modulator of the activity of one or more members of the adiponutrin family to be used in the treatment of at least one cardiovascular and metabolic disorder.

Methods for Identifying Proteins Involved in Nutrient and/or Energy Homeostasis and/or the Development of Cardiovascular and Metabolic Disorders

The present invention provides evidence that adiponutrin, desnutrin/ATGL, GS2 and GS2-like proteins are regulated by one or more metabolic paradigms (e.g., these proteins are regulated by diet, adipocyte differentiation, genetic obesity, etc.) and have enzymatic activity related to nutrient and/or energy homeostasis (e.g., enzymatic activity associated with lipogenesis and/or lipolysis such as esterase, lipid acyl hydrolase, and/or lipase activity). These findings suggest that adiponutrin, desnutrin/ATGL, GS2, and GS2-like proteins help adipocytes regulate nutrient and/or energy homeostasis, and therefore, may be involved in the development of at least one cardiovascular and metabolic disorder. Additionally, the present invention is based on the findings that these proteins belong to the adiponutrin family of proteins. Consequently, the present invention provides methods of identifying other proteins that may also be involved in nutrient and/or energy homeostasis of animals (preferably vertebrate animals, more preferably primates, and most preferably human) and/or the development of one or more cardiovascular and metabolic disorders in these animals.

In one embodiment of the invention, a protein is identified as being involved in nutrient and/or energy homeostasis and/or the development of at least one cardiovascular and metabolic disorder because the expression of the protein is regulated by one or more metabolic paradigms and results in enzymatic activity related to lipogenesis and/or lipolysis.

Demonstration that a protein is regulated by one or more metabolic paradigms may be accomplished by methods well known to one of skill in the art. For example, several well-known methods for determining the presence, and/or the level of expression, of a protein include methods that detect and determine the level of MRNA encoding the protein (e.g., reverse transcriptase PCR, real-time PCR, Northern blot analysis, microarray analysis, etc.) and methods that detect and determine the level of the protein itself (e.g., Western blot analysis, flow cytometric analysis, etc.). Additionally, one of skill in the art will recognize that methods for determining whether expression of the protein is regulated by a metabolic paradigm include, but are not limited to, demonstrating the expression of the protein is enriched in or specific to adipose tissue, demonstrating the expression of the protein is upregulated or downregulated by factors that affect nutrient and/or energy homeostasis (e.g., adipocyte differentiation, genetic make-up, and/or diet), and demonstrating the expression of the protein is regulated in the liver.

For example, to demonstrate that the expression of the protein (or cDNA encoding such protein) is enriched in or specific to adipose tissue, the expression level of the protein (or cDNA encoding the polypeptide) may be determined using any of the methods described above for a panel of biological tissues, which includes adipose tissue, and comparing the expression levels of the protein in each tissue. A protein is enriched in adipose tissue if its expression level is significantly higher in adipose tissue compared to several other tissues in the panel. A protein is specific to adipose tissue if it is expressed primarily and/or exclusively in adipose tissue. One of skill in the art will recognize that a panel should include more than one biological tissue in addition to adipose tissue, and that exemplary panels are commercially available from, e.g., Clontech (Palo Alto, Calif.) and United States Biological (Swampscott, Mass.). Several cell lines are available to demonstrate that the expression of a protein is regulated by adipocyte differentiation, including but not limited to the white adipocyte cell lines, 3T3-L1 (as described above) and F442A, and the brown adipocyte cell line Hib I B. In addition, adipocyte precursor cells may be isolated from human or animal brown or white adipose tissue and differentiated in vitro. Primary human adipocytes as well as reagents and protocols for differentiation are available from a variety of commercial sources, e.g., Zen-Bio (Research Triangle Park, N.C.) and Cambrex (East Rutherford, N.J.). To demonstrate that expression of a protein is regulated by adipocyte differentiation, the expression levels of the protein may be determined in a first sample of cells that have not differentiated into adipocytes, e.g., 3T3-L1 cells, and compared to the expression levels of the protein in a second sample of the cells that have differentiated into adipocytes, e.g., 3T3-L1 cells subject to culture conditions that promote their differentiation into adipocytes. Expression of the protein is regulated by adipocyte differentiation if its level is either upregulated or downregulated in, e.g., the second sample compared to the first sample.

Several animal models of obesity exist (e.g., genetically obese fa/fa rats, genetically obese ob/ob mice, etc.) that may be used to determine whether the protein is regulated by a genetic predisposition for dysregulated nutrient and/or energy homeostasis. A protein is regulated by a genetic predisposition for dysregulated nutrient and/or energy homeostasis if its expression level is either upregulated or downregulated in a genetically obese animal compared to a wild-type animal.

One of skill in the art will recognize other methods that may be used to determine whether the expression of the proteins is regulated by a metabolic paradigm. For example, a first sample containing a biological tissue expressing the protein of interest (e.g., an animal) may be subject to a stimulus affecting nutrient and/or energy homeostasis (e.g., nutrient starvation) and the expression of the protein in the biological tissue may be determined. The expression of the protein is regulated by a metabolic paradigm if its expression is upregulated or downregulated in response to the stimulus.

To identify a protein as being involved in nutrient and/or energy homeostasis and/or the development of at least one cardiovascular and metabolic disease, the protein must be regulated by a metabolic paradigm and also exhibit enzymatic activity. Methods for demonstrating that a protein has enzymatic activity are well known in the art and are dependent on the type of enzymatic activity that is sought to be demonstrated. For example, the protein may be sequenced and analyzed for the identification of conserved domains known to have enzymatic activity. The methods that may be used to demonstrate the enzymatic activity are dependent on the identified domains. For example, if the identified domains are associated with esterase activity, an appropriate method for testing the enzymatic activity of the protein would include methods that subject the isolated protein to a substrate that is susceptible to esterase activity, e.g., an ester, and determining whether the ester is cleaved by the isolated protein. The appropriate substrates for testing particular enzymatic activities are well known in the art.

In one embodiment of the invention, a protein is identified as being involved in nutrient and/or energy homeostasis and/or the development of a cardiovascular and metabolic disorder because it is a member of the adiponutrin family of proteins.

Several bioinformatics tools, including the use of computer programs, such as SignalP, Sigcleave, TMHMM, Pfam, programs that perform phylogenetic analyses, and sequence prediction algorithms were used to identify a novel member of the adiponutrin family of proteins (i.e., PNPLA1), and to classify adiponutrin, desnutrin/ATGL, GS2, GS2-like and PNPLA1 as members of the adiponutrin family of proteins (see Example 1). As such, the invention provides methods for identifying members of the adiponutrin family and methods for identifying a protein as a member of the adiponutrin family. The criteria that must be met for a protein to be classified as a member of the adiponutrin family include the following: 1) expression of the protein must be enriched in and/or specific to adipose tissue and/or expression of the protein must be regulated by a metabolic paradigm (as described above), 2) the protein must contain, at its N-terminus, an active patatin-like domain that is conserved in both primary and tertiary structure to the patatin-like domain of a protein chosen from the group consisting of adiponutrin, desnutrin/ATGL, GS2, GS2-like and PNPLA1, and 3) the protein must have a close evolutionary relationship to adiponutrin, desnutrin/ATGL, GS2, GS2-like and/or PNPLA1. The methods for identifying a protein with the above characteristics, and or determining that a protein exhibits the above characteristics are well known in the art and include the methods described in Example 1 and methods similar to those described in Example 1.

Polynucleotides and Polypeptides Belonging to the Adiponutrin Family

The present invention is based on the finding that members of the adiponutrin family, e.g., adiponutrin, desnutrin/ATGL, GS2, GS2-like and PNPLA1 polypeptides, are regulated with different metabolic paradigms and have enzymatic activity. As such, the present invention is related to isolated and/or purified polynucleotides and polypeptides of members of the adiponutrin family. The invention also provides the isolated and/or purified polynucleotides and polypeptides a novel member of the adiponutrin family, PNPLA1.

For example, the invention relates to isolated polynucleotides encoding adiponutrin, desnutrin/ATGL, GS2, and GS2-like proteins. Preferred DNA sequences related to the invention include genomic and cDNA sequences and chemically synthesized DNA sequences.

The nucleotide sequences of cDNAs encoding human adiponutrin cDNA, human desnutrin/ATGL cDNA, human GS2 cDNA, and human GS2-like cDNA are set forth in SEQ ID NOs:1, 4, 7, and 10, respectively. Polynucleotides related to the present invention also include polynucleotides that hybridize under stringent conditions to SEQ ID NOs: 1, 4, 7, and 10, or complements thereof, and/or encode polypeptides that retain substantial biological activity of full-length human adiponutrin, human desnutrin/ATGL, human GS2, and human GS2-like polypeptides. Polynucleotides related to the present invention also include continuous portions or fragments of the sequences set forth in SEQ ID NOs:1, 4, 7, and 10, comprising at least 21 consecutive nucleotides. Preferred polynucleotides related to the present invention comprise the nucleotide sequences of SEQ ID NO:1 from nucleotide 201 to nucleotide 711, SEQ ID NO:4 from nucleotide 230 to 740, SEQ ID NO:7 from nucleotide 146 to nucleotide 659, or SEQ ID NO:10 from nucleotide 34 to nucleotide 544.

The amino acid sequences of human adiponutrin protein, human desnutrin/ATGL protein, human GS2 protein, and human GS2-like protein are set forth in SEQ ID NOs:2, 5, 8, and 11, respectively. Polypeptides related to the present invention also include continuous portions or fragments of the sequences set forth in SEQ ID NOs:2, 5, 8, and 11, comprising at least seven consecutive amino acids. A preferred polypeptide related to the present invention includes any continuous portion of the sequences set forth in SEQ ID NOs:2, 5, 8 and 11 that retains substantial biological activity (i.e., an active fragment) of full-length human adiponutrin, human desnutrin/ATGL, human GS2, or human GS2-like protein. Preferred polypeptides comprise the amino acid sequences of SEQ ID NO:2 from amino acid 10 to amino acid 179, SEQ ID NO:5 from amino acid 10 to amino acid 179, SEQ ID NO:8 from amino acid 6 to amino acid 176, or SEQ ID NO: 11 from amino acid 12 to amino acid 181.

The nucleotide sequences of genomic DNA encoding human adiponutrin protein (designated human adiponutrin genomic DNA), human desnutrin/ATGL protein (designated human desnutrin/ATGL genomic DNA), human GS2 protein (designated human GS2 genomic DNA), and human GS2-like protein (designated human GS2-like genomic DNA) are set forth in SEQ ID NOs:3, 6, 9, and 12, respectively. Polynucleotides related to the present invention also include polynucleotides that hybridize under stringent conditions to SEQ ID NOs:3, 6, 9, and 12, or complements thereof, and/or encode polypeptides that retain substantial biological activity of full-length human adiponutrin, human desnutrin/ATGL, human GS2, and human GS2-like proteins. Polynucleotides related to the present invention also include continuous portions of the sequences set forth in SEQ ID NOs:3, 6, 9, and 12 comprising at least 21 consecutive nucleotides.

Polynucleotides related to the present invention also include, in addition to those polynucleotides of human origin described above, polynucleotides that encode the amino acid sequences set forth in SEQ ID NOs:2, 5, 8, and 11, or continuous portions thereof, and that differ from the polynucleotides of human origin described above due only to the well-known degeneracy of the genetic code.

The invention is also related to the murine homologs of the polynucleotides and polypeptides described above. The nucleotide sequence of cDNAs encoding murine adiponutrin cDNA, murine desnutrin/ATGL cDNA, and murine GS2-like cDNA are set forth in SEQ ID NOs:13, 16, and 19, respectively. Polynucleotides related to the present invention also include polynucleotides that hybridize under stringent conditions to SEQ ID NOs:13, 16, and 19, or complements thereof, and/or encode polypeptides that retain substantial biological activity of full-length murine adiponutrin, murine desnutrin/ATGL, and murine GS2-like polypeptides. Polynucleotides related to the present invention also include continuous portions of the sequences set forth in SEQ ID NOs:13, 16, and 19, comprising at least 21 consecutive nucleotides. Preferred polynucleotides related to the present invention comprise the nucleotide sequences of SEQ ID NO: 13 from nucleotide 66 to nucleotide 576, SEQ ID NO:16 from nucleotide 108 to 618, or SEQ ID NO:19 from nucleotide 96 to nucleotide 606.

The amino acid sequences of murine adiponutrin protein, murine desnutrin/ATGL protein, and murine GS2-like protein are set forth in SEQ ID NOs:14, 17, and 20, respectively. Polypeptides related to the present invention also include continuous portions of the sequences set forth in SEQ ID NOs:14, 17, and 20, comprising at least seven consecutive amino acids. A preferred polypeptide related to the present invention includes any continuous portion of the sequences set forth in SEQ ID NOs:14, 17, and 20 that retains substantial biological activity (i.e., an active fragment) of full-length murine adiponutrin, murine desnutrin/ATGL, or murine GS2-like protein. Preferred polypeptides comprise the amino acid sequences of SEQ ID NO:14 from amino acid 10 to amino acid 179, SEQ ID NO:17 from amino acid 10 to amino acid 179, or SEQ ID NO:20 from amino acid 12 to amino acid 181.

The nucleotide sequences of genomic DNA encoding murine adiponutrin protein (designated murine adiponutrin genomic DNA), murine desnutrin/ATGL protein (designated murine desnutrin/ATGL genomic DNA), and murine GS2-like protein (designated murine GS2-like genomic DNA) are set forth in SEQ ID NOs: 15, 18, and 21, respectively. Polynucleotides related to the present invention also include polynucleotides that hybridize under stringent conditions to SEQ ID NOs: 15, 18, and 21, or complements thereof, and/or encode polypeptides that retain substantial biological activity of full-length murine adiponutrin, murine desnutrin/ATGL, and murine GS2-like proteins. Polynucleotides of the present invention also include continuous portions of the sequences set forth in SEQ ID NOs:15, 18, and 21, comprising at least 21 consecutive nucleotides.

Polynucleotides related to the present invention also include, in addition to those polynucleotides of murine origin described above, polynucleotides that encode the amino acid sequences set forth in SEQ ID NOs:14, 17, and 20, or continuous portions thereof, and that differ from the polynucleotides of murine origin described above due only to the well-known degeneracy of the genetic code.

The invention also provides purified and isolated polynucleotides encoding a novel member of the adiponutrin family, designated PNPLA1. Preferred DNA sequences related to the invention include genomic and cDNA sequences and chemically synthesized DNA sequences.

The nucleotide sequence of a cDNA encoding the novel member of the adiponutrin family, human PNPLA1, designated human PNPLA1 cDNA, is set forth in SEQ ID NO:22. Polynucleotides of the present invention also include polynucleotides that hybridize under stringent conditions to SEQ ID NO:22, or its complement, and/or encode polypeptides that retain substantial biological activity of full-length human PNPLA1. Polynucleotides of the present invention also include continuous portions of the sequence set forth in SEQ ID NO:22 comprising at least 21 consecutive nucleotides. A preferred polynucleotide of the present invention comprises the nucleotide sequence of SEQ ID NO:22 from nucleotide 46 to nucleotide 529.

The deduced amino acid sequence of human PNPLA1 is set forth in SEQ ID NO:23. Polypeptides of the present invention also include continuous portions of the sequence set forth in SEQ ID NO:23 comprising at least seven consecutive amino acids. A preferred polypeptide of the present invention includes any continuous portion of the sequence set forth in SEQ ID NO:23 that retains substantial biological activity (i.e., an active fragment) of full-length human PNPLA1. One such preferred polypeptide comprises the amino acid sequence of SEQ ID NO:23 from amino acid 16 to amino acid 176.

The nucleotide sequence of a genomic DNA encoding this novel human PNPLA1, designated human PNPLA1 genomic DNA, is set forth in SEQ ID NO:24. Polynucleotides of the present invention also include polynucleotides that hybridize under stringent conditions to SEQ ID NO:24 or its complement, and/or encode polypeptides that retain substantial biological activity of full-length human PNPLA1. Polynucleotides of the present invention also include continuous portions of the sequence set forth in SEQ ID NO:24 comprising at least 21 consecutive nucleotides.

Polynucleotides of the present invention also include, in addition to those polynucleotides of human origin described above, polynucleotides that encode the amino acid sequence set forth in SEQ ID NO:23 or a continuous portion thereof, and that differ from the polynucleotides of human origin described above only due to the well-known degeneracy of the genetic code.

The nucleotide sequence of a cDNA encoding the novel member of the adiponutrin family, murine PNPLA1, designated murine PNPLA1 cDNA, is set forth in SEQ ID NO: 25. Polynucleotides of the present invention also include polynucleotides that hybridize under stringent conditions to SEQ ID NO:25, or its complement, and/or encode polypeptides that retain substantial biological activity of full-length murine PNPLA1. Polynucleotides of the present invention also include continuous portions of the sequence set forth in SEQ ID NO:25 comprising at least 21 consecutive nucleotides. A preferred polynucleotide of the present invention comprises the nucleotide sequence of SEQ ID NO:25 from nucleotide 426 to nucleotide 933.

The deduced amino acid sequence of murine PNPLA1 is set forth in SEQ ID NO:26. Polypeptides of the present invention also include continuous portions of the sequence set forth in SEQ ID NO:26 comprising at least seven consecutive amino acids. A preferred polypeptide of the present invention includes any continuous portion of the sequence set forth in SEQ ID NO:26 that retains substantial biological activity (i.e., an active fragment) of full-length murine PNPLA1. One such preferred polypeptide comprises the amino acid sequence of SEQ ID NO:26 from amino acid 16 to amino acid 184.

The nucleotide sequence of a genomic DNA encoding this novel murine PNPLA1, designated murine PNPLA1 genomic DNA, is set forth in SEQ ID NO:27. Polynucleotides of the present invention also include polynucleotides that hybridize under stringent conditions to SEQ ID NO:27, or its complement, and/or encode polypeptides that retain substantial biological activity of full-length murine PNPLA1. Polynucleotides of the present invention also include continuous portions of the sequence set forth in SEQ ID NO:27 comprising at least 21 consecutive nucleotides.

Polynucleotides of the present invention also include, in addition to those polynucleotides of murine origin described above, polynucleotides that encode the amino acid sequence set forth in SEQ ID NO:26 or a continuous portion thereof, and that differ from the polynucleotides of murine origin described above only due to the well-known degeneracy of the genetic code.

The invention also provides purified and isolated polynucleotides encoding a novel rat adiponutrin. Preferred DNA sequences related to the invention include genomic and cDNA sequences and chemically synthesized DNA sequences.

The nucleotide sequence of a cDNA encoding the novel rat adiponutrin, designated rat adiponutrin cDNA, is set forth in SEQ ID NO:52. Polynucleotides of the present invention also include polynucleotides that hybridize under stringent conditions to SEQ ID NO:52, or its complement, and/or encode polypeptides that retain substantial biological activity of full-length rat adiponutrin. Polynucleotides of the present invention also include continuous portions of the sequence set forth in SEQ ID NO:52 comprising at least 21 consecutive nucleotides. A preferred polynucleotide of the present invention comprises the nucleotide sequence of SEQ ID NO:52 from nucleotide 28 to nucleotide 538.

The deduced amino acid sequence of rat adiponutrin is set forth in SEQ ID NO:53. Polypeptides of the present invention also include continuous portions of the sequence set forth in SEQ ID NO:53 comprising at least seven consecutive amino acids. A preferred polypeptide of the present invention includes any continuous portion of the sequence set forth in SEQ ID NO:53 that retains substantial biological activity (i.e., an active fragment) of full-length rat adiponutrin. One such preferred polypeptide comprises the amino acid sequence of SEQ ID NO:53 from amino acid 10 to amino acid 179.

The nucleotide sequence of a genomic DNA encoding this novel rat adiponutrin, designated rat adiponutrin genomic DNA, is set forth in SEQ ID NO:54. Polynucleotides of the present invention also include polynucleotides that hybridize under stringent conditions to SEQ ID NO:54 or its complement, and/or encode polypeptides that retain substantial biological activity of full-length rat adiponutrin. Polynucleotides of the present invention also include continuous portions of the sequence set forth in SEQ ID NO:54 comprising at least 21 consecutive nucleotides.

Polynucleotides of the present invention also include, in addition to those polynucleotides of rat origin described above, polynucleotides that encode the amino acid sequence set forth in SEQ ID NO:53 or a continuous portion thereof, and that differ from the polynucleotides of rat origin described above only due to the well-known degeneracy of the genetic code.

The invention also provides purified and isolated polynucleotides encoding a novel rat PNPLA1. Preferred DNA sequences related to the invention include genomic and cDNA sequences and chemically synthesized DNA sequences.

The nucleotide sequence of a cDNA encoding the novel rat PNPLA1, designated rat PNPLA1, is set forth in SEQ ID NO:55. Polynucleotides of the present invention also include polynucleotides that hybridize under stringent conditions to SEQ ID NO:55, or its complement, and/or encode polypeptides that retain substantial biological activity of full-length rat PNPLA1. Polynucleotides of the present invention also include continuous portions of the sequence set forth in SEQ ID NO:55 comprising at least 21 consecutive nucleotides. A preferred polynucleotide of the present invention comprises the nucleotide sequence of SEQ ID NO:55 from nucleotide 46 to nucleotide 532.

The deduced amino acid sequence of rat PNPLA1 is set forth in SEQ ID NO:56. Polypeptides of the present invention also include continuous portions of the sequence set forth in SEQ ID NO:56 comprising at least seven consecutive amino acids. A preferred polypeptide of the present invention includes any continuous portion of the sequence set forth in SEQ ID NO:56 that retains substantial biological activity (i.e., an active fragment) of full-length rat PNPLA1. One such preferred polypeptide comprises the amino acid sequence of SEQ ID NO:56 from amino acid 16 to amino acid 177.

The nucleotide sequence of a genomic DNA encoding this novel rat PNPLA1, designated rat PNPLA1 genomic DNA, is set forth in SEQ ID NO:57. Polynucleotides of the present invention also include polynucleotides that hybridize under stringent conditions to SEQ ID NO:57 or its complement, and/or encode polypeptides that retain substantial biological activity of full-length rat PNPLA1. Polynucleotides of the present invention also include continuous portions of the sequence set forth in SEQ ID NO:57 comprising at least 21 consecutive nucleotides.

Polynucleotides of the present invention also include, in addition to those polynucleotides of rat origin described above, polynucleotides that encode the amino acid sequence set forth in SEQ ID NO:56 or a continuous portion thereof, and that differ from the polynucleotides of rat origin described above only due to the well-known degeneracy of the genetic code.

The nucleic acids related to or provided by the present invention may comprise DNA or RNA and may be wholly or partially synthetic. Reference to a nucleotide sequence as set out herein encompasses a DNA molecule with the specified sequence, and encompasses an RNA molecule with the specified sequence in which U is substituted for T, unless context requires otherwise.

The isolated polynucleotides related to or provided by the present invention may be used as hybridization probes and primers to identify and isolate nucleic acids having sequences identical to or similar to those encoding the disclosed polynucleotides. Hybridization methods for identifying and isolating nucleic acids include polymerase chain reaction (PCR), Southern hybridizations, in situ hybridization and Northern hybridization, and are well known to those skilled in the art.

Hybridization reactions can be performed under conditions of different stringency. The stringency of a hybridization reaction includes the difficulty with which any two nucleic acid molecules will hybridize to one another. Preferably, each hybridizing polynucleotide hybridizes to its corresponding polynucleotide under reduced stringency conditions, more preferably stringent conditions, and most preferably highly stringent conditions. Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1

| | | | Hybridization | |
|---|---|---|---|---|
| Stringency | Polynucleotide | | Temperature and | Wash Temperature |
| Condition | Hybrid | Hybrid Length (bp)¹ | Buffer² | and Buffer² |
| A | DNA:DNA | >50 | 65° C.; 1X SSC -or- | 65° C.; 0.3X SSC |
| | | | 42° C.; 1X SSC, 50% | |
| | | | formamide | |
| B | DNA:DNA | <50 | T_{B}*; 1X SSC | T_{B}*; 1X SSC |
| C | DNA:RNA | >50 | 67° C.; 1X SSC -or- | 67° C.; 0.3X SSC |
| | | | 45° C.; 1X SSC, 50% | |
| | | | formamide | |
| D | DNA:RNA | <50 | T_{D}*; 1X SSC | T_{D}*; 1X SSC |
| E | RNA:RNA | >50 | 70° C.; 1X SSC -or- | 70° C.; 0.3X SSC |
| | | | 50° C.; 1X SSC, 50% | |
| | | | formamide | |
| F | RNA:RNA | <50 | T_{F}*; 1X SSC | T_{F}*; 1X SSC |
| G | DNA:DNA | >50 | 65° C.; 4X SSC -or- | 65° C.; 1X SSC |
| | | | 42° C.; 4X SSC, 50% | |
| | | | formamide | |
| H | DNA:DNA | <50 | T_{H}*; 4X SSC | T_{H}*; 4X SSC |
| I | DNA:RNA | >50 | 67° C.; 4X SSC -or- | 67° C.; 1X SSC |
| | | | 45° C.; 4X SSC, 50% | |
| | | | formamide | |
| J | DNA:RNA | <50 | T_{J}*; 4X SSC | T_{J}*; 4X SSC |
| K | RNA:RNA | >50 | 70° C.; 4X SSC -or- | 67° C.; 1X SSC |
| | | | 50° C.; 4X SSC, 50% | |
| | | | formamide | |
| L | RNA:RNA | <50 | T_{L}*; 2X SSC | T_{L}*; 2X SSC |
| M | DNA:DNA | >50 | 50° C.; 4X SSC -or- | 50° C.; 2X SSC |
| | | | 40° C.; 6X SSC, 50% | |
| | | | formamide | |
| N | DNA:DNA | <50 | T_{N}*; 6X SSC | T_{N}*; 6X SSC |
| O | DNA:RNA | >50 | 55° C.; 4X SSC -or- | 55° C.; 2X SSC |
| | | | 42° C.; 6X SSC, 50% | |
| | | | formamide | |
| P | DNA:RNA | <50 | T_{P}*; 6X SSC | T_{P}*; 6X SSC |
| Q | RNA:RNA | >50 | 60° C.; 4X SSC -or- | 60° C.; 2X SSC |
| | | | 45° C.; 6X SSC, 50% | |
| | | | formamide | |
| R | RNA:RNA | <50 | T_{R}*; 4X SSC | T_{R}*; 4X SSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ²SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete. | | | | |
| T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base | | | | |
| # pairs in length, Tₘ(° C.) = 81.5 + 16.6(log₁₀Na⁺) + 0.41(% G + C) − (600/N), where N is the number of bases in the hybrid, and Na⁺ is the concentration of sodium ions in the hybridization buffer (Na⁺ for 1X SSC = 0.165 M). | | | | |

Additional examples of stringency conditions for polynucleotide hybridization are provided in Sambrook et al., Molecular Cloning: A Laboratory Manual, Chs. 9 & 11, Cold Spring Harbor Laboratory Press, Cold Spring Harbor, NY (1989), and Ausubel et al., eds., Current Protocols in Molecular Biology, Sects. 2.10 & 6.3-6.4, John Wiley & Sons, Inc. (1995), herein incorporated by reference.

The isolated polynucleotides provided by the present invention (i.e., of the present invention), or related to the present invention, may be used as hybridization probes and primers to identify and isolate DNA having sequences encoding allelic variants of the disclosed polynucleotides. Allelic variants are naturally occurring alternative forms of the disclosed polynucleotides that encode polypeptides that are identical to or have significant similarity to the polypeptides encoded by the disclosed polynucleotides. Preferably, allelic variants have at least 90% sequence identity (more preferably, at least 95% identity; most preferably, at least 99% identity) with the disclosed polynucleotides.

The isolated polynucleotides related to or provided by the present invention may also be used as hybridization probes and primers to identify and isolate DNAs having sequences encoding polypeptides homologous to the disclosed polynucleotides. These homologs are polynucleotides and polypeptides isolated from a different species than that of the disclosed polypeptides and polynucleotides, or within the same species, but with significant sequence similarity to the disclosed polynucleotides and polypeptides. Preferably, polynucleotide homologs have at least 50% sequence identity (more preferably, at least 75% identity; most preferably, at least 90% identity) with the disclosed polynucleotides, whereas polypeptide homologs have at least 30% sequence identity (more preferably, at least 45% identity; most preferably, at least 60% identity) with the disclosed polypeptides. Preferably, homologs of the disclosed polynucleotides and polypeptides are those isolated from mammalian species.

The isolated polynucleotides related to or provided by the present invention may also be used as hybridization probes and primers to identify cells and tissues that express the polypeptides related to or provided by the present invention and the conditions under which they are expressed.

Additionally, the function of the polypeptides related to or provided by the present invention may be directly examined by using the polynucleotides encoding the polypeptides to alter (i.e., enhance, reduce, or modify) the expression of the genes corresponding to the polynucleotides related to or provided by the present invention in a cell or organism. These “corresponding genes” are the genomic DNA sequences related to or provided by the present invention that are transcribed to produce the mRNAs from which the polynucleotides related to or provided by the present invention are derived.

Altered expression of the genes related to or provided by the present invention may be achieved in a cell or organism through the use of various inhibitory polynucleotides, such as antisense polynucleotides and ribozymes that bind and/or cleave the MRNA transcribed from the genes related to or provided by the invention (see, e.g., Galderisi et al. (1999) J. Cell Physiol. 181:251-57; Sioud (2001) Curr. Mol. Med. 1:575-88). Such inhibitory polynucleotides may be useful in preventing or treating at least one cardiovascular and metabolic disorder (e.g., cardiovascular disease (i.e., any disease that affects the heart or blood vessels, e.g., by restricting the flow of blood), obesity, insulin resistance, type 2 diabetes, dyslipidemia, nonalcoholic fatty liver disease, metabolic syndrome (i.e., a syndrome involving the simultaneous occurrence of a group of health conditions, which may include insulin resistance, abdominal obesity, dyslipidemia, hypertension, chronic inflammation, a prothrombotic state, etc. that places a person at high risk for type 2 diabetes and/or heart disease), etc.).

The antisense polynucleotides or ribozymes provided by the invention can be complementary to an entire coding strand of a gene related to or provided by the invention, or to only a portion thereof. Alternatively, antisense polynucleotides or ribozymes can be complementary to a noncoding region of the coding strand of a gene related to or provided by the invention. The antisense polynucleotides or ribozymes can be constructed using chemical synthesis and enzymatic ligation reactions using procedures well known in the art. The nucleoside linkages of chemically synthesized polynucleotides can be modified to enhance their ability to resist nuclease-mediated degradation, as well as to increase their sequence specificity. Such linkage modifications include, but are not limited to, phosphorothioate, methylphosphonate, phosphoroamidate, boranophosphate, morpholino, and peptide nucleic acid (PNA) linkages (Galderisi et al., supra; Heasman (2002) Dev. Biol. 243:209-14; Micklefield (2001) Curr. Med. Chem. 8:1157-79). Alternatively, these molecules can be produced biologically using an expression vector into which a polynucleotide related to or provided by the present invention has been subcloned in an antisense (i.e., reverse) orientation.

The inhibitory polynucleotides of the present invention also include triplex-forming oligonucleotides (TFOs) that bind in the major groove of duplex DNA with high specificity and affinity (Knauert and Glazer (2001) Hum. Mol. Genet. 10:2243-51). Expression of the genes related to or provided by the present invention can be inhibited by targeting TFOs complementary to the regulatory regions of the genes (i.e., the promoter and/or enhancer sequences) to form triple helical structures that prevent transcription of the genes.

In one embodiment of the invention, the inhibitory polynucleotides of the present invention are short interfering RNA (siRNA) molecules. These siRNA molecules are short (preferably 19-25 nucleotides; most preferably 19 or 21 nucleotides), double-stranded RNA molecules that cause sequence-specific degradation of target MRNA. This degradation is known as RNA interference (RNAi) (e.g., Bass (2001) Nature 411:428-29). Originally identified in lower organisms, RNAi has been effectively applied to mammalian cells and has recently been shown to prevent fulminant hepatitis in mice treated with siRNA molecules targeted to Fas mRNA (Song et al. (2003) Nature Med. 9:347-51). In addition, intrathecally delivered siRNA has recently been reported to block pain responses in two models (agonist-induced pain model and neuropathic pain model) in the rat (Dorn et al. (2004) Nucleic Acids Res. 32(5):e49).

The siRNA molecules of the present invention can be generated by annealing two complementary single-stranded RNA molecules together (one of which matches a portion of the target MRNA) (Fire et al., U.S. Pat. No. 6,506,559) or through the use of a single hairpin RNA molecule that folds back on itself to produce the requisite double-stranded portion (Yu et al. (2002) Proc. Natl. Acad. Sci. USA 99:6047-52). The siRNA molecules can be chemically synthesized (Elbashir et al. (2001) Nature 411:494-98) or produced by in vitro transcription using single-stranded DNA templates (Yu et al., supra). Alternatively, the siRNA molecules can be produced biologically, either transiently (Yu et al., supra; Sui et al. (2002) Proc. Natl. Acad. Sci. USA 99:5515-20) or stably (Paddison et al. (2002) Proc. Natl. Acad. Sci. USA 99:144348), using an expression vector(s) containing the sense and antisense siRNA sequences. Recently, reduction of levels of target MRNA in primary human cells, in an efficient and sequence-specific manner, was demonstrated using adenoviral vectors that express hairpin RNAs, which are further processed into siRNAs (Arts et al. (2003) Genome Res. 13:2325-32).

The siRNA molecules targeted to the polynucleotides related to or provided by the present invention can be designed based on criteria well known in the art (e.g., Elbashir et al. (2001) EMBO J. 20:6877-88). For example, the target segment of the target MRNA preferably should begin with AA (most preferred), TA, GA, or CA; the GC ratio of the siRNA molecule preferably should be 45-55%; the siRNA molecule preferably should not contain three of the same nucleotides in a row; the siRNA molecule preferably should not contain seven mixed G/Cs in a row; and the target segment preferably should be in the ORF region of the target MRNA and preferably should be at least 75 bp after the initiation ATG and at least 75 bp before the stop codon. Based on these criteria, or on other known criteria (e.g., Reynolds et al. (2004) Nature Biotechnol. 22:326-30), siRNA molecules of the present invention, targeted to the MRNA polynucleotides related to or provided by the present invention, can be designed by one of ordinary skill in the art.

Altered expression of the genes related to or provided by the present invention in an organism may also be achieved through the creation of nonhuman transgenic animals into whose genomes polynucleotides related to or provided by the present invention have been introduced. Such transgenic animals include animals that have multiple copies of a gene (i.e., the transgene) of the present invention. A tissue-specific regulatory sequence(s) may be operably linked to the transgene to direct expression of a polypeptide related to or provided by the present invention to particular cells or a particular developmental stage. Methods for generating transgenic animals via embryo manipulation and microinjection, particularly animals such as mice, have become conventional and are well known in the art (e.g., Bockamp et al. (2002) Physiol. Genomics 11 :115-32).

Altered expression of the genes related to or provided by the present invention in an organism may also be achieved through the creation of animals whose endogenous genes corresponding to the polynucleotides related to or provided by the present invention have been disrupted through insertion of extraneous polynucleotide sequences (i.e., a knockout animal). The coding region of the endogenous gene may be disrupted, thereby generating a nonfunctional protein. Alternatively, the upstream regulatory region of the endogenous gene may be disrupted or replaced with different regulatory elements, resulting in the altered expression of the still-functional protein. Methods for generating knockout animals include homologous recombination and are well known in the art (e.g., Wolfer et al. (2002) Trends Neurosci. 25:336-40).

The present invention also provides constructs in the form of plasmids, vectors, transcription or expression cassettes which comprise at least one nucleic acid related to or provided by the invention as above. The isolated polynucleotides related to or provided by the present invention may be operably linked to an expression control sequence and/or ligated into an expression vector for recombinant production of the polypeptides of the present invention. General methods of expressing recombinant proteins are well known in the art.

An expression vector, as used herein, is intended to refer to a nucleic acid molecule capable of transporting another nucleic acid to which it has been linked. One type of vector is a plasmid, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., nonepisomal mammalian vectors) can be integrated into the genome of a host cell upon introduction into the host cell, and thereby are replicated along with the host genome. Moreover, certain vectors are capable of directing the expression of genes to which they are operably linked. Such vectors are referred to herein as recombinant expression vectors (or simply, expression vectors). In general, expression vectors of utility in recombinant DNA techniques are often in the form of plasmids. In the present specification, plasmid and vector may be used interchangeably as the plasmid is the most commonly used form of vector. However, the invention is intended to include other forms of expression vectors, such as viral vectors (e.g., replication-defective retroviruses, adenoviruses and adeno-associated viruses) that serve equivalent functions.

The recombinant expression vectors of the invention may carry additional sequences, such as sequences that regulate replication of the vector in host cells (e.g., origins of replication) and selectable marker genes. The selectable marker gene facilitates selection of host cells into which the vector has been introduced. For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids or viral, e.g., phage, or phagemid, as appropriate. For further details see, for example, Molecular Cloning: a Laboratory Manual: 2nd ed., Sambrook et al., Cold Spring Harbor Laboratory Press, 1989. Many known techniques and protocols for manipulation of nucleic acids, for example, in preparation of nucleic acid constructs, mutagenesis, sequencing, introduction of DNA into cells and gene expression, and analysis of proteins, are described in detail in Current Protocols in Molecular Biology, 2nd ed., Ausubel et al. eds., John Wiley & Sons, 1992.

The present invention also provides a host cell, which comprises one or more constructs as above. The present invention also includes a method of producing the encoded product. The method comprises expression from the encoding nucleic acid. Expression may be achieved by culturing recombinant host cells containing the nucleic acid under appropriate conditions.

A number of cell lines may act as suitable host cells for recombinant expression of the polypeptides related to or provided by the present invention. Mammalian host cell lines include, for example, COS cells, CHO cells, 293 cells, A431 cells, 3T3 cells, CV-1 cells, HeLa cells, L cells, BHK21 cells, HL-60 cells, U937 cells, HaK cells, Jurkat cells, as well as cell strains derived from in vitro culture of primary tissue and primary explants.

Alternatively, it may be possible to recombinantly produce the polypeptides related to or provided by the present invention in lower eukaryotes such as yeast or in prokaryotes. Potentially suitable yeast strains include Saccharomyces cerevisiae, Schizosaccharomyces pombe, Kluyveromyces strains, and Candida strains. Potentially suitable bacterial strains include Escherichia coli, Bacillus subtilis, and Salmonella typhimurium. If the polypeptides related to or provided by the present invention are made in yeast or bacteria, it may be necessary to modify them by, for example, phosphorylation or glycosylation of appropriate sites, in order to obtain functionality. Such covalent attachments may be accomplished using well-known chemical or enzymatic methods.

The polypeptides related to or provided by the present invention may also be recombinantly produced by operably linking the isolated polynucleotides of the present invention to suitable control sequences in one or more insect expression vectors, such as baculovirus vectors, and employing an insect cell expression system. Materials and methods for baculovirus/Sf9 expression systems are commercially available in kit form (e.g., the MaxBac® (kit, Invitrogen, Carlsbad, Calif.).

Following recombinant expression in the appropriate host cells, the polypeptides related to or provided by the present invention may then be purified from culture medium or cell extracts using known purification processes, such as gel filtration and ion exchange chromatography. Purification may also include affinity chromatography with agents known to bind the polypeptides of the present invention. These purification processes may also be used to purify the polypeptides of the present invention from natural sources.

Alternatively, the polypeptides related to or provided by the present invention may also be recombinantly expressed in a form that facilitates purification. For example, the polypeptides may be expressed as fusions with proteins such as maltose-binding protein (MBP), glutathione-S-transferase (GST), or thioredoxin (TRX). Kits for expression and purification of such fusion proteins are commercially available from New England BioLabs (Beverly, Mass.), Pharmacia (Piscataway, N.J.), and Invitrogen (Carlsbad, Calif.), respectively. The polypeptides related to or provided by the present invention can also be tagged with a small epitope and subsequently identified or purified using a specific antibody to the epitope. Preferred epitopes are the V5, 6His, 10His, Flag, Myc, and HA epitopes, or any combination thereof.

The polypeptides related to or provided by the present invention may also be produced by known conventional chemical synthesis. Methods for chemically synthesizing the polypeptides related to or provided by the present invention are well known to those skilled in the art. Such chemically synthetic polypeptides may possess biological properties in common with the natural, purified polypeptides, and thus may be employed as biologically active or immunological substitutes for the natural polypeptides.

The polypeptides related to or provided by the present invention also encompass molecules that are structurally different from the disclosed polypeptides (e.g., which have a slightly altered sequence), but which have substantially the same biochemical properties as the disclosed polypeptides (e.g., are changed only in functionally nonessential amino acid residues). Such molecules include naturally occurring allelic variants and deliberately engineered variants containing alterations, substitutions, replacements, insertions, or deletions. Techniques for such alterations, substitutions, replacements, insertions, or deletions are well known to those skilled in the art.

Thus, a further aspect of the present invention provides a host cell comprising a nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection, and transduction using retrovirus or other virus, e.g., vaccinia or, for insect cells, baculovirus. For bacterial cells, suitable techniques may include calcium chloride transformation, electroporation, and transfection using bacteriophage. The introduction may be followed by causing or allowing expression from the nucleic acid, e.g., by culturing host cells under conditions for expression of the gene.

Screening Assays for Agents That Modulate the Activity of One or More Members of the Adiponutrin Family

The present invention is based on the novel finding that members of the adiponutrin family are biologically active esterases and may have lipid acyl hydrolase and/or lipase activity (Example 3). This finding provides for the development of enzymatic assays involving members of the adiponutrin family. Thus, the polynucleotides and polypeptides related to or provided by the present invention may be used in screening assays to identify pharmacological agents or lead compounds for molecules that are capable of modulating, in a cell or organism, the activity of one or more members of the adiponutrin family, e.g., adiponutrin, desnutrin/ATGL, GS2, GS2-like, and PNPLA1 (i.e., a modulator of one or more members of the adiponutrin family), and are thereby potential regulators of nutrient and/or energy homeostasis. For example, samples containing one or more members of the adiponutrin family (either natural or recombinant) can be contacted with one of a plurality of test compounds (either biological agents or small molecules), and the esterase activity of one or more members of the adiponutrin family in each of the treated samples can be compared with the activity of the same one or more members of the adiponutrin family in untreated samples or in samples contacted with a different test compound(s). Substrates for esterase activity, lipid acyl hydrolase activity, and lipase activity have been described in the literature and may include, but are not limited to, triacylglycerol, derivatives of triacylglycerol containing radiolabeled, chromogenic or fluorogenic moieties, such as 1,2-o-dilauryl-rac-glycero-3-glutaric acid-(6′-methylresorufin) ester (DGGR), 1,2-dioleoyl-3-(1-pyrenedodecanoyl)-rac-glycerol, triacylglycerols containing fluorescently labeled fatty acids (e.g., BODIPY-, NBD-, pyrene or dansyl fatty acids), sphingolipids containing radiolabeled, chromogenic or fluorogenic moieties (e.g., BODIPY-, NBD-, pyrene-labeled sphingolipids), esters of fatty acids containing radiolabeled, chromogenic or fluorogenic moieties (e.g., 2-nitrophenyl dodecanoate; 6,8-difluoro-4-methylumbilliferyl octanoate). A wide variety of radiolabeled, chromogenic or fluorogenic esterase substrates are available from various vendors (e.g., Invitrogen-Molecular Probes Inc., Eugene, Oreg.; Roche Diagnostics, Indianapolis, Ind.). Such comparisons will determine whether any of the test compounds (i.e., candidate modulators) results in: 1) a substantially decreased level of expression, or of esterase, lipid acyl hydrolase, and/or lipase activity, of one or more members of the adiponutrin family, thereby indicating an inhibitor of one or more members of the adiponutrin family, or 2) a substantially increased level of expression, or of esterase, lipid acyl hydrolase, and/or lipase activity, of one or more members of the adiponutrin family, thereby indicating an activator of one or more members of the adiponutrin family. The ability of the test compound to inhibit or increase the level of expression or enzymatic activity of one or more members of the adiponutrin family indicates that it may be capable of restoring nutrient and/or energy homeostasis in an animal, e.g., it may be useful in treating disorders associated with dysregulated nutrient and/or energy homeostasis, such as a cardiovascular and metabolic disorder. In one embodiment, the identification of modulators of the activity of one or more members of the adiponutrin family is performed using high-throughput screening assays, such as BIACORE® (Biacore International AB, Uppsala, Sweden), BRET (bioluminescence resonance energy transfer), and FRET (fluorescence resonance energy transfer) assays, as well as ELISA and cell-based assays.

Small Molecules

Modulated activity of one or more members of the adiponutrin family in an organism afflicted with (or at risk for) at least one cardiovascular or metabolic disease (e.g., cardiovascular disease (i.e., any disease that affects the heart or blood vessels, e.g., by restricting the flow of blood), obesity, insulin resistance, type 2 diabetes, dyslipidemia, nonalcoholic fatty liver disease, metabolic syndrome (i.e., a syndrome involving the simultaneous occurrence of a group of health conditions, which may include insulin resistance, abdominal obesity, dyslipidemia, hypertension, chronic inflammation, a prothrombotic state, etc. that places a person at high risk for type 2 diabetes and/or heart disease), etc.), or in an involved cell from such an organism, may also be achieved through the use of small molecules (usually organic small molecules) that modulate the activity of one or more members of the adiponutrin family. In other words, a modulator of the activity of one or more members of the adiponutrin family may be a small molecule. The term “small molecule” refers to compounds that are not macromolecules (see, e.g., Karp (2000) Bioinformatics Ontology 16:269-85; Verkman (2004) AJP-Cell Physiol. 286:465-74). Thus, small molecules are often considered those compounds that are, e.g., less than one thousand daltons (e.g., Voet and Voet, Biochemistry, 2^{nd} ed., ed. N. Rose, Wiley and Sons, New York, 14 (1995)). For example, Davis et al. (2005) Proc. Natl. Acad Sci. USA 102:5981-86, use the phrase small molecule to indicate folates, methotrexate, and neuropeptides, while Halpin and Harbury (2004) PLos Biology 2:1022-30, use the phrase to indicate small molecule gene products, e.g., DNAs, RNAs and peptides. Examples of natural and synthesized small molecules include, but are not limited to, cholesterols, neurotransmitters, siRNAs, and various chemicals listed in numerous commercially available small molecule databases, e.g., FCD (Fine Chemicals Database), SMID (Small Molecule Interaction Database), CHEBI (Chemical Entities of Biological Interest), and CSD (Cambridge Structural Database) (see, e.g., Alfarano et al. (2005) Nuc. Acids Res. Database Issue 33:D416-24). Small molecules known to modulate the activity of one or more members of the adiponutrin family can be used in the treatment methods of the present invention. For example, the small molecule troglitazone has been shown to modulate adiponutrin mRNA expression (e.g., Polson and Thompson (2003) Horm. Metab. Res. 35:508-10). Small molecules that modulate the activity of one or more members of the adiponutrin family include but are not limited to those approved for treatment of disease, as well as others in clinical trials, and also include both natural and artificial small molecules. These molecules can be used directly or can serve as starting compounds for the development of improved modulators of one or more members of the adiponutrin family. Alternatively, novel small molecules (preferably isoform specific) identified by the screening methods described herein may also be used.

Antibodies

Modulated activity of one or more members of the adiponutrin family in an organism afflicted with (or at risk for) at least one cardiovascular and metabolic disorder, or in an involved cell from such an organism, may also be achieved through the use of antibodies that bind to and modulate the activity of one or more members of the adiponutrin family. In other words, a modulator of the activity of one or more members of the adiponutrin family may be a modulating antibody. Additionally, antibodies that bind to but do not modulate the activity of one or more members of the adiponutrin family (i.e., detecting antibodies) may be used to detect the presence of such polypeptides, e.g., as part of a kit for diagnosing, prognosing or monitoring a cardiovascular or metabolic disease.

One of skill in the art will recognize that as used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (“FR”). The extent of the FRs and CDRs has been precisely defined (see, Kabat et al. (1991) Sequences of Proteins of Immunological Interest, Fifth Edition, U.S. Department of Health and Human Services, NIH Publication No. 91-3242, and Chothia et al. (1987) J. Mol. Biol. 196:901-17, which are hereby incorporated by reference). Each VH and VL is composed of three CDRs and four FRs, arranged from amino-terminus to carboxy-terminus in the following order: FR1, CDR1, FR2, CDR2, FR3, CDR3, FR4.

The antibody can further include a heavy and light chain constant region to thereby form a heavy and light immunoglobulin chain, respectively. In one embodiment, the antibody is a tetramer of two heavy immunoglobulin chains and two light immunoglobulin chains, wherein the heavy and light immunoglobulin chains are interconnected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (C1q) of the classical complement system.

Immunoglobulin refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd, or 214 amino acids) are encoded by a variable region gene at the NH2-terminus (about 110 amino acids) and a kappa or lambda constant region gene at the COOH-terminus. Full-length immunoglobulin “heavy chains” (about 50 Kd, or 446 amino acids), are similarly encoded by a variable region gene (about 116 amino acids) and one of the other aforementioned constant region genes, e.g., gamma (encoding about 330 amino acids). The immunoglobulin heavy chain constant region genes encode for the antibody class, i.e., isotype (e.g., IgM or IgG1). The antigen binding fragment of an antibody (or simply “antibody portion,” or “fragment”), as used herein, refers to one or more fragments of a full-length antibody that retain the ability to specifically bind to an antigen (e.g., CD3). Examples of binding fragments encompassed within the term “antigen binding fragment” of an antibody include (i) an Fab fragment, a monovalent fragment consisting of the VL, VH, CL and CH1 domains; (ii) an F(ab′)₂ fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment, which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)). Such single chain antibodies are also intended to be encompassed within the term “antigen binding fragment” of an antibody. These antibody fragments are obtained using conventional techniques known to those skilled in the art, and the fragments are screened for utility in the same manner as are intact antibodies.

One of skill in the art will recognize that the methods disclosed herein for generation of antibody molecules to the polypeptides related to or provided by the present invention, e.g., members of the adiponutrin family, may also be used to generate antibody molecules to other proteins, e.g., other proteins containing the Ser-Asp catalytic dyad. Consequently, the methods for generating antibody molecules apply not only to the polypeptides of the present invention as disclosed, but also to, for example, patatin and proteins with patatin-like domains.

Antibody molecules to the polypeptides related to or provided by the present invention, e.g., antibodies that inhibit the esterase, lipid acyl hydrolase, and/or lipase activity of one or more members of the adiponutrin family, including but not limited to human and murine adiponutrin family members, and homologs thereof, may be useful in screening assays for identifying modulators of the activity of one or members of the adiponutrin family, and preventing or treating at least one cardiovascular and metabolic disorder (e.g., cardiovascular disease (i.e., any disease that affects the heart or blood vessels, e.g., by restricting the flow of blood), obesity, insulin resistance, type 2 diabetes, dyslipidemia, nonalcoholic fatty liver disease, metabolic syndrome (i.e., a syndrome involving the simultaneous occurrence of a group of health conditions, which may include insulin resistance, abdominal obesity, dyslipidemia, hypertension, chronic inflammation, a prothrombotic state, etc. that places a person at high risk for type 2 diabetes and/or heart disease), etc.). Such antibody molecules may be produced by methods well known to those skilled in the art. For example, monoclonal antibodies may be produced by generation of hybridomas in accordance with known methods. Hybridomas formed in this manner are then screened using standard methods, such as enzyme-linked immunosorbent assay (ELISA), to identify one or more hybridomas that produce an antibody that specifically binds with the polypeptides related to or provided by the present invention. For example, an adiponutrin family protein related to or provided by the invention may also be used to immunize animals to obtain polyclonal and monoclonal antibodies that specifically react with the protein and which may inhibit the enzymatic activity (e.g., esterase, lipid acyl hydrolase, and/or lipase activity) of the protein. The peptide immunogens additionally may contain a cysteine residue at the carboxyl terminus, and may be conjugated to a hapten such as keyhole limpet hemocyanin (KLH). Additional peptide immunogens may be generated by replacing tyrosine residues with sulfated tyrosine residues. Methods for synthesizing such peptides are known in the art. A full-length polypeptide of the present invention may be used as the immunogen, or, alternatively, antigenic peptide fragments of the polypeptides may be used. An antigenic peptide of a polypeptide of the present invention comprises at least seven continuous amino acid residues and encompasses an epitope such that an antibody raised against the peptide forms a specific immune complex with the polypeptide. Preferably, the antigenic peptide comprises at least 10 amino acid residues, more preferably at least 15 amino acid residues, even more preferably at least 20 amino acid residues, and most preferably at least 30 amino acid residues. In one embodiment of the invention, the antigenic peptide of a polypeptide of the present invention comprises the patatin-like domain of one of the members of the adiponutrin family.

Monoclonal antibodies may be generated by other methods known to those skilled in the art of recombinant DNA technology. As an alternative to preparing monoclonal antibody-secreting hybridomas, a monoclonal antibody to a polypeptide of the present invention may be identified and isolated by screening a recombinant combinatorial immunoglobulin library (e.g., an antibody phage display library) with a polypeptide related to or provided by the present invention, e.g., adiponutrin, desnutrin/ATGL, GS2, GS2-like or PNPLA1, to thereby isolate immunoglobulin library members that bind to the polypeptides related to or provided by the present invention. Techniques and commercially available kits for generating and screening phage display libraries are well known to those skilled in the art. Additionally, examples of methods and reagents particularly amenable for use in generating and screening antibody display libraries can be found in the literature. For example, the “combinatorial antibody display” method has been developed to identify and isolate antibody fragments having a particular antigen specificity, and can be utilized to produce monoclonal antibodies; descriptions of combinatorial antibody display are known in the art. After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and PCR.

Polyclonal sera and antibodies may be produced by immunizing a suitable subject with a polypeptide related to or provided by the present invention. The antibody titer in the immunized subject may be monitored over time by standard techniques, such as with ELISA using immobilized marker protein. If desired, the antibody molecules directed against polypeptides related to or provided by the present invention may be isolated from the subject or culture media and further purified by well-known techniques, such as protein A chromatography, to obtain an IgG fraction.

Fragments of antibodies to the polypeptides related to or provided by the present invention may be produced by cleavage of the antibodies in accordance with methods well known in the art. For example, immunologically active Fab and F(ab′)₂ fragments may be generated by treating the antibodies with an enzyme such as pepsin.

Other protein-binding molecules may also be employed to modulate the activity of an adiponutrin family member. Such protein-binding molecules include small modular immunopharmaceutical (SMIP™) drugs (Trubion Pharmaceuticals, Seattle, Wash.). SMIPs are single-chain polypeptides composed of a binding domain for a cognate structure such as an antigen, a counterreceptor or the like, a hinge-region polypeptide having either one or no cysteine residues, and immunoglobulin CH2 and CH3 domains (see also www.trubion.com). SMIPs and their uses and applications are disclosed in, e.g., U.S. Published patent application Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties.

Additionally, chimeric, humanized, and single-chain antibodies to the polypeptides related to or provided by the present invention, comprising both human and nonhuman portions, may be produced using standard recombinant DNA techniques and/or a recombinant combinatorial immunoglobulin library. Humanized antibodies may also be produced using transgenic mice that are incapable of expressing endogenous immunoglobulin heavy and light chain genes, but that can express human heavy and light chain genes. For example, human monoclonal antibodies (mAbs) directed against members of the adiponutrin family may be generated using transgenic mice carrying the human immunoglobulin genes rather than murine immunoglobulin genes. Splenocytes from these transgenic mice immunized with the antigen of interest may then be used to produce hybridomas that secrete human mAbs with specific affinities for epitopes from a human protein; such techniques are well known in the art.

Human antibodies to polypeptides related to or provided by the invention may additionally be produced using transgenic nonhuman animals that are modified so as to produce fully human antibodies rather than the animal's endogenous antibodies in response to challenge by an antigen. See, e.g., PCT publication WO 94/02602. The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. One embodiment of such a nonhuman animal is a mouse, and is termed the XENOMOUSE™ as disclosed in PCT publications WO 96/33735 and WO 96/34096. This animal produces B cells that secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

A chimeric antibody (e.g., an antibody in which the portion that encodes the Fc constant region of an antibody gene from a first species is replaced with the equivalent portion of a gene encoding an Fc constant region from a second species, such that translation of the modified antibody gene results in a chimeric antibody), including chimeric immunoglobulin chains, may be produced by recombinant DNA techniques known in the art. An antibody or an immunoglobulin chain may also be humanized by methods known in the art. For example, humanized antibodies, including humanized immunoglobulin chains, may be generated by replacing sequences of the Fv variable region that are not directly involved in antigen binding with equivalent sequences from human Fv variable regions. Such methods are well known, and include isolating, manipulating, and expressing the nucleic acid sequences that encode all or part of immunoglobulin Fv variable regions from at least one of a heavy or light chain. Sources of such nucleic acid sequences are well known to those skilled in the art and, for example, may be obtained from a hybridoma producing an antibody against a predetermined target. The recombinant DNA encoding the humanized antibody, or fragment thereof, can then be cloned into an appropriate expression vector.

Humanized or CDR-grafted antibody molecules or immunoglobulins may be produced by CDR grafting or CDR substitution, wherein one, two, or all CDRs of an immunoglobulin chain can be replaced using well-known methods, e.g., a well-known CDR-grafting method may be used to prepare the humanized antibodies of the present invention. All of the CDRs of a particular human antibody may be replaced with at least a portion of a nonhuman CDR, or only some of the CDRs may be replaced with nonhuman CDRs. It is only necessary to replace the number of CDRs required for binding of the humanized antibody to a predetermined antigen.

Monoclonal, chimeric and humanized antibodies that have been modified by, e.g., deleting, adding, or substituting other portions of the antibody, e.g., the constant region, are also within the scope of the invention. For example, an antibody can be modified as follows: (i) by deleting the constant region; (ii) by replacing the constant region with another constant region, e.g., a constant region meant to increase half-life, stability, or affinity of the antibody, or a constant region from another species or antibody class; or (iii) by modifying one or more amino acids in the constant region to alter, for example, the number of glycosylation sites, effector cell ftunction, Fc receptor (FcR) binding, complement fixation, etc.

Methods for altering an antibody constant region are known in the art. Antibodies with altered function, e.g., altered affinity for an effector ligand, such as FcR on a cell, or the C1 component of complement, can be produced by replacing at least one amino acid residue in the constant portion of the antibody with a different residue; similar types of alterations to the murine (or other species′) immunoglobulin may be applied to reduce or eliminate these functions. Such alterations are known in the art. For example, it is possible to alter the affinity of an Fc region of an antibody (e.g., an IgG, such as a human IgG) for an FcR (e.g., Fc gamma R1), or for C1q binding by replacing the specified residue(s) with a residue(s) having an appropriate functionality on its side chain, or by introducing a charged functional group, such as glutamate or aspartate, or an aromatic nonpolar residue such as phenylalanine, tyrosine, tryptophan or alanine.

Antibodies of the invention may be useful for isolating, purifying, and/or detecting a member of the adiponutrin family in supernatant, cellular lysate, or on the cell surface. Antibodies disclosed in this invention may be also used diagnostically to monitor adiponutrin family protein levels as part of a clinical testing procedure, or clinically to target a therapeutic modulator to a cell or tissue comprising the antigen of the antibody. For example, a therapeutic such as a small molecule, or other therapeutic of the invention can be linked to an antibody directed to an adiponutrin family member in order to target the therapeutic to the cell or tissue expressing the adiponutrin family member. Modulating and detecting antibodies of the invention (preferably monoclonal antibodies) that bind to a protein belonging to the adiponutrin family may also be useful in the treatment of conditions involving nutrient and/or energy homeostasis, e.g., one or more cardiovascular and metabolic disorders. These modulating monoclonal antibodies may be capable of downregulating or upregulating the enzymatic activity (e.g., esterase, lipid acyl hydrolase, and/or lipase activity) of one or more members of the adiponutrin family. The present invention further provides compositions comprising an antibody that specifically reacts with one or more members of the adiponutrin family. Similarly, the antibodies may be useful in isolating, purifying and/or detecting one or more members of the adiponutrin family, diagnostically monitoring expression levels of one or more members of the adiponutrin family, or clinically targeting a therapeutic modulator to a cell or tissue comprising one or more members of the adiponutrin family.

Methods for Diagnosing, Prognosing, and Monitoring the Progress of Cardiovascular and Metabolic Disorders

It is well known in the art that disease mechanisms studied in animal models, particularly murine models, may be and often are translatable to the related human diseases. As such, although the Examples disclosed herein demonstrate differential expression of adiponutrin family members, e.g., by liver and adipose tissue in a murine model, the disclosed methods for diagnosing, prognosing, and monitoring disorders related to dysregulation of nutrient and/or energy homeostasis, e.g., a cardiovascular and metabolic disorder, will be particularly useful for diagnosing, prognosing and monitoring such disorders in humans. In practicing the disclosed methods, a skilled artisan will recognize that the human homologs of members of the adiponutrin family, as well as modulators thereof, may be used in the claimed methods of diagnosing, prognosing, and monitoring such disorders in humans.

The present invention provides methods for diagnosing, prognosing, and monitoring the progress of a cardiovascular and metabolic disorder in a subject (e.g., disorders that directly or indirectly involve increases or decreases in the levels and/or activities of one or more members of the adiponutrin family) by detecting the level of expression of a member(s) of the adiponutrin family and/or activity(ies) (e.g., esterase, lipid acyl hydrolase, and/or lipase activity) thereof, including but not limited to the use of such methods in human subjects. One of skill in the art will recognize that these methods can apply to several cardiovascular and metabolic disorders. These methods may be performed by utilizing prepackaged diagnostic kits comprising at least one of the group comprising the following: 1) a polynucleotide, or fragments thereof, encoding a member of the adiponutrin family, 2) a polypeptide, or portions thereof, belonging to the adiponutrin family, 3) a modulator of one or more members of the adiponutrin family (e.g., a small molecule, a modulating antibody, etc.), or 4) a detecting antibody to a member of the adiponutrin family, or derivatives thereof, any or all of which may be conveniently used, for example, in a clinical setting. In addition, one of skill in the art will recognize that the levels of a member(s) of the adiponutrin family, e.g., adiponutrin, may also be detected by indirect methods, such as determining the levels of adiponutrin substrates or products or the expression of genes regulated by adiponutrin function.

“Diagnostic” or “diagnosing” means identifying the presence or absence of a pathologic condition. Diagnostic methods include detecting the expression level of a member of the adiponutrin family, or activity (e.g., esterase, lipid acyl hydrolase, and/or lipase activity) thereof, by determining a test amount of an adiponutrin family gene product (e.g., mRNA, cDNA, or polypeptide, including fragments thereof), or activity thereof, in a biological sample from a subject (human or nonhuman mammal), and comparing the test amount with a normal amount or range (i.e., an amount or range from an individual(s) known not to suffer from a given cardiovascular and metabolic disorder) for the adiponutrin family gene product, or activity thereof. Although a particular diagnostic method may not provide a definitive diagnosis of a cardiovascular and metabolic disorder, it suffices if the method provides a positive indication that aids in diagnosis.

The present invention also provides methods for prognosing such a cardiovascular and metabolic disorder by detecting the level of expression of a member of the adiponutrin family, or activity (e.g., esterase, lipid acyl hydrolase, and/or lipase activity) thereof. “Prognostic” or “prognosing” means predicting the probable development and/or severity of a pathologic condition. Prognostic methods include determining a test amount of an adiponutrin family member gene product, or activities thereof, in a biological sample from a subject, and comparing the test amount to a prognostic amount or range (i.e., an amount or range from individuals with varying severities of a cardiovascular and metabolic disorder) for the adiponutrin family member gene product, or activity thereof. Various amounts of the adiponutrin family member gene product, or activity thereof, in a test sample are consistent with certain prognoses for cardiovascular and metabolic disorders. The detection of an amount of an adiponutrin family member gene product, or activity thereof, at a particular prognostic level provides, or aids in determining, a prognosis for the subject.

The present invention also provides methods for monitoring the progress or course of such cardiovascular and metabolic disorders by detecting the expression level of a member of the adiponutrin family, or activity (e.g., esterase, lipase, and/or lipid acyl hydrolase activity) thereof. Monitoring methods include determining the test amounts of an adiponutrin family member gene product, or activity thereof, in biological samples taken from a subject at a first and second time, and comparing the amounts. A change in amount of the adiponutrin family member gene product, or activity thereof, between the first and second times indicates a change in the course of cardiovascular and metabolic disorders. Depending on the adiponutrin family member, a decrease in amount may indicate, e.g., remission of the cardiovascular and metabolic disorder, and an increase in amount may indicate, e.g., progression of the cardiovascular and metabolic disorder; for another member of the adiponutrin family, such indications related to monitoring may be reversed, e.g., an increase may indicate remission. Such monitoring assays are also useful for evaluating the efficacy of a particular therapeutic intervention in patients being treated for cardiovascular and metabolic disorders.

Increased expression of members of the adiponutrin family, or activities thereof (e.g., esterase, lipid acyl hydrolase, and/or lipase activities) in methods outlined above can be detected in a variety of biological samples, including bodily fluids (e.g., whole blood, plasma, and urine), cells (e.g., whole cells, cell fractions, and cell extracts), and tissues. Biological samples also include sections of tissue, such as biopsies and frozen sections taken for histological and other purposes. Preferred biological samples include blood, plasma, lymph, tissue biopsies (e.g., adipose tissue biopsies), urine, and bile. It will be appreciated that analysis of a biological sample need not necessarily require removal of cells or tissue from the subject. For example, appropriately labeled agents that bind adiponutrin family member gene products (e.g., antibodies, nucleic acids) can be administered to a subject and visualized (when bound to the target) using standard imaging technology (e.g., CAT, NMR (MRI), and PET).

In the diagnostic and prognostic assays of the present invention, the adiponutrin family member gene product is detected and quantified to yield a test amount. The test amount is then compared with a normal amount or range. Depending on the adiponutrin family member, an amount significantly above the normal amount or range may be a positive sign in the diagnosis of cardiovascular and metabolic disorders. Particular methods of detection and quantitation of adiponutrin family member gene products are described below.

Normal amounts or baseline levels of adiponutrin family member gene products can be determined for any particular sample type and population. Generally, baseline (normal) levels of an adiponutrin family member protein or MRNA are determined by measuring the amount of the adiponutrin family member protein or MRNA in a biological sample type from normal (i.e., healthy) subjects. Alternatively, normal values of an adiponutrin family member gene product can be determined by measuring the amount in healthy cells or tissues taken from the same subject from which the diseased (or possibly diseased) test cells or tissues were taken. The amount of the adiponutrin family member gene product (either the normal amount or the test amount) can be determined or expressed on a per cell, per total protein, or per volume basis. To determine the cell amount of a sample, one can measure the level of a constitutively expressed gene product or other gene product expressed at known levels in cells of the type from which the biological sample was taken.

It will be appreciated that the assay methods of the present invention do not necessarily require measurement of absolute values of an adiponutrin family member gene product because relative values are sufficient for many applications of these methods. It will also be appreciated that in addition to the quantity or abundance of adiponutrin family member gene products, variant or abnormal adiponutrin family member gene products or their expression patterns (e.g., mutated transcripts, truncated polypeptides) may be identified by comparison to normal gene products and expression patterns.

Detection of Adiponutrin Family Members

The diagnostic, prognostic, and monitoring assays, and other methods of the present invention involve detecting and quantifying adiponutrin family member gene products in biological samples. Adiponutrin family member gene products include adiponutrin family member mRNAs, cDNAs, and genomic DNAs, and adiponutrin family member polypeptides, and both can be measured using methods well known to those skilled in the art.

For example, the MRNA of a member of the adiponutrin family can be directly detected and quantified using hybridization-based assays, such as Northern hybridization, in situ hybridization, dot and slot blots, and oligonucleotide arrays. Hybridization-based assays refer to assays in which a probe nucleic acid is hybridized to a target nucleic acid. In some formats, the target, the probe, or both are immobilized. The immobilized nucleic acid may be DNA, RNA, or another oligonucleotide or polynucleotide, and may comprise naturally or nonnaturally occurring nucleotides, nucleotide analogs, or backbones. Methods of selecting nucleic acid probe sequences for use in the present invention are based on the nucleic acid sequences of the members of the adiponutrin family and are well known in the art.

Alternatively, mRNAs of members of the adiponutrin family can be amplified before detection and quantitation. Such amplification-based assays are well known in the art and include polymerase chain reaction (PCR), reverse-transcription-PCR (RT-PCR), PCR-enzyme-linked immunosorbent assay (PCR-ELISA), and ligase chain reaction (LCR). Primers and probes for producing and detecting amplified adiponutrin family member gene products (e.g., MRNA or cDNA) may be readily designed and produced without undue experimentation by those of skill in the art based on the nucleic acid sequence of an adiponutrin family member. Amplified adiponutrin family member gene products may be directly analyzed, for example, by gel electrophoresis; by hybridization to a probe nucleic acid; by sequencing; by detection of a fluorescent, phosphorescent, or radioactive signal; or by any of a variety of well-known methods. In addition, methods are known to those of skill in the art for increasing the signal produced by amplification of target nucleic acid sequences. One of skill in the art will recognize that whichever amplification method is used, a variety of quantitative methods known in the art (e.g., quantitative PCR (also referred to as “Q-PCR,” “real time PCR”, “quantitative real time PCR,” “quantitative real time reverse transcriptase polymerase chain reaction,” “quantitative real time RT-PCR,” and the like)) may be used if quantitation of the adiponutrin family member gene products is desired.

Adiponutrin family member polypeptides (or fragments thereof) can be detected using various well-known immunological assays employing the antibodies described above. Immunological assays herein refer to assays that utilize an antibody (e.g., polyclonal, monoclonal, chimeric, humanized, scFv, and fragments thereof) that specifically binds to a polypeptide (or a fragment thereof) belonging to the adiponutrin family. Such well-known immunological assays suitable for the practice of the present invention include ELISA, radioimmunoassay (RIA), immunoprecipitation, immunofluorescence, fluorescence-activated cell sorting (FACS), and Western blotting. A polypeptide of the adiponutrin family can also be detected as enzymatic activity using the esterase, lipase, and/or lipid acyl hydrolase assays described in Example 3. In addition, an antibody of the invention (i.e., an antibody directed against a polypeptide of the invention) and/or an antibody directed against at least one member of the adiponutrin family, can be labeled with a radioactive biomarker whose presence and location in a subject can be detected by standard imaging techniques.

Each adiponutrin family member of the invention (i.e., human PNPLA1, murine PNPLA1, rat PNPLA1, and rat adiponutrin) may be considered individually, although it is within the scope of the invention to provide combinations of two or more adiponutrin family members of the invention and/or adiponutrin family members related to the invention for use in the methods and compositions of the invention to increase the confidence of the analysis. In one embodiment, the invention provides panels, e.g., of polynucleotides and/or polypeptides, for the detection of the expression and/or activity of at least one adiponutrin family member of the invention. In one embodiment, a panel of the invention detects the expression and/or activity of at least two adiponutrin family members of the invention. In one embodiment, a panel of the invention detects the expression and/or activity of at least three adiponutrin family members of the invention. In one embodiment, a panel of the invention detects the expression and/or activity of at least four adiponutrin family members of the invention.

In addition to providing panels, e.g., of polynucleotides and/or polypeptides, for the detection of the expression and/or activity of at least one adiponutrin family member of the invention, it is within the scope of the invention to provide a panel conveniently coupled to a solid support. For example, polynucleotides and polypeptides of the invention for the detection of at least one adiponutrin family member of the invention may be coupled to an array (e.g., a biochip for hybridization analysis), to a resin (e.g., a resin that can be packed into a column for column chromatography), or a matrix (e.g., a nitrocellulose matrix for Northern blot analysis) using well-known methods in the art. Methods of making and using such arrays, including the use of such arrays with computer readable media and/or databases, e.g., a relational database, are well known in the art.

By providing such support, discrete analysis of the presence or activity in a sample of each adiponutrin family member of the invention selected for the panel may be detected. For example, in an array, polynucleotides complementary to each adiponutrin family member of the invention included in a panel of the invention may be individually attached to different known locations on the array using methods well known in the art. The array may be hybridized with, for example, polynucleotides extracted from a from a subject. The hybridization of polynucleotides from the sample with the array at any location on the array can be detected, and thus the presence, quantity, and/or activity of the adiponutrin family member of the invention in the sample can be ascertained. Thus, not only tissue specificity, but also the level of expression of a panel of adiponutrin family members of the invention in the tissue is ascertainable. In a preferred embodiment, an array based on a biochip is employed. Similarly, ELISA analyses may be performed on immobilized antibodies specific for different polypeptide biomarkers hybridized to a protein sample from a subject.

In another embodiment, a reporter nucleic acid is utilized to detect the expression of one or more adiponutrin family members of the invention. Such a reporter nucleic acid can be useful for high-throughput screens for agents that alter the expression profiles of peripheral blood mononuclear cells. The construction and use of such reporter assays are well known.

For example, the construction of a reporter for transcriptional regulation of an adiponutrin family member of the invention generally requires a regulatory sequence of an adiponutrin family member of the invention, typically the promoter. The promoter can be obtained by a variety of routine methods. For example, a genomic library can be hybridized with a labeled probe consisting of the coding region of the nucleic acid to identify genomic library clones containing promoter sequences. The isolated clones can be sequenced to identify sequences upstream from the coding region. Another method is an amplification reaction using a primer that anneals to the 5′ end of the coding region of the adiponutrin family member polynucleotide of the invention. The amplification template can be, for example, restricted genomic nucleic acids to which anchor bubble adaptors have been ligated.

To construct the reporter, the promoter of the selected adiponutrin family member of the invention can be operably linked to the reporter nucleic acid, e.g., without utilizing the reading frame of the selected adiponutrin family member polynucleotide of the invention. The nucleic acid construct is transformed into tissue culture cells, e.g., peripheral blood mononuclear cells, by a transfection protocol to generate reporter cells.

Many of the well-known reporter nucleic acids may be used. In one embodiment, the reporter nucleic acid is green fluorescent protein. In a second embodiment, the reporter is β-galactosidase. In other embodiments, the reporter nucleic acid is alkaline phosphatase, β-lactamase, luciferase, chloramphenicol acetyltransferase, or other reporter nucleic acids known in the art. The reporter nucleic acid construct may be maintained on an episome or inserted into a chromosome by, for example, using targeted homologous recombination. Methods of making and using such reporter nucleic acids are well known.

One of skill in the art will understand that the aforementioned methods can be applied to one or more cardiovascular and metabolic disorders. One of skill in the art will also recognize that the aforementioned methods or variations thereof can also be used for diagnosing, prognosing, and monitoring the progress of various cardiovascular and metabolic disorders in a subject (e.g., that directly or indirectly involve modulation in the levels of one or more members of the adiponutrin family) by detecting modulation of activities associated with the adiponutrin family members, e.g., by detecting the downregulation of an adiponutrin family member or activity thereof, including but not limited to the use of such methods in human subjects.

Methods of Treating Cardiovascular and Metabolic Disorders

The notion that members of the adiponutrin family play critical roles in nutrient and/or energy homeostasis and may be key players in one or more cardiovascular and metabolic disorder (e.g., cardiovascular disease (i.e., any disease that affects the heart or blood vessels, e.g., by restricting the flow of blood), obesity, insulin resistance, type 2 diabetes, dyslipidemia, nonalcoholic fatty liver disease, metabolic syndrome (i.e., a syndrome involving the simultaneous occurrence of a group of health conditions which may include insulin resistance, abdominal obesity, dyslipidemia, hypertension, chronic inflammation, a prothrombotic state, etc. that places a person at high risk for type 2 diabetes and/or heart disease), etc.) has recently been established. The art has demonstrated preferential expression of some members of the family (e.g., adiponutrin and desnutrin/ATGL) in adipose tissue, differential expression of these members between genetically obese and wild-type animals, and regulation of this expression by nutritional control (e.g., starvation). The inventors have confirmed this data (Examples 1-2 below) and have shown that members of the adiponutrin family have enzymatic activity (e.g., esterase, lipid acyl hydrolase, and/or lipase activity) (Example 3). These findings by the inventors further support the idea that members of the adiponutrin family are involved in nutrient and/or energy homeostasis and/or the development of cardiovascular and metabolic disorders. Accordingly, modulators of the activity of one or more members of the adiponutrin family, identified as described herein, may be used to treat such cardiovascular and metabolic disorders.

Thus, the present invention also provides methods for modulating the enzymatic activity of one or more members of the adiponutrin family in a subject comprising administering a modulator of the activity of one or more members of the adiponutrin family, e.g., a small molecule identified as described above, an inhibitory antibody generated as described above, etc. In another embodiment, the present invention provides methods for treating cardiovascular and metabolic disorders comprising administering to a patient a modulator of the activity of one or more members of the adiponutrin family. In some embodiments, the invention comprises selecting a subject in need of a modulator of the enzymatic activity of one or more members of the adiponutrin family of proteins, and administering to the subject a therapeutically effective amount of such a modulator. Additionally, it should be noted that the polynucleotides and polypeptides of the present invention may also act as modulators of one or more members of the adiponutrin family. For example, the enzymatic activities described for the polypeptides of the present invention may be provided by administration or use of such polypeptides, or by administration or use of polynucleotides encoding such polypeptides (such as, e.g., in gene therapies or vectors suitable for introduction of DNA), resulting in, e.g., enhanced enzymatic activity. Additionally, the enzymatic activities described for the polypeptides related to or provided by the present invention, e.g., lipid acyl hydrolase activity, may be inhibited by administration or use of inhibitory polynucleotides as described above.

Administration of a modulator of the activity of one or more members of the adiponutrin family may be accomplished using methods known to those of ordinary skill in the art. The administration may, for example, be intravenous, intraperitoneal, intramuscular, intracavitary, subcutaneous or transdermal. Additionally, one of ordinary skill in the art would know that the modulator may be administered as part of a pharmaceutical composition formulated to be compatible with the intended route of administration. For example, it may be possible to obtain compositions which may be topically or orally administered, or which may be capable of transmission across mucous membranes. Administration of a modulator of the activity of one or more members of the adiponutrin family used in a pharmaceutical composition to practice the method of the present invention can be carried out in a variety of conventional ways, such as oral ingestion, inhalation, or cutaneous, subcutaneous, or intravenous injection. Administration by oral ingestion is often preferred.

As used herein, the term “therapeutically effective amount” means the total amount of each active component of the pharmaceutical composition or method that is sufficient to show a meaningful patient benefit, e.g., amelioration of symptoms of, healing of, remission of, or increase in rate of healing of or remission of, a given condition(s). When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of a modulator of the activity of one or more members of the adiponutrin family in a pharmaceutical composition of the present invention will depend upon the nature and severity of the condition being treated, and on the nature of prior treatments that the patient has undergone. Ultimately, the attending physician will decide the amount of the modulator with which to treat each individual patient. Initially, the attending physician will administer low doses of modulator and observe the patient's response. Larger doses of modulator may be administered until the optimal therapeutic effect is obtained for the patient, and at that point the dosage is not generally increased further.

The duration of therapy using a pharmaceutical composition comprising a modulator of the present invention will vary, depending on the severity of the disease being treated and the condition and potential idiosyncratic response of each individual patient. Ultimately the attending physician will decide on the appropriate duration of therapy using the pharmaceutical composition of the present invention.

In practicing the method of treatment or use of the present invention, a therapeutically effective amount of a modulator of the activity of one or more members of the adiponutrin family is administered to a subject, e.g., a mammal (e.g., a human). A modulator of the activity of one or more members of the adiponutrin family may be administered alone in accordance with the method of the invention. In another embodiment, the modulators of the invention, e.g., pharmaceutical compositions thereof, are administered in combination therapy, i.e., combined with other agents, e.g., therapeutic agents, that are useful for treating pathological conditions or disorders, such as cardiovascular and metabolic disorders. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment.

For example, the combination therapy can include one or more modulators of the activity of one or more members of the adiponutrin family, coformulated with, and/or coadministered with, one or more additional therapeutic agents, e.g., insulin-sensitizing agents, insulin secretagogues, insulin, appetite-suppressants, cholesterol-and/or lipid-lowering agents, or agents that lower blood pressure or otherwise improve cardiac function, as described in more detail below. Furthermore, one or more modulators identified as described herein may be used in combination with two or more of the therapeutic agents described herein. Such combination therapies may advantageously utilize lower dosages of the administered therapeutic agents, thus avoiding possible toxicities or complications associated with the various monotherapies. Moreover, the therapeutic agents disclosed herein act on pathways that differ from the adiponutrin family pathway, and thus are expected to enhance and/or synergize with the effects of a modulator of the activity of one or more members of the adiponutrin family.

Therapeutic agents that are beneficial for type 2 diabetes and work by improving insulin sensitivity may used in combination with a modulator (of the invention) of the activity of one or more members of the adiponutrin family. In one embodiment, one or more modulators of the activity of one or more members of the adiponutrin family described herein may be coformulated with, and/or coadministered with, one or more additional agents, such as metformin, modulators of peroxisome proliferator-activated receptors (PPARs), such as PPARalpha, PPARgamma and/or PPARdelta modulators, glucagon-like peptide 1 (GLP1) and GLP-1 derivatives, inhibitors of dipeptidyl peptidase IV (DPPIV), adiponectin and adiponectin derivatives, beta3 adrenergic receptor agonists as well as other therapies currently being investigated for the treatment of type 2 diabetes that have insulin-sensitizing action (including, but not limited to, PTP1B inhibitors, 11beta-HSD1 inhibitors, SHIP2 antagonists, DGAT antagonists).

In other embodiments, one or more modulators of the activity of one or more members of the adiponutrin family can be coformulated with insulin or insulin-derivatives (oral, inhaled and/or local injection), or agents that improve insulin secretion. Nonlimiting examples of the drugs or inhibitors that can be used in combination with a modulator of the activity of one or more members of the adiponutrin family described herein include, but are not limited to, one or more of: sulfonylurea drugs, e.g., glyburide, glibenclamide, gliclazide; insulin secretagogues of the “glinide” class, e.g., nateglinide, repaglinide or other insulin secretagogue drugs, e.g., glucagon-like peptide 1 (GLP1) and GLP-1 derivatives or inhibitors of DPPIV.

In other embodiments, one or more modulators of the activity of one or more members of the adiponutrin family can be coformulated with agents that inhibit hepatic glucose output. Nonlimiting examples of the drugs or inhibitors that can be used in combination with a modulator of the activity of one or more members of the adiponutrin family described herein, include, but are not limited to, one or more of: fructose-1,6,-bisphosphatase inhibitors, glucokinase activators, glucagon-receptor antagonists and others.

Additional examples of therapeutic agents that can be combined with a modulator of the activity of one or more members of the adiponutrin family include agents which decrease body weight through a variety of mechanisms, e.g., by inhibiting fat absorption (e.g., orlistat), by decreasing appetite (e.g., sibutramine or the cannabinoid-receptor antagonist rimonabant), or by increasing energy expenditure (e.g., beta-3-receptor agonists). Additional examples of therapeutic agents that can be combined with a modulator of the activity of one or more members of the adiponutrin family include drugs that improve blood lipid profiles, e.g., the statin class of drug (e.g., simvastatin, atorvastatin, lovastatin, or pravastatin), cholesterol absorption inhibitors (e.g., ezetimibe), cholesterol ester transfer protein inhibitors (e.g., torcetrapib), famesoid X receptor modulators, Liver X Receptor modulators or ileal bile acid transporter inhibitors. Additional examples of therapeutic agents that can be combined with a modulator of the activity of one or more members of the adiponutrin family include one or more of: antithrombotic agents, antihypertensive agents, and anti-inflammatory agents.

Pharmaceutical Compositions

Administration of a modulator of the activity of one or more members of the adiponutrin family via an intradermal or subcutaneous route typically requires the agent be in a solution or suspension that may also include one or more of the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; agents for the adjustment of tonicity such as sodium chloride or dextrose; and agents for the adjustment of pH. Such preparations may be enclosed in ampoules, disposable syringes or multiple dose vials made of glass or plastic. A pharmaceutical composition of the invention may be in the form of a liposome in which a modulator of the invention is combined, in addition to other pharmaceutically acceptable carriers, with amphipathic agents such as lipids that exist in aggregated form as micelles, insoluble monolayers, liquid crystals, or lamellar layers while in aqueous solution. Suitable lipids for liposomal formulation include, without limitation, monoglycerides, diglycerides, sulfatides, lysolecithin, phospholipids, saponin, bile acids, and the like; preparation of such liposomal formulations is within the level of skill in the art.

Pharmaceutical compositions suitable for injection include sterile aqueous solutions or dispersions and sterile powders for the extemporaneous preparation of sterile injectable solutions or dispersion. For intravenous administration, a modulator of the activity of one or more members of the adiponutrin family may be combined with suitable carriers including physiological saline, bacteriostatic water, Cremophor™ EL (BASF, Parsippany, N.J.) or phosphate buffered saline (PBS). In all cases, the composition must be sterile and should be fluid to the extent that easy syringability is desired. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Oral compositions generally include an inert diluent or an edible carrier. They can be enclosed in gelatin capsules or compressed into tablets. For the purpose of oral therapeutic administration, a modulator of the activity of one or more members of the adiponutrin family may be incorporated with excipients and used in the form of tablets, or capsules. Pharmaceutically compatible binding agents, and/or adjuvant materials can be included as part of the composition. The tablets, pills, capsules, and the like can contain any of the following ingredients, or compounds of a similar nature; a binder such as microcrystalline cellulose, gum tragacanth or gelatin; an excipient such as starch or lactose; a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring. When administered in liquid form, a liquid carrier such as water, petroleum, oils of animal or plant origin such as peanut oil (taking into account the frequency of peanut allergies in the population), mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol.

For administration by inhalation, a modulator of the activity of one or more members of the adiponutrin family may be delivered in the form of an aerosol spray from a pressured container or dispenser that contains a suitable propellant, e.g., a gas such as carbon dioxide, or a nebulizer.

Systemic administration can also be by transmucosal or transdermal means. For example, in case the modulator of the activity of one or more members of the adiponutrin family is an antibody that comprises an Fc portion, compositions may be capable of transmission across mucous membranes (e.g., intestine, mouth, or lungs) via the FcRn receptor-mediated pathway (see, e.g., U.S. Pat. No. 6,030,613). Transmucosal administration can be accomplished, for example, through the use of lozenges, nasal sprays, inhalers, or suppositories. For transdermal administration, the active compounds are formulated into ointments, salves, gels, or creams as generally known in the art. For transmucosal or transdermal administration, penetrants appropriate to the barrier to be permeated are used in the formulation. Such penetrants are generally known in the art, and include, for example, detergents, bile salts, and fusidic acid derivatives.

A modulator of the activity of one or more members of the adiponutrin family may be prepared with carriers that will protect the agent against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions containing a modulator of the activity of one or more members of the adiponutrin family may also be used as pharmaceutically acceptable carriers, and are known to those skilled in the art.

It may be advantageous to formulate oral or parenteral compositions in dosage unit form for ease of administration and uniformity of dosage. Dosage unit form as used herein refers to physically discrete units suited as unitary dosages for the subject to be treated, each unit containing a predetermined quantity of active compound calculated to produce the desired therapeutic effect in association with the required pharmaceutical carrier. The specifications for the dosage unit forms of the invention are dictated by and directly dependent on the unique characteristics of the active compound and the particular therapeutic effect to be achieved, and the limitations inherent in the art of formulating such an active compound for the treatment of individuals.

Another aspect of the present invention accordingly relates to kits for carrying out the combined administration of a modulator of one or more members of the adiponutrin family of the invention with other therapeutic compounds. In one embodiment, the kit comprises one or more modulators of the activity of one or more members of the adiponutrin family formulated in a pharmaceutical carrier, and at least one agent, e.g., a therapeutic agent, formulated as appropriate, in one or more separate pharmaceutical preparations. In another embodiment, the kit is for using a modulator of the invention as a research tool to determine the presence of a member of the adiponutrin family (e.g., an ELISA kit comprising an antibody of the invention) and/or its level of enzymatic activity in a biological sample.

The contents of all of the references and patent documents cited herein are hereby incorporated by reference in their entireties.

### EXAMPLES

The following Examples provide illustrative embodiments of the invention and do not in any way limit the invention. One of ordinary skill in the art will recognize that numerous other embodiments are encompassed within the scope of the invention.

The Examples do not include detailed descriptions of conventional methods, such as real-time PCR, Northern and Western hybridization, or those methods employed in the construction of vectors and plasmids, the insertion of genes encoding the polypeptides into such vectors and plasmids, the introduction of such vectors and plasmids into host cells, and the expression of polypeptides from such vectors and plasmids in host cells. Such methods are well known to those of ordinary skill in the art.

### Example 1

### Identification of Members of the Adiponutrin Family of Proteins

### Example 1:1

### Materials and Methods—Bioinformatic Analysis

Two bioinformatics gene lists of human proteins were merged to obtain gene sequences of proteins enriched in or specific to fat tissue. The first gene list was derived from a GeneLogic Bioexpress database. The GeneLogic Bioexpress database, which comprised a random set of expressed genes obtained from microarray analysis, was further narrowed using statistical algorithms to include only genes that were enriched or specific to adipose tissue and omentum fat tissue. The second gene list, which comprised gene sequences encoding proteins that either were secreted or contained a transmembrane domain, was derived from public databases using several bioinformatics tools (e.g., SignalP, Sigcleave, and TMHMM) to identify a signal peptide or a transmembrane domain within the included sequences. Merging of the two gene lists filtered out most non-adipose-tissue-enriched genes, and identified genes specific to or enriched in fat tissue that encoded for either secreted or transmembrane proteins. Twelve genes that encoded for fat-enriched secreted proteins and 19 genes that encoded for fat-enriched transmembrane proteins were obtained (FIG. 1).

Almost all of the well-known fat-enriched secreted genes, e.g., leptin, adiponectin, and adipsin, (e.g., Polson and Thompson (2003), supra), were found in the fat-enriched secreted protein pool, indicating that the approach and criteria used were feasible. Several proteins of unknown function were also identified within the fat-enriched secreted protein pool.

One of the identified proteins was desnutrin/ATGL, which contains a patatin-like domain and is strongly homologous to murine adiponutrin. It should be noted that desnutrin/ATGL encompasses the proteins previously identified as TTS2.1 and TTS2.2 since these two proteins and the genes encoding them have identical coding sequences. Two other genes, GS2 and GS2-like, also reported to be homologous to murine adiponutrin, were identified as also containing the patatin-like domain. Since primary sequence and tertiary structure alignment of adiponutrin, desnutrin/ATGL, GS2 and GS2-like amino acids to patatin indicated that all of the identified proteins contained a conserved patatin-like domain, a bioinformatics analysis using Pfam profile was applied to identify other members of the adiponutrin family and to study the potential fuiction of the patatin-like domain.

Pfam is a database of multiple alignments of protein domains or conserved protein regions. The alignments represent some evolutionarily conserved structure, which has implications for the proteins' functions. Profile hidden Markov models (profile HMMs) built from the Pfam alignments can be very useful for recognizing that a new protein belongs to an existing protein family, even if the homology is weak.

A profile HMM was generated using desnutrin/ATGL (NP_{—}080078), adiponutrin (NP_{—}473429) and the patatin family Pfam alignment as a seed alignment. The profile HMM was used to search public databases to generate a list of potential family members (Sonnhammer et al. (1997) Proteins 28:405-20). CLUSTALW (Thompson et al. (1994) Nucleic Acids Res. 22:4673-80) was used to align the patatin-like domain-containing sequences identified by the profile HMM, which were in turn added to the profile HMM for subsequent searches (Sonnhammer et al. (1998) Nucleic Acids Res. 26:320-22).

It should be noted that the human PNPLA1 sequence available from public databases contained only half of the patatin-like domain and that no EST evidence supports the 5′-end of the human PNPLA1 gene sequence. Several gene-prediction, protein, and genomic sequence comparison analyses were applied to analyze the sequence of human PNPLA1. The results indicated that the first exon in human PNPLA1 was misannotated in the public databases. Using gene-prediction algorithms and EST data, two isoforms of human PNPLA1 that differ at their 3′-ends were obtained. Additionally, the murine and rat PNPLA1 homologs were predicted by comparative genomic analysis.

To confirm whether the identified genes were members of the adiponutrin family, the identified genes containing the patatin-like domain were further classified by phylogenetic analysis using the amino acid sequence of the catalytic domain of patatin as a base sequence. The final alignment, which included predicted as well as confirmed sequences, was used as the input for the phylogenetic tree with the neighbor-joining method and 1000 bootstrap trials (see, e.g., Li and Godzik (2002) Trends Biotechnol. 20:315-16; Yang and Honig (2000) J. Mol. Biol. 301:691-711). Table 2 lists reference number (e.g., public accession numbers or SEQ ID NOs) and the amino acid ranges for the patatin-like domain used in the analysis.

TABLE 2
Amino acid sequences used for phylogenetic analysis

| | | Amino acid |
|---|---|---|
| | | range for |
| Gene | Reference Number | patatin-like domain |
| Potato patatin | AAK56395 | 32-196 |
| Human adiponutrin | NP_079501 | 10-179 |
| Mouse adiponutrin | NP_473429 | 10-179 |
| Rat adiponutrin | SEQ ID NO: 53 | 10-179 |
| Human GS2 | NP_004641 | 6-176 |
| Rat GS2 | XP_343791 | 6-175 |
| Human GS2-like | NP_620169 | 12-181 |
| Mouse GS2-like | XP_128189 | 12-181 |
| Rat GS2-like | XP_235538 | 12-178 |
| Human IPLA2 | XP_374515\|XP_291241 | 473-668 |
| Mouse IPLA2 | NP_080440 | 439-634 |
| Rat IPLA2 | XP_234092 | 444-639 |
| Human IPLA2-like | ENST00000329749 | 443-604 |
| Human NTE | NP_006693 | 933-1099 |
| Mouse NTE | NP_056616 | 933-1099 |
| Rat NTE | XP_341026 | 987-1148 |
| Human NTE-like | NP_689499 | 928-1094 |
| Mouse NTE-like | NP_666363 | 924-1090 |
| Rat NTE-like | AAM44077 | 280-446 |
| Human PLA2G6 | NP_003551 | 481-665 |
| Mouse PLA2G6 | NP_058611 | 427-611 |
| Rat PLA2G6 | NP_446344 | 426-610 |
| Human PNPLA1 | SEQ ID NO: 24 | 16-176 |
| Mouse PNPLA1 | SEQ ID NO: 27 | 16-184 |
| Rat PNPLA1 | SEQ ID NO: 56 | 16-177 |
| Human desnutrin/ATGL | NP_065109 | 10-179 |
| Mouse desnutrin/ATGL | NP_080078 | 10-179 |
| Rat desnutrin/ATGL | XP_341961 | 10-179 |

### Example 1.2

### Results—Identification of Adiponutrin-related Proteins as a Subfamily of Patatin-like Domain-containing Proteins

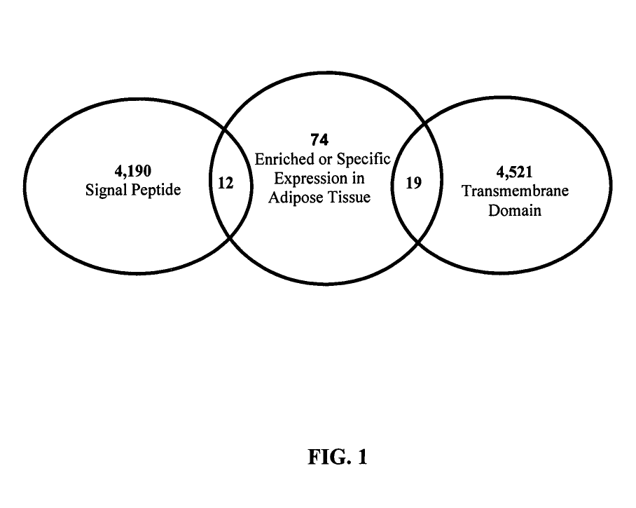

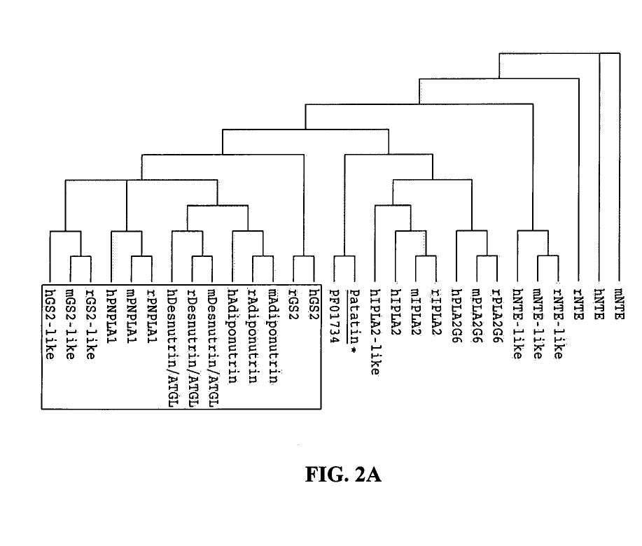

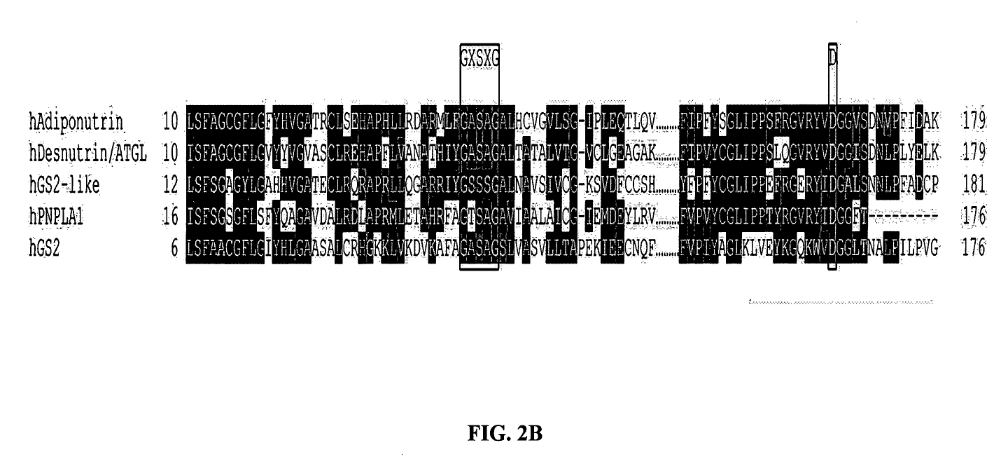

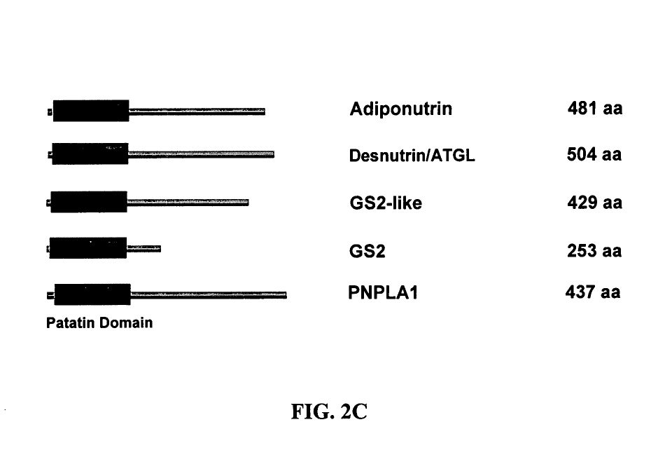

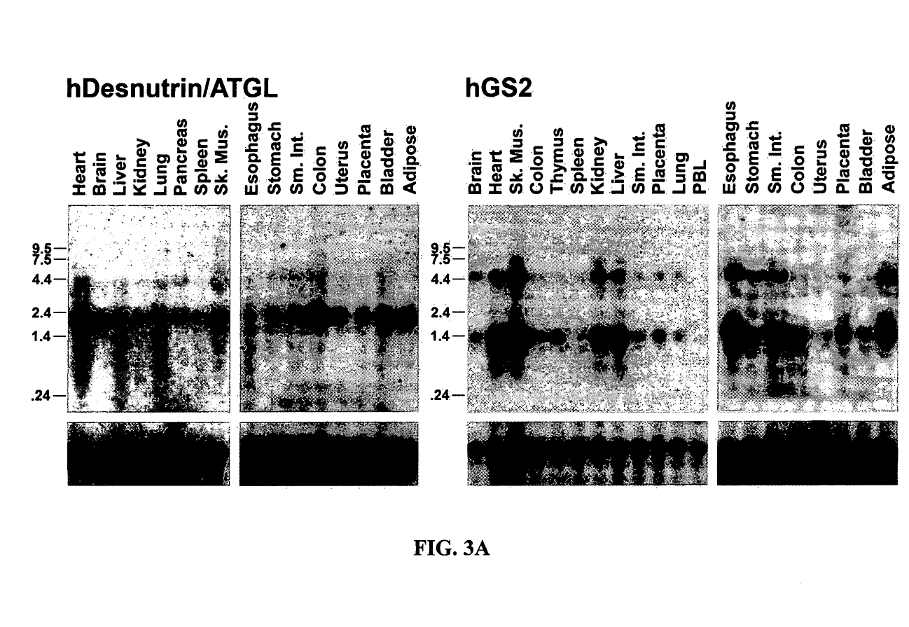

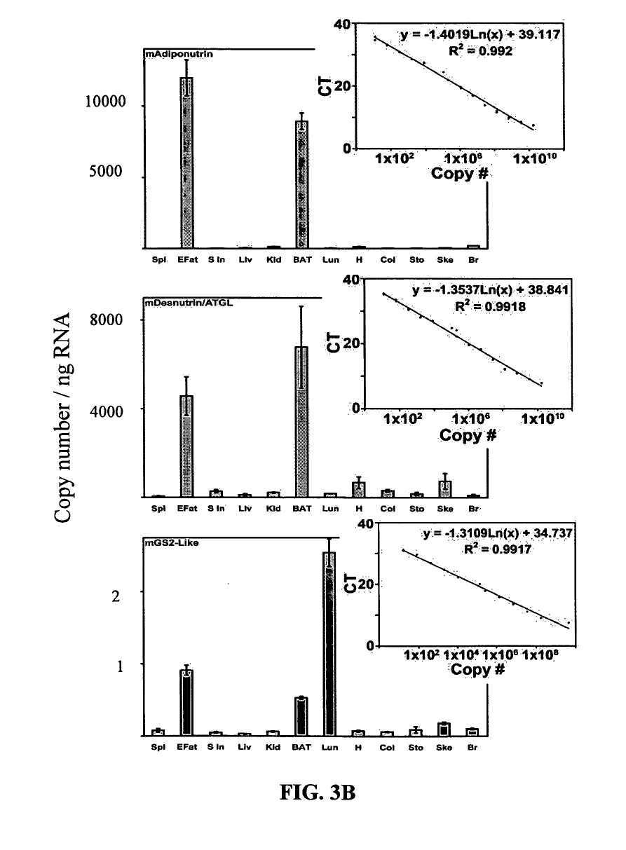

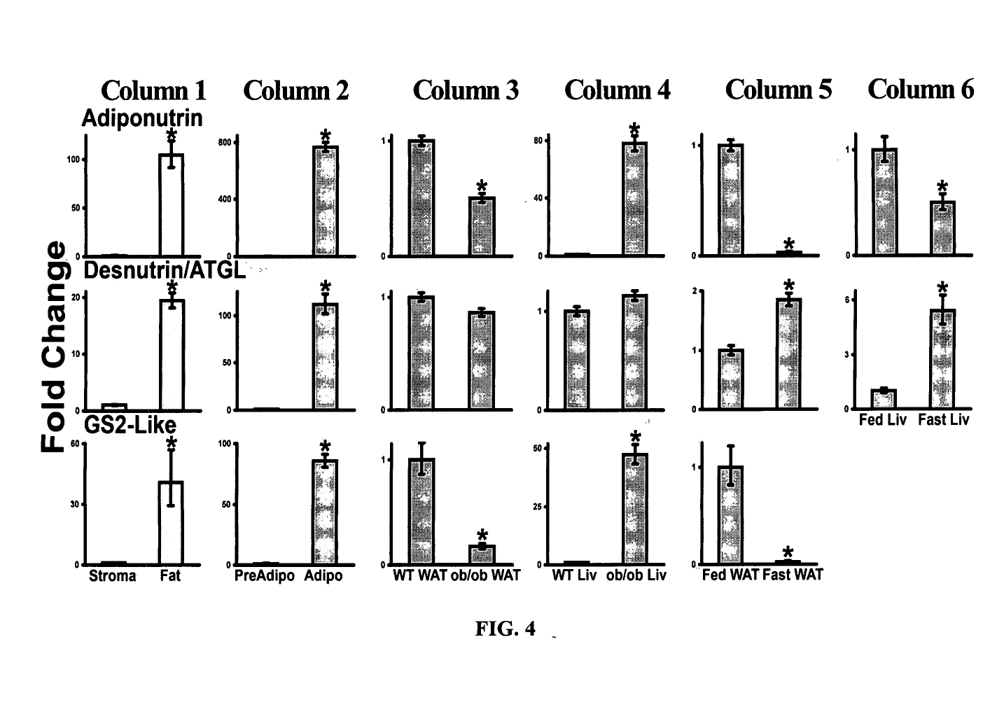

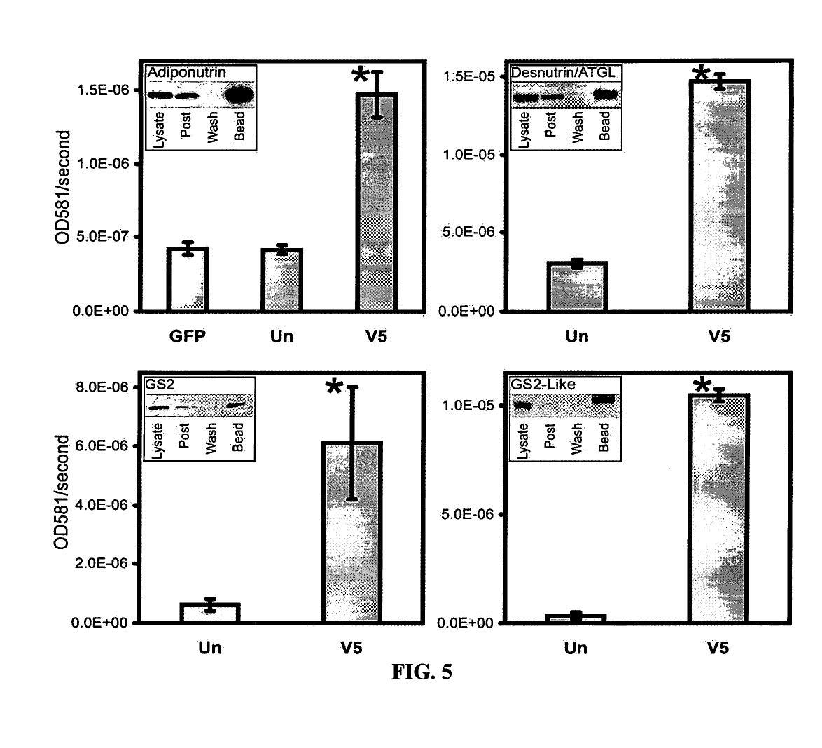

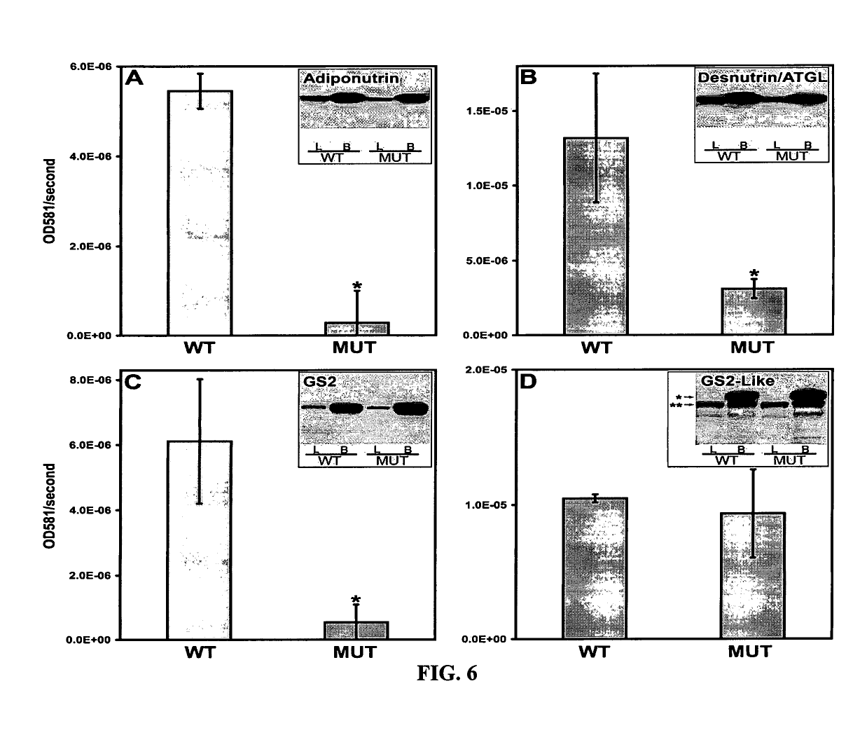

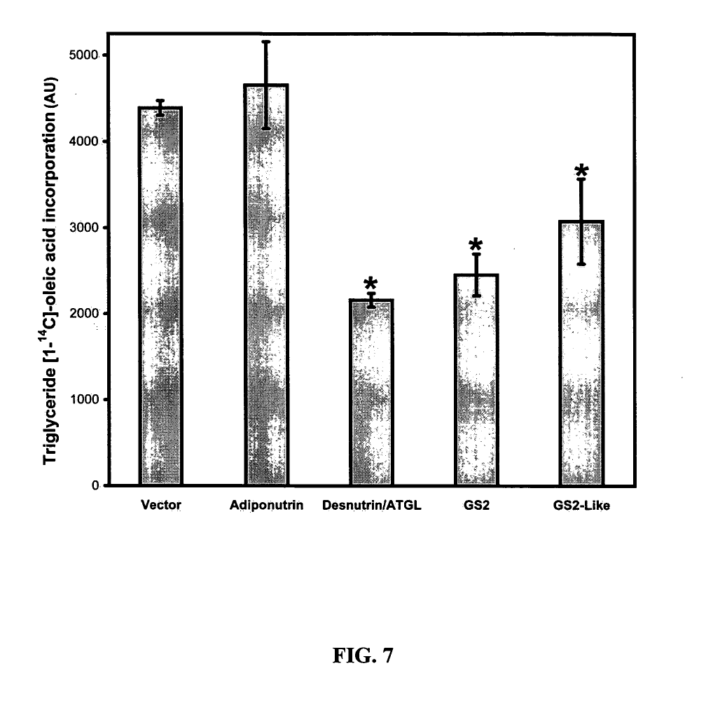

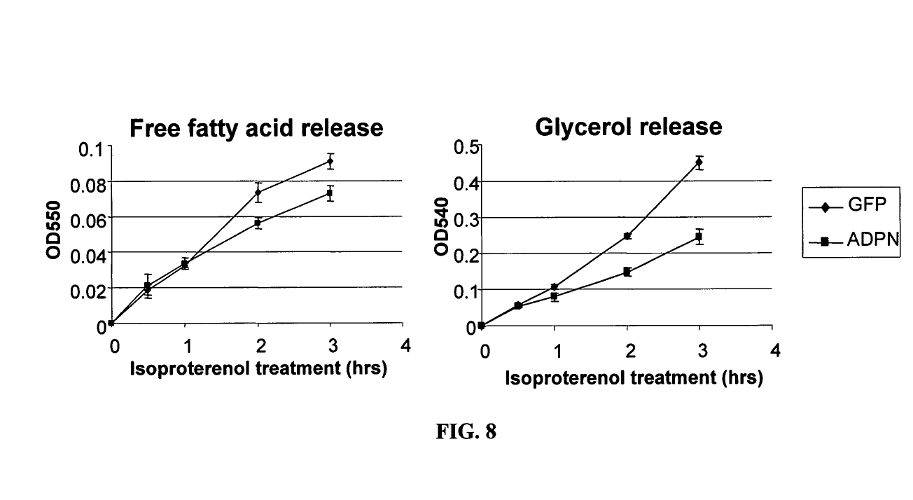

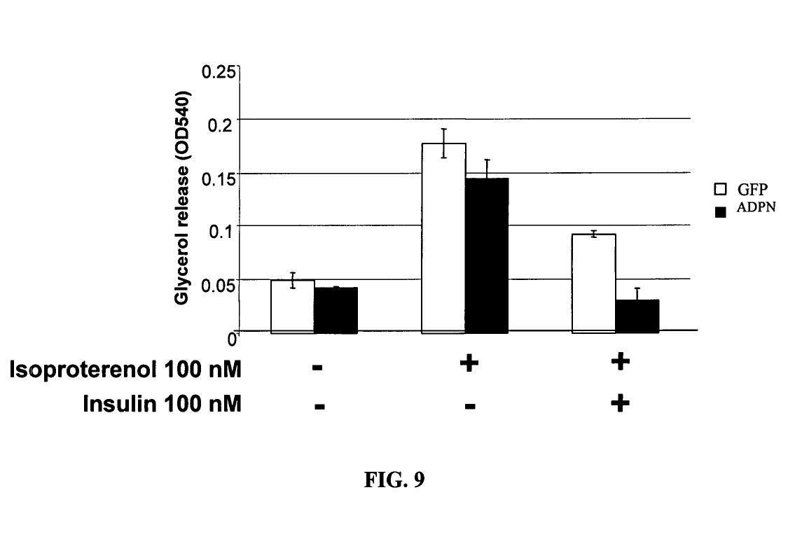

Adiponutrin, desnutrin/ATGL, GS2 and GS2-like are characterized by the presence of a patatin-like domain (Gly-X-Ser-X-Gly and Asp-X-Gly/Ala motifs) (Rydel, supra). To identify additional patatin-like domain-containing proteins, a profile HMM was generated and used to search public protein, EST and genomic databases. A total of 10 ortholog patatin families were identified (FIG. 2A). Phylogenetic analysis using patatin as the base sequence clustered adiponutrin, desnutrin/ATGL, GS2-like and GS2 in one branch of the phylogenetic tree (FIG. 2A). The inventors term this the adiponutrin family. A newly identified gene, PNPLA1 (in silico), also clustered with the adiponutrin family (FIG. 2A). The conserved residues of the patatin-like domain are present in all adiponutrin family members (FIG. 2B). Additionally, N-terminal patatin-like domains and variable C-terminal domains characterize all of the adiponutrin family members (FIG. 2C). In contrast to the adiponutrin family, the other patatin-like domain-containing proteins, including the phospholipases IPLA2, IPLA2-like, PLA2G6, NTE, and NTE-like, clustered on separate branches of the phylogenetic tree (FIG. 2A). In addition, these proteins were characterized by a C-terminal patatin-like domain and by a variable region at the N-terminus of the protein (data not shown).

With the exception of GS2, human, mouse, and rat orthologs for all members of the adiponutrin family were unequivocally identifed. Searches of EST and genomic databases failed to identify a mouse ortholog of GS2. Syntenic analysis of mouse, human and rat genomic regions shows that the region expected to contain the GS2 gene is absent in published mouse genomic sequences, allowing for the possibility that a gene corresponding to mouse GS2 remains to be discovered. The absence of any cDNA sequences corresponding to mouse GS2 in public or internal databases makes it unlikely that this gene is expressed at significant levels in major tissues.

Gene and protein prediction algorithms support the existence of PNPLA1 in the genome. However, a full-length cDNA for either the mouse or human predicted gene has not be isolated to date. Northern blotting of multiple human tissues using a human PNPLA1 EST (B1257213) or Q-PCR analysis of mouse tissues using multiple primer-probe sequences for the predicted mouse PNPLA1 failed to detect any appreciable levels of transcripts (data not shown). Consequently, the expression and physiological significance of PNPLA1 remains to be determined.

In conclusion, the bioinformatics analysis of adiponutrin, desnutrin/ATGL, GS2, GS2-like and PNPLA1 proteins indicated the following: 1) desnutrin/ATGL is enriched in or specific to fat tissue, 2) adiponutrin, desnutrin/ATGL, GS2, GS2-like and PNPLA1 have a close evolutionary relationship (FIG. 2A) and 3) the homology among adiponutrin, desnutrin/ATGL, GS2, GS2-like and PNPLA1 is due, in part, to each protein comprising at its N-terminus a patatin-like domain that is conserved in primary sequence (FIGS. 2B and 2C). These data indicate that adiponutrin, desnutrin/ATGL, GS2, GS2-like and PNPLA1 likely belong to the same protein family and that their patatin-like domains are likely to fuiction as esterases, e.g., lipases and/or lipid acyl hydrolases. As such, members of the adiponutrin family are likely to be crucial players in cardiovascular and metabolic disorders.

### Example 2

### Expression and Regulation Profiles of Adiponutrin Family Members

### Example 2.1

### Materials and Methods—Northern Blot Analysis

Human tissue Northern blots containing poly(A)-enriched mRNA were purchased from Clontech (Palo Alto, Calif.). Blots were prehybridized in Quickhyb (Stratagene, La Jolla, Calif.) for 30 minutes at 68° C., followed by hybridization in Quickhyb for 1.5 hours at 68° C. with gene-specific ³²P-labelled probes. Blots were washed to high stringency in 0.1×SSC, 1% SDS at 65° C. Human total RNA Northern blots (United States Biological, Swampscott, Mass.) were incubated in hybridization buffer (6×SSC, 5× Denhardts, 10 mg denatured salmon sperm DNA, 50% formamide, 0.5% SDS) for 3 hours 42° C., followed by hybridization with gene-specific probes for 16-24 hours at 42° C., and washes in 0.1×SSC, 0.1% SDS, 50° C. Random-primed ³²P-labeled, double-stranded cDNA probes were generated using the Stratagene Prime-It kit and protocol (Stratagene, La Jolla, Calif.) and purified using Amersham Biosciences' NICK column and protocol. Probes were made from gel-purified restriction fragments of I.M.A.G.E. Consortium [LLNL] cDNA clones as follows: human adiponutrin: ˜800 bp EcoRI/Xhol, 6081351; human desnutrin/ATGL: ˜1200 bp ApaI, 6598433; human GS2:˜800 bp EcoRV/SalI, 6109547; human GS2-like: ˜2300 bp SalI/NotI, 4778605; human PNPLA1: ˜700 bp SalI/NotI, 5123005. Autoradiography was performed at −80° C. using Optex L-plus intensifying screens. Blots were subsequently hybridized with a β-actin probe.

### Example 2.2

### Materials and Methods—Sample Acquisition and RNA Isolation

Normal mouse tissues were collected from 9-week-old male C57B1/6J mice fed ad libitum and euthanized by CO₂ asphyxiation. For all tissues with the exception of liver, two pools containing tissues from two individual animals each were analyzed. For liver, two individual animals were analyzed. The mouse preadipocyte line 3T3-L1 was obtained from the American Type Culture Collection (ATCC) and maintained according to ATCC guidelines. For differentiation, cells were grown in basal medium (Zen-Bio, Research Triangle Park, N.C.) for 4 days, and then transferred to adipocyte differentiation medium (Zen-Bio) for 4 days followed by culture in adipocyte medium (Zen-Bio) for 2 days prior to harvest. Undifferentiated control cells were harvested after 2 days in basal medium only. Three independent samples were analyzed for both undifferentiated and differentiated cells. Primary adipocytes were obtained from epididymal adipose tissue of ad libitum fed male C57B1/6J mice at 8-12 weeks of age. Adipose tissue was cut into small pieces, rinsed in isolation buffer (120 mM NaCl, 0.5 mM KCl, 1.2 mM KH₂PO₄, 0.6 mM MgSO₄.7H₂O and 0.9 mM CaCl₂.6H₂O, 10 mM HEPES, 200 nM adenosine, and 2.5% BSA), and digested with collagenase Type I (1 mg/ml/g fat; Worthington Biochemical Corporation, Freehold, N.J.) for 30-60 min with gentle shaking at 37° C. The digested material was passed through a 400 μM nylon mesh (Tetko, Kansas City, Mo.), and stromal cells and adipocytes were separated by centrifugation, with adipocytes floating on the surface. Adipocytes were transferred to clean tubes, and washed four times with the isolation buffer. The pellet containing stromal vascular cells was resuspended in red blood cell lysis buffer (0.83% NH₄Cl, 0.05 mM Na₂ EDTA, 0.1% KHCO₃, pH 7.3) to dissolve the red blood cells and was then pelleted for RNA extraction. One pool of stromal cells and three pools of primary adipocytes representing at least 15 mice each were analyzed. To examine regulation of gene expression in a mouse model of obesity and type 2 diabetes, the indicated tissues were obtained from 10 week old, ad libitum fed, male ob/ob or control mice (n=6, average body weight at 9 weeks of age: wt≈25 g, ob/ob≈49 g; fasting blood glucose at 9 weeks of age: wt≈60 mg/dl, ob/ob≈140 mg/dl). To examine regulation under conditions of altered lipid metabolism, 8 week old male C57B1/6J mice were either fed ad libitum or fasted for 24 hours prior to sacrifice. Samples from individual animals (n=6) were analyzed and data were expressed as mean±SEM. All mouse tissues were flash frozen in liquid nitrogen and stored at −80° C. until RNA isolation.

RNA was isolated from mouse tissues as follows. Tissue (˜5 mm×5 mm) was placed in a cold mortar containing liquid nitrogen and pulverized to a fine powder with a pestle. The frozen powder was transferred to a 1.5 ml centrifuge tube containing 800 μl of extraction buffer (TRIzol, Invitrogen, Carlsbad, Calif.). Next, the extraction buffer was passed through an 18 G needle 5-10 times followed by passage through a 23 G needle 20 times. Chloroform (160 ml) was added, the sample mixed, and phases separated by centrifugation. An equal volume of 70% ethanol was added to the aqueous phase and mixed. Samples were then processed using the RNeasy Mini purification kit and protocol, including DNaseI treatment (Qiagen, Valencia, Calif.). RNA quantity and quality was assessed by OD 260/280 and by visualization on an ethidium bromide-stained agarose gel. RNA was isolated from cell cultures by harvesting cells and immediately resuspending them in TRIzol. Extraction and purification was performed as described above.

### Example 2.3

### Materials and Methods—TaqMan Real-time Quantitative Reverse Transcription PCR (Q-PCR) Analysis

Murine adiponutrin, desnutrin/ATGL, GS2-like, and PNPLA1 MRNA expression was measured by Q-PCR analysis on panels of cDNAs from a variety of murine tissues and cell lines, which are described in Example 2.2.

Oligonucleotide primers and fluorescently-labeled TaqMan probes were designed using Primer Express 2.0 software (Applied Biosystems, Warrington, UK) based on the following GenBank or ENSEMBL reference sequences: adiponutrin, ay037763; desnutrin/ATGL, ak002826; GS2-like, xm₁₃ 128189; PNPLA1, ENSMUST00000056866. Sequences for primers and probes were as follows:

| | |
|---|---|
| Adiponutrin forward: | |
| 5′-CGAGGCGAGCGGTACGT-3′ | (SEQ ID NO:28) |
| Adiponutrin reverse: | |
| 5′-GTGACACCGTGATGGTGGTTT-3′ | (SEQ ID NO:29) |
| Adiponutrin probe: | |
| 5′-FAM-ACGGAGGAGTGAGCGACAACGTCC- | (SEQ ID NO:30) |
| TAMRA-3′ | |
| Desnutrin/ATGL forward: | |
| 5′-CAGCACATTTATCCCGGTGTAC-3′ | (SEQ ID NO:31) |
| Desnutrin/ATGL reverse: | |
| 5′-AAATGCCGCCATCCACATAG-3′ | (SEQ ID NO:32) |
| Desnutrin/ATGL probe: | |
| 5′-FAM-TGGCCTCATTCCTCCTACCCTCCAA- | (SEQ ID NO:33) |
| TAMRA-3′ | |
| GS2-like forward: | |
| 5′-CTCTGATCATGGTATTGACTGCTCTAA-3′ | (SEQ ID NO:34) |
| GS2-like reverse: | |
| 5′-TCCTCACTATCACAGGGATCAATC-3′ | (SEQ ID NO:35) |
| GS2-like probe: | |
| 5′-FAM-TGGGCTCCTTTCTCTGACCCACACTTATC | (SEQ ID NO:36) |
| T-TAMRA-3′ | |
| PNPLA1 forward: | |
| 5′-ACTGAATGCAGCGTACCTTGACT-3′ | (SEQ ID NO:37) |
| PNPLA1 reverse: | |
| 5′-GGCGACCTCTATCTGGCAGTATAC-3′ | (SEQ ID NO:38) |
| PNPLA1 probe: | |
| 5′-FAM-TCCCAGCAAGAGAGTGATTTTCCCGA- | (SEQ ID NO:39) |
| TAMRA-3′ | |

Q-PCR analysis was performed in an ABI PRISM 7000 Sequence Detection System (PE Applied Biosystems). Reactions were performed in a 25 μl volume with a final concentration of 1× Taqman PCR master mix (PE Applied Biosystems), 450 nM forward primer, 450 nM reverse primer, 250 nM probe primer, 10 ng of reverse transcribed total RNA (TaqMan Reverse Transcription Reagents kit, first-strand cDNA synthesis system protocol; Roche, N8080234), and 1× Eukaryotic 18S rRNA Endogenous control (VIC/TAMRA; PE Applied Biosystems). The thermal cycler conditions were as follows: 2 minutes at 50° C., followed by 10 minutes at 95° C., followed by two-step PCR for 40 cycles of 95° C. for 15 seconds and 60° C. for 1 minute. Threshold cycle (Ct) values were obtained for mouse adiponutrin, desnutrin/ATGL, GS2-like, and PNPLA1 and the values were normalized relative to the 18S internal control. Q-PCR reactions were performed in duplicate and the average values were used for quantification. Data analysis was performed as recommended by the manufacturer, and values were assigned based on standard curves, which were generated for each probe-primer set. Plasmid DNA containing the gene of interest was serially diluted and amplified by Q-PCR as described above. The following image clones were used for the Q-PCR standard curves: adiponutrin, 1265861; desnutrin/ATGL, 225573; GS2-like, 1150147. PCR efficiency was evaluated for each gene by examining the slope, which was ˜3.0 for all genes in this study.

### Example 2.4

### Results—Expression Analysis of Adiponutrin Family Members

To elucidate the expression patterns of the adiponutrin gene family, Northern analysis was performed using gene-specific probes. Both desnutrin/ATGL and GS2 transcripts were easily detectable by Northern blotting and both genes showed highest expression in metabolically active tissues, such as adipose tissue, heart, skeletal muscle, and portions of the gastrointestinal tract (FIG. 3A), consistent with a role in lipid metabolism. As reported previously, two desnutrin/ATGL (˜2.2 kb, ˜4.3 kb) (Zimmermann et al. (2004) supra) and two GS2 transcripts (˜1.4 kb, ˜4.4 kb) (Lee et al. (1994) Genomics 22:372-76) are detectable by Northern blotting and appear to be coordinately expressed in most, but not all, tissues (FIG. 3A). Human GS2-like or human adiponutrin transcripts were undetectable by Northern blotting, presumably due to lower levels of expression (data not shown).

To assess the mRNA expression of mouse adiponutrin family members, quantitative real-time PCR (Q-PCR) analysis was used. As expected, both adiponutrin and desnutrin/ATGL are most abundant in both brown adipose tissue (BAT) and white adipose tissue (epididymal fat; EFat). In addition, it was found that GS2-like was expressed predominantly in adipose tissue (both EFat and BAT) and lung. Notably, multiple probes for GS2-like consistently required very high cycle numbers, suggesting poor expression of GS2-like MRNA in the tissues examined. To verify that this was indeed the case, Taqman analysis was quantitated using standard curves generated on known quantities of plasmid containing the indicated gene (FIG. 3B, insets). These standard curves were used to convert cycle time (CT) into copies of cDNA normalized to the amount of starting RNA. FIG. 3B shows that GS2-like cDNA was much less abundant (˜10,000-fold) than adiponutrin or desnutrin/ATGL-cDNA. Identical data were generated with an independent set of primer/probes (data not shown). In conclusion, GS2-like is expressed at very low levels, likely explaining the inability to detect expression by Northern Blotting in either mouse or humans (data not shown).

### Example 2.5

### Results—Regulation of Adiponutrin Family Member Expression in Metabolic Paradigms

To further examine the expression of adiponutrin, desnutrin/ATGL and GS2-like, primary adipocytes were separated from the stromal fraction of mouse adipose tissue and examined for expression during 3T3-L1 adipocyte differentiation. As expected, both adiponutrin and desnutrin/ATGL were detected almost exclusively in the adipocyte fraction and were highly upregulated during adipocyte differentiation (FIG. 4, columns 1-2). Similarly, GS2-like was found to be an adipocyte-expressed gene that is upregulated during adipocyte differentiation (FIG. 4, columns 1-2).

Desnutrin/ATGL has previously been reported to be downregulated in the adipose tissue of genetically obese (ob/ob) mice (Villena, supra), while adiponutrin was reported to be strongly induced in the adipose tissue of obese (fa/fa) rats (Baulande, supra). The expression of adiponutrin family members in adipose and liver tissue from ob/ob mice and from fasted mice was examined. Contrary to previous reports, a significant change in desnutrin/ATGL mRNA expression in adipose tissue of ob/ob mice was undetectable and adiponutrin mRNA was significantly (˜2-fold) decreased (FIG. 4, column 3). GS2-like mRNA was downregulated ˜5-fold in the adipose tissue of ob/ob mice, again closely paralleling the regulation of adiponutrin (FIG. 4, column 3). It is interesting to note that tissues from ob/ob mice were collected under fed conditions. It is possible, therefore, that the previously observed decrease in fasted ob/ob adipose tissue (Villena et al. (2004) supra) reflects a lack of upregulation of desnutrin/ATGL upon fasting. Consistent with previous data, adiponutrin expression was strongly suppressed upon fasting (˜80-fold), while desnutrin/ATGL expression is increased ˜2-fold in adipose tissue from fasted mice (FIG. 4; column 5). GS2-like MRNA was regulated in a manner similar to adiponutrin and decreased strongly in adipose tissue upon fasting (FIG. 4, column 5).

The regulation of adiponutrin and GS2-like in adipose tissue is reminiscent of regulation found for genes involved in lipogenesis rather than lipolysis. Since genes expressed in lipogenesis (e.g., SCD1 (Ntambi and Miyazaki (2004) Prog. Lipid Res. 43:91-104; Ntambi et al. (2004) Lipids 39:1061-65; Ntambi et al. (1988) J. Biol. Chem. 263:17291-300) and DGAT2 (Cases et al. (2001) J. Biol. Chem. 276:38870-76; Suzuki et al. (2005) J. Biol. Chem. 280:3331-37)) are often upregulated in the livers of animals with hepatic steatosis, such as ob/ob mice, and downregulated in livers from fasting animals, the expression of adiponutrin family members in the livers in these models was examined. Both adiponutrin and GS2-like expression were strongly induced in ob/ob livers compared to control litter-mates, while desnutrin/ATGL expression was unchanged (FIG. 4, column 4). During fasting, desnutrin/ATGL expression increased significantly, while adiponutrin expression decreased below starting levels (FIG. 4, column 6). GS2-like expression was essentially undetectable in the liver under both fasting and fed conditions (data not shown). The upregulation of both adiponutrin and GS2-like in the liver of ob/ob mice suggests a role for these proteins in hepatic steatosis.

### Example 3

### Enzymatic Activity of Adiponutrin Family Members

### Example 3.1

### Materials and Methods—Expression Vectors

Human adiponutrin, desnutrin/ATGL, GS2 and GS2-like expression constructs encoding only the open reading frame with or without epitope tags were constructed by subcloning PCR amplification products into the mammalian expression vector pADORI1.2 (CMV promoter (generated at Wyeth, Cambridge, Mass.)) only, or into the gateway entry vector pDONR (Invitrogen, Carlsbad, Calif.) followed by recombination into the Gateway destination vector pDEST40 (Invitrogen). PCR amplification was performed using Invitrogen's Platinum Taq DNA polymerase, I.M.A.G.E. Consortium [LLNL] cDNA clones 5243623 (adiponutrin), 6598433 (desnutrin/ATGL), 6109547 (GS2) and 4778605 (GS2-like) and the following primers:

| | |
|---|---|
| Human adiponutrin forward: | |
| 5′-TATATACTAGTACTAGTCGGACCATGTACGACG | (SEQ ID NO:40) |
| CAGAGCGCGGCTGGAGC-3′ | |
| Human adiponutrin no tag reverse: | |
| 5′-ATATAAAGCTTAAGCTTTCATCACAGACTCTTC | (SEQ ID NO:41) |
| TCTAGTGAAAAACT-3′ | |
| Human adiponutrin carboxy-V5 tag | |
| reverse | |
| 5′-ATATAAAGCTTTCACGTAGAATCGAGACCGAGG | (SEQ ID NO:42) |
| AGAGGGTTAGGGATAGGCTTACCCAGACTCTTCTCT | |
| AGTGAAAAACT-3′ | |
| Human desnutrin/ATGL forward: | |
| 5′-GGGGACAAGTTTGTACAAAAAAGCAGGCTTCGA | (SEQ ID NO:43) |
| AGGAGATAGAACCATGTTTCCCCGCGAGAAGACG- | |
| 3′ | |
| Human desnutrin/ATGL no tag reverse: | |
| 5′-GGGGACCACTTTGTACAAGAAAGCTGGGTCCTA | (SEQ ID NO:44) |
| CAGCCCCAGGGCCCCGAT-3′ | |
| Human desnutrin/ATGL carboxy-V5-6His | |
| tag reverse | |
| 5′-GGGGACCACTTTGTACAAGAAAGCTGGGTCCAG | (SEQ ID NO:45) |
| CCCCAGGGCCCCGAT-3′ | |
| Human GS2 forward: | |
| 5′-ATATAGAATTCCGGACCATGAAGCACATCAACC | (SEQ ID NO:46) |
| TATCATTTGCA-3′ | |
| Human GS2 no tag reverse: | |
| 5′-ATATAAAGCTTTCATTCAAACCAATTTTCTTTA | (SEQ ID NO:47) |
| AGTAA-3′ | |
| Human GS2 carboxy-V5 tag reverse: | |
| 5′-ATATAAAGCTTTCACGTAGAATCGAGACCGAGG | (SEQ ID NO:48) |
| AGAGGGTTAGGGATAGGCTTACCTTCAAACCAATTT | |
| TCTTTAAGTAA-3′ | |
| Human GS2-like forward: | |
| 5′-ATGGGCTTCTTAGAGGAGGAGG-3′ | (SEQ ID NO:49) |
| Human GS2-like no tag reverse: | |
| 5′-GGACTAGTTCAATGGTGATGGTGATGATGGGTA | (SEQ ID NO:50) |
| CGCGTAGAGTCGAGACCGAGGAGAGGGTTAGGGATA | |
| GGCTTACCGGCCTGGTGGGTGG-3′ | |
| Human GS2-like carboxy-V5-6His tag | |
| reverse: | |
| 5′-GGACTAGTTCAATGGTGATGGTGATGATGGGTA | (SEQ ID NO:51) |
| CGCGTAGAGTCGAGACCGAGGAGAGGGTTAGGGATA | |
| GGCTTACCGGCCTGGTGGGTGGGCCC-3′ | |

Each expression vector was used to transiently transfect HEK293 cells and expression was confirmed using Western blotting.

### Example 3.2

### Materials and Methods—Generation of Adenovirus

The pADORI1.2 expression plasmid comprising human adiponutrin was digested with AscI and BstZ17I and the fragment containing the expression cassette and adenoviral genomic sequence was isolated. Recombinant adenoviral genomic DNA was linearized with PacI. The isolated pADORI1-2 fragment was cotransfected with adenoviral genomic DNA into early passage 293 cells using Fugene 6 (Roche). Ten to fourteen days post-transfection, the recombinant adenovirus was harvested and subjected to another two rounds of viral amplification in 293 cells. The resulting virus was used to infect 293 cells at a large scale followed by purification by CsCl gradient ultracentrifugation.

### Example 3.3

### Materials and Methods—Patatin-like Domain Mutant Generation

The C-terminal-V5-tagged hAdiponutrin and hGS2 expression vectors, and the C-terminal-V5-6His tagged hDesnutrin/ATGL and hGS2-like expression plasmids, were used to generate site directed patatin-like domain mutants. Mutant design was based on previously reported mutants for patatin (Rydel et al. (2003) Biochemistry 42:6696-708). Using the QuikChange® XL Site-Directed Mutagenesis Kit and protocol (Stratagene, La Jolla, Calif.), the serine of the Gly-X-Ser-X-Gly motif (FIG. 2B) was changed to an alanine for each family member. The resulting plasmids were sequenced.

### Example 3.4

### Materials and Methods—Cell Culture and Transfection

HEK293 cells were grown in DMEM media supplemented with 10% fetal calf serum at 37° C. in an atmosphere of 5% CO2. Ten cm tissue culture dishes with 90% confluent HEK293 cells were transfected using Lipofectamine 2000 according to the manufacturer's instructions (Invitrogen, Carlsbad, Calif.). Ten μg of plasmid DNA containing the following expression constructs were used: GFP, human adiponutrin, human adiponutrin-c-term-V5, human desnutrin/ATGL-c-term-V5-6His, human GS2-c-term-V5, human GS2-like-c-term-V5-6His, and patatin-like domain mutants of c-term-V5 and c-term-V5-6His tagged constructs. Three days post-transfection, cells were washed with ice cold TBS and harvested. Cells were pelleted by centrifugation at 1000×g for 5 minutes. Supernatant was discarded, and pellets stored at −80° C. until used for assays.

### Example 3.5

### Materials and Methods—Preparation of Cell Lysates and Immunoprecipitations

Frozen cells, as described above, were resuspended in 1 ml of lipase reaction buffer (20 mM Tris-HCl, pH 8.0, 150 mM NaCl, 0.53% sodium taurodeoxycholate, 1.33 mM CaCl₂) containing complete mini-protein inhibitor tablets (Invitrogen, Carlsbad, Calif.; 1 tablet per 7 ml). The resulting suspensions were then sonicated on ice with 4 bursts of 10 seconds from a probe sonicator. Homogenized lysate was then centrifuged at 1000×g for 10 minutes to remove cell debris. A 50 μl aliquot of the resulting lysate was saved for analysis and the remaining lysate was used for the immunoprecipitation. Anti-V5 mouse monoclonal antibody (7.2 μg; Invitrogen, Carlsbad, Calif.) was added to each lysate and then placed at 4° C. overnight with tumbling. Next, 20 μl of protein-A beads (Repligen) was added to each sample and tumbled at 4° C. for 2 hours. Beads were then pelleted with gentle centrifugation and 900 ml of supernatant was saved for analysis. The beads were washed 4 times with 900 μl of lipase reaction buffer followed by resuspension in 100 μl of lipase reaction buffer.

### Example 3.6

### Materials and Methods—Lipase Assay

The lipase assay (Lehner and Verger (1997) Biochemistry 36:1861-68) uses 1,2-o-dilauryl-rac-glycero-3-glutaric acid-(6′-methylresorufin) ester (DGGR) as substrate. DGGR is cleaved by lipase, resulting in an unstable dicarbonic acid ester that is spontaneously hydrolyzed to yield glutaric acid and methylresorufin, a bluish-purple chromophore with peak absorption at 581 nm. The rate of methylresorufin formation is directly proportional to the lipase activity in the sample. Ten μl of whole cell lysate or IP beads were added to the wells of a 96-well plate. After diluting the samples up to 125 μl in lipase reaction buffer, 125 μl of reaction buffer containing DGGR (final concentration 36 μg/ml in a final assay volume of 250 μl per well). After mixing, OD 581 was monitored at 5 minute intervals for 2 hours to assess lipase activity. Lipase activity is plotted as delta-OD 581/sec. All samples were assayed in triplicate.

### Example 3.7

### Materials and Methods—[1-¹⁴C]-Oleic Acid Incorporation into Triglyceride

HEK293 cells were cultured in Dulbecco's modified Eagle's medium (DMEM) that contained 10% FBS at 37° C. with 5% CO₂. HEK293 cells grown in 12-well plates to 60-70% confluence were transfected with 0.5 μg of human adiponutrin, desnutrin/ATGL, GS2, or GS2-like expression vector using FuGene (Roche). Empty vector, pADORI1.2 was used as a control. After 48 hours, cells were treated with 4 μM [1-¹⁴C]-oleic acid (204 μCi/ml total; Perkin Elmer) and 16 μM cold oleic acid (Sigma) in serum-free DMEM. After 4 hours, cells were washed twice and incubated further with DMEM with 10% FBS. After 16 hours, lipids were extracted and separated by TLC using hexane/ether/acetic acid (80:20:1). Radioactive lipids were detected and quantitated by Molecular Imager FX (Bio-Rad).

### Example 3.8

### Materials and Methods—Effect of Adiponutrin Overexpression on 3T3-L1 Lipase Activity

For adenovirus infection, 3T3-L1 cells were cultured in DMEM with 10% FBS at 37° C. with 5% CO₂. Two days after the cultures were confluent, the cells were induced to differentiate (defined as day 0) by incubation in DMEM supplemented with 10% FBS, 1 μM dexamethasone (Sigma, St. Louis, Mo.), 0.5 mM isobutylmethylxanthine (Sigma) (MIX), and 1 μg/ml insulin (Sigma). After two days, the medium was replaced with DMEM supplemented with 10% FBS and 1 μg/ml insulin, and cells were then fed every 2 days with the same medium. At day 8 of differentiation, cells grown in 24-well plates were incubated with 500 MOI of adenovirus in OPTI-MEM (Invitrogen, Carlsbad, Calif.) for 2 hours and then DMEM with 10% FBS. After 48 hours, cells were incubated with DMEM with no serum overnight. The next day, cells were washed three times with PBS and incubated with PBS with 2% fatty acid-free BSA (Sigma, St. Louis, Mo.) with 10 μM or 100 nM of isoproterenol (Sigma). When needed, cells were preincubated with 100 nM insulin 30 min prior to isoproterenol treatment. Conditioned medium was taken at the indicated 0-4 hours after isoproterenol treatment and analyzed for free fatty acids and glycerol. Free fatty acids release was measured by using a NEFA-c kit (Wako Chemicals, Richmond, Va.) and glycerol release was measured by using a lipolysis kit (Zen-bio, Research Triangle Park, N.C.).

### Example 3.9

### Results—Adiponutrin Family Members Are Lipid Hydrolases

Lipase and transacylase activity for a subset of adiponutrin family members was recently demonstrated (Zimmerman, supra; Jenkins, supra). Here, the ability of adiponutrin family members to hydrolyze a commonly used lipase substrate, 1,2-o-dilauryl-rac-glycero-3-glutaric acid-(6′-methylresorufm) ester (DGGR) was examined. V5-tagged human adiponutrin, desnutrin/ATGL, GS2 and GS2-like proteins were expressed in HEK293 cells and partially purified by immunoprecipitation. GFP or untagged versions of the same protein, processed in an identical manner, were used as controls. Western blots confirmed that the proteins were expressed, and present in the immunoprecipitate (FIG. 5, insets). All four adiponutrin family members tested showed a significantly higher rate of hydrolysis compared to immunoprecipitates from cells expressing GFP or untagged controls (FIG. 5).

The patatin domain is characterized by a conserved GXSXG motif, the conserved serine (S) of which is part of the Ser-Asp catalytic dyad and thought to mediate catalysis. To determine whether this serine is required for the catalytic activity of adiponutrin family members, this residue was mutated to alanine. Equivalent amounts of wild-type (WT) and mutant (MUT) proteins were assayed as confirmed by Western blotting (FIG. 6, insets). For three family members, adiponutrin, desnutrin/ATGL and GS2, the rate of lipid hydrolysis was significantly higher when wild-type rather than mutant protein was used, demonstrating that the lipase activity is due to the overexpressed protein and depends upon an intact active site serine (FIG. 6). The activity of preparations containing GS2-like was not affected by mutating the active-site serine. It is possible that one of the two other serine residues within the GXSXG motif of GS2-like (i.e., GSSSG) can function as a catalytic serine; alternatively, GS2-like preparations may contain a coprecipitating lipase that masks the decreased activity of mutated GS2-like.

### Example 3.10

### Results—Overexpression of Adiponutrin Family Proteins Modulates Fatty Acid Metabolism

To further assess the ability of adiponutrin family members to function as lipases, the effects of overexpression of these proteins on intracellular triglyceride levels were examined. Overexpression of desnutrin/ATGL in mammalian cell lines has previously been shown to decrease the incorporation of radiolabeled fatty acids into triglycerides by increasing the release of fatty acids during a pulse-chase experiment. Adiponutrin, desnutrin/ATGL, GS2 and GS2-like were examined in a similar experiment using overexpression in HEK293 cells. Consistent with previous results, overexpression of desnutrin/ATGL caused a significant decrease in intracellular triglycerides compared to control cells (FIG. 7). GS2 and GS2-like also decreased intracellular triglycerides significantly. In contrast, the inventors were unable to demonstrate a decrease upon adiponutrin overexpression; in fact, in several experiments, adiponutrin showed a trend toward increased triglyceride incorporation (data not shown). Thus, desnutrin/ATGL, GS2 and GS2-like can all function to decrease intracellular triglycerides, at least when overexpressed, while adiponutrin behaves differently in cells.

### Example 3.1 1

### Results—Overexpression of Adiponutrin Inhibits Lipolysis

To evaluate the effects of adiponutrin on adipocyte lipolysis, human adiponutrin was overexpressed in 3T3-L1 adipocytes using an adenoviral expression system. Overexpression of adiponutrin significantly decreased the release of free fatty acids and glycerol into the medium after treatment with 10 μM isoproterenol (FIG. 8), indicating that adiponutrin overexpression blocks lipolysis induced by adrenergic receptor agonists. To evaluate whether adiponutrin acts synergistically with insulin, the effect of adiponutrin overexpression on lipolysis induced by a submaximal concentration of isoproterenol in the presence or absence of insulin was evaluated. Adiponutrin overexpression had no effect on basal lipolysis (FIG. 9). As expected, the presence of insulin decreased isoproterenol-stimulated lipolysis (FIG. 9). Adiponutrin overexpression significantly enhanced the inhibition of lipolysis by insulin (FIG. 9).

### Example 4

### Discussion

Controlled storage and release of fatty acids is a central function of the adipocyte. A patatin-like domain containing protein, desnutrin/ATGL, has recently been demonstrated to have triglyceride lipase activity and to mediate lipolysis in mouse adipocytes (Zimmermann, supra; Jenkins, supra; Villena, supra). Desnutrin/ATGL is believed to be similar, in some regards, to adiponutrin, an adipocyte-specific protein of unknown function that is dramatically downregulated upon fasting in mouse white adipose tissue (Polson and Thompson (2003) supra; Liu (2004) supra; Polson and Thompson (2004) supra; Bertile and Raclot (2004) supra). Recently, desnutrin/ATGL, adiponutrin and a third patatin-like domain-containing protein, GS2, were expressed in insect cells and shown to have triglyceride lipase as well as transacylase activities (Jenkins, supra). The bioinformatic analysis provided herein identified two adiponutrin family members that have not been studied to date, GS2-like and PNPLA1. As such, the present studies evaluated adiponutrin family members (with respect to tissue distribution, MRNA regulation, and activities in enzymatic and cell-based assays) to determine the possible contributions of different adiponutrin family members to adipocyte lipolysis. Since evidence of PNPLA1 expression in any of the tissues examined was difficult to obtain, efforts focused on GS2-like in parallel with the three other adiponutrin family members, GS2, adiponutrin and desnutrin/ATGL.

DNA sequences corresponding to GS2-like have been identified as possible adiponutrin homologs in several publications; however, to date no studies have been conducted on GS2-like itself. The present invention demonstrates that GS2-like is indeed a member of the adiponutrin family; similar to adiponutrin and desnutrin/ATGL, GS2-like has an N-terminal patatin-motif followed by a C-terminal variable domain. Human, mouse and rat orthologs of GS2-like are clearly identifiable in the respective genomes, demonstrating evolutionary conservation of this gene. Interestingly, GS2-like is located adjacent to adiponutrin in human, rat and mouse genomes (unpublished observations), and may therefore be the result of an adiponutrin gene duplication event. While GS2-like is qualitatively expressed and regulated in a manner similar to adiponutrin (FIGS. 3B and 4), the absolute expression levels of GS2-like are dramatically lower, at least at the RNA level in the mouse (FIG. 3B). While the relative expression of GS2-like in human adipose tissue was not quantitated, the inability to detect expression by Northern blotting analysis is consistent with low levels of expression of GS2-like in the human adipose tissue as well. The cell-based data show that GS2-like can decrease triglyceride incorporation when overexpressed in cells in a manner similar to desnutrin/ATGL, suggesting it can function as a lipase in cells. Given the low level of expression of GS2-like in mouse adipose tissue, it is unlikely that GS2-like contributes significantly to lipolysis in this tissue. It is possible, however, that GS2-like becomes functionally more important under particular pathophysiological conditions, especially in tissues that do not express large amounts of desnutrin/ATGL. For example, GS2-like expression, but not desnutrin/ATGL expression, is upregulated significantly in the liver of ob/ob mice (FIG. 4, column 4).

GS2 is an adiponutrin family member that was originally identified as part of the genome sequencing effort, and was shown to be expressed in several tissues (Lee, supra). Recently, GS2 has been shown to be expressed in the human liposarcoma cell line SW872 and to have triglyceride lipase and transacylase activity. Here, it is demonstrated for the first time that GS2 is expressed in human adipose tissue, and that adipose tissue as well as other tissues with significant lipid metabolism, such as heart, skeletal muscle, liver, kidney and sections of the gastrointestinal tract, are the major sites of GS2 expression in humans. Given the relatively broad pattern and high levels of expression of GS2 in human tissues, the complete absence of sequences corresponding to GS2 transcripts in mouse databases is surprising. Since the genomic region predicted to contain GS2 is lacking in the mouse genomic sequence, there is currently no evidence of a mouse GS2 gene. One possible explanation is that mouse GS2 is expressed at much lower levels compared to human GS2 making the identification of transcripts more difficult; alternatively, GS2 may be lacking in the mouse genome. Purified GS2 has previously been shown to have triglyceride lipase and transacylase activities that are comparable to or stronger than those observed with adiponutrin and desnutrin/ATGL. Here, overexpression of GS2 in 293 cells lowers triglyceride incorporation in a manner similar to desnutrin/ATGL (FIG. 7). Thus, GS2 can act as a lipase in cells, at least upon overexpression. The expression pattern of GS2, coupled with its ability to act as a lipase, raises the possibility that GS2 may contribute significantly to adipocyte lipolysis in humans. GS2 is unique among adiponutrin family members in its gene structure: GS2 only contains a patatin-like domain and lacks the C-terminal variable region found in all other adiponutrin family members. The ability of GS2 to act as a lipase in the context of a cell suggests that the variable region is not required for triglyceride lipase activity. The function of this variable domain is currently unclear.

he serine within the Gly-X-Ser-X-Gly motif has been suggested to be the active-site serine for adiponutrin family members; however, to date, this has not been demonstrated. Here the inventors show for the first time that for three of these enzymes (adiponutrin, desnutrin/ATGL, and GS2), the predicted active-site serine is indeed crucial to the lipase activity (FIG. 6). Interestingly, for GS2-like, activity was not abolished in protein-A beads derived from cells transfected with patatin-domain mutants. Since beads from cells expressing untagged GS2-like protein did not show lipase activity, it is possible that the mutant V5-tagged protein is coimmunoprecipitating with some other protein, which could also contribute to the GS2-like lipase activity observed. Another possibility is that other serine residues at the active site Gly-X-Ser-X-Gly motif can also contribute to the activity of the enzyme. GS2-like is the only family member with additional serine residues adjacent to the active site (FIG. 2B). The fact that the mutant GS2-like protein retains lipase activity, does not, however rule out the possibility that GS2-like protein has lipase activity of its own as evidenced by the triglyceride hydrolysis results (FIG. 7).

Desnutrin/ATGL and adiponutrin have both been shown to have triglyceride lipase activity in vitro (Zimmermann, supra; Jenkins, supra); however, only desnutrin/ATGL is thought to contribute to adipocyte lipolysis (Jenkins, supra; Zimmermann, supra; Villena, supra). The data herein show that GS2 is highly expressed in human adipose tissue and, similar to desnutrin/ATGL, can reduce triglyceride accumulation when overexpressed. This raises the possibility that in human adipose tissue, GS2 may mediate a significant portion of lipolysis. While adiponutrin, desnutrin/ATGL, and GS2 have both transacylase and lipase activity in vitro (Jenkins, supra), it has been previously suggested that adiponutrin, in the context of a cell, acts primarily as a transacylase to incorporate fatty acids into triglycerides, rather than as a lipase to release fatty acids. The regulation and cell-based assay data provided herein further support this hypothesis.

Previous studies have shown that adiponutrin and desnutrin/ATGL are both highly expressed in adipose tissue, but are differentially regulated during fasting (Zimmermann, supra; Polson and Thompson (2003) supra; Liu, supra; Polson and Thompson (2004) supra; Villena, supra). Desnutrin/ATGL expression increases ˜2-fold upon fasting, while adiponutrin expression becomes virtually undetectable (Zimmermann, supra; Villena, supra). The MRNA expression data provided herein confirm the expression of desnutrin/ATGL in adipose tissue and primary adipocytes, its upregulation during adipocyte differentiation, and its differential regulation upon fasting. Contrary to previously reported results, a significant downregulation of desnutrin/ATGL in ob/ob mice was not observed. Since this study used fed animals while the previous study relied on fasted animals, it is possible that this difference reflects a dysregulation of desnutrin/ATGL mRNA in ob/ob mice specifically upon fasting. Interestingly, it was found that adiponutrin expression is downregulated in the adipose tissue of ob/ob mice. It is observed that many lipogenic genes (e.g., G3PAT, DGAT2, SCD1) are downregulated in the adipose tissue of ob/ob mice under the conditions used in this study (unpublished data); thus, this downregulation of adiponutrin in the present model is consistent with its proposed anti-lipolytic role. Previously, adiponutrin mRNA was shown to be dramatically upregulated in adipose tissue of genetically obese fa/fa rats (Baulande, supra), which carry a mutation in the leptin receptor (Koteish and Mae Diehl (2002) Best Pract. Res. Clin. Gastroenterol. 16:679-90). It is possible that differences in species, diet or mutation account for the differential regulation observed in the obesity model herein compared to others.

The regulation of adiponutrin family members in the liver had not been examined previously. Similar to adipose tissue, the liver undergoes cycles of lipogenesis and lipolysis. Increased triglyceride accumulation in the liver (hepatic steatosis) is commonly observed in genetic and diet-induced obesity (Koteish and Mae Diehl, supra; den Boer et al. (2004) Arterioscler. Thromb. Vasc. Biol. 24:644-49) and is accompanied by increased expression of genes involved in lipid storage, including PPARgamma (Gavrilova et al. (2003) J. Biol. Chem. 278:34268-76), DGAT2 (Suzuki et al. (2005) supra), SCD1 (Ntambi and Miyazaki (2004) supra; Cohen et al. (2003) Curr. Drug Targets Immune Endocr. Metabol. Disord. 3:271-80), etc. In the present studies, it was found that the regulation of adiponutrin and desnutrin/ATGL in the liver during fasting mirrored their regulation in adipose tissue. In addition, a dramatic upregulation of both adiponutrin and GS2-like, but no increase in desnutrin/ATGL expression in livers of ob/ob mice, was observed. The regulation of adiponutrin in the liver is consistent with the proposed role in lipogenesis, and further accentuates the difference between desnutrin/ATGL and adiponutrin in terms of MRNA regulation.

While adiponutrin acts as a lipase in vitro (FIG. 5; Jenkins, supra), its function in vivo has been unclear. The data in HEK293 cells (FIG. 7) clearly show that adiponutrin does not function as a lipase in cells and is functionally distinct from the other adiponutrin family members. Also, overexpression of adiponutrin in 3T3-L1 adipocytes inhibits lipolysis and potentiates the anti-lipolytic action of insulin (FIG. 9). This is of interest, since adiponutrin mRNA is known to be upregulated in response to insulin in human and mouse adipocytes in vitro (Baulande, supra; Johansson (2006) Diabetes 55:826-33) and is upregulated in obese individuals which have high levels of plasma insulin (Johansson, supra). Interestingly, analysis of obese individuals shows that adiponutrin mRNA is positively correlated with insulin sensitivity suggesting a protective fuiction for adiponutrin (Johansson, supra). Thus, modulators that increase the activity or expression of adiponutrin may be beneficial for the treatment of type 2 diabetes. On the other hand, agents that increase lipolysis have been suggested as treatments for obesity (e.g., beta3-adrenergic receptor agonists, inhibitors of perilipin). Thus, modulators that inhibit adiponutrin expression or activity may be protective for the treatment of obesity. Furthermore, the experiments described herein (e.g., Examples 3.10 and 3.11) are the first to demonstrate activity of adiponutrin in a cell-based system. The system described may be used to identify and characterize modulators of adiponutrin function.

It is unclear why adiponutrin behaves as a lipase in vitro, but has anti-lipolytic activity in cells. One possibility is that, in cells, adiponutrin interacts with additional proteins or cofactors that stimulate its transacylase activity while inhibiting its lipase activity, thus promoting anti-lipolytic function. Another possibility is that adiponutrin sequesters molecules (e.g. substrates or cofactors) from desnutrin/ATGL, thus blocking lipolysis. The materials and methods provided herein, e.g., to measure the activity of adiponutrin in a cell-based system, etc., provide novel tools for further studies aimed at understanding the molecular basis of the anti-lipolytic action of adiponutrin.

In summary, the data presented herein are consistent with the proposed role for desnutrin/ATGL as the major adipocyte lipase and provide evidence that adiponutrin, while exhibiting lipase activity in vitro, functions to inhibit lipolysis in cells. While GS2-like can ftunction as a triglyceride lipase in vitro and in cells, its low levels of expression make an important ftunction in adipocyte lipolysis unlikely. Interestingly, it was found that GS2 is highly expressed in human adipose tissue and has triglyceride lipase activity both in vitro and in cells, suggesting that GS2 may function as an important mediator of lipolysis in humans. Together, our data characterize adiponutrin, GS2, and GS2-like as new modulators of lipolysis.

What is claimed is:

1. An isolated nucleic acid molecule having the nucleotide sequence of SEQ ID NO:22.

2. An expression vector for inhibiting esterase activity, wherein the expression vector comprises a nucleic acid sequence selected from the group consisting of SEQ ID NOs:1, 4, 7, 10, 13, 16, 19, 22, 25, 52, 55, and portions thereof.

3. A cell exhibiting an inhibited esterase activity, wherein the cell comprises the expression vector of claim 2.

4. An expression vector for enhancing esterase activity, wherein the expression vector comprises a nucleic acid sequence selected from the group consisting of SEQ ID NOs:1, 4, 7, 10, 13, 16, 19, 22, 25, 52, 55, and portions thereof encoding active fragments of the polypeptides encoded by said SEQ ID NOs.

5. A cell exhibiting an enhanced esterase activity, wherein the cell comprises the expression vector of claim 4.

6. A method for identifying a modulator of the esterase activity of a member of the adiponutrin family of proteins, the method comprising the steps of contacting the member of the adiponutrin family with a candidate modulator and determining whether the candidate modulator modulates the esterase activity of the member of the adiponutrin family of proteins.

7. The method of claim 6, wherein the esterase activity is lipid acyl hydrolase activity.

8. The method of claim 6, wherein the esterase activity is lipase activity.

9. A method for modulating esterase activity in a subject comprising the steps of:
selecting a subject in need of a modulator of the esterase activity of one or more members of the adiponutrin family of proteins; and
administering to the subject a therapeutically effective amount of such a modulator.

10. A method for identifying a modulator of the enzymatic activity of a member of the adiponutrin family of proteins for the treatment of a disease selected from the group consisting of cardiovascular disease, obesity, insulin resistance, type 2 diabetes, dyslipidemia, nonalcoholic fatty liver disease, and metabolic syndrome, the method comprising determining whether a candidate modulator modulates the enzymatic activity of one or more members of the adiponutrin family of proteins.

11. A method for treating a disease selected from the group consisting of cardiovascular disease, obesity, insulin resistance, type 2 diabetes, dyslipidemia, nonalcoholic fatty liver disease, and metabolic syndrome, the method comprising the steps of:
selecting a subject in need of a modulator of the enzymatic activity of one or more members of the adiponutrin family of proteins; and
administering to the subject a therapeutically effective amount of such a modulator.

12. A pharmaceutical composition comprising a modulator of the activity of one or more members of the adiponutrin family of proteins and a pharmaceutically acceptable carrier.

13. An expression vector for inhibiting lipase activity, wherein the expression vector comprises a nucleic acid sequence selected from the group consisting of SEQ ID NOs:1, 13, 52, and portions thereof encoding active fragments of the polypeptides encoded by said SEQ ID NOs.

14. A cell exhibiting inhibited lipase activity, wherein the cell comprises the expression vector of claim 13.

15. The method of claim 6, wherein the step of contacting the member of the adiponutrin family of protein with the candidate modulator comprises contacting a cell with the candidate modulator, wherein the cell comprises an expression vector comprising a nucleic acid sequence selected from the group consisting of SEQ ID NOs:1, 4, 7, 10, 13, 16, 19, 22, 25, 52, 55, and portions thereof encoding active fragments of the polypeptides encoded by said SEQ ID NOs.

16. The method of claim 15, wherein the cell is an adipocyte.

17. The method of claim 16, wherein the adipocyte is a 3T3-L1 cell.

18. The method of claim 15, wherein the step of contacting the cell with the candidate modulator comprises contacting the cell with isoproterenol.

19. The method of claim 15, wherein the step of contacting the cell with the candidate modulator comprises contacting the cell with insulin.

20. A method for identifying a modulator of a member of the adiponutrin family of proteins, the method comprising the step of contacting a cell with a candidate modulator, wherein the cell comprises an expression vector comprising a nucleic acid sequence selected from the group consisting of SEQ ID NOs:1, 4, 7, 10, 13, 16, 19, 22, 25, 52, 55, and portions thereof encoding active fragments of the polypeptides encoded by said SEQ ID NOs.

21. The method of claim 20, wherein the expression vector comprises the nucleic acid sequence of SEQ ID NO:1.

22. The method of claim 20, wherein the cell is an adipocyte.

23. The method of claim 22, wherein the adipocyte is a 3T3-L1 cell.

24. The method of claim 20, wherein the step of contacting the cell with the candidate modulator comprises contacting the cell with isoproterenol.

25. The method of claim 20, wherein the step of contacting the cell with the candidate modulator comprises contacting the cell with insulin.


SEQUENCE LISTING (57 sequences)

SEQ ID NO: 1 - DNA, 2235 bp, Homo sapiens
cgcttgcgggcgccgggcggagctgctgcggatcaggacccgagccgattcccgatcccgacccagatcctaacccgcgcccccgccccgccgccgccgccatgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccggtatcccgctggagcagactctgcaggtcctctcagatcttgtgcggaaggccaggagtcggaacattggcatcttccatccatccttcaacttaagcaagttcctccgacagggtctcggcaaatgcctcccggccaatgtccaccagctcatctccggcaaaataggcatctctcttaccagagtgtctgatggggaaaacgttctggtgtctgactttcggtccaaagacgaagtcgtggatgccttggtatgttcctgcttcatgcctttctacagtggccttatccctccttccttcagaggcgtgcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgctgggagagatatgccttcgaggatatttggatgcattcaggttcttggaagagaagggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacagcactgagtgaagaaatgaaagacaaaggtggatacatgagcaagatttgcaacttgctacccattaggataatgtcttatgtaatgctgccctgtaccctgcctgtggaatctgccattgcgattgtccagagactggtgacatggcttccagatatgcccgacgatgtcctgtggttgcagtgggtgacctcacaggtgttcactcgagtgctgatgtgtctgctccccgcctccaggtcccaaatgccagtgagcagccaacaggcctccccatgcacacctgagcaggactggccctgctggactccctgctcccccgagggctgtccagcagagaccaaagcagaggccaccccgcggtccatcctcaggtccagcctgaacttcttcttgggcaataaagtacctgctggtgctgaggggctctccacctttcccagtttttcactagagaagagtctgtgagtcacttgaggaggcgagtctagcagattctttcagaggtgctaaagtttcccatctttgtgcagctacctccgcattgctgtgtagtgacccctgcctgtgacgtggaggatcccagcctctgagctgagttggttttatgaaaagctaggaagcaacctttcgcctgtgcagcggtccagcacttaactctaatacatcagcatgcgttaattcagctggttgggaaatgacaccaggaagcccagtgcagagggtcccttactgactgtttcgtggccctattaatggtcagactgttccagcatgaggttcttagaatgacaggtgtttggatgggtgggggccttgtgatggggggtaggctggcccatgtgtgatcttgtggggtggagggaagagaatagcatgatcccacttccccatgctgtgggaaggggtgcagttcgtccccaagaacgacactgcctgtcaggtggtctgcaaagatgataaccttgactactaaaaacgtctccatggcgggggtaacaagatgataatctacttaattttagaacacctttttcacctaactaaaataatgtttaaagagttttgtataaaaatgtaaggaagcgttgttacctgttgaattttgtattatgtgaatcagtgagatgttagtagaataagccttaaaaaaaaa

SEQ ID NO: 2 - protein, 481 aa, Homo sapiens [sequence omitted]

SEQ ID NO: 3 - DNA, 25830 bp, Homo sapiens
gaactgtgagacagagaaataagccaggcgaagtggctcacccctgtaatcccagcattttgggaggctgaggtgggtgatgacttgaggtcaggagttctagactagcctggccaacatggtgaaaccccatctctactaaaaatacaaaaaattagtcaggcatggtggcgcatgcttgtaatcccagctactcgtgaggctgaggcaggagaatcccttgaacccgggaggaagaggctgcagtgggccaagatcgtgccattgcactccagcctggggggaaaaagatagaaataaatctcttgtttgtaagcccctccgctttaaggtgttttgttttcgcacctgaacggactaagggactaagggaccaggaatcatccaggcctcagcttcccaaagcactccccactccccagttcctacagggccccgaccccagccccagcacccgtcattcacattttttacttacaaaggaaactttgaaatggacacttcctgttactttcattcaatgaaaaaaatccccagcacttgaaggcaatcatacagaaagcctgcaacaacgcagagagtagactcttcaggtggtggtgcctgctttaggctggctctccccgggtcaaaagggacgtgataaagtgttaaagtgatgttaaggatcaagtgggcaccaactgcgactcccctcccaccccccgacttaacagatgtcaaggaaaacagaaggaagccgtttctctccaggacatgcagtgtctgctggagttttggaaaaatgctttctctcgagtcgctgcggggagctcccaggctggacccccccgcacaggtcctccgccatcgccctcccagcccccgcccccaatccccctcccagacccggtccccgcccccatcccccgccagacgtcgtccccacccccatccccctccaagccccgcccgggagggcgtcctgcccgctcattgggcctcccgccggctcatttgcatggtccgaggggggcggggctgacgtcgcgctgggaatgccctggccgagacactgaggcagggtagagagcgcttgcgggcgccgggcggagctgctgcggatcaggacccgagccgattcccgatcccgacccagatcctaacccgcgcccccgccccgccgccgccgccatgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccggtatcccgctgggtgcgtctggggacgctgcccgggctccacgtgcggagtgggtgccccctaggccggggagcgggggatccccaggggtcgcggggccctggaggagcgggcatcggacgcggacacggcggggtgcatcccgagggccccctccgaggcagatgcttcctgcgggggcgctgttcctgggcccgggaagggggcgttggaaccccgagcggtccgggccgaagcctgggactctcgtgcgtccccacccctacccccatcaggcgcccgtgcatgaagggagaccctcacctccggactgagagtcggagcgtctcggagcgacggggagtagggagcgggacccggggcggagggtagtgctggcccctgcggactccgggtcccctgtgtcctctcgggaggggctggacgggctgagctgccgaggggccgatttgccctgggccggacaaagagtggggctttggccggtcccccacggtgggctccttccctctggggattgagggactcaagacaccccgcgcctgcgcttttcttttctttttttctttttttttttttgagacggagtttcgctcagtcgcccaggctggagtgcagtggcgtgatctcaactcactgcaagctccacctcccaggttcacgccattctcctgcctcagcctcccgagtagctgggactacaggcgccagccaccaagcccggctaattttttgtattttttagtagagacggggtttcaccgtgttagccaggatggtctcgatctcctgacctcgtgatctgcccacctcggcctcccagaatgctggggttacaggcgtgagccactgctccctgctgcctacgctctctgggtcgcagcccagccttctgggggctgggtagcctcccagaagggcaaccctgggcatcctccagggcaggctaactggagtctagtggggaggggtaccttgaaagaggaaagttgtttcctcctcctcctcctcctccagtgtttgggacccttcctgggggctggagtgcatccctggacaccccccaatcccatcctcttctctagtttccactgacctaggcccaccctcccctctccggctcagtactcctggaaatgagattccgtacatttgaatcttgtcctaatgaaatatttgtccatgtgggtacctgtgtgtgtgtggtgggggtgcagacggagggtttgtttctcactagctggaactactggggtgtggtatgcttcctgggaatttgtgtgccacagtcctggaggcgaggagggggttgtgagccagtaggcaggggctggggcaagtagcattgtgaagctattgacacccagacgtccccaggcaggagattatgcccccattagcccccttttatctgggcttccttaacaatggactctttgccctgcctgccagagccagcagggagtgactgttcagtggtgaggaagcgggcagaggaagccctgccattgggtaggagcagtgggcagcccctgggctgactgggaggtggggattagggattagacagtcctggctgtctgccttcccctaagccagggggagaggagcaaagggcacgaaatgtggcctccaggaggattagaccgccacatgatcatttgcacaccctggggtttagcaacaataaaagtcagcttttttgtatcccaaggtggcctgtggacacccacatggacaaatgtttacactgggacagaattcaaatgcagaggtcccaggagcctaaagtacactcactctggtatagaaaggattccttactgggcagaggacaggtgcagcctggggctttcccaggcaggacacagggaggctcaggaaccaccaagtccctggaaggtggatctggaggcgttggcaggagccactccctgggttccagggctccaggttcctgctttaaccccctgtctcacagagggctgtgcacttgggggctgctgagcatgtcccagaggctgcatcctggacacagcacctcagtgcatctgagctgaggctaacttggcaggagggacaggcagaacctgccagccacgtgcaattccacccctctggccactcagggaaggagagctgtgagtcaagatcagatttgggtcaggacaggctggggcctgcctgtccctgtgcatcccaagatttatggctggccaggggttgggctgggaggggtggtcttgcatgccaggagagtgcagatcagcctgagaggccaggccagtaagtgaggtcagatctcctgcacctgatagcattaaggccatctacaccaaagctctaatgctgatatgttcctggcctctatgtggggcatggaggtggggcatggaggtgaggcctgctcgcctgggcttctggaagtgggagactcattcctgtggctgaggcctacagcagtgctgtgtggtaggaatacactggaagccatgatgtcattgtgcattttctagaagccacattgaataaagtaaaagacacaggtagaattaatttcattgagcccaatatatccaaaataatatcattttcacatctattcaatataaaaatttactaatgagatatttcatactaagccactgaaatccagtttgtatcttacacatctcagttttgacgagccacatttcaagggcgtgatagccacatgtggctcccatagtagacagtactggtctagagaaatgttggtggcatccttgctgtctggtttctggccttgccaaaagtattaccatcccagtgtggtacattctttcatgtatttgtctcctgtccccagagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaataggcatctctcttaccagagtgtctgatggggaaaacgttctggtgtctgactttcggtccaaagacgaagtcgtggatgtaagcagtttgcttatctggacgttgtcaagttagaaaagctgttttgggatgggtgtggtggctcatgcctgtcatcccggcactttgggaggccgaagcgggtgggttgcttgagcccaggagctcgagaccaacatgatgaaacccagtctctacaaaaattacagaaaaattagctaggcatggtgttgtgggcccatagtcccagctactagggaggctgaggcaggagaattgcttgagcctgggaggtggaggttgcagtaagtcatgatcatgccactgtactccagcccgggtgacagtgagatgctgtctggaaaaaaaaaaaaaagaaagactgttttgttttggaagcaacacaggcagttgtaggccccctgtgccagagtgacataaactctgtacacctccagtgatttggtccatgtttgtaaaccctgaatgttccagggcagtttcttttcttcactttttatctcttttttttgggtgggggggcggggtacagagtcttgctctgtctcccaggctggagtgcagtggcgcaatctcaacctcccgaggagctgggactacaggcacaggccatcacaccttgctaatgtttgtactttttgtagagacggggttttgccctgttgcccaggctggtcccaaactcctgcacccaagtaatctgcccacctctgcctggcagttacaatttcaaataattcctccctttccttcaacacttggctcatgaccgtccagtccaaggaacctgtcctgcaggtgtgcctctcccgagcttcctctatgcatcttccataatgaagatgccttctcactggaaaccctacaagggtgggaacgtgccttatttgcctgtatcctcagggtctagcagagagaagataatttgtaataccaaaacaccattaaattcagctgatgctttcataagcgctccttggaggaaggactccatttacttgacagatctgtgcaagacagcagcctggcgcgtctaacctgcagccagttgcatcctctgtttaaccttgtttgcggaagctttctctaaacagccagcacttgtctgttcccacatgggtccgttctcccagtgaatcaccgtggtgcctgctgactgctctgtagcacagtgcttcgcaaagtgtgatcctgggaccagcagagcagcagctcctttgagcttattggaatggcagaccctcaggtcccacctctgacctgctgcatgggaattctggggagggacgcagaatctctggttccacaggctctccggtgatgctaatgaataccggcatttgaacagcaccgatctagcccctttcagtccatgagccaacaacccttggtcctgtctgtggtgacccagtgtgactctcatggggagcaaggagaggaagttgaagttcactgacagggttgttaaggggattatgcaatagatgagacccatgggcctgaagtccgagggtgtatgttagttccccgttcttttgacccatggattaacctactctgtgcaaagggcattttcaagtttgttgccctgctcacttggagaaagcttatgaaggatcaggaaaattaaaagggtgctctcgcctataacttctctctcctttgctttcacaggccttggtatgttcctgcttcatccccttctacagtggccttatccctccttccttcagaggcgtggtaagtcggctttctctgctagcgctgagtcctgggggcctctgaagtgtgctcacacatctcctgcctgcagggcactggtgtcgggcacctcagggtctgtcccatggtggagccccatgcctcactgcctttcagacagagtagccacagctggccctatttccaggctacccgggcagcaaaacttactgcatgtgtaattaattatttggctatctgtaaggtaaactggctggttcacttaatctgcaccttaagcatcagatagcttctcagtgatctagttaaactatatgatgttggccaggcgcggtggctcatgtctgtaatcccagcactttgggagcctgaagcaggcagatcacttgaggtcaggagttcgagaccagcctggccaacagtgtgaaactctgtctctcctaaaaatacaaaaattagctgggcatggtggtgtgcacctgtaatcccagctgctcgggaggctgaggcaggagaattgcttgaacttgggaggcggaagttgcagtgagccaagatcgcaccactgcactccatcctgggtgacagagcgagactctatctcaaaaagaaaaaaaaaaaaaaggtaaataaagtatatgacactgaagaatctgttacccctggaaggtggagctttactcttagggggaactataacagtcatatatatatatttttttcttttctttttttttttttttgagatggagtctggctctgtcgcccaggctggagtgcagtggtgcaatctcggctcactgcaacctccacctcacaggttcaggcaattctcctgcctcaacctcccgagtagctgggattacaggtgcctgccgtcacgccaagctaatttttgtatttttagtagagacagggtttcatcatattggccaggctggtctccaactcctgacctcaggtgatccgcccgccttggcctcccaaagtgctgagattacaggcgtgagccatggtgcccggccaacaatcacatgtgttgtaaacaacaacaaaaatctgtcagcctggtctaacctagatttgtgctttgttttgttttgccactttgtgatgcacaggaggaagtttaggctgtaaaatactagccttttagggtaatttttgaactcacaagagcagcagcggaacctttgatgcaatcctgtatgtagcaccagcagagccacgtggcagagggactcgcattaggagcctcccattacagactacgtgctcctgtgcgttatcttatagggtccccacaaccaaggggagatgtgattattcatcctgtgtggctgtggggaacttgagagtcatacttgcccaaagagcacggccagcgagcttgcacccaggtcactctctgctcctctgtcagaacagggcatgtcttggttcactgcagggcggctcttctcattctctgtagtttggggtccaggatagtggtccacggagccactggagtgcccagctactgagtgaccaaagcatattttggatttccgacattgccacagcatggttgggcatcagcaggaccccaaccccttgttatgctggtggctttatgtggttatttgatcttccccagaactcagcaggagtgcacccagcagcaccgtagtgatgctctctggctccccagtgcacggttctggctttccttcctggtcgagagtttcaagccctctgggtcctactctgtccttttcagcccatagctttgttcaaaagctgctggcagtgttcagatttggctgagttcagtgaatatgtgcattggctgatttctgagccatgccagggggatggagaagccgaagcaggagtgtttgttctgcaggctctggagtaggcattgggtctgtgccggctcacttgctagtcttgcatccttccccaaccccctctggggatgtctggccacatcagaagacagtttgggttgtcagaactgggggagtaccaggccgaggtgggtggatcatgaggtcaggagatcgagaccatcctggctaacacagtgaaacctcatctctactaaacatacgaaaaaaattagctgggcgtggtggcgggcgcctgtagtcccagctactcgggaggctgaggcaggagaatggtgtgaacccggggggcggagcttgcagtgagctgagatcctgccactgcactccagcctgggcaacaaagcgagactccgtctcacaaacaaaacaaaacaaaacaaaacaaaatctgggggagtgccactggcatctgatgtatagaggcccgagatgctgtgtcatcacccgttgagtgcgctcataggcatcttcctgacaattagaacccattattcttcaaattcaatgcaagcaaattcaaagcattactgtgtacataccgcatgctaatcaattgcaccactggagctcctaaattcaaaacattactataaaaaagttcaaaatgcatggaaaagttgtacatggcaggagaatatttgggcttctgactaccccttgaatgaagatgatccaccagccgccttcctccttggtcttcactccagattcctagcatttcattctgtgtctctttatgcagtgaggtttttgtttgttttttgagacagagtctcactgtatcacctaggcctggagtgcagtggcgcgatctcagctcactgcaaccctcggctcctgggtttaagcgattctcctgcctcagcctcccgagcagctgagattacaagcacacatccccatgcccagctaatttttgtatttttagcagagacagggtttcaccatgttgcccaggctggtctcgaactcctggcctcaagtgatccatgtgcctcagccttccaaagtgctgggattacaggcgtgagccaccatgcccagctcctagtgaggtttttgatgccttgctacatctgccctagaaattgtgtgactacgattttggaaatgttgctgtgtaaacttgtgatcatttctggactccaggcaagaatcttgatggctaaggtgtggctgaacatgtctgattctctcctggacctgttttaggccaaactctgctctgaaattcctccgtgtggaagggcgggctggggagagcctcccagctggaatcttttggatgcctttctctgtgggtatctgatggctggctctgatggctggctgtgatggctgtggctggaaatcattgttgacatgagtttcacagatgcaggctctgtccaaattgtagcaaaagctgcctgccccagccgagctatgggcaataaggtggtttaaggatatagatgaaggaaaactcacccttagaataatttatccaaaatgctgctgtgttgtgggttagaggacattttctgaggtcccaggttcattgtttcatttaagtctcaaaagtccctccaggtgttggttctaattgtcaaagcatggggggagatgggctcatgggttaaaggtcttatcccagatttctgtatcctccttgcaagcagcaaaggggtctggatttgaatccatgaccatgtttctcctttgggtttccatcacactctgtccccgtgcactgagcaccctttagttcatatgacccccttaggcatgttacatgggcactcctataggtgcccatctggccctaggacttggccaacacaacatggactccagtttccatctgcctctttgccaggcacttttgtgcagtgcacacactgtacaacagtagacggcaaccctgagagccagagtagagcctgtcctagcaccggaatgctcggtaaggatttgtcgcaggagtgattccaaagccaatgtcctccctccatatcagcctgtttgtggctctgagaagctctgcccacatgtgaaagcttgttaagcacttaagcactaacccagagcttcagacagtgccagtcctttttccccttctttaaaagcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgagttggtggtgagggggcaggtgttctggggtgcagctcttctttgcctccctgattgccaggagctaccagttactgtctgcacaatcaaacagaaatagacctgtccttgatggttaacggaaataaaaggcgcttgtcccagaagctcaggtgaggcaccaccctgattatgggaatcacctgggaacatatacccagacctaaaactcagatccacttcccaggctgtggttatatagtcaggggggtgcagtatgggtattaggattttttattttttagttataaagatttttttttggtttgtttttgagacagggtcttgctctgccgcttaggctggagtgcagtggtgcaatcatagctcactgaagcctcagactcctgggttcaagcagtcctcccacctcagcctcctaaggagctgggacccacaggcatgcagcaccacacctggctaatttttaaaaattttgtggagtgttgcccaggctggtctcacactcctggcctcaagcgatcctcccaccccagcctcccaatgtgttgggattacaggcatgagccattgtacccagccactaagatgattcttatttggaaacacggtcaagaacaactgcgttcggtagtttaaccttttttgattgtggtggttttagtatgccttaccactctaccatagtaagaaatttgcagaccatgtacaccaacctttggtgctcctggggagaaagaaagaaggctatgcaatgcaatgcatgctcacagtccaagggagagggaaagctgtctaacaggattggttttcccgtgtgctttataagcagatgagtagaggagacagctcttattgtcctagtggcaattgggataggctgcaaagtttgttagggtggaggcttattccgggaccaagggagcccaaagaaacaagctcctgccaggcgcggtggctcacgcctgtaatcccagcactttgggaggctgaggcaggtggatcacctgaggtcaggagtttgagaccagcctggccaacatggtgaaaccccgtctccatgaaaaatacaaaaattacccgggcatggtggcgggcacctgtaatcccagctactagggaggctgaggcaggaaaatggcttgaacctcggaagtggaggtggccgttagccgagatcacgccactgcactccagcctgggcaacagagcaagactctgccttaaaaaaaaaaaaaaaaaaaagaaaagtaaaaggaaaaaaaagaggctctggcctgctggggtgcctgcaaagtctccgtggaagggtgacattcaagccgagacctccagggaactgtctcctgggagcacagagccctttgctcagcccccaggtggctcagtgcccccagccagcagactcagagcttgcatgattctttggtgctctctgcggtcttccaatgatgctgaaataaatggtgcttggtgtctccctgctgtagtccccttgcttgctttgctcacaggtgctgggagagatatgccttcgaggatatttggatgcattcaggttcttggaagagaagggtatgtatgggctgggaggatcagccatgcccttttgacaagcatttactagcggtcttggtaaagacttgagatttgccttagttctaacacttagtgcccaacgccttccttgtgttgctcaacctactcatgagcccaggagataggaaatctccgtcccattgtacagatggggaaacagaattttggaaaggagagccaagcagcacacacccctccctgaggggcagagccgagatttgaactgggatgtcatgactccagggccctctccctccccagggtccccttatctgaaggcggtttttctttccagctcgacctcttgtgacccttagtttaacaagggccgaagttaaagagtttctgcgcctggaccccaaatgaagcaatcagatttctcatctccagtcaggtgtgggtccaagcccactagacaagtttgctcttcccagagcacatttctgccttcaagtcatcctggcttgtcagggctgggggagttctgctgtagaaatattagagtggaaggaaaaagatgtgttgggagctatttttctttaatactaaaagttggttgatgaatttgtcgttggccaagaccaaggagactgcatttttaaggacatatgtgtatttatctgctcagaaaatgttcattgctgtgtgctagggatactgcagtgaacacagaggtgtgacccttgccagccttgtgagagaagtgagcagataagtaagcagaagggtgatgctgtgtcgatgggaaagtacaggtgccaatgagaaggcacaggtgtcaaggagaagacacaggatgctggaggctcatgcaggatggatctccaaggcccaggggaagaagggcctctcggaggacgtgaatccacattaagactttggggataagtaggagcgccttaggcatggggacccatggatgcgaggcctgtaggacacagacaggatggcatgaaggcctgtgcaactggaggggtggggatggggacactaagagatggctggaagtgtgggggtggggacactaagagatgactggagaagagggggtcaggagtggtgaaaaatgggagaggagggcaggctgggccttttggatacagggggattgcatcctgcagtggtagggagccactgagggctgctgcagtaggagtgaggggatcagaggagagctttggaagccccctggatgcgggacaggaagcgagataccagtgtctaggaggccagtgaggcagccacaggctccaccaggatcagggctgcgagggtcatgaggaggaaaccaatttgaaggagtccaggggaataggacttggaaatgaccgatgggacatttgggaagaggaagacagaagagcgcagtcccggcttctggctttagcagttgggcaaggggagatggggagatgtgcccatgggttgagggttgaggacattaggagggagccggtatggcaggaagagctggtgtgccagagatgctggaagcagcatctgcctgagaacagatacctggcaatattcctaagggaaagtgacatctcggagggtgaggagggcatctgatagggcctggaaagagccggggcaagcatgaatgtgaggttatcttggggggcaaggctcaggcgttgaggagcagcccctggtctcttcagcctgaagttggaagccagagttgggccaggtgcagctgtggttgtctgaagtccccctcccccagcccagtgtgccaatgctgtaagagcaagggccgctcactggtgctggtggctgagtcccagcacccaggacagggcctggcacatactggtgcccaatcctcccttctgggtgcttcttccaaggccttgtgatggaagtgagtaccctcttcgacatcagacccagcttcaaatcctggctctgctatgtattggctgcgtggctttagacaagtcttttaaccttgctgtgcttctgatttctcagctgaaaaatggagatgatgatagtggtttctgtaaggccttatggtgaagcacctagctcagggcctggaaggcaggtgtaaccagtggttcagttgttataaaccaacactaaccctcgcctttgcacctcatgaaaccagatatgtagatggagcccacaaagctagcaggagccaagctcacgtgtgtcctgctttaaagccccatacccctttctccgggtgacaaacacctgtgctcgttctcttcccttcccctcttccccttgcatttggctaataacaggccagctgcctgcctccctgcagtttggtagatgggtgggtaacgaccaccactcccacgttcgcctgatgggcttgttttccgtgcccttcacaggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacaggtacccactcctcggggtgagcacgggcagcaccttgttttctttcttgtgcattatggaggaagatggtactgccacatgggagcgatagggtgaggcaaccatgacaggtggttgggaacatctccttccatgtgtacagcctgggctgctgccatcactcccagcacagcccccaacccccccaatcctggaaccttgccaagtctcccttcccatggggtcatgaccaggaggaaaacaaactccagctgagccccttggggttccccatataggctcctgcctgtggcagctgggccctctgtacccctttccaactctgtctccctaacatggcacctgagctcctgccatcctggatttcatggaccccaaggatgggggtcctgcatctgggacttggcctattactcggagctccttttcagccgcctccctccacctgtccacccacctcaaggctcctttcttgagacctctcctaatttctcccttcccctaaacccacaattttgaacctccatcgaatggtgctgtattttataatgtcatcaaatatcaaatggagacagtgctatggtccaaatgattgtgtaccccccagaatttgtcttttgaaatcctaacccccaacatgatggtcttaggaggtggggcctttgggaggagattaggtcatgaggaaagggctgtcatgaatgggattggtgcccttattaaacagacccaagagaggtcccttgtcccttctactgtgtgaggactcagaaggtggtgtctatgaagaagcaggccctcaccagacaccaacatgtctgctgccccttgatctgggaccttgcagcctctagaactctgaaaaatcgatgtttgttgttttataagccactcagttggtggcattttgttagagtagcctgaacacggactaagtcaaacagaagaacccacaaaccagctacagagttgggcatttggagaaattcaaaaatgagtcagacataactccttattcttgaggtgccctaagagatgggacacagcagctgcccaggtgcattagtttgttctcacattgctataaagaaatacctgagactgggtaactcataaagaaagaggttgaattggctcacagttgcacaggctggacaggaagcatggtgctggcatctgctcagcttctggggaggcctcaggaaacttacaatcatggcagaaggtgaacgggaagcatgcacatcccatgactggagcaggagtgagagagagagggaaatagagggaaggtgccatacacttttaaacaaccagatctcatgagaacacattcactatcaagagaacagcaccagtggggaaatccgcccccatgatccaatcacctcccatcaggctccgcctccaacactgggaattacaatttgacatgagatgtgggcagggacacagatccaaaccatatgaccagattaatacgatttgaggcatcacgaggtcattaaagagagggaataaaagactggggctccaggaagaaggctctggaatccagcagagggtcaaggaccagcttgtaaagctggtggtgcctgagaagtacctaggagaacatagatgctgtgacgtttgatgtagctgttttttgttttgtgttttggtttttgagacagagtctcactctgttgcccaggctggagtgtgcagtggcgtgatcttggctcactggagcctccatctcccaggttcaaatgatcctcatgcctcagcctcctgagttgctgggattacaggtgcacaccaccacgcctggctaatttttgtgttttcagtagagacagggtttcaccatgttggccaggctggtcttgaactcctgacctcaagtgatccaacaacttcagcctcccaaagtgctgggatgacaggcatgagccaccatgcccagcctgatgtagctgtttctgtgcacattatttgctgtggggtatattcagatttcttaatacaagatgattctttgcctcatgacttacacaccattttctatttaatttcagctatgatattggaaatggacatgtcttttcaaggaaaataaaagcaggctttctggaatggcgacttccaaacatatttgtcaatttaaaggagctgggagtggggaccctatgctccgtaagcactctcttagctgttcttggctgtgctccccgcttcagcttcacactgcccttgctgtgaagggagcagcctgggccgggcgcggtggcttacacctgtaatcctagcactttgggaggccgaggtgggtggatcacctgaggtcaggagttcaagaccagcctggccaacatggtgaaactccatctctactaaaaatacaaaaaattagctgggcatggtggcaggtgcctgtaatcccagctacttgggaggctgaggcagaagaatcgcttgaacccaggaggcggaggttgcagtgagccgagattgcgccattgcactccagcctgggggcaacaagagcaaaactctgtctggaaaaaaaagaaaggagcagcttggcaaaccccaccttgtcgcttttgtgagtgcctctgaccctttggctgccaggacgggcgtattttatggaaatgctaagcaccaacagagtaaagtggtttggtttttcacagtggtgggagataatagctccaaattgtctttttcagcactgagtgaagaaatgaaagacaaaggtggatacatgagcaagatttgcaacttgctacccattaggataatgtcttatgtaatgctgccctgtaccctgcctgtggaatctgccattgcgattgtccagaggtgagcattttaggtggctccgtgtcttcctcacagggttgatatgaggatgaaacaagatgatagatcatggtggcatgtagtctgggacctggattgtcgtgccacagatcacagctcacagtctatgtgcaatgcccctgaatgttgcccacctgtcctcaagccacacatgcacctgtaactcagtgcaagcccagaaactccccgtggggactcctagagctgtcagtggcctcacatagcagctggtccagtctcttgtgattgcccaaggaaactgaggcctggagagcttggggtcactgctctgaggccatagagatgcctagtagaagggccaggcctagaagcaggatccttgctgcccctctgagctgtttccatttaaaatcacatgaaggccggcgccgtggctcacggctgtaatcccagcattttgggaggccaaggtgggtggatcatgtgaggtcaggagtttgagaccagcttggccaacatggtgaaatgccatctgtactaaaaatacaaaaattagtggagcatggtggcacgtgcctgtactcccagctacttggaaggctggggcagaagaatcgcttgagcctgggaggcagaggttgtagtgagccaagattgtaccactgcactccagcctgggtgacaggagagaaaccctatctcaaaataaaatgaaaggtaatgaaatgaataaaataataaatcaagtcacggccgggcacggtggctcacacctgtaatcccagcgctttgggaggccgaggtgggtggataatgaggtcaggagttcaagaccagcctggccaacatggtgaaaccatgtctctactaaaaatacaaaaattagctgggcatggtggtgcatgcctgtaatcccagctactccggaggctaaggcaggagaattgcttgaagcaggacctaggaggcggaggttggttgcagtgagccgagatcatgccactgcactctagcctgggctacagagcgaaactccgactcaaaaaaaaaaaaaaaaaaaaatcaaatcacatgaaagtagaacatagggaattccatctttcgttctaggcatagtttgttaatatgattcagagccagcagttaggagaacacagtgtgactctcctagaacttcttgattgggcttcctctgattgggtttcctctgattgggcttcctctgaaagtgggggggatggggggtggggagcagaatggtcagagcttggctcagcagtcagactgctcttcttcaaatcctggctgcattgcttactacagctgtgtgactccagatgactgaatccacctctctgtgctgcagcttcccgtctagagagatcacctggagcagagggtggtcaggagactcaatctggttactgactcacagtgcaggagtactcatcccatagtaagcatccagctagagatgttgatttctattttcaggtaataatgatgatcgtaaaattagagacagataaaaggtatgggcattaggccagggcactgcaatttctaagctgtgtgacctcaggcaagttactcgacttctctgagcctcagcggtttcatccgcaatatatggataggaaaaccgacctcagtgggttgtctgacagtggagggcacttgattaaaaaaaaaaaaattaccctggtctgaatattaccctggactgaaagaaaaatattgagctaatacaggcatcaggaatggggctgcagggagtccagggaagggagaacgaagagcctgaaggtgtgaggaggtgcgagtgctgatctgtctgctacaaagaggctgctgagcctcctgtggatgtggccctggacttggcagtttaatacctgagctgttaaaataacctcagatgctgtgttctttaaggggtaggattcagattcctgctgaaatgcttctgaaagggagggaatgagccagcccatccccagttgctttttaagatcattgggaagttctggtcttgccatttgtccctggaccactcttaggtcctcctgccccacttccatctgggtgtgtgccctgggctgtccaccacacagctacatcctgccatcttccctcctggagccactgtgccatgcatggatctgtagcttcatttttcttggcttttccctggtttttctggagcagagtctctagtaaactcccaaggaagaaaacgtttgactttatgtgtgttgggaaacgtgctttttttctattacatctcagtgataggttggccatgtctagaattgcaggttgaaaatcatttcctctcagtatattggttagtgagaagcctgggactgagacagtcacattctcacttctttgcaggtgagtgctcttaggactgtctttttatcccttatactctgaaatgtcatatgtcttggtgtaagtccttatttcagttattgagctggacaagtactggagaccccttcagtcaaagccttctgtcattctccagctctaggaaattatcttctattgttatttctgttattccttcccttccattttcttttttctttttttttttttttttttgagacagggtcttactctggtgcccaggctggaatgcagtgacctgatcatggtacactgcagcctgaacctcccagactcaagtgatcctcccacctcaacctcctaagtagctgggactgcaagcacacatcaccacacccaacaaatattttttaaaaattttgtaagatgggatcttactatgttgcccagactttttcttcctcttcctggggctcttattaggaagatgtttgacttcctgggttggattcctgtctccgtgtctgactttctctctttgtcatatttttcatcactcgttgtctttttgcgtctgctctgacagatttcctcaaattttgtcttctagtcctatcctacagtttttactttcagcaaatataatttaatctccaagagtactctcttgttcttttttcttagcattctgttcttgttttatggatgtaacattctcttggaatatttgctgtcctctagatcatcccttctccatttcttcttgggctagtttttctgtttcttcatctttctcttttatgctacttattctgggcgtgttcttggtgggttttttcccatatagcaacagaggacttggagctcagggagaaaagggtaggtgcatcacctggcagagctcccagacagtgacaggcaggctgcgggaaggatgtctacttggcggtgctaccgctttcctagaaaccctttccctggagctggttgaactgttgggttttgccctggtggtgaacgctggctccccgtgctctgcctgtttcatcaccagccccctccccttctgcctggggtccagtaatctgttgaaatatatatcttgctcattggtgagctcctgctccttcctcgttgctcttgcagatttatcacttctcgtaaggctgcgcttgtacttcgggattttctctgtgccacactgggaaacatagggtggttgcatgctgcagtcctgagcacttatttcactcacatctttacacgaagatttggtgggtgtttactttgtttttagtaagttagtctgtcatgtcctttgatcctttttttttgttttttgagatggagtctctctgtgtcctccaggctggagtgcaatgtcgcgatctcagctcactgcaacctccacctcctgggctcaagagattctcctgcttcagtctcctgagtagctgggattacaggcatgtgccaccacacctggctaatttttgtatttttagtagaggtggggtttggcatgttggccagcctggtctcaaactcctgacctcctgacctgcctgccttggcctcccaaagtgctgggattacaggtgtgagccaccacacctggccctgattaatcttttaatgcccagtctctccttcaaaagccggctcctttctctccctcgccttcctagattccttctccactccccaggatcagcctcctcctccccaccccaccactgccggggggatgtctgtggtcaggcatttatcagagaccctgaggtgggggtcctttatgtgtctgggggatggagagtctagaggaggtagcgttcagacctctccatggtgcctctgctgggctcacatgtgaccaagcacagcaaaccatgaggcaggggatggtcttgaccatgagagcccttgcagcagctgccatgggcctcagctcctctccaagctgggaagagccctgaaaagccaaggtgtttttttttccctctttatttcagtgtaagtcccttgagctttcttgaaccagaagtgggctcattttgctttagagatttcaggtgggcttgtccttgtcctagcatcccagatccaccttctgggaagtcatcagattggaggtgatgttggcagcttttgtaaacaaagggtagtgttgtaagctgttgtgtctgcctatgtgtgtgtttgtgtacttggtctcatctctgcagactggtgacatggcttccagatatgcccgacgatgtcctgtggttgcagtgggtgacctcacaggtgttcactcgagtgctgatgtgtctgctccccgcctccaggtaaatactttggctgtgggtgtgtgggccggacgggcacctctctcatctgatgaggcctcacacgacattctagaaacagctggctgaacaccaagcaaggagcttgcccttgggtgtggggaccctgtctcatgggaggcagctgagtcagtcagaggtcctggcacacctgctgagagctgccacccaggccaacctgaaccggagcctgggaagacttcccgtcggatgagtctctttgagtgcagcattgatggtggaagagcagagaggccccagataagcagggaaaggtgcttcagacagagtggctgggatgaggactggggagtgtcagatagcgctggcgtgtctgagcgaaggagctctggcacccatggcacaggaaggaggtgggaccctggaggggcagggctagcagagctcctcggagcgtgtggctaggtgcctggtaatgcaagccccctgtcctccaccctctgttgtactgagtcacagtctccggggtgaagcctagcagtctgcgttgacaggccccaggggatgccgctacttcctgaattctgaattctggaaactgagccggagttcagggcctggctcccattaccagggttgggcgttatcctgaaaatcataggccttggtttcctcacttggctaacaggggtgatccccatcccctcaatgggtttccgtgagctcctgagagcccgtagcatggtacttggcacatgctgggcatcaggaggtatggcctctcttgctattgttgttattggtagacacagaaggatttaaaagtaggggaatgcaaagatccgatttgctagggaagagggcagtagtggccaagtagagggtggatcctgggccctggctggcagcaggcagcaaggggggctgccagggcccaggcagggacgatctgtagaccgagaggcttcctaaggctcttggacaggaggaggtgtcggttccaagcctaaggagtggggcagccctggtgactggtggtcagtggtgccaggcggtgggtggtaggacaccctggcaggcaagtaggtttgtgtgggggaaactgataggcccctccagggattcgttggtggacaacacctgtgatgtccagtgggaggtgtccaggtagctgggagggccacaggcttggaagacctaggtggtgacatcagcccagcactgagggctagaagaagctgtgtctctggctgtgacggcaccctagagtgtgtgtggtgccctctactggccggcaatgtgggtccaccgtagctcagactgcacactgcagcagcgggaacggcctctaagccaacttcctccatgtgtttcaggtcccaaatgccagtgagcagccaacaggcctccccatgcacacctgagcaggactggccctgctggactccctgctcccccaagggctgtccagcagagaccaaagcagaggccaccccgcggtccatcctcaggtccagcctgaacttcttcttgggcaataaagtacctgctggtgctgaggggctctccacctttcccagtttttcactagagaagagtctgtgagtcacttgaggaggcgagtctagcagattctttcagaggtgctaaagtttcccatctttgtgcagctacctccgcattgctgtgtagtgacccctgcctgtgacgtggaggatcccagcctctgagctgagttggttttatgaaaagctaggaagcaacctttcgcctgtgcagcggtccagcacttaactctaatacatcagcatgcgttaattcagctggttgggaaatgacaccaggaagcccagtgcagagggtcccttactgactgtttcgtggccctattaatggtcagactgttccagcatgaggttcttagaatgacaggtgtttggatgggtgggggccttgtgatggggggtaggctggcccatgtgtgatcttgtggggtggagggaagagaatagcatgatcccacttccccatgctgtgggaaggggtgcagttcgtccccaagaacgacactgcctgtcaggtggtctgcaaagatgataaccttgactactaaaaacgtctccatggcgggggtaacaagatgataatctacttaattttagaacacctttttcacctaactaaaataatgtttaaagagttttgtataaaaatgtaaggaagcgttgttacctgttgaattttgtattatgtgaatcagtgagatgttagtagaataagccttaaaaaaaaaaaaatcggttgggtgcagtggcacacggctgtaatcccagcactttgggaggccaaggttggcagatcacctgaggtcaggagttcaagaccagtctggccaacatagcaaaaccctgtctctactaaaaatacaaaaattatctgggcatggtggtgcatgcctgtaatcccagctattcggaaggctgaggcaggagaatcacttgaacccaggaggcggaggttgcggtgagctgagattgcaccatttcattccagcctgggcaacatgagtgaaagtctgactcaaaaaaaaaaaatttaaaaaacaaaataatctagtgtgcagggcattcacctcagccccccaggcaggagccaagcacagcaggagcttccgcctcctctccactggagcacacaacttgaacctggcttattttctgcagggaccagccccacatggtcagtgagtttctccccatgtgtggcgatgagagagtgtagaaataaagacacaagacaaagagataaaagaaaagacagctgggcccgggggaccactaccaccaatgcgcggagaccggtagtggccctgaatgtctggctgcgctgttatttattggatacaaagcaaaaggggcagggtaaagagtgtgactcatctccaatgataggtaaggtcacgtgggtcacgtgtccactggacaggggtcccttccctgcctggcagctgaggcagagagagagaggagacaaagagaaagacagcttacgccattatttctgcatatcagagacttttagtactttcaataatttactactgctatctagaaggcagagccaggtgtacaggatggaacatgaaggtggactaggagcgtgaccactgaagcacagcatcacagggagacagttaggcctccagataactgtgggcgagcctgactgatgccaggccctccacaagaggtggaagagcagagtcttctctaaactcccccagggaaagggagactctctttcctcgtctgctaagtagcgggtgttgttccttgacactttttgctaccgctagaccacggtccgcctggcaatgggcgtcttcccagatgttggtgtcaccgctagaccaaggagccctctgatggccctgtccaggcataacagaaggctcgcactcctgtcttctggtcacttctcgctacgtcccctcagctcctatctctgtatggcctggtttttcctaggttatgattgtagagtgaggattattataatattggaataaagagtaattgctacaaataatgattaatgatattcatatataatcatatctaagatctatatctggtataactattcttgttttatattttattatactggaacagctcgtgtcctcagtctcttgcctcagcacctgggtggtttgccacccacaattttccacatgcttctggtctgcgttctttttttctccttgcatcttctcattctctgatcacccc

SEQ ID NO: 4 - DNA, 1662 bp, Homo sapiens
gcggggaccccgagctagagccgcagcgggacctgcccggcccccggctccagcgagcgagcggcgagcaggcggctcacagaggcctggccgcccacggaacccggggcccggcggccgccgccgcgatgtttccccgcgagaagacgtggaacatctcgttcgcgggctgcggcttcctcggcgtctactacgtcggcgtggcctcctgcctccgcgagcacgcgcccttcctggtggccaacgccacgcacatctacggcgcctcggccggggcgctcacggccacggcgctggtcaccggggtctgcctgggtgaggctggtgccaagttcattgaggtatctaaagaggcccggaagcggttcctgggccccctgcacccctccttcaacctggtaaagatcatccgcagtttcctgctgaaggtcctgcctgctgatagccatgagcatgccagtgggcgcctgggcatctccctgacccgcgtgtcagacggcgagaatgtcattatatcccacttcaactccaaggacgagctcatccaggccaatgtctgcagcggtttcatccccgtgtactgtgggctcatccctccctccctccagggggtgcgctacgtggatggtggcatttcagacaacctgccactctatgagcttaagaacaccatcacagtgtcccccttctcgggcgagagtgacatctgtccgcaggacagctccaccaacatccacgagctgcgggtcaccaacaccagcatccagttcaacctgcgcaacctctaccgcctctccaaggccctcttcccgccggagcccctggtgctgcgagagatgtgcaagcagggataccgggatggcctgcgctttctgcagcggaacggcctcctgaaccggcccaaccccttgctggcgttgccccccgcccgcccccacggcccagaggacaaggaccaggcagtggagagcgcccaagcggaggattactcgcagctgcccggagaagatcacatcctggagcacctgcccgcccggctcaatgaggccctgctggaggcctgcgtggagcccacggacctgctgaccaccctctccaacatgctgcctgtgcgtctggccacggccatgatggtgccctacacgctgccgctggagagcgctctgtccttcaccatccgcttgctggagtggctgcccgacgttcccgaggacatccggtggatgaaggagcagacgggcagcatctgccagtacctggtgatgcgcgccaagaggaagctgggcaggcacctgccctccaggctgccggagcaggtggagctgcgccgcgtccagtcgctgccgtccgtgccgctgtcctgcgccgcctacagagaggcactgcccggctggatgcgcaacaacctctcgctgggggacgcgctggccaagtgggaggagtgccagcgccagctgctgctcggcctcttctgcaccaacgtggccttcccgcccgaagctctgcgcatgcgcgcacccgccgacccggctcccgcccccgcggacccagcatccccgcagcaccagccggccgggcctgcccccttgctgagcacccctgctcccgaggcccggcccgtgatcggggccctggggctgtgagaccccgaccctctcgagg

SEQ ID NO: 5 - protein, 504 aa, Homo sapiens [sequence omitted]

SEQ ID NO: 6 - DNA, 8315 bp, Homo sapiens
gtgcagtggcacaatctcagttcactgcaacctctgcctcccgggttcaagtgactctcttgcctcagcctcccgagtagctaggactacatgcgtgcactaccacacctggcaaatttttgtattttcagtagagacagggttttgccatgttggcctggctggtctcaaactcctgacctcaggtgatctgccctcctcggcctcccaaagtgctgggattacaggcatgggccactgtgcccggcctctttttaggacggagcctcgctctgccccccaggctggagtgcagtggtgcgatctcagctcactgcaacctccgcctcccaggttcaagtgattctcctgcctcagcctcccgagtagctgggattacaggcgtgtgccaccctgaccggctaatttttgtatttttggtagagacagcgtttcaccagccaggcggtctcaaactcccgactttctgtgatccgcccgtcttggcctccccaagtgctgggattacaggtgtgagcttccacacccggcctcctctcattttgagcttatgtaaatgcctaccggcccccagtagggctggggcagggttgactttccagtgctgtctgggatggctgggagcaaagaggctgaggactgggcagcatccccctagatggccccatcctgttctggccctgttctggcttccctaactcagcttgggttgggggcagccccaagccccatcccagaccctctgcagctctggcaacagctaggtccagagatgacctcagcctggggagccagtgttcctggggagacagaatctcattgcctcccaccccaacctgagaaagcctctcgtttgggggtggggtgaggttcagggagggatcctggatctactccaagccctgtttgctccactgaccaggagaaaaatgcggaaaaggggtgaaagggtgacatcacccaggattgctccacctctggggcaattatcgggagcagggcggccccagtcagacgcaggcagccccaaagcctgaacaggcagggccagacccaggtaagaagccaaggtccggaagggtcgcgggcgcggggcagggctgaggccccacgtcaggtcgcgtgggtctgcggagctggggtcccccacgaagggtttgcgggactcgcatccggccttggccttggatgtttatgggtctgtctgtgggtgagcctgtgggtcccggggctcccgaggcctgtgcggctttcaggtctgggggtgctgccggtgcgcctctgtccagggaccagctcctctgtcccgggtgggtgtgggaggcggacccggtggggaggggcggtggctgccctgaaaccagagctccctctcgccggtacccaggtctgtgtgaggcttccggcccccaggctcgggcctgcgcccagcccaccctacacccctaggcgtgtgcccccagccgtgcccagcaggggcggagggagggcagaagctttctccgggcggcagcggagggcggggctccagcgcggggggccgggtcaaggacgggggcgggtcccgagggcacggccgttccctgcgcgccccgattggtcttcgtgtgccggccccgcccccgccgtgagtcccacacttaaaagcgccgcctccgcgccgcccgcgtagcttcttcgcctccgccagcggggaccccgagctagagccgcagcgggacctgcccggcccccggctccagcgagcgagcggcgagcaggcggctcacagaggcctggccgcccacggaacccggggcccggcggccgccgccgcgatgtttccccgcgagaagacgtggaacatctcgttcgcgggctgcggcttcctcggcgtctactacgtcggcgtggcctcctgcctccgcgagcacgcgcccttcctggtggccaacgccacgcacatctacggcgcctcggccggggcgctcacggccacggcgctggtcaccggggtctgcctgggtgagcggggccgggggcggcaggcggggggctggcgggaaggccgtgcggggccgggggatggtctccgtgagcggaggggccccgcggcgagtcggtgggtaccgtggggagggttccggtccgcgcctggggaacccggcgaggatccaatccgggtcgctggcctgggggcggggcctggtttgttttgctccgagggtgcccggggcccgcctgtctggcgcaaaggtttgggggcgggcaggtgcagcaaaagctgcttaattggacagaaaaagtaactcgtgggcgggaccgtgagaatccgaggagggacctaagtcaccccgtggccctggatattacaaccggtgccagcgatcggtcacagtgtcctgggcttccgcagttgcaggctgctatcagggggcctggcatgggggtagcctcttgagaggcgtctgggaaccgtgacctggtccttcccaagggcccggaggaggagccccgctgaggtcagggctgggcgcgaaggggctctcctgccctctccttgcacacggtgccctgtggcctggctccccgctgcggcccaccgcgtttgcacacttcatgggtgagggtgcttctgggctctggtgcctgggtcaggagtggatgggtctctgtgtgctgggctggcctcggctcgcaccatcggctgccatgagggagtgatgtttacagcacacgacttcaggagcctgtgaggacacccaagatgacaggggcactctgctcagcaggagcctgtccggggctcacccctgccctcttcctctgaactttgtcctgggagggagggggctggaccacagaagtgaacctctcaggtcccaataactagagctattattgggaacagccccctccagccccctccctctcatggaggccttcagacaagcgcctcagtcggccccctggcttcaagatacatcaaggtccctgctacccgagccaaggacacctctcccacagtgcttccccaccctgctcccaacaagactccttctcacgggtggtcttgcacagctggagcccatagtgcagcaggtgctggctgaagagcccctggctccacactgccccactcctgaccagggtgatgcactggaggagggcttggacctcagctcctccctcagtgctcccgaccacttccagggactatccccaagctggccagcactcctgcgccccaagaggagtgtttggggtgcttcctcttggctgcagtgggacacaggtgtgccttcctaggaactgggccctgactacttccagcccaacactcccgggcctgtgaactgtgacctgtgtgccgggatgggttttgtgggtctgccccatccccgcactgctggatctggccaagtgggtgaaggctaaggccggtcagagttgagtttctgccttgtcccctctcctgggctagatgccacaccaggcccagtgactcatagggcaggcagttgggaaataccaggcagagggcaggtcctggtctcagctggccagcctctgctgtctgccatcccaggggaggtggccaaagtcccaactgtgagccaggccccacattcactgggcctcctccagggtctgtatgccatggaaccctggacatggggctatgaaggaaggtgggtgttgctaagcccaggagcatgggcccctaaccttggccctgtgccccaggtgaggctggtgccaagttcattgaggtatctaaagaggcccggaagcggttcctgggccccctgcacccctccttcaacctggtaaagatcatccgcagtttcctgctgaaggtcctgcctgctgatagccatgagcatgccagtgggcgcctgggcatctccctgacccgcgtgtcagacggcgagaatgtcattatatcccacttcaactccaaggacgagctcatccaggtggggcctggtggagccatgctgggtggcggtggggggggcagtgggaacctcaaggcctctgctcattctctcccactctgtccctgccctgaaggccaatgtctgcagcggtttcatccccgtgtactgtgggctcatccctccctccctccagggggtggtgagtattcctagccctggacaccttctatggggtgggccgtgggatgagggacagaggaggaggccgcctagagccatccttcaggcccttgctctgccaccgcctgttacccacttcccctgtgttactcaagaaacagctgtggcaacgcacgcttcctggccccccatcccttcctccgtccctgccctcccccgtctaccatctgctcagtgcccaggctggcccacagccagtgcccagtgggtaaaacgctcaaatgaggtagccactgaatggggcccttggtggccgggtggggtggctggggtgggtggccagtgcagccacaggccctcacatacggtcctgtctgtgtgtcccgtggaagcgctacgtggatggtggcatttcagacaacctgccactctatgagcttaagaacaccatcacagtgtcccccttctcgggcgagagtgacatctgtccgcaggacagctccaccaacatccacgagctgcgggtcaccaacaccagcatccagttcaacctgcgcaacctctaccgcctctccaaggccctcttcccgccggagcccctggtgagctctgctccgaggactgtggccttcccagccactcctcactgggcaccaagggagaacactgatcctttgacttctgagtgcccagggtagggtggtgggagcctcttctgagggctggggaaagcgcacaacctttctaggggcagatggcccatccaacctctctgtctagctgctctgtccaggctccctgtccagtctctctctcttttttttttttttttttgtttgagacggagtctcgctctgttgccaaggctggagtgcagtggcaggatctcagctcactgcaacctctgcctccaaggttcaagctattctcctgcctcagcctccccattagctgggattacaggcgcctgccaccatgcctggctactttttgtatttttagtaaagaaatagggtttcaccatgttggccaggctggcctcaaactattttatttttttattttttttttgagatgaagtctcacactgtcacccaggctggagtgcagtggctggatctcctctcactgcaagctccacctcccgggttcctgccattctcctgcctcagcctccccagtagctgggactacaggtgcccaccaccacgcctggctaattttttgtatttttagtagagacggggtttcaccgtgttagccaggatggtcttgatctcctgacctcgtcatccgcctgcctcagcctcccaaagtgctggggttacaggtgtaagccactgcgcccggctttctgtccaacctccctgtacagctttgatcttgttctggatctaggataggttttggggttgggagctgcgggtagtgaagggaggtggctgttgggggtgccagggattccaggggcagaacgggcatccctggctccctccgctccatttaaccctcctcactgcaggtgctgcgagagatgtgcaagcagggataccgggatggcctgcgctttctgcagcggaacggtgcgcggacccgggcgggagagggcggggtgggctcggctctgctaccccctgcgggccgcggccgcgctgatgaactgagaatcccttctctccccaaccccaggcctcctgaaccggcccaaccccttgctggcgttgccccccgcccgcccccacggcccagaggacaaggaccaggcagtggagagcgcccaagcggaggattactcgcagctgcccggagaagatcacatcctggagcacctgcccgcccggctcaatgagggtgcgcacctgggggacgggaggggaggaggggaggcaggagggaaagagagagaggagaggacggtgagcaggggagggaagccgagcgggtcctgggccccgctgcctccactggccgccgacctcccgcccacccgcagccctgctggaggcctgcgtggagcccacggacctgctgaccaccctctccaacatgctgcctgtgcgtctggccacggccatgatggtgccctacacgctgccgctggagagcgctctgtccttcaccatccggtgtgagggctggggggtcgggagaggggcccaggggacggactttgggatcatccgcgagtgatggttaccagggaaggtggggtggggtgagcagtgccaagtcagggcacagacagggcccggcgggtgggcatgtgggtctggccaagtccctgaacgcgccgctcgccctgtccccagcttgctggagtggctgcccgacgttcccgaggacatccggtggatgaaggagcagacgggcagcatctgccagtacctggtgatgcgcgccaagaggaagctgggcaggcacctgccctccaggtgagccgccgaccgcgcgtccacccggggcccgcggtgctagcgccgggagctgaagccctccctgccgcatccctgccccgcaggctgccggagcaggtggagctgcgccgcgtccagtcgctgccgtccgtgccgctgtcctgcgccgcctacagagaggcactgcccggctggatgcgcaacaacctctcgctgggggacgcgctggccaagtgggaggagtgccagcgccagctgctgctcggcctcttctgcaccaacgtggccttcccgcccgaagctctgcgcatgcgcgcacccgccgacccggctcccgcccccgcggacccagcatccccgcagcaccagctggccgggcctgcccccttgctgagcacccctgctcccgaggcccggcccgtgatcggggccctggggctgtgagaccccgaccctctcgaggaaccctgcctgagacgcctccattaccactgcgcagtgagatgaggggactcacagttgccaagaggggtctttgccgtgggccccctcgccagccactcaccagctgcatgcactgagaggggaggtttccacacccctcccctgggccgctgaggccccgcgcacctgtgccttaatcttccctcccctgtgctgcccgagcacctcccccgcccctttactcctgagaactttgcagctgcccttccctccccgtttttcatggcctgctgaaatatgtgtgtgaagaattatttattttcgccaaagcacatgtaataaatgctgcagcccagcctctgcccactttgtgtgtatgtgaccgcctgcttacttgcagtgagagcctggtggccagggtctggccctaccttggctgaccagcctctccacagctgcaggccaggtctcccagcgtcgcactcctgggcctggcatttggaacctgccaggctggcctgggaacacccccctacaggcacatatgaacgtactgcattcctgccgacccccctgtctaggatgcatccacaccccccccaattttgcccagcagcctcctggctgacccttggccacagccttctgagggccaatggaaatatttgggaccaagattcttggtaaataaaaacgaaaatgtttgcaaaaggtgtcgcctgcccctcccctcacacggtgacacagtggggtgtgtgactgttttggggcttggccagctgtgggggctgaggttcctgaaaccaagcacaggggacctgggaaagctgggcccctgctccacttgggcgtttggcccaaagttctctgtccccccaccctcacaagcctctctggcctggtgcacataccttcttgttcccctgcccagcctcctgccagtacacagggcagggctgggatatggaggcaggtgaggtcctagctgaaggggactcagccctgtgctggggaagtggtttcctgagggcatcctcttcatggcagggaggccatgaggggagaaggccagccctccttggcaaccaaaaaggccccatggcctgagggtgctggagccagcccccacctgctcctcccagaaggcgtggggtggctgtggtggcagccccctgccaacaataacggttggttctgggatctgggactgggcgagccggccaggcaggtccctgatgtgcctgtgccagaggccaccccccttggtctcaaggtcctgtcctataagcccagccttttgtggttcagctgggtcgcacgctggatcccaggccccttccttgaaaagcagagg

SEQ ID NO: 7 - DNA, 2809 bp, Homo sapiens
ccggctcgcggagagcgtagcgcggccttggtggcggaatggcgttgagtgacggcccggccccgccatctggttaaagggactcgttcaacacggaagtgtcccggggctgcattgtgctacagctagaatgaagcacatcaacctatcatttgcagcgtgtggatttctgggcatttaccacttgggggcagcatctgcactttgcagacatggcaaaaaacttgtgaaggatgtcaaagccttcgctggggcgtctgcgggatcgttggttgcttctgttctgctaacagcaccagaaaaaatagaggaatgtaaccaatttacctacaagtttgccgaagaaatcagaaggcagtctttcggggcagtaacgcccggttatgacttcatggcccgactaagaagtgggatggagtcgattcttcctcccagcgctcacgagctggcccagaaccgactgcacgtatccatcaccaacgccaaaaccagagaaaatcacttagtctccactttttcctccagggaggacctcattaaggtcctcctagccagcagttttgtgcccatttatgcaggactgaagctagtggaatacaaagggcagaagtgggtggacggaggcctcaccaacgctcttcccatcctgcccgtcggccggacagtaaccatctcccccttcagtggacgactggacatctccccgcaggacaaagggcagctagatctgtatgttaatatcgccaagcaggatatcatgttgtccctggcaaacctggtgagactcaaccaagccctttttcccccaagcaagaggaaaatggaatctttgtatcagtgtggttttgatgacactgttaagtttttacttaaagaaaattggtttgaataaaatgcataaaagtttataatgcaaaacacgttttagatagtttttgatggaagtttctaatcaaatcctttttagaaaatctatcatgactcaattgtattactccttgtaatatttgtgtttatttttaatatttgaattattacttagcacatggatataagaggcactttattagaatttgacaacgtcattaagaaggatacacttaggtgataaggaatcatttttggtgaactgttgtgtcctttaaaaaatggaggaggataggttctttgggtaaattgaggaacatggaaaatggagggccaccacttaggttccatggagaatatgtggcatgatcttgacgtgaagcatgggtgtttccgtgacttagctgtgtccctgtagctctggaactgaaggaactaggtaggagaggaaggatttgagaaataggcaactcatggttaaagacctttgatcaggactggaggaacaaaggtttgtgtattagtccattttcacactgctgagaaagacatacccaagactgggcaatttacaaaagaaaggtttaatgggctcacagttccacatgactgaggaggcctcacaattgtggtggaaggtgaaaggcatgtctcacatggcagcagacaagagaagaaaacttgtgcagggaaactcccctttataaaaccatcggatctcttgagacgcgttcactatcatgagaatagcatgggaaagacctgcctccatgattcagttacctcccactgggtccctcccacaacgtgtggcaattgtgggagctacatacacttcaagatgagatttgggtggggacacagccaaaccataacagtttgctttccacaggtgtgtgcagttgctcccagtgcagttaggggcagatatccagaagcctaggagattttgaagaggcttatcccctctcatcttcctcccctaaaccccaccccagtttaggagattatcaagctggttgtcattcattctctctctctctctctctctctctctctctctctctctctctctctcgccatacctctctgagtatttagctattgttctatatactggatacatctattatgggaacttaaatagataaaatactactaaacacaaagaaccaagtagcctagtaaataagaattaaatccagaataatcattcacttctcatttttaaatcacgttgtactgatcccaggttttttactttctgagtagttgtcgatcactttgaggacttgtgtatgggcttgtaccaatttactgacactgtcttaaatcctaaatctgtttttatgaggttttgccaagcaagaactcttgattactaagaggcagttagagacaaaccaatttaattccatgtcttcaattgtcacgcatttcttcttttactctttgaaactattctttgagtcaattttatggttctcagaagccaaaatacacaacttttagcacataaacaccaacgatggcctctttttgaggagttatgcatagacccactctagagtaatgatggtccctgtggtatatactttctcctactctagcaaacatgtagtttaatcttaatgtgttgtttccataagtgacatgaagtggatagattctcaattgttatgtccacttattcactaggtaaattttcagttttaatacttttctccttaccccttcctttgatcatttcatgtgaatattctatttgtatgatacactgtatttcaataaattccttgttgatgtacccttaaattgaagaaatttaagctgcaaaaccaaatctcattgtataagacttttttgaagtatcttgtatcgactacatatgtatttgaccctgtgggaggatggtacttttctttttaacttttattttaag

SEQ ID NO: 8 - protein, 253 aa, Homo sapiens
MKHINLSFAACGFLGIYHLGAASALCRHGKKLVKDVKAFAGASAGSLVASVLLTAPEKIEECNQFTYKFAEEIRRQSFGAVTPGYDFMARLRSGMESILPPSAHELAQNRLHVSITNAKTRENHLVSTFSSREDLIKVLLASSFVPIYAGLKLVEYKGQKWVDGGLTNALPILPVGRTVTISPFSGRLDISPQDKGQLDLYVNIAKQDIMLSLANLVRLNQALFPPSKRKMESLYQCGFDDTVKFLLKENWFE

SEQ ID NO: 9 - DNA, 30640 bp, Homo sapiens
aacagaaagaaaaagcccaaaccagaggcaatttggagagctccacccctcctccctgttttgtctaaggtagagacattctcgcagacgctcctttgtagcctcagagaaagtaaatctctgcccttaaactaggggacctggagagccttcaatcacgaagtcagggattcctccaggcttggttattaggattagataaggaaaacaccgtttggggagagaaagaattatcagagtcctttaaatcttggtgggcatggcaggcatcttggcggtcgttgaagctgcttaagtctgagccaaaggtgccagaggacaaaacagccacctgctcacggccccctatgtggaaaaagctttccaatttagctctctcattttagcatctcaatctgcacataaggcacgcgggaaactcagagaggttatgctacctgctcaaggtcacacagcctaaggtcaactgaggatatgaaagttccccagagtcatagtccagcttttcctttagatagcacttcttaaaaaaaccaaaggcacgccttgaaaagctgcgggatgtccagccccaaaatagccgctggagttggcccctaggggcagctggaccgcccaggcgggcgaaagggtggagagggagaagaaccacttccgggaaacagacgcgaagagggtaaggcacttccggggcacagaaaatgaggattattaaaggtcagttgctcgcaggtaagggccaagtagcgttaaaagacacgtaaggattacaacgcctctctagggtgtgggttaaaatgtctctctaggtactcgaagataacatgactctaaacgaggaatccttgcctgcgctggaggacgctccggtctagcggccaggttgagtccagaagtttaaatagagcaggcggggcctagcgggcgccttcgtcggcgcgatgacgtcacatacctgcgcgggaggcggtaccaacccctgccgcaggactttcctgggccggctcgcggagagcgtagcgcggccttggtggcggaatggcgttgagtgacggcccggccccgccatctggttaaagggactcgttcaacacggaagtgtcccggggctgcattggtacggagccctggggcccagcaagggtgtgctcctgcgggactgatgcggggaccaccccggccctgccggcctccaagccgcgggagggcactgagcctgtgctggctgggcctggccgcctggttgctacacacccaacccccttcccgtccccatttgcagaagtggaatcaaaactggggcttgaccccggatcgatgactgggactcagcctacgcggacgccagcgcagcctgcggcggccgagccacctgccctcacagggcctggccgggtgcatcaggaacaggcggtcttaggtccctaaattcgaggccctgtaatgtgcctcatcttggggtccctgaatggggcccttgggcccccaaggaaaccctgagaccttcagaatggacagccttgtggaggcacaatttcattcacttgggcaaattgtgcgcactgaggcataggaaaacatgtaattttgtatttagatgtgttccaagtataaagtggcgtggctgacatttcatagagccttaaattgccttcaccaggcatgacctcagtttgtgttgagatacttggcaaagaaatgtattgcattgttgagtgattgcttattgcgttactgagtaattgtattggcgttcataaaagcatgggtgctcacaatagaaattgggaaggggagtagggaactgtatttgaaatcttctgatacacttaccaagaacaaatgtattgttcatttggaatttgatacggtctctaataattttctgtttcataatggtagtgccgcccccacccgcaaaacgtttcaaagggtcaagagaaaagaccaaatgcccttgtcaactggaatttatacgtctgtttcctgccaagtaattaaatcgtagattcataatctatgtaccgaataaatgtggaatgtttttgttttgttttgttttaatgtataagacttgggtcaaggtgaggagggaaagaaagggtttccttgaaagattcttgtgtttaattagaattaattgaaaccactgtgcttaaaacagagaatgatgttagaagaaagccttggaacgatgatcaacataagactacacagtatgtgggataaactattccaacttacagcatttttgttttttgtattgccagtgctacagctagaatgaagcacatcaacctatcatttgcagcgtgtggatttctgggcatttaccacttgggggcagcatctgcactttgcagacatggcaaaaaacttgtgaaggatgtcaaagccttcgctggggcgtctgcgggatcgttggttgcttctgttctgctaacagcaccagaaaaaatagaggtaattaagtagtaacttaggctgtgttcctcatcaagaaattaagacccaaaggcaaacaccagcattgactctactaagttaacactgcagagaaataatcgtttgcttggaaacagaaaggattaggacttttctcttaagatgtaattattagggatctgtgctctggttgtggaaaagattattttgggtgatagtgaaataaggaaataaaatgacttatattactcttgagtttttatttatccttaagattccagctctgttcctttcaagcaaaggatatttcttctttctatgatcatgagaagagcatggggcctgttttcaaggccttgaacatgtgggcaacaagtcttacctcactgctacttagaaatagatacataaattaaaatgtgggcatgcagtttctattaagtggaatggtataggaatttaattcttttttaattctcacagtaagaggaagctctgagttccaagcaatttatccagaagtaatacaaataattattcattaagtactttatttaatgcaatctatgttttaaaatctgttttctgctatccagacagtgatcaatcatttgctgactgtgagagctggattccgtagttggagaatattatatttgttaatcattacgtactctagcagctttgaacaataatgaaataacaatttaacaccctcatacccaagaatgaatttcagtcacatttaaaattccaaaactatgcaatgttaaacatcagaatctcatttcagatcttttgtgagctgcttatcaaagggaagctagatttgaggccaatcattcagaaatccacctatcttcctgtctttatatggttgatttacagggaactagaaaagtgatttaaaaactgcagagattaaacaggaccagaaccttaagccatgcaaggtataaatgaggtcggaaaattccccctaaactagcagattcataagctttgaattacaagctacctccttcccataactgtgtttcgtttttgaagcagagggctttcagggtgtcacttcttcctttttactgcagtcttttcttgagctgtggcaaatcctaagctttggagttatgtagacttggtttccaattatcagcaaattaaaaaatgtattgtatgatttagaattaacctgatttggaaatactgcactagtacagccatttatacttaactaaaattctgccatgttaagacattgaataatttacctgaattggagacttgatgtcatcatattctcagatcagtttacctgttgttatttattttttgcactaaatgagagactgggttgtgttgtctgatggtgctggtatgatgaaaaggtgtgtgtctttatctggtgctcccatgggaaggtagaaaataattttcttagtttccttagggcatcctcatttgttcttcttcaaaataatttttcagccaaagtaaccctttgacttaaggttattttagttcctttatttttagcatctgaaataatccacttgtaggatagccttgagttttttttagcaaatgtttctacctactcagattcgtgttcgctcggttctcgctgatgatataagttcctcaagctttttactgttgtgacaaatacgtgtgtaattaatcttctgagcttatgcatgaataattaagaggagcacctgatactgggcatattttaagtggtaggcaaatttagacattgatgtggaagagctcaatgctgcaagactttcctcagccctcacaatttgcgaggaagccatggtgctagactcagtggggaactcaaagatgaataagtccatctttaataagtttacagtctggtccgggctatggagagtattgaatataactaatattagtacaaagaaataggctagatgggcagtctagctcagtagaatgaacatacatttggtggggctacccccatttctcacccaggagcttcccacagcctatatcaattcaagctccctacaatccacctacctcatagcgcttagagtgatgtcacccaaaggcacatctgatcacctcattacccccggggcaggggctgtctgtggacttaggacaatgtgacccaggtttctcagcatgtatcacaaggccccatgtgacttagccctacttgtggctcgccatagggacacatttcagctctgtgcccctctcagaattgttcttgctgttgtttttactatcctctgctttcaccttcgtcctctgcccaactcctgtttatatttaagtcccagcctgggtgtcaaattggagggaaacctttggcaacccatgaggttggaggttgacacacactgtattcatttcccgttgctgatttgacaaaggaccttcagcgtagtggcttaaaacagcatgtgtttatcatcttccagctctggcggtcttaagtctaaaagggtctcactgagctaaaatcaaaatgtccatgctcactctctccagaggctccaggggagaatccatttccttaccttttccagtttctggagggtgcccagactccttggctcctgaccccttccttgatttctaaagccataagcacagcatcttcaaatctctccaactctgcctctcctacctccctccttcccttacaagaaaccttgtgatgatattgggcctacccaggtcatccaggattatctcctcatctcaaggtccttaactggatctacaaagtcccttttaacatagaaggtctcctagtcacaggttccagggactagaatgtggacatctttgtggggagaggagagactgctgcaataatatagccacttaactgaaattctgcaatgttaagacattgaatgatttacctgaatttgagacttgatgtcctcatattctcaaaacagtttacctgttgttatttattttttgcactaaatgagagccaaacacacccttgtgggggctcttatagccctctatgacctgctcttggaggcacttatgactctcatgagcacagggtctttgtatcttgtcctctccaagtcccagtgcctagccaagtacctggtgcccagttggttgccaaattaattcattatttaatacaaaatagacttagatgtaaattctggctctgagactcatttttaatgagatattgggatagacacttgaactctccaatcctccgttttcttgtctgtcaagtgggatggttatacccaccctgcagggtgatgtgaagatgaactgacagtgtcacctctgtggatcccagtccagggtagacatgagcatccctgaaagactttataaaataccagtgcccaagatgaatctccgaagactctgattcagtctgtgcaaaccatagacgtcactactcctatttctgtcactggtgctgtcatttgtaatcagctttgttgcacggttgtaatcaaactgctgctgagaccacaaaataggggtgggagagaaggttaattgcgctgaggaaaccttttgaagaatgtgctcaattaaaattcagattgagccttcaaggatttttcagatggggcaaaggccatttaaggtgctttacaaaggaaaaaacaaaaaaagattaggattcttgagagtgtataccttgcaaagggcctgagagtggtgggtgaggccccggccaggtggggaatttgcacacctttaataacatgacaccctacatttgctgttgggtctatagaatcccaagttttagaagtggaatgattgttttctttacaacacctagggtcacttcttaattttctttttttaggaatgtaaccaatttacctacaagtttgccgaagaaatcagaaggcagtctttcggggcagtaacgcccggttatgacttcatggcccgactaaggtatacagagtacattttgcccaaaagtacatgaagacagaatgttgacgactctctgtacaaactttggcaagctcagcattctctcttgtaaaatatttacagggagttcaataattaaaggtgagtaattcagattgctttttcttctttagaagtgggatggagtcgattcttcctcccagcgctcacgagctggcccagaaccgactgcacgtatccatcaccaacgccaaaaccagagaaaatcacttagtctccactttttcctccagggaggacctcattaaggtaacaaagccatgtttcctatttgaagaaatcagcaagtgctgatttcaatacttcatataaagtaatgagcattaccacgttctttagatcctgtgtctcttaccaggcccgatagatggttgagtgctcagtaaattacttgtttgatgaaagtattcgccaggatccctcctgaggccgccttatgatgcactgtgatccaaatgctccctgtacttgttcagtgttttcctgcaatgttcagatctcacgttgattatgtgaagcaagtcgctgatgcagtttagtagcaataataattcctgaaatttatgtggctaatactttggagtgtatgtagggtttttaacatgtactctcttattcagtccttcaagtccttctgtaaatcaagtagggtaggtgttattgtgacccttttatgtttttgtttttgttttgagatggagtctcgctcgtttgcctaggctggagtgcagtggtgcaatcttggctcattgcaacctctgcctcctagattcaagcaattctcctgcctcagcctcctgagtagctaggattacaggcacgtaccaccatgtccacctaatttttgtatttttagtagagacagggtttcactgtgttggccaggccagtcttgaactactgacctcaaatgatctgcccacctcagccccccaaagtgctggaattacaggcatgaaccaccacacctgatcccttttatgtttttaacagagaagtgaatggaaagattttatgtgatgtacgtacctatgtacccaagattatcctgcgtggcagtgggagctaattctggtacccaggtctcctaacttttcaatctaatgttccattttgtttcttccattttgttctgatgtctttctatccaaacacatttgaaagttggttggttgagaattcatcatctctgggaaatacttcagctggttgatttccaaataccaatgtgcctgctgtggtaatgcactgaatatttgtagggtggaaagggtgtacttctcttggaattttttcttgtttttccctctagtgcaaatcataacaagcttcaggtaatatggagaatgtatgttttcgaaagcagaaggtaatgggttgagagtaagtggtaagattccatatattcggatgctgtcatttgaaatacttaatgaaatgttgaattgacaagtaaaattactttgccacttaatagattagaatattttccctcgatatccctattacgtcattctctcacttacaagagtctatcttataaaaagacattattctataatcttattacaatattatgtagacttgacagtgtcaagggaagagctgggcaacccatgctaagtggtcctaagccatcaatgtccaatacagacagataaggcttgcggtgggcagacatgcatcctcttgtttacctaggaacagactgtgtgctcttgtgacataaaatgagggggattctgccccagatctctctcacggggtccatccctggtttttctgtctacaatgacagaactctcagcccctgatctccccacatccctcctgatttacatatcactctttagcatgctacctcttttactgctgtcctgttcagctttcattgtccatcctccttcaccagaatgtaagctccaagaggcaggggtttctctggtttgctcactgccgcagcccagtacctgccacactgccagccattgtgtaggcactccgtaatatgcgtgaaacgaatggatatgaaccaaggttgcatctgttacactggctcgatttaaaataaaagaaaagaaagaagctgggccctccatactgtatgacctgggcaaaatcacatagctggctggggcccagctgtgactgtggttcatgtgtctagcattcttccctaactatatataccccacactggataaaatgctcatcctccctatccatcccttttatgtggcagagagaagaatgacacccaaaactgtccttaccctaatccccagaacctgtccagatataaccttacctggcaaaagggattctgtagatgtgattaaattaaaggtctgagataggaggatgatcccatattatccaagtggacccaaactaagtacatgaatcttgaaaagtggacaccttttctggattctgtgtcatggctggtttggagatgtagcaaccacatgccagggaagcacagaggcttctcggaaagcaggtccccactgacagccagcaaagaaaagggggcatctgccctttaacctatggaactggattctcctaacacctgactgagcaaggagacacattctcccccagagcctccagaaagagatgcagccctgccgacacctggattttccccagtgggacccataccagactttggaccctcagactgtgtgacaatacatgtgtcttgtttgaaggggttaaatttgtagtaatttgttataacggcaatagcaaactattgctgttaagaaaacatgacgtttttctagttcctttgttttggataataactattcagagaatttgtgtaattacttgagttcattgaacagaagagaaattgttgacacaaatgtgtaatactttgtaaggagaatatggaataattgacatttattctaggttctacaagaaacattccacgtgtttaggaggatgttttaacatttttcagaactaaaaaggaaggatgaatagttccttgatagcagatggtgattatcaaactgtgaaacctatgcctggaatactcatgaagcatttaggggaaaagagtgagagtgcgggaagactagggatgatgtagcaaaggaagaagtgtgaagaggaagtaaagtaaaccccctgccaggtagggacatgatcaagtatatatgttccggtgaaagaagtggctggtgggagaatgttgtctgaggtggtgccatgggaggatggtgcttccgacctttttggttgggactggggaggctgcctgaaggtcaccccactccccttaggtggagacaggaggctctgaagccatgcattgaagcagctcgatgatgggtagctgtgggtctcccagggcttggcacacctcgctctgaacatacagcctggggtcttgatatggtttgcctgtgtccccacccaaatctcaccttgaattgtagttcccataattcccatgtattgtgggagggacccagtgggagataattgaattatggggatggttttccccatcccattctgttctcctggtagtgaataagtctcacaacatctgatggttttataaggggtttctgcttttgcttctctctcattctctctcttgcctgctgccatgtaaaacgtgcctttcaccttccaccacgattgtgaggcctccccagccacatggaattgtgaggccattaaacctctttttctttataatttacccagtctcgggtatgtctttatcagcagtgtgaaaatgaactaatacaggtctacttcccagtatctggatattctgtagatgggaggggatgggagacagagagaaagagagaggttgattaaaactcaagttagagtaaaagagcatgggagagcatgaaatactaaacactggtgattttcaccttctttaaactttgctccatgttgtattttctgtttttatcattaactcatttattctgctacttaatagttgaattttttaaaaaatgcctagtttttcattatccactagctgactgatgctgtttgttacagagtgcctatgtacctgaaaatccaactagaaacctgtagcttagaatatgtgagagtgtgtgcttgtgtgtttgtgtctgtgtgtgtattttgacgtgtttttgaatctcctcaagaatgattacttgttttatgagtgtcccggtgatcatgtctaggcctgtcgtttcctgacgttgtcctcttggactgattacttttgagtctcagttttctcatctctgaaatggagtaattcacacagtaatactaatgagtgttactgtacagaataaataagataaagcatgtgaaagtgtgttttaaatgataaagccctttatgcatgctgaatggagctaatatttcataaataatgaacgaataacataataacccacacacacccctgctcagcacccttgtatgaatctgtcatgaaattttaaagccatattaaatcacaactttcttttcttcaggctgaataggattactttttcttatcagtgttattctttaaccccttgcttattacctgtcttcatgaacaatgtgctaaccgatatctgtaatacttcaggaaatggttgctactgagttcttgctacctttaggcatctgattttacagctcatcattataagccttcttttttaaaataaataattttgtgtgatgattcatacttaatttatcttaatgagcttcacctttagtagcatggaaaaatcactcatttgatagctgcaatgctagttgattgttttcttgaaacccttgtggactgtgtggtccaactattcattttatttggggttgaatttccacagaattttgaatcttacgttcatgaggatttaactcagtcttttctactttataaactgttataagacaggtgcaggccttaaggcattcctttgttcagaatttattggtgccaaccattcccctcctcctttatagtaactgtactgacaaagggaaacacagatgatgtctgctcaactacttgaaggcagcctgcagaggggagacccctttatagagttaaccagagccccaggaggcccccctccaaaatcatgtctttatttcccgtctgagagatttatttccaggtgccacacccagaaaaatgttacccccaaggatgaccctttatgtctgtgggtcgccagcagatcaacaccaagctgagatcagttcctgcaaatgcagataatttagggaattgaaaagagtcatggaggggtggtacacatccacctaaccctgttgttggtgggctctgggacccttggcgagtctcaacttcttggagccttggtttcttcatctttaagatggagaccttcagaacaaggtcaaatgaagtatgtatgaaagtatttcatcagctcttaagtacacaccagatgtgcatggtcaccttcaagtgcctccactctccagacatccatcccttcatactgttcattatttagcatgattccttcctgattcctctcctggtgtgccgctctacacagtatcccccgagtgtcaagtgtgtgcattttgcataccaactcattcatttcgagttccagattttattgtgggctctttttctttagaagcatgcatgcaactaggaaatccaaacacattttaatgttagccttcagcaaagcctaactagggaggtgagcccctaggttaaaagaaagtcatatgcaacttccaaggctgagctgaaatgatgcataaattaacagtgcaatggcgtcgtagagttccactacttccctgaattggtacaaatcattgaattcttcatgtttaagactgatgcacaagtgttttccgaaagggtcttttgacacgtgtgaatgctctagggtagaggtacccaggaagcctttcctgcaccatagtgtccctggccgtcttgagaaaattctgccctgtctctgttgcagtgatgtcctgaatttctttgcatgtgtgagaatgctcttaacaagatcctctcacatttagccttgtgaaggtgaaataaaaactgaatttaagattggtcattatcttggaaaaactgaggaagataaatttgaatctgtctttgaatttgaactacagcagcaagttctgtataaacagttaattctcattaaagtccacagtaaactttaggcataaatgaaacttgctgagaatttaatccctaaggaagctaggccacataattttctaaatacctcttttctgtaaaatgcaaaatgaagagagtcaatcagaacgtgtcgggaatgcgcacctggcgatgttttatacagttgctacaggaagacactagctctcatttttatcattgtgtgtttagtaagaacacaaatcagtggtttcacatatgtccaacatttaaagtgctgtaccaagtgatagggtgtgaaaagtatacccctgctttccacatcagcgttctcaaccacaaacactgcactcctggcagtaagggcccagattattacccagatagttatgtattgtgacagctgtctgtcctttgtaggaatttgagcagcatccctggtctgtgttcagtagatgctagcaggacacacccccccccccaccctccaccccaggttatgacagacacattgctaaatgtccctagcgggtaaaatcacctccagttgagaacaagtgctgtagacgtgttgaggttcattggcgaggtgaatttcactgggaatattgtgaggtgagatacactaaaagcacatgtttaggcataatattttattacatgaccttagaatgtcagtctttatctcctttcttaggaaattctatttgcctgcaataaaatggcagtaagctgactacaaataagaaaagctgcttagctcttaactatctaaagctaatttcccaactaaacttggtttgatatacaaatcatcagtccatataaagggagagtcagctatgacaaaaacaaatacattgatttggagaagtgacaagtattttccagttcattgcacttttcatatcatttaggaggattcttactgttattatctgtaggaaagtaattggatagcatttttcatccaaatatctcaaaatagttgcaagcattaacttgtgaactctacattatactttgacacgggggaaaagaaaaaaaaaacataattctaaggttagtgatttttttgcagtccacatgtctctctgcagaatagtgaaataaatcattatatttatgtattgtctttggttctttaaagacctctaagtactgtaatttcacaatatgtacgctatctacaaattgctctagttattgctttgcagtgttgcatttgctgaataacttcattaaatagctacattgtgcttgcgaaacatacctagggtctgtgattatttttttaaaagcatagccaattgatgataaggtcaggtttagttagggtgaatgcagttaccttaaccgtaaacaacctttctctgaagccaatgatgtcattgctaccggtggactgagagggttggagttgaagatgcctgctttctgtcctctctctcctattgatttattaagcgatcttcaccaaatcacccaaggtgctactcactcctgctttgcctctttgtgaaagagacatgattgctctaatgtcacagtattgcattatgcgggccatttaaagttgagattttgagaagaatgtcacagtgcgggcatgtccacattgacattttgtagaaggttggcagaccttgcgtgaggttattattatgccttgtaataaataaataaacaagcacataaaaaataaatagaagtgcatgctttcctttgggaaacagtgctgcaaattagcatgctttcaatatttcaataaacaagcgattgagatagatgtgcatgaaattgaccaaaagatgaaaaaaagacccattgtgagaaatctgacgtgtatgttggctctggtaaaaatatacattttatattaaattgattctggggacagtctcaaccacgtattagcagagagacctttgagcaactttatctttttgagacagggtcgagaaataattaaatgagacactatatttgaggcaaggtctacttcatgaatgtatgacctgtgcatttgcacgtggctccactatccaaagggcctcaccattggtttaatgctctgctgtcaacatcttaatattctccatactttctgaacagggggccctgcatttatcttattttggacaggacccagaaattatataactgattctggcacctagcaccagcatggtgcaatagaaccctttcacacttggctatttcttaccttctttcctgctggagtccttgtgttgtgctgttacccttgaatttgccactgctgaaacctgaggccccagaagcttctgtgggcactcgtcaggagcctgttgctataggagctgttacaaccaggcctgtgcctttgcaaccaacatgccaccaacagcactgggcaaccttctttagtgccactattgaggtaggtggaggagtgttgggtccattcccatcacagagaagggagagagatacccaacagatgtttgttgagtactaactctgccaagggctggacataccaggagagacacaagtccctctcctttcagtgggttttttggtaatgaggaaacaggagacatgtccaaggaagaaccactgcataagaaagctatggaagttccctgtacctggtgcagaaataaacatggcaagagtggattccagctgtgtcgggtcacagctcaaaagaggattttcctatggaaattgttatgctggtgactgagtctgtaatttagttgcagttcagaataaataggcttttgagagctgacccaaaaaccatacctataacattgatttttggaggaaatgccttctgagttctaaataattgtaagtggatgatctgcaagtcagcatttgagccataatccatttctgaataatgtatgatgaaaaaaaactcatgaaatctttgacaacaaacagccagagtctttcacttatcagttgaaagattatagcttaaaaaatattcagtatctaatgctgtatgttttcaaatagaacactagtgatttggaaactacacggcaaaataaaacatttacatttcagaaaagctatcagagcccaacttcccatgatgtatggaatggtaatttcctaaatgacaatctttttcaactgtttaaatgggagataaagcataattgatgtcactgcttgccagggttccattacatatcctgaccacgtcattggaacataaaatatgccattccctttgcaaatcctggtgacataaatgagaacatctctgagctgcggactaatctctcctgtcataatcggagggctttgttcctccagaaagcaggagtgcaactgtaacatgatttttacagtatatgatggaacattacatataatttatctgtaacctttacatactcaggaacttagacttaccttaaatttcattttgtagtaacttgtgtgtctactattaaaatgctgatttttacctaggtgaagttgattgattcagagtatgtcaacttgtacctatctgtttagagtcttttccgatataaagtgttatcttttatgtaattacgtgcagtgctgcacttgaagtctgtggggcttatttattgagattttgctagtcaatactacatcaactgtattgtctgaatgttcatgcctgtaatcatgagctgcttctttattttatccatctaggtcctcctagccagcagttttgtgcccatttatgcaggactgaagctagtggaatacaaagggcaggtaagcttgttataactgaggaattaccctcctccctatattaatatgtttcttttgaaacatccaattttataattattctagtggcttcaaatcagtttatgtttttttcctcctaaatagctttgtttctctttaagagggaaagaatatacagggctagtgataacttatcatcaagatcttgtgaatgggaatgattgactgtgatgctcttattttcatcatatatatatattctgttgttgttagcagtgaacgattttttttccacctagtaaagtccatactcacagccccttctaagcagagttgtaccaccgtcagggtgagaaaaggctttagggctccacaccttgtcctcccccagacaatgcggcataggttccttccagcctgtcctcagcccctctgctaggggacgacccactcccctccaaggctctctacttacccagacacgttgatcactgcaaggccaaaacccattttgtttgaggagagtgctcctcacttggagctggcacttaagacaggatgccagttcttttagggaaatttagacatgggcttccacctgaagtctgcgctgactgtgacccagcttccttctgtgatctacacagagtttgttttgagtattcaccattcgacccaaggcagctcctccattgcatggacacaggttcaacatgagcattttattctcaaatatagtgtctatttcacatatgtcacagatacgtattttataaccaaatatagtatttggtttaatgtgtttatttaacgtatactggaaacatgcattttataattaatacagtagctattttacagacaccagagatatgtattttataatcaaatagagtatctgtttagcacataccagagatatgcattttgtaattaaatgcagtatctatttaacacataccagaaatacgcatcttataatcaaatggaatagctatttaacacatagcagagatatatattttgtaatcaaatagagtatctgtttagcacatacaccagagatatgtattttataatcagttatctaacaattactagagatatgtatcttttaatcaaatattatacctatttaacacattccagagatatatattttataattaaatagagtagatatctaactactagatatctaaatagagtagatatctatttataattaaatagagtaggtatctaacacataccagagatatgtattttataatcaaatagggtatctgtttagcacataccagagatatgtattttataatcagagtatctgcttaacacgtaccagagatatgtattttttaatcaaatatagtacctatttaacacatgccagagatatgttttataatcaaatagagtagatatttcatacatatcagagatatatattttataataaaagtatttagcatatatctaataaagtatctatttagcatatatcagagatcaagcatatattcaggatgaataaattacatttattttatatatatgtaatatatatttatttattaataaaagagtacatacaaagtgctttaaccatctttcattcattcattcacccactcaccttgagggcccagcaccaggcctgtgctcaaggaattgatggtctctcaacaaaagaactgagttttacaaaccctttggctaataggatcgaggcagtgtctctgttctgatctcatcggtcctcatcagtgtctttccttgagctttgctgagctttggaattttttcttttctctctagtgcaaatcataataggcttcaggtaatatggagaatgtatgttttgatagagcagaaggtaatgggttgagagtgagtggtaaaattccacatattcagatgctgtcatttgaaatacttaatgttgaattgacaagtaaaattactttgccacttaggagattagaatatttttcctctatgtccctattacgccagtctctcacttacaaattaagagtgtttctatcttagaaggagaggcaggtggtagaggctccaggttctcttccaatatacactttcttcttctttcttagtaacagaatcccaatttttatctgaacctgtggctgtgcagctaggttatacatcccagcttcccttgcagttaggtgtggccatttatttcattgttgggcaatgacatgtgagccgaataactgtgggacttacagtacaaggaaagttgtatatctttccatttttcctgttgcccagaacttagatgtgattactggagctccagcagctcttgttcatcatgaggtcaaggatcacaccctgaagatggccaaatagtgagctggaaatacccaggtcctaaatgatttgaggagtctaccttaccagcctgccctcgatattcctgttaccagaaaaaagaagtaaaactttttttttttttttgagatggagtctcactctgtcacccaggctggagtgcagtggcacgatctcagctcactgcaagctccacctcccgggttcacgccattctcctgcctcagcctcctgagtagctgggactacaggtgcatgccaccaagcccggctaattttttgtgtttttagtagagatggggtttcaccgtgttagccgggatggtctctatctcctgacctcgtgatccacccgccttggcttccgaaagtgctgggattacaggcgtgagccaccgtgcccagcctaaaacttttgttattattgtatacaccagaatccagtgctatctgctgtagggattcttacttccaattttacatgtgaggaaattgagatgtaaagacatcagtctacttcactgaagtcactgagcttggaaggggtcaacttccccaagatagtccttaatgtttctatctttgctatgcctcttactctgctttgccctttagttcttttcaaacaatgcgttccttgttttcttcctgtccagccgcagcgccatgttgtgggtgtgtgacaggaagactagcaatgtttaaaagggcatgaatgaggaatcagtttctagtgaactttccatgatgtgattaagtactttggacatggacatagagtgacagccacaattaaacagcagggaacgcacaggcccagaactgtgaggaagatggaaagcgacccacccaagccccttgaccctggacaatgtcccaggacagtgtcttgctgatcatgtggtgtctgtgcataggaggcacaccaataattacttgtagagttgaaccttatgtctcctgaaagcattcagttttcctctgttgtttaatggagtccacaataagggggcctcatgcacatgattgacagagagccacagcggccttgcattgtttataacaccagaaagggacaatttagaagtgccattctctgcttaacactaactctctttaagcctgatcacctcccacattctaatagggcttccatgccgagttgttttctagaatctttcctttccattttcagggaagcgtgaatgttgctttaaatgcagtgttttaatgtgggtataagctttttatgtgacttaaattacataaacatttcagttgtgctgaatacacctcttattttctagattttcatgttttcatacagctcaggttttgatgtatttgttgtctttagttgtttttctgaagtgacgaaagtggaggtggcagaaggggaaaaagggacttagcgttacttatatttgtggcactgagtcaggaaaggcagagaaagtgggactcagccaacaggggcagagtctagtctctccacttgtgacttttgttaaatacacagacctcctcatttctacattgcgggtattactggattttattaagatatatttgtggatccaatatgtgcattaaaaaatgtttttattaatcgcttagaagaatcataccttaataagacttaagattaatttcaaccacagcaaataccagtatgagtcctttcaaaaccctctaccagtgatttaaagtggaatgatgtctttgaaaattgctttttctttttaacttgggaaacatggcaaaaccctgtctctacaaaaaaaaaaaaacaaaaattagccaggcatagtggcatgtgcctatagtcccagctactttggaggccgaggtgggaggatcacctgagcctggggaggttgaggctacagtgagctatgatcacaccaccgtactccagcctgggcaacagagtgaaaccctgtttaaaaaaaaaaaagaaagaaagaaaactgctttttaaaaaatattattttaaaaagttctgtagctttctctggcttcatcttatgactattttgataccttatagtacatactcattaatagatttgtcagtgaatgaatggaaataaattgaagtttagtcctatatttttgactaatgatttttctttactacatatgaactctttccagtggtagaataaatccacaggaatgttcacacatttaggccatgttttcacagattcttgtcaatttggaataagaatgaaaattttcaagaggaattcttaactcatttgttttaagagatagaattagcattggcaggtgactaaaatatcagaataaaatcatctccagaactggcacatgcagatcagaagaaaagctgtagggttggctgggcacagtggctcacgcctgtaattctagcactttgggaggctgaggcgggcggatcacctgagatcaggagttcgagaccagcctggctaacgtggtgaaaccctgtttctactaaaaatacaaaaaattagccgggcatggtggcgcatgcctgtaattccagctactcgggaggctgaagcaggagaattgcttgaacccaggaggcagaggttgcagtgagcagagatcatgccatggcactccagcttgggcaacaagagtgaaactctgtctgaaaaaaaaaagaaaagaaaagctgtaggattttgcattgtttctttcatctgagttggaattcaagtaatattttgactgtgtagtattacaagatcgcttcttggccttttggttaagattaagtatatactattacaggaacgtgaaaatgcactgttgtattaccctagtccatgcggatatgggtcatttttcctttcattattgacacttgtgtgggaaagagaattgtggaatactccatgtaactgtgcttttatatgaggttgatactgtggtccttaggcttctatttcctcgtggcttagactttttacatggaaatgcagctctgtcactgtcatagcaaacagtggtagctggccttgccaccagctagatttggctggatggaaacctggatatcaatttctagttattctgaaatagacgctcttattcttcagtcactttttagatttttttgcactgtctgtttaatttttgaaacaacatgttagtcctctaggaaaaattttctgtatggtgtttatagatggtcaactgttaaccctttagagccttctcaaaagtagatatctggagtcatcatttttaatcatccttcacacatgcaaaaaccaagaaagcaatgcaccaagaatcaatgcttagtagaggttagtatggcaggtttctcaacctctcttttagaattgtatttttacttgtctttctttgattaaaaaatctcatctgtctcttcttcccaccgtaattggaagattctcacctgtgtctaaggaagtggatatctctttgtctctttccctctcccttctattttaggaattgctaatcaagagcattgttgggcattgctgtctgtaggctatctcatgaaagcaatgcagatatgtttgcaaataagaaattattgttcaaagcacatttacttctttctcacccctcagaaatcagttctaatttgccctgccccatcgagcatccctgaattttacagtgtgcgagaaaaaaaacctgaagtgaaaagacaactatcccttcttgcctgtatattctatagcaatagatgttttattctctgtaaagtatcagatagcaaatgctttcaacattgtctctgtcaacacacctactcaactgtgctgtagtagagggaagcagccgcaggcaatatctcaacaaaaggtcctggtcatgtgccaataaaattttatttactgaagcagatggtgggtcgtagtttaccaccccgtgaactatagcaaggaaacctgcaagcctcagtttcctcttctgtaacatgagggaactgaatatacaagcatgacctttctgccttacagggttcgcatattaaattatacatgtgaaatcacttttaaactactaggccctctgccatggtatttataataattatagatagcaacattcattcttccagcaaagtattttttattcacttgttttttgagcaaaatataccttaagaaaatggagcccttcatggaagttctatcaaaaacatatccccagcatctaccatacatccagacacacactagatgctcagggatcacttgctgaatgaataaactggccctttcagtacaggttaactcatcaggtcctttcttaagttttccacttgcgttatgtaatagcagttacttgaattcataaggcatgttctcaatagctacccataacacatgaatgtctaaagcgaagtttaacaagtactgccagatgaatattctagcctgtttaggtgtaatgggtagccttatctctttagaagtgattctattaattccagttttcagtgctataactttcagagaacaatcacagtagatgtctgccccacaattgggcatgagtttttttgttttgtttttggtttttctattgagcagttgcttatttctatgtcttaggactcaggcattgacttgaatgtggaacacatttagaatatggccaaagattagagaaaagagtagtaggtctagcagtgggactgagaggtggtcactgtacagcctgtaacgagtatgattttgaaactattttgccttggaatttttatgtacttgctctcccttttcctttgaggatgatggtgatatgtgatgaaaactgacaaacacacaggaaaactctaacggggtagaccagcagctatttttcttaaggacaattttggagtcacagaagctcatggaagtagctaccagcatgtaaaatgctgcccatgaagcaagaaggtgggaatgactcacagccatatgttgtgaacagccagagaattcatgggcagaagtgtttactatgcaagattttaaactatcagtagagcacaagatataaaatgtttctacttttaatttggaggttgaagaatatttgttgttgtgacggttgtttccagaaactgctggaatctgtatgcccgaatgaatgtgtgattgtcctaagtgatacagctcgtgagagcataaatcaggagtaagaggtaccacatttagcatatctgagcagtggggctatagcatcaagggttgtgaaggagccggtatcattgcaagtttaatcaagtgatttgataagctgctatacattttcccttgtcatttatctaatcagaataagctaacaagagtgaacaaaatttggaaaaaggattttcgattccctaaaacagcagtcattaaccccacagtaaattagaacactttttaatttactgagccagattgcccttcttttcattaagttgaataaatcagaagagctgttacttaaagttgcatatctttaatttttcttgatgaaacctgtatgaatttttacatcgttgatgacagcttctgttgacaatatgaaataattttggaagctgtctttcttttcccttctgggtattatcctattccagaatttgatatttacacctaaagcaatcagggaaaaaaagcaggttctgggacagaatcagatattttggagcctttttttttgcaaacctcccttttagatcatacagttacataaccagctatattttatgtatacatatgggtacatatataaacacacatacacaaacacatgtgtacatgcatatacacatacacatataaataagtatgtgtaatacacataacacataatatatcataatctttatatcttttaaaaataatgacaatatgcaaattatgcaggtcagttttattttaatccaatctacttatactctctcctccaccaaaatgcaattctctcaaaaggaattttcaacccatttttatcttttatttatttacttttatttcctttggaggagtttgtggctgtccagagcctttgtaaataatgtaataccactcattttgtcatcagctttagaaataaacacaattggccaggcgcagtgactcatgcctgtaatcccagcactttgggaggccgaggcgggcagatcacgaggtcaggagattgagaccatcctggctaacccggtgaaaccccgtctctactaaaaatacaaaaaattagccgggcatggtggcgggcgcctgtagtcccagctactcgggaggctgaggcaggagaatggcgtgaacctgggaggcggagcttgcagtgagctgagatcacaccactgcactccagcctgggcgacagagcgagactctgtctcaaataaataaataaataaataaataaataaataaataaataaacacaagaaggacatgaaaaagacacattggaagaaataaatgcatggttttaagtgagttgagaacattttattttggcctgtttctccctcctcttgccacctttatgttgaataacaaaggtttgttttatctgcattgcattttacacattgaagcaaaactcagtttacaaacttcactaaagattcctgcatacaggactttcaactgttaccagactttttgtttttttgtatattatctgaaggctatttttaataacagataaacttagacaatttgggtgttatttccatgtgtgttgtgtttattgagtgctatagctgatcatgtgaatacatcagatcatggctgcattgagactgtttggccttggaatagacagcagaaggaatattgtctgtgtggttcttcagtcaccatttctcattgtgcgcttctgtagagctaggggctgaggctgcacacaccttgagcacctcattgtccgtcctcattgccccaagactctttcattttcttccttgggaggcttctgtcctccgccttctccatcactcactcccctttgcggtgggcttgagagcactgagtgcctactgaggaatgtgtgctttgatgcagcactcccattccttatggcttcaggacatgccccttgcttcaagagcacaggaaccactttggaattagaagcatttctagttgctgtatttactttttaagtttgccacagaaaatgccagtttccaattcatataataaaaatggaacctcctctgctggtggatgttttgttcagcatccacagtaaatttgactttttagtttgattaaataatgaaccccaatctaattacacatggcttcttaagcatgcactgtagtcagtaagaattcaagtcttacagacaataatctagttaaattctgtccagagttgttcttatattggtaagtgatatgtgtaccaagatttgttttacccccagaacagatccccaatgggattagtcctcaaaaatagcaatttaaaagcatatccaaggcacacatattattagaagctgcttttctttgtgcaggatgtgttgttgacgtgtgctgacgtgagatgtttctttccacagaagtgggtggacggaggcctcaccaacgctcttcccatcctgcccgtcggccggacagtaaccatctcccccttcagtggacgactggacatctccccgcaggacaaagggcagctagatctgtatgttaatatcgccaagcaggatatcatggtgagtaattatgttcagtgatatttgaagagttctgatctactaaaaattcccacagaattcaacgtgtttcccaagcactggaagttcattgataattgtaggtaggagttgcatcttctccttagtgtgttgtttaatactttataacatgtatcagccataggaacggggctaatttttttatcttttgtagagacagggtttcactaggttgcccaggctggtctcaaactccctggctcaagtgattcttccaccttggcctcccaaagcactaggattacagacatgcaccacgcccacccatttttgctgcttattaactgtcactgctttttcttcacttcttttttacatcaataaacttggtaggatatcagagcaatttttttcttctttgagagagggtgttgctctgtcccctaggctggagtgcagcggcaagatcatgtctcactgcagccttgaccgcccaggctcaggtaatcctgccacctcagcctcccaagtacctgggactgcaggcgcgtgccaccacacccaactacttttttaaaaaaattatttgcagagacaaagtctcactgtgttgcctaggctggtcctgagctcctgggctcaagtgatccacctgcctcggcctctcaaagtgctgggattacaggcctcagccaccacgcctggcccagagcaattttaaataaaatcatttatttgcttagaaagatggaggtaatgaaatatataaaaatagtggagaaaaacttagggacccaaatgaataattgacataaaaatactgattcccttaatctagtctaatagttttccacatgggatgatattgtactagacatttttggttatcatgtcccagtggtgctactggcatctggtgggtagaggccaggaatggtgttaaacgtcctgcagtgcacatgactgtcccacagttaagacgtcgtggccccaaatgtcagcagtaccaaggctgagaaactctcctttagtgtatcagtggaggtaacttctgaagtttaaatttttcctactgattttttgttactgctaattgtatctgtgataagaaaacctagattataaaaatagtgtatatttacctgtactgcaatatcctacagcttaaattttggtctttttattctagttgtccctggcaaacctggtgagactcaaccaagccctttttcccccaagcaagaggaaaatggaatctttgtatcagtgtggttttgatgacactgttaagtttttacttaaagaaaattggtttgaataaaatgcataaaagtttataatgcaaaacacgttttagatagtttttgatggaagtttctaatcaaatcctttttagaaaatctatcatgactcaattgtattactccttgtaatatttgtgtttatttttaatatttgaattattacttagcacatggatataagaggcactttattagaatttgacaacgtcattaagaaggatacacttaggtgataaggaatcatttttggtgaactgttgtgtcctttaaaaaatggaggaggataggttctttgggtaaattgaggaacatggaaaatggagggccaccacttaggttccatggagaatatgtggcatgatcttgacgtgaagcatgggtgtttccgtgacttagctgtgtccctgtagctctggaactgaaggaactaggtaggagaggaaggatttgagaaataggcaactcatggttaaagacctttgatcaggactggaggaacaaaggtttgtgtattagtccattttcacactgctgagaaagacatacccaagactgggcaatttacaaaagaaaggtttaatgggctcacagttccacatgactgaggaggcctcacaattgtggtggaaggtgaaaggcatgtctcacatggcagcagacaagagaagaaaacttgtgcagggaaactcccctttataaaaccatcggatctcttgagactcgttcactatcatgagaatagcatgggaaagacctgcctccatgattcagttacctcccactgggtccctcccacaacgtgtggcaattgtgggagctacatacacttcaagatgagatttgggtggggacacagccaaaccataacagtttgctttccacaggtgtgtgcagttgctcccagtgcagttaggggcagatatccagaagcctaggagattttgaagaggcttatcccctctcatcttcctcccctaaaccccaccccagtttaggagattatcaagctggttgtcattcattctctctctctctctctctctctctctctctctctctctctctctctctctctcgccatacctctctgagtatttagctattgttctatatactggatacatctattatgggaacttaaatagataaaatactactaaacacaaagaaccaagtagcctagtaaataagaattaaatccagaataatcattcacttctcatttttaaatcacgttgtactgatcccaggttttttactttctgagtagttgtccatcactttgaggacttgtgtatgggcttgtaccaatttactgacactgtcttaaatcctaaatctgtttttatgaggttttgccaagcaagaactcttgattactaagaggcagttagagacaaaccaatttaattccatgtcttcaattgtcacgcatttcttcttttactctttgaaactattctttgagtcaattttatggttctcagaagccaaaatacacaacttttagcacataaacaccaacgatggcctctttttgaggagttatgcatagacccactctagagtaatgatggtccctgtggtatatactttctcctactctagcaaacatgtagtttaatcttaatgtgttgtttccataagtgacatgaagtggatagattctcaattgttatgtccacttattcactaggtaaattttcagttttaatacttttctccttaccccttcctttgatcatttcatgtgaatattctatttgtatgatacactgtatttcaataaattccttgttgatgtacccttaaattgaagaaatttaagctgcaaaaccaaatctcattgtataagacttttttgaagtatcttgtatcgactacatatgtatttgaccctgtgggaggatggtacttttctttttaacttttattttaagttcaggggtacgtgtacaggtttgttataggtaaacttgtgtcacgggggtttgttgtaaagattaatttatcacccaggtgttggtacttttctttaaaaatactgtaagatcattgacgtatcagtctgcagcaactttcctacatgaggggaatacagcggtgtaatgaaacacatacttggtgcacactaggcgctctccggggctggtgcaccaaattctcctgtgagcatgctaccaagcatgctctgcttacctttgctggagtgtgtgctcatgcagccatactaattgaaaacaggcattcacaacaggatctgcctcacttgtgggatcaaaattttgaggcattgtagaactggcatatagaaaatctgaaaggagtttctgacccagatactgatatgtttgcagggtaattactgtgattggcagcttgataaattcccaattccctgtgggggcaatttccttatatatttgaatctgtcattaaaaatgtcattagtcttccttttcttcttttcaataagacactttactatgtaactatggtaacccagtgcttccattgacaattagagaaaataggtttggttttgaagttcaatagtgctctaaaggaacaagccatatattaaaaagtaagaaagaaaaaaaaactgttaattctgggttcttaatatcaggtgcagggtgtgtatactagaagcttgcaaagatacagtctcaccactctgggcattgaattttgattctaaacaagcatgagcactcaaaatacacagatattagtgattatgcttttaattttttggaagattaaaaaagataaatacaattacataaaaacatgtttataaatattagtcgtagaaagtttttggatccagtctattacttttacaattgaggagcctaactatggaagatcagtgttttgggatttttcttgcctaaattttgtctacgtgatagcaaaataaaatcagaagtt

SEQ ID NO: 10 - DNA, 1290 bp, Homo sapiens
atgggcttcttagaggaggagggcagatggaacctgtccttctccggcgccggctacctgggcgcccaccacgtgggcgccaccgaatgcctgcgccagcgagccccgcgcctcctccagggcgcccgccgcatctacggttcctcgtctggggcgctcaacgcagtcagcatcgtctgcggcaagtcggtcgacttctgctgctcccacctcctgggcatggttgggcagttggagcggctgagcctaagcatcctgcacccggcctacgcgcccatcgagcacgtcaagcagcagctgcaggatgctctgccccccgacgcccacgtcctggcctcccagcggctgggcatttcgctgacccgctggcctgacggacgcaacttcttggtcactgacttcgccacctgcgatgagctcatccaggccttggtctgcaccttatactttcctttctactgcgggctgatcccccccgagttcagaggggagcgctacatcgatggggctctgagcaacaacttgccctttgcagactgcccctccaccatcacggtgtcgcccttccatgggacagtggacatctgcccccagagcacctcccccaacctgcatgagctgaacgtcttcaacttcagcttccaaatctccactgagaacttcttcctggggctcatatgtctcataccccccagcctcgaggtagtggccgacaactgcagacaaggctacctggatgccctgaggttcctggagagacgtggactcaccaaggaaccagtgctatggacgctggtgtctaaggaacccccagccccggctgacggaaactgggatgctggctgtgaccaacgctggaaggggggcctgtctctcaactggaaagtgccccatgtgcaagtcaaggatgtacccaactttgagcagctctcaccagagctggaggctgcactgaagaaagcatgtacgagggatcccagccggtgggcccgcttctggcactcggggcctggacaggtgctgacgtacctgctgctaccctgcacactgcccttcgagtacatctacttccgcagcagaaggttggtggtgtggctgcccgatgtgccggcggacttgtggtggatgcagggcctgctgaggaacatggccctcgaggttttctccaggaccaaggcccagctccttgggcccatcagccctccggccactcgcgtcctggaaacaagccccctccaaccccagatagctcctcatagagaggagctcgggcccacccaccaggcctga

SEQ ID NO: 11 - protein, 429 aa, Homo sapiens
MGFLEEEGRWNLSFSGAGYLGAHHVGATECLRQRAPRLLQGARRIYGSSSGALNAVSIVCGKSVDFCCSHLLGMVGQLERLSLSILHPAYAPIEHVKQQLQDALPPDAHVLASQRLGISLTRWPDGRNFLVTDFATCDELIQALVCTLYFPFYCGLIPPEFRGERYIDGALSNNLPFADCPSTITVSPFHGTVDICPQSTSPNLHELNVFNFSFQISTENFFLGLICLIPPSLEVVADNCRQGYLDALRFLERRGLTKEPVLWTLVSKEPPAPADGNWDAGCDQRWKGGLSLNWKVPHVQVKDVPNFEQLSPELEAALKKACTRDPSRWARFWHSGPGQVLTYLLLPCTLPFEYIYFRSRRLVVWLPDVPADLWWMQGLLRNMALEVFSRTKAQLLGPISPPATRVLETSPLQPQIAPHREELGPTHQA

SEQ ID NO: 12 - DNA, 13086 bp, Homo sapiens
ttaaataaataaataaataacatagcggttgccattgattgtgcgccacttgattaaaacaatcaccgtagtaaccattgtttgtgtgcttatccagtgccgggcgctattccaaacacgttacttgcattaactctaatccttaccggtcctatggagtaagtaatgttattatccccacttgatggatgagaaaactgaggcacagatcagttaaattatccagggtcaccagccaagacattctggagccagaattggagcccagacagccctaatcactacaaacccaagttcttggagctttgcagtttataaagcactttccaatggattgattaggggaggagggaggaggtgggatgtttgagcaggctgtagtggtaggtgaggcttggagagcgggtgagccaggggaaggtgcagtggtgctggctggagcggaggctggccccgagacctctctttccctcaagtccctccctggaaccagcacagctgcggacttaagcctggtttgaccccaaagaccaggtgtcctgggtccgactctgccacttcctggctgtcaccatcctgggctgcgcctcagttgcctcatttgcgtaatgaggactggaataacatctgcccagactaccagagatcaggcaagatttggtcccactcccctgccgtgtcacccagaaatctatgacccgatgcggatccggggcgggcgcgtgggggcggcgccagcgtatacacagcgcgcgggcacttctggggcacacctggcgcacaactggagtgggggctgatcgcgacccggggacttcattggctggggcgcaggtgacgcagcccgggggcggcgcagggagcaccagcaccagccccagcccggctccctccagagcccacatttgggcccagccccgtccaaggctgcatcccggccctatcccgggtcacggagtgacccttcctcgtcccgatcaccccgcccggtccacccgccatgggcttcttagaggaggagggcagatggaacctgtccttctccggcgccggctacctgggcgcccaccacgtgggcgccaccgaatgcctgcgccagcgagccccgcgcctcctccagggcgcccgccgcatctacggttcctcgtctggggcgctcaacgcagtcagcatcgtctgcggcaagtcggtcggtgagtcccggaccgcgggggcgaaaaggggcggggacagcttaatgaaagcggatcgagcccagagacgctggtgcctggcgctgtgctctgcgccctctacccccggcacagcctcatttaatcccatgagaacaccgagtgctggggactaggtgacccccatttgcagaggagggatcgtggctcagagccagaaactctgtttcccaggtcggcggggcaaaaagtgaaaaggtgaacggatcatttgaatccggggccgagcccagaaattctgagctggagtggggaggaccccgctcccactgcacctaggagggcagtgggggtgtggagggactggcaggatccctgggcgctgaggtctccctttccctgcccccactgcagacttctgctgctcccacctcctgggcatggttgggcagttggagcggctgagcctaagcatcctgcacccggcctacgcgcccatcgagcacgtcaagcagcagctgcaggatgctctgccccccgacgcccacgtcctggcctcccagcggctgggcatttcgctgacccgctggcctgacggacgcaacttcttggtcactgacttcgccacctgcgatgagctcatccaggtgtggggcggggggtgcccagggcttggctggtgggcgggctccatctagtccagagtgacatccaggtacttccccgtcagacgtttctgcaggggcggaaggagggagggcgggtggacctgtttgtaggtgaggaaactgaggcccagagagggtcggcagggtgggtctggagactttccaggttgctgcacacagaagggtctggctggggcccggcagagtcaggctgctgagtcagggcccccacagggactgctctttccagagggcagctggctagccccagcctccaccccactccatcctcccgccgctttttggcggtgggttgacatttattgaacacctattatgtgccagtcactattctaggcactgtgactgtaacagggagcaaaacagacagaaacccttgccttcctggggcttctatgctcagtggttctggggggcaggcggtatcgataatgaactcatctccaagtaaggaggtaaggaaccatccagtattgaaatgaaataggatgtctaaaaaagatgcttcacatacaccctgacactattatttgccaatagtgtgtacgcgcacgctcaagtgcaggcacacagtgtgacaaccaaaaatgtctctgacattgttaggtgtcctgtaggggagaaaatcactcctagttgagaaccaccaacccagagcttgcaaacttttgaatataggactttgtctcatggaacccttggaaacgtcccaactctctctgggaggtgcagaacccatcctggccctgaccagatggagatgcaggtgacagttttgttcccacagactgataacagtcacggcagctaatgtgtactgagcacctactatgtgccaggccccatcccaactgctacacaggcaaggagtctgggccactctttggggtccccggctggcttcctcaggagcctgcacctggcctccttccctgcacacccacagaaagaggaggtgggtgtcaagacagaggtgctccaatccctgcttcccttggagagcttggctgggaggtgatgcctcagctccctcatctggacgtgggactgatgacacctcctttgcaggctgttgggattagaaaccgtgcctaggaaggttctgaatgcaagaagcatgcacagttgtgttcctgctgactcttctcttcctaggccttggtctgcaccttatactttcctttctactgcgggctgatcccccccgagttcagaggggaggtgagtgcactctggaaccccgttctgtgctctgggcagcctggggtgcacaccgtggagaggtgcttgggtggaggaaaccagcgctccttgagcacctactgtgtgccaggccctccccctgctcctggggggcgttcagcctcctgggctggagtggccctggtgactcagggacagctggagggtcaccggctgcagccagtgtgggaggccctctgagagggcagcaccacccacctgctccccacaaattgcagcgctacatcgatggggctctgagcaacaacttgccctttgcagactgcccctccaccatcacggtgtcgcccttccatgggacagtggacatctgcccccagagcacctcccccaacctgcatgagctgaacgtcttcaacttcagcttccaaatctccactgagaacttcttcctggggctcatatgtctcataccccccagcctcgaggtaggtctggccctgtgatggtgttagaggcttggccaaggtcaagggccccgtgagtcagactctgtgtgctcctaacccccactgccccccgagcctgaaggcagttacaacacatgtcccgagtctcacagccaaacacctgaatcctaagcttggggttcagttgtgtccttactgttcttgctgccctgagccagtgatgaatgaggactggccccatgtcaatgggacacactcacgccaactctgcttagctggcctttgagcagggcctttgcctgtgctcctctgggccagggtctgtaggcaggtaccgttatcatttccatttctcaggggaggatgctatggttcagagaggttaagcaacttgttcaaagtcacacagactggaagcagcaaaaccaggattcagacctagagctggctcaggcccatgctcttgaccaccgtccttgactgtggccaaattgaccatccattgtagaggcaaggcacaaatgcaaagaaaaaaatgaaggaagcgtttttttaccccttggtatttctggaaaacagataatttgacaactattgaaatatactcctcctggccgggtgtggtggctcatgcctataatcccagcactttgggaggccaaggctggcagatcacttaaggtcaggagtttaagaccaacctggccaatatggtgaaaccctgtgtctactaaaaatacaaaaattagctgggcatggtggcgcatgcctgtaattccagctactcaggaagctgaagcaagagaattgcttgaacctgggaggtggagattgcagtgagccgagatagtgccattacactctagcctgggtgacagagtgagactccatctcaaaaaaaaaaaaaaaaaaaaaaaaaagaaggaaagaaagaaacacacacacacacacacacacacacacacacacacacactcctctgccccatccacatcctggctcttccttgtcccttctacctctcaggtttgtgtagtgctttctaagtcacgagacaaagtcatgttcatttactccggtactccccacacccctgggcgggcaggaaaccccagccccattttctagataaggaagctgaggcactcagagagttggcactgccatgctgaagtctcacagcaaggatgtgagggggacagcaccctttgccaccagtgggactttaatcagacaggcaagtggggctcctggttcctcagcccacctcagtctggtactggaagtaaacatcgaaggggcaaagggaacccccatccactgccctacaaggggccagacaaagtcccagccatcccaggccttgttatcccattttccagagacagaaactgaggcaagagaggggaggggctggggcaagagcagccagggtgtctcaggggggagatgcaatggtcctgcagggacagaactagccaagcagatctgttctgcctgcaggcctctggcccctcctgcccagtgctaaggtcctgcccaggctcctgccctgagccctgttgaatggtcacttggggagaataggctgggctgggactatttccagttgaagataagtctctggcctgtgacatccactcgaggggtaggtccaggccttgcccatctcacccttgccctgtgtgttggcaggtagtggccgacaactgcagacaaggctacctggatgccctgaggttcctggagagacgtggtaggtggcagtcccacagtggctggggatcatgtcccacatggtgccaggtagaccctgcctcttgggcctttgagctgctagggacatgggctaactcagtctcttcctcatcatctgctctgtgctggacctgagctgagtactggggactcagagaagaatcacatacaagttccactgttgagcagcccgcagcctggtacagggggacagactcagagagagacaacccagggagatgggtgtagtgacagagggatgctcaaggctgtgggagtccagaggaggcattggactcagcctgggggttgggggtaggcaaggagggcttcctggaggcagtgacagataagctgggttttgactagttatgaagacaccagcaaaattgaagattccagcaaaagaagagggcattctaggcagagggaaaaacaagagagaagggacagaggcaggcaccttgagggcacataactcaatatggcaggaggggtgtgtgtgtgtgtgtgtgtgttcatggggcagtgggtgtgccagagaggtctgggctggagagcctgaacatcatccacaggacagagctcttcaaattggtgcaatagacattcagacacaggccacctggctccagaactcatgtgctcaagtcctacgttacaaccaaaaacacacaatcaggaggcatttggcgagcgcttaatatgtgcccagacgtgagcttaagctttatgtatttatcttgtttaattctcagagtaactgtataaagcaggtcttacttttccttttttacagagagggaaactgaggcaaagggcattgggtcacatagcttgcaggtggtggagccagaattcgaactctggattgtgtctttggggtctgggctctgatgagccactcgaaagtgggcagggagcagggcagagcagggccaggggaaggcagaagggagtcatccggaataagctgtgggtccgtcccacagcacagtggatccccagaactgggtttgggctctgagggatgtgggcagcatggggcgccttggggaggctcagatgcttgacttatcctctcccttcccctgacccaggactcaccaaggaaccagtgctatggacgctggtgtctaaggaacccccagccccggctgacggaaactgggatgctggctgtgaccaacgctggaaggggggcctgtctctcaactggaaagtgccccatgtgcaagtcaaggatgtacccaactttgagcagctctcaccagagctggaggctggtagggactctacctgcatttgctcacctaccagggccctgcactcaggggcccgggctcagggaccctgccttgaaggcactcgtggtctggaagaggacaggactcatttactgagcacctactaaatgccaagtgtggtactatgggcttttcacatgtggcatcgttcattcttccccacaaccctgggcctaggcattattcgtacccacttcactgatgaggaaaccaaacctcagagaaaggaagggactcaccaagggctcagagcctgcaactaggggatttgaacccaagcctgtgcctgcgaaagccaggtctttgcccactgccccggaaagggcgggagtgttgacagacataggagaggcacacacagagggttctggattccccagatgaaaatgaactcatttcatccttccaacaaccatctgaggatagtacagaattgttctcattgtacagatgaggaaaccatagcacagagaagttaagcgacttgcccaaggtcacacagtagcaaatggcagaactaggggttctactcggaaccatttagtctgcccgcccgctgtgtgactaaatgtcacaactgcaacaacaggaaagtacagagtcccacaggcacatgtaacgatggtggggggtgactttgagcagcatctaagtgaagctggaaaagagatgttggccaggccaagaggcagaagagcatcggggagagggcatggcacgtgcaaaggtcctggggttggtgagggacagccaggaggtccctgtggctgcggcatgagggacaggcagcagaggtgagcttgggaggctggcagggtgggcaggtggtctcaaaagctgtgggaggagcaagtgaggtgggcgggagggggtggtggacccagctgggtgggaagaggctgaaggggtgggaggctgggaggagagggatggcggtcgaggccagatcagggagagtggggtgggcaaggggatgaggaagggggcccctttccatgccggagaccacacgaggacgtgcagaaatgggcggggacctgtgcggaacccctgaggtccaagcttgggattctggaacccggcagtgtacctgtagccacacgggggcaccagagagctgcctcgggtggagcaccaactcctgggcgctgcgccctcgccatttgggaagggaattgtgaatctcatctctaatcctcacctgcctgcccagggaggaatgtcctgctgggatccccagtttatggagaaggaagctaggactcgtcctaggacactcggcaggtggatgtggagcagggtctctggcctgtgctatctgtgaatccttgtaatcctccgtctccagagggcagtggccagagtggacgtggtggcctgagctgtggcctgggctgtgtctggaggctgggatttgggctccggctctgtcccagcccagatgctggtcccttccactctggtcaggtcagtgaatagagcacccaggaaatggttgctgcggtcatagttgtggctgtggttattaataacactgtcgtgttactgttatgagagagtgtggtgagagcatctgtcccagcctagcaggccacagactttctagaggggcagaagaggtagaaacaactcaggagcctgagagtcctcaagtccatcctggccctgccacttcctgttgatggtccttaggcaagtcacttctccactctgaacctcagtctcctcatctacgagagggcatacaccccttgtctcacggacaacgatgagcgcagagctacctgctggcctgtggtgggggcttgctcagtgttgcttcttcatctgcagaaggtggagggggtgcagtctcttactgtgtgaaatgggggggctcaggttgctggcccacagacttcaggtgggcagggccatgccagggcttctcttgcagcactgaagaaagcatgtacgagggatcccagccggtgggcccgcttctggcactcggggcctggacaggtgctgacgtacctgctgctaccctgcacactgcccttcgagtacatctacttccgcagcagaaggtaaggctcggccagggggcctgggaagggcctggcttgtggcggagactggtgcccatggcaggaggggcggctggcacactgggtgggtagggctcggcggggtggggggagcccggctggtgtccccacactgtgtggtgttgactgcatgactcagagcctctgtgatctcctctgcaaaatgagactggtggagaagtggcatggtgggtaggattggaagtcgggggcttcctggtcggcctcctgggccctacactgtctccgtgacctttgaccattgctccacccatcagaatctcaggttggctgtgtggctgggtctgagtctcttgacctctctgggctacaggtttcttttctcttctcttttctttcttttcttttctttttttttttctgacagagtctcactctattgcccaggctggagtgcaatggcatgatcttggctcactgcaacctctgcctcccaggttcaagcgattctcctgcctcagcctcctaagtagctgggaccacaggtgcccaccgccacgcccggctaattgttttgtattttagtagaggcaggatttcaccttgttgcccaggctggtggcaaactcctgagctcaggcaatccgcccgcctcgccctcccaaagtgctgggatgacaggcatgagccaccgctacaggttccttttctgtaaaacaagccccttccaagccaggccggctgtggttaggctctcagtaacattctgacttgctggttaatgtgcccgtctgcatcagttgttcattcatcccaccagcagttgctgaggacctctcaggggccaaggcctggctgggggccatggaagcagccccgacgcagacccagctcctggcctggtggagctcacgacctacaaattatcacacactcacctgtaactacagcgccacctagaggttccctgggagaggtctacactggcttccagctgcacagctcaatctagggactctctggccagggaaggctgcccggaggagatgactcttaggctataatctgaaggatgagtcagcgtccaccaggcaaagaggacaaggagaatgttctcggcagatggaacagcatgtgcaaagatccagaggcaggagggagaaaggcccattcgaggagcaggaagggaggccagtaaagctggagcctcagggagtgtgccgtacacgacgcaggggaggctggtggggcagacgcactgagctctggagccagctgagcgtggtctttgtctggagagtggtggaaaggctgggaagggtttaagcagggaggtgtcatggggggacctgtattttcaaaaattctctggaagaaggtctatgatggtgaaccacaggtctctgtcctgctcaatgttgagttaatgactgggtcagccctgctgtttaaattgccagtggcccaaacccaggagagagctttctaccgacagattctcctcacgctgagctgtttacggaaaccacaagttgaaatccaaaagtataaatgtaaagtccagttttcagttaaaaaaagaaactcattgtgtgagccccaaaggggagcctgcccagaccacagggaagttcactcttggcatatttcacaatacatatgttcaacacataggttgcattaatagaggcaggaggtccagagcaagggaggtgacagtccagcttatgtcttgccgtcacaccacactctctggactgcatgcctagtttgaggtctggtgtaggggcccctccaagggtgctgtggtgcaggttgaggccctgcagtggccagtgtctgaggtgtccggccagggctttctcaggcctggccccacagggctgtggctctgcagacagactcatgctgtctctcctctgtgctgggataacacgcagttccaaggaccctgaatctggggctggctccatcatggaggcagtggggaaccacggggacatcttgagcaagagggggctctgtcagctgttttcggagggccagcagcttctgagaggagagtggacgaaggggcatggggtagggagggcaggggctagctgggagagaagagagagaaatggacggcgataaggaggacattaggaactatgggtgaggtgactgtcctcatttgacaggttccctgacctgtgcaggtcacccagctgatgggcagggaggctgggctgtagcctcaagccagcctgaccccaaaggtccaccctctccctccctgcgcttcctacttgtttcctctggagtcaaggaacctaaggcctggccacctctgccaagttcaaagtccattttggatctttccaaccctgcaggtgtccctgtggctctgacctgctctgcaacagtggccaacgctgctggcccttcttccctaacctgggcagccctcaggagcagggctcccggctgtgctctgggcctatggggagggtgggcttggtggggccacaccccaggctcacctgggcaaagctgacctgccccttcctccaggttggtggtgtggctgcccgatgtgccggcggacttgtggtggatgcagggcctgctgaggaacatggccctcgaggttttctccaggaccaaggcccagctccttgggcccatcaggtgaggtgggcagagggcagggatggagagggggtggctgtgctggggggaacgcaccagctgcttccctggaccctgggtaggccggccatgccatctgcctgcccacctgcttacctgcgtccctctggcctactcatgcagccattggcacgtgtgttggtcactgctgtgcgcccgacctgtgctagacgctctatgacagtcaccttggtcagtctgcatgacctgccaggtgcagtcactgggccacccatgggacaggtgcctcttgcggcttcaccagctccctgccagtcctcctgccattgttccagacgtccccacgtgaccttggacgaggctggccctgtgtgtggtctggcctgcagtggcctggtccctcttctctgaccagccctccagacttggggggtcctgtgtggccctcccccacccactcgacagttatctgtttagcagggacatcagtgcagttctctggggagtggccatcccaggacctccctgacacttccagctcagccacactgaggtccacatgtcctccctcagagcaggatttgggggaagagtgacctgggttcactgtggctgcgcctgcacccacgtggtttcacctacccttcccgagccccaggcatttacccgtggcttctcctccctccacagccctccggccactcgcgtcctggaaacaagccccctccaaccccagatagctcctcatagagaggagctcgggcccacccaccaggcctgagggggccgagcagggccagcccagtgactggtgtcccttgattcctgctctggcctttgtcctgtgtgcccacatctgccccagcgggaagaggccctgccaatctccgtggagaccctgcagcttggagcccgttccttggggcgcagcctgtcagcatgtccccggtcactgtgtacccaggtcacctgccctccctcttgccccgtctcatcagggagagaaaggaggacagccacagagacacatgtcttactttcagaatttccacggggggactaggagggcagcattgctggggagaggggctcctgttctgccgtggagctgcccacggagcacctcacagaagattgagaaagtgcctgatgcccgcgtcagagaccagaggagccaactgagatgatggggtgggggtgctccccccagactttctcagtcctgcagcggcccttgggggtgacccccggcatctaccccactcaggaaaccttgacttctgaattcctatcaggtccagagggtggaaggccctggcaggaagcaggaggaggaggagaggggggtccaggtcttttcaggatcccagctgtgggtgcttctgttgggcccagtggcccctgctatccccacagcctcattcctgcagaaggtttgtgacgtagctgtgcctgtacctggggcagagaggatgccagggacaccaggcctctggcctctccacgagtcactgctctccccattggcagcacgggacccatgctgtgcccccggccaggccctgaggaaatgctcagcctcagtggagatggggaggttagagagatggaggcgtgagtgagaacgggaagcaaaggaatacacgtgggaactaaagagaaaaatgaccagcctgggcagcatagcaagaccctgtctctaaaaaaaataataaaaacttagctgggcatggtggtgcatgcctgtggtcctggctacttgggaggctgaggatggagga

SEQ ID NO: 13 - DNA, 3007 bp, Mus musculus
ggagctgaactgcagcgccgcccggagcttcaagcaccatgtatgacccagagcgccgctggagcctgtcgtttgcaggctgcggcttcctgggcttctaccacgtcggggctacgctatgtctgagcgagcgcgccccgcacctcctccgcgatgcgcgcactttctttggctgctcggccggtgcactgcacgcggtcaccttcgtgtgcagtctccctctcggccgtataatggagatcctcatggacctcgtgcggaaagccaggagccgcaacatcggcaccctccacccgttcttcaacattaacaagtgcatcagagacgggctccaggagagcctcccagacaatgtccaccaggtcatttctggcaaggttcacatctcactcaccagggtgtcggatggggagaacgtgctggtgtctgagttccattccaaagacgaagtcgtggatgccctggtgtgttcctgcttcattcccctcttctctggcctaatccctccttccttccgaggcgagcggtacgtggacggaggagtgagcgacaacgtccctgtgctggatgccaaaaccaccatcacggtgtcacctttctacggtgagcatgacatctgccccaaagtcaagtccaccaacttcttccacgtgaatatcaccaacctcagcctccgcctctgcactgggaacctccaacttctgaccagagcgctcttcccgtctgatgtgaaggtgatgggagagctgtgctatcaagggtacctggacgccttccggttcctggaggagaatggcatctgtaacgggccacagcgcagcctgagtctgtccttggtggcgccagaagcctgcttggaaaatggcaaacttgtgggagacaaggtgccagtcagcctatgctttacagatgagaacatctgggagacactgtcccccgagctcagcacagctctgagtgaagcgattaaggacagggagggctacctgagcaaagtctgcaacctcctgcccgtcaggatcctgtcctacatcatgctgccctgcagtctgcccgtggagtcggctatcgctgcagtccacaggctggtgacatggctccctgatatccaggatgatatccagtggctacaatgggcgacatcccaggtttgtgcccgaatgacgatgtgcctgctcccctctaccagatcccgagcatccaaggatgaccatcgaatgctcaagcatggccatcacccatctccccacaagcctcaaggcaactctgctggtttgtaaattgctggtctccgtgcttccagtgaacttggacattcttctcatggttggtccaggagaggccaaagctgagggcaccctgccttccacccccagtccagcttgaccttttatctggagcaacagtgtctagatgatgggtgggtgaggggtgctatactgtctgtccctctgggaagggttctgttacttttggaggcagctaggaagtttctctgtgcagctgccccctggtgctgtgtggtgacctcattgcctgtgaccccaggatcacaggatctgggctaaagtggtagtccatagaaaccaaagacaatgatttggtgtttagaaagctactcttggtctgggtgaagtctggtgcttaagggctatcacaaagagcgtgtcaaaccatctctcagcctgtgagtcagtggggagcccaagggcatcagtgtttggaaactggaatccaaaccgggcaatctcggaaggaaactgtttaggaattgtgatgggacgggccgtggctgtctctgaaaagggcctgccagataacttattacttttaaggacacctttggctcttactactttataaagcattttatataaacacaccagggagtgcatggtgaactacacgtatgatcagttaagtggggctagaattaggtagggagagcatcggacctctgcctcctcaacctcaacttgcttgctttctccactggctccaaatctttgtatagtcatcagccatgaccacctctctccctccccatctactaccagcagcgttaatgggaataagtacccacttctctcaggtgtactatacagctgtgggtgtggtgtgtgtttcctgtaattcacactttagaaaggaaacaagcaaacaaaagaaaccaggtgctgcccatagtcctaagtgtagacagtgaaggtgtgtgtctcccatgcctgagtctcctggaggcctagtgagctccaggttcatgcaagcacatcaggaggaatcatataatctcagcacggttgatccagatgggataagaaaggactctcggagagagaatgtggttctagagacaaagtgtctaggctacacagaagataagactgtcccaaggaaagaaaagaaaccaggaactagggtgcagctcagttgtcagaggaattctctaggcttgaagcccagagtccaatctcagcaccttataaactgtggagtgacaggcagtgacatcggcctgtaatcccaacactcaagcagtagaggcaagaggatcataagttcaaggtcttccttggctatttagggagttggaggttagctctggctacatgagaccctgtctcaaaaaaaaaaaaaaaaaaaaagtagaaacttctgccttgctttgagctgcccctttctggacgtttctcatcagtagagaatattcctgccaccctatcagacaaaactcccactggtttggagtctctccattctcaggaacacctcaggagtcagacagtgagcagcagggagcaattttttgacttgtaagccccttaccaagcctggttcatttgtttattaaaagcaggtgtgggtgaatttatgcaaatgagtatgcaaactagtggaacagcagaaggattgaatggatacaccaaaaataaccacaactgtttaagggaaaagggtccataataaatgtggggaacaaaaaacaaataaatgtgattttttttag

SEQ ID NO: 14 - protein, 413 aa, Mus musculus
MYDPERRWSLSFAGCGFLGFYHVGATLCLSERAPHLLRDARTFFGCSAGALHAVTFVCSLPLGRIMEILMDLVRKARSRNIGTLHPFFNINKCIRDGLQESLPDNVHQVISGKVHISLTRVSDGENVLVSEFHSKDEVVDALVCSCFIPLFSGLIPPSFRGERYVDGGVSDNVPVLDAKTTITVSPFYGEHDICPKVKSTNFFHVNITNLSLRLCTGNLQLLTRALFPSDVKVMGELCYQGYLDAFRFLEENGICNGPQRSLSLSLVAPEACLENGKLVGDKVPVSLCFTDENIWETLSPELSTALSEAIKDREGYLSKVCNLLPVRILSYIMLPCSLPVESAIAAVHRLVTWLPDIQDDIQWLQWATSQVCARMTMCLLPSTRSRASKDDHRMLKHGHHPSPHKPQGNSAGL

SEQ ID NO: 15 - DNA, 23678 bp, Mus musculus
tagctcccagctttccttactcctgccttcattggcaccggccaatcacatattttatttataagtgacacttgtacatggaaacttcaggtcctccctgcctggtgttgtcttcctgtagtcttagttacttaggaggctgtggcaaaggggccagaggctgaagtccaggctgagtcactgaaggaggcagctggttaaaagaaaaaggaacccagggcagctcagtggtagagcctttgcctagcatgcaggagggccgaggttcagttcttagtactgtaaaattaaacaaggcgctgaaaggatggttcagcggtagaggccacaactactcttacagagcaccagagtggagtccaagaatccacgcgatggctcacaatcctctgcaactccagttctaggaggtttaacgtcctcttatggcctccttggtcactgcacacccatggtgcatagacatacatgcagacaaaatactcataaacatgggggcggggagcctaagtatctttttaagaggaaggaaggaaggaaggaaggaaggaaggaaggaaggaagggagggagggagggaaggagggagggaggaaggaagggaggaagaaaacctctctccatgaattaaaaacgaacagaaagcaaaataaataactgcaaaaacagacaccctacatgaaaataaaggaaagacagcatgtggatggtagcatctctcgtgtaataaagaaaaggagagagtgccaccggcgtgccatccagaacctgaaagaaaagggtattcaaagagccattctgcccagcaagcatccaagcctaggatttgggaaagcccacgcgggagggacatggggcggggcatgagtccagtacgcccattgggcctcccactggcttatttgcatggtcctagtggggcggggcctcagctgacgtcacccgaagacagcttaggcggctgcggctctttaagctcagagcagcaacaccgggagcagagctgaactgcagcgccgcccggagcttcaagcaccatgtatgacccagagcgccgctggagcctgtcgtttgcaggctgcggcttcctgggcttctaccacgtcggggctacgctatgtctgagcgagcgcgccccgcacctcctccgcgatgcgcgcactttctttggctgctcggccggtgcactgcacgcggtcaccttcgtgtgcagtctccctctcggtgcgtccacggccactacccaggccccgcgtgcggggaggggttgctacacctggggaatcggtaacactttccggggtgcccgaagaaacctgtccggagagctctcatccttcccggtgccgttgagcactcagctggagaccctgggcctgtcacctggcgtgggatttcccggggccggctggagcagacgcgctccgagcatcttcttcctgacccgctctggcgccctggtcctgtcagctgggtcatccctgagcagcagggaacgacgcaggtttgccgggtcctctggtccctgagccgcagaaggtttgtcctgtgtggctctggctagtgtcccatggctggtgccgccaggaagggcagaaggcacccagactagaacacccttagtggcagtccaatgctccggttgcctttagaggtggctggtgggggcggggtacgtggattagagaaatatttggaacactccctagaagctggaatgggttcccagacagatccagttccactctctcctgtcttcactagcctagataagcggcccttcccagatcgatgtcactgacattcagggcatttgaatcttggcctaattaaacatttgcccatgcacccatgagcgagaggaggcgagtactctcctctcccccactaaactactaggatgtgatgccttctcttcccagtgtcatagccggatgcttttggggttgtgggcagggcttggctaagtgcccacgggggggggggcggactgctgacacccaaaggtccccaggctgcatgacaatgtcccacatcaaccctctttgatctggggcctctttaacactttactctttgctctctgctggccacagccagcagggagagactttggcaggcaggggaaagcccctgctgatgagcggagctctgccattggatgggatcggtgagcagaccctgagccgatgagaatcagggattagaagacaagcaaatctggctgtgtgcctgcttgtctgggtttcccagtgaggagggagccaggagtaaagggcagtgagagtggcctctggaaagactagattagtccagtggccattgcacaccctgggcttcggcactaaaggcagttagttcttaaatggagggctctgagcccaatagcgatggatgattattgggacatggtttaaatgcattgactccaggaatcttaagtacacccactttcatatagagcaatggttctctcaacctgtgggtcacagcctctctgggggtctaatgaccctttcacaggagtcatcaaagaccattgggaaacagatacttatgattcataacagcaaaattacattatgaagtatcatcgaaaataattgtatatttgggggggcaaccacaacatgagggactgtattaaagagttccagcactgggaagattgagaaccaatgataaagtggggtgtttgtaactgctcagaagacagggacagcctggggctatcctgtcaggtcacagagagggcacaaaattacagattatctctggtgggagctgctaaggtgcattgcccagggcttggaacgggcacaggtgtttggggcatctgagcatttggctggacttgacagaggaggtaggtgcaattggctaccagtgtgtgagttcaccctcaagccaggctcagcaggctggcttggggtagggtttgctgggagtgagagttagagttggatcagaaaaggctgggatatgcctggcctcatggaaccaggattcatgttgtcataagtgctagggcagagtggattagtcaaagagggctggtctggagagtgagggcagatggactgtgaatactactgtgtacctactgtgcgatctgggaagtcttggcttagaggtttacacggagcctagccgcttacacagtcttttctggactgagcattttatagaccttattttacacaatcctttctgcctccagaagtgaatccatttttactctccttgaaaaaaggcagtcgctgggcgtggtggcgcacacctttaatcccagcactcgggaggcagaggcaggcggatttctgagttcaaggccagcctggtctacaaagtgagttccaggacagccagggctacacagagaaaccctgtctcaaaacccaaaaaaaaaaaaaaaaaaaaaagaaagaaagaaaaagaaaaaaggcagtctagcagctggagagatgtctcagtggttaagatcgcttactgctcttacagagaaccagcttggcaacttactgaccacctataattctagatcctgggaatgtgatgctgtcttctggcctctgcagggttttgcacacatgtggtgcccataagctcctacacacatatgcatatatatataatcgtttgtttgtttgtttgtttgttttaaattaaagtcaatctgtggtatagacagatggcagagtgctttcctctgtaaggggcaggggtgagatctacatttaggtccaggcctacattatttcttctgtgctgtctgatgttgagtccattgtgccaaggctggaatgctgatgtgttcgtggcttgtggctttatagacaggcaggtggaattctcatgagcatgcgtcaccggaagacctgccccagcaccatccaggaaataccttatacaacaggctattatatatatatatatatatatttttttttagttgctcccttgaataaagttagacaagggtgaaattgatttttctttttgagtcatacccagaatattaccactttgtaatgactgttaattactgttaatgagataatgtcacctagcaagcctttggaatatagggtgtcacctctagctaccttgaatgtacccagctctgagcaaaacacagggtgtgctttatggtatcttaattggccacagttcacgagttctgcgtctgcctggtttctgttcgccagtgtggtaccctgttctatgtgtttatatctgtcacaggccgtataatggagatcctcatggacctcgtgcggaaagccaggagccgcaacatcggcaccctccacccgttcttcaacattaacaagtgcatcagagacgggctccaggagagcctcccagacaatgtccaccaggtcatttctggcaaggttcacatctcactcaccagggtgtcggatggggagaacgtgctggtgtctgagttccattccaaagacgaagtcgtggatgtaagccctttgcctctctgggtgtggtcccatgaaagcacaacaacgcagggctgtcattgtctcactggactggggccatgggaatgctatacattcagggactgtgtctgttaacccggaagccttactatgccaggactcttgaattctcttcatttttccagtgctgggagaggagagagggccttggtcatatcaggcaaggacttgatggtagggctgcaccctaccacaagtttttttttttttccctcctttaagactcagagaactggttctgggtgtgccccacacctcaatatgcatgttatgcagtaatgttggctttttgaatgaggcttctccagggtggatccattcttatttgcttctatcaccagatcttgcacagagaagatcatttgtgccatatcaaaatgccacatttggcgcatacacacacacacacatatacatatatattgtagtatatatcctttctatagttcttgaatgaatggctgcatgtgactgaatgtgactccctgtgaggcatcctgtgtccagcatccctgcaggcagtctgggcctccatttgaatttctagtagcttctgaacagcggacacctatctgttctcagtggacagtgtgctgttcagaacagtggttctcacagtgtgctctagggaaccactgttctatatgacccctaactggcttcaggagagctctgagggagggcccaggtccgtgcttcctgtaatgcctgagtgagatgtgtgattccagctccctatgccaccaagtcaacggtgtggtcaacctgggggtcaaggacagcaagattgaactgtgcgattagtaaagctaaggacctgccattgctgcaaagacttatgagcctcaaatacaagcgtgagggtcattcattcactcagggattaaaaaaataaaatgggctggagagatggctcagtggtttaagaacactgaccactcttccacaggtcccgagttcaattcccagccaccacattgtggctcacaaccatctgtaatgggatctgatacactcttctggcgtgtgtctgaagacagctacagtgtactcatataaataaaaataaataatatttaaaaacaaaaacaacctactgtcacccaggaggcagaggcaggcagatctctgtgagttcgattgccagcctggtctatagagcaagttccaggatagccagggctacaagaagaaacactgtcttggaaaaaacaaaacaaccaaacaacaatccccagccccccgtgcccccccccgcccgcaaaaaaacccctactgtgtgcagagaagcatctcacttacactgctcacttgggaggcgctgtgccctgtgactgactactcccttttttctcacaggccctggtgtgttcctgcttcattcccctcttctctggcctaatccctccttccttccgaggcgaggtaagtgttgggggtcgttgggcagctgagtgtttggggctgctatttgtaatgagctggggtcaggtccccatgactgtatctaactgcagagctgggatctgagtcttgaggacagaagccatacatccccagcagactgaccacagacttatacccccaggagtttcctcacctcctgcctgctgtctgttggatacactggcctggttcccttaatctggtcctgagcaccctagggagcctcctggtggcctaattaaagtgtaagatgcttaggaattaaattactcttgttaggtagttgattttcatgagaatacaggctagctgtctatttcttttttaatcttttagacagggtattactatgtgactttggctagaattctctaggtagaccaggctggccttaaactcacagaacatttgcctctgcttcactctgccacctaagtactaagactaaaagacaagtgtcatcacacccaagagtgaaacaatctacattttcttttgttgttgttgttttatgtttcttgataaatatgaggatatgcctgagtgtacaaagaccctagcgattaagcacatctttgatcttgatactggtccccagtcttcttgtgatgtaatttaggctaagtggaagggactcggagcattatggcgatgtccctacactccctagaatgcacgcctagattttcctctagacaggagagtcaagagttcaaggccagccttagcttcataagactctatcatgacacagtgactttcacaggtaatcccagcaatcagggactgagactggagggtcatgagttcaaggccagcccgtaaccccctctccccaaaagaaaagtattgccgggcactggtggcgcatgcctttaattccagcacttgggaggcagaggcaggtggatttctgagtttgaggccagcctggtctacagagtgagttccaggacagccagggctatacagagaaaccctgtctcaaaaatccaaacaccaaaccaaacaacaacaacaaaaacaaacaaaaaaaaaacccaaaaaacaaaagaaaagtattacaagaaaacaggctcaattgaatatcttgtcctagatctccaaagggagtctggttttgaacctatggccatgtgtctccttggtgcagggttcatatcacatactatccccagtacatgtaacacccttcgtttatatggcccctttatgcatacagtagccactcacctatgggtgaccacccggcattaggatgtaagaagcacaccatggagtggactctcatctccatgtgtctttgcctggcacttttgcatggggggcatcctttgcaacagtacccgtaaccttgaaagtcaggaagaaactcctagaagggtggttcccagaccaccatattggctgcagtgtcttgtaaaaacacttaagcacggcctcctccctttccccttccccacagcggtacgtggacggaggagtgagcgacaacgtccctgtgctggatgccaaaaccaccatcacggtgtcacctttctacggtgagcatgacatctgccccaaagtcaagtccaccaacttcttccacgtgaatatcaccaacctcagcctccgcctctgcactgggaacctccaacttctgaccagagcgctcttcccgtctgatgtgaaggtgagccaggtgctagggtgccactctctgcccctacccccagatagctggggataccagttagtccctccatgagcagacagaaatagattatgccatgatggtgacttagaaaaggttgctatcgtaaagctgagtaatcacgccatagcctttaccaggggtcacacggacagaccccgagttcagactcaccttctagggattcggggctaagaagtcaggggtaatgaacggtgtctaagtttcttttcctgttgttaagattttctttttaaaaaaatcctgacaagggaaagttttctgaaaaaaaaaaaagaaaatttattcaggctcacagttcaaggtacagtccacgatagcagggaagccagttgtcgggagcttgtagcaagcagttggtcacatggagcccagaatcaggaagcagggtgtgatggatgtgagctacagcccagctccctttcccacaacatgtaaaatccaggatgccggccagagagcggagccactcacagtgggtgggtcttcctgccccagttaacgccatcatgataaactcccacaggcatgcttagaggtccatctcctaggtgaggctagatggtatcaagtcaacagttggcactacccacagaagggtaggtgggggtggtatatacctgtattccagcacccaggagactgaggcggggggatagagagcttgagttgagtctgggctttctagaaagacccttccttacaaaacaaaaaacaaataagcaaattgtggatggagagatggctcggcagttaagaacaccggctactctttcagaggtccagcacccacatggcagctcacaacaataataggatcagacaccctcttctggcatgcagacatacaagtaggcagagcactcatacataaaatacataataagtaaagttttaaaaagcttttaaaaaattaactaggaagatggcttagaggtgaagaacattctctgttctcgtagacgacttgaatttgattcctaatacccataccggtacaggactgcttgtaacttgaacactaatgcctgtggcttctgtgaccacctgtatacacatgaacatacctatatatgctgggcagtggtggcgcacgcctttaaccccagcacttgggggtgggggtggaggcagaggcaggaagatctctgtgaatttgaggccagcctggtctacagagctagttccaggacagctagggctacacagagaatctctgtctcaaaaacaaaacaaaacaaaacccaaaacaaacaaacaaacaaaaaagtaaaacaacccccttcagagaaaatacctatataaattaaataaataaaaatataaaaaataaatcttaaaaacacacccacaaacaaggtgggtttctttaatctcagctctcaggagacagaagcaactagatctttgtgagtttaagctagggctatagaaagagaccctgcctcaaaaacaaaaaccaacaaaaacctatgggcccttaagatggctcagtgtgtaaaggctgccaagcctgaggacccagctcatttttgggaacccacagagcaagagaaagtaacaatttcctataaattgtcctctgctctccatgtgcacacgtggacacacacatatgcacacgcacacacattaaatattttgaaataaaacagcagcatggccccgccatttggattttctttttttgtgttatcgaggatggaagccacggcctcaggtatgccaggcaaagagagagcattgaatcatgaccccaacactcactggtggattatgggtaaggcctcacagcccagccatgccttcatatcctcactggtgggttcttggcaagagctatactgatgagccatgcccccaattcagagctcttttaattttggttcctgacgtgattctgattagcaccgtgacaaggagtcaccacaatcgggggtttccttttctaatgggtcttttagtgcagcgaccgtgtctccacagagctattgagagaacctggccacctgtacccgagttgctcaccaatcatgatgctcaatgatgattggccagggacagggtagtactttgttagctacagtgacaaggctgagccccaggccgaggctgcatgaaacactgcttctgctggcagagtgtctgtagagaagacattggcgctgagacctggggaagcacagagttaagtggaccctctcagggggcctccaagtcttgaatgtcctaggtcctcagagggcatggcagttgcagccccagtggttcacctgcccaatttgctcactcacaggtgatgggagagctgtgctatcaagggtacctggacgccttccggttcctggaggagaatggtaggtcctggctggggggttactacagagtctttcagttctgtcatccagacttgtggccttcccagagcctcacttcacctgcttctcagccctgtggaataggaaacctttgcccatgttatagatggctggggaggagaggccagcagggcaggggcagccatcatgatacagagctgggatttgaacctgggaagcctgagagcaccgtgtcgtcatcccccccccccccgcacctccccccccccccgcaccccccccccctgcaggcaacttgcttctctttctttgagctctcttgcgagcccttagcataatgagctctttgacagggttcagggcagtttccagattagggtgtcaaaatctgctccctagccaaatgtgggttccacccagacgagacttcttcccagcaggtcctgagtcatccaagctcacctggcaaccaggttgcgctgtgcatacttagtcacacagtgggatggcattttgacatcatgactgtctcagttttcacgggcacatcctataatcactcggcgaggaaagctagcccttcttaaaacatgcccatgtcataagctgagcctggtagcatggtcctgtcgtctcaggtactggggaggccgatgcagggggattgcagtttaagacctagcttggttgccagcctgggctactttgcaatgtggggggtgggggtggggggagagctacttagtgagactttgtgtgtatgtgtgtgcgtgtgtgtgtgtgtatatatatgtgtgtgtgtatgtgtgtgtgcacgtctatgtacgtgtgtgttatatatatatgtgcatgtatgtatgtgtgttatatgtttgtgtgtgttgtgtatgtatgtatatatatagccatatacatacaaggctagggctatagcttagtgatagtgctagtgtgtgtgcctagtatatagaaggccctgggttcagtcctcaggcccatcaggaaaagaaaggaagagaagaaaaagagggaaaggagaagaaaacaaaaagaggattagtagaacatttgcccagcatgtgtggctagaagcacattatggagacagaggagatacttttgtaaggacagatgtgcattcgttacagaaagctgtatgctgaaccagatcactacagggccatggcaggagcctttgcagccttagagccagcctgagctatatagtgagaccttggctcaaaggaaagaaacagaaagcacaagagaagaagaaagacaaagtcaaagagagaaaaccagatgggaattggatgtgtctctgccggcctcttggtgcttgacagccacgtgacagagggttttatcttggaccccagagctggctctgccaagtaatggctgtgtgactctgaacgagcatcccgtgcttgtgagtcctcagctgacagttgtcttagttggtgttttattgctgtgaagaaacaccgtgaccatcgcaagtctcatgaaggaaaacatttaacttccttcggaaaacattctggttttctgaattggcttgtagttcagcggttcacagtccattatgtcatggcaggaagcatggccactgtccaggcagacatggtgctggagatggagaggaggtttctacgtctggatcaccaggcagcaaggagagactgtcacactgggcagtgcattgcttgcactgcccagtgtgacatattgcactgagcatatatgacctcaaagcccacctccgcgatgacatacttcccgtaaaaagggcactcctactccaacaaggccacactcgtgtcacacacctgagtctatggggggccgttcctattcaaaccaccacacaagtagacctgctgattccggtacctgttgaatggccactgactgctgttgttcgaggcctggagtttgtgctcatcctgtgagaggagagttattggttcactgtagtattttcctttaaagcttatgaacagttgcagagagactcccacacagctagagagtgagctagcgatctggggcggaaccacaaaccacactcccatctctgcctgactgcgcacaccgcactcctcatcctctctgttctgctgaggaaggccaagcacagatgctcgttcagagtcgcacagccattacttggcagagccagctctggtgtccaaggtgaaacccttaacccacaccctgcatgggatcatagcaagtgtgaagtgctgtgttggctcaccttccccgctgtctcccatctatctttcccttcacgggtatctctgtgttctctgcaggcatctgtaacgggccacagcgcagcctgagtctgtccttggtggcgccagaagcctgcttggaaaatggcaaacttgtgggagacaaggtgccagtcagcctatgctttacagatgagaacatctgggagacactgtcccccgagctcagcacaggtactgctattctggggcaggatgagctcagggcatgccagccagtgcaagggactgggccacctgctgtttgtcatgtattttatctcattggctgttaagtagattctaaatgttctttatgtctgtgtccctcgaaattcctgcactgacagcagaatggggggaggggtgtccttaggaggtgaagccaggggtccaaggctagcctcagctacatagtgaattctgatggtcagcctgggctacatgaactcctatctcaggaacaaaaataattctcatactgaaatccaagcccccagggggatgatgttgggaggtaggcaagcactggaacaactcctgagggcaaggccttccaaatgagattggtgcagtaaagagcaacccatccaggcggtggtggcgcacacttttagtcccagcgcctgagaggcagaggcaggggaatctctgatttcaaggccagcctggtctatgggatgatttctgggacagccagagttatatagagaaagcctgtcttagaaagaaagaaagaaagaaagaaagaaagaaagaaagaaagaaaggaaggaaggaagaaagaaagagggaggagggagggaggaagggagagagagagagagagagagagagagagagagagagagagagagaaagaagccaggcagtggtggcgcatgcctttaatcctagcatttggaaggcagagcaggcggatttctgagttccaggccagcctggtctacaaagtgagttccaggatagccagggctacacagagaaaccctgtctcaaaaaaaaaaagaaagaaagaaagaaagaaagaaagagagagagagaaagagagagagagagagagaaagagagagagaaagagagagagagaaagaagaggatcagtagatatcctccagtccttcagcccacagagatgcaatctgtgaccagggcctccaccagacaccagtgactgctggcaccttaacacttggccttccagcatccaagtctgtgaggaggccgtgttttttggttatagaagtggctgggtaacggtgtgacagtggtctgatccaccaagtcagacagactagtccacaaaccagcttcctgcttggcccaggacagattcagccatgaatgggacccagtgtctgccctccaggcagtagctgttcaattggaggtatccggaggtcatcaggggaagagtgagccagctgaggagtgcgggtgttgtcagggaagctgtgtgctccctcaaacttatctggaaacactgaggagtcatagacttaaacgtgtgccaggatcggggagaggggagagtctcaccgtgtagctcacactggtcacaaggatcccaagtgctgggattagaaccaccacacttggcttgagttggacctcatcaccctcactgtgggagagacaactcacaagccccagctgcctgccctttggctccaagcttcctgtatttatttcaaagcccatctgctcgaagggcttgaaccctggtgttctcaggagccattcggacttgtggttccaaactgccttttttccccccagctctgagtgaagcgattaaggacagggagggctacctgagcaaagtctgcaacctcctgcccgtcaggatcctgtcctacatcatgctgccctgcagtctgcccgtggagtcggctatcgctgcagtccacaggtgagcatcaaggcatggtgtgatagttcagtgagctgatgatagaggctttagcccaggctggccgcaggccacaagatggagcccctgggagaggcttccctttgtagaaattgaagataagattgcctgtgaggtcctgagtgtggagtagtgggctcatgtgtccagttcgcaggggggatttgtttgcatgttgacttccctcgttggaaacttcagagggtttggttttgttgttgctacttgggaattcggggtgtttgcacaggcttaccagttaagcaagttcaaacaggtaaacatctcagacgtgttttgagcatcacaccaacgctcagaaggcttctgacttttacactgggtgtggcggcacacacctgtggtcccagcactgtggatggaggctgaggcagaagagtcaggaattcaaacctatcctgggccacagagttagtcaaggccagcttgggctttgtatagagaccatgtctcataactaagatgtgtctggggacgtaactcagtggatagtttgcatggttcaatccctgtgctaccattaggaaggagaagggagggaaggagaaagagaggaagggaaagagggaaaggggggaaagagagagaagaaaggagagggggagaagaagagagggaaggagagagggagaggggaaaaacagaaagggcaagaggaagagagtgagagggagggaaggagagagggaagaagggggagaggaagggagaaaacaagaggggaagagagagtaggagggagggaaggagggagaggaaggaagggagaagaaggaagggaaggaaggagagaaaaagaggaaaggagaggggagagaaggagagagagaggaagaagaggaggaggaagaggaggaggaggaggaaagaaagaggagggtgtgttatctagaggctctggatgtaccttctgccctggactactcctggtctccttatcctgtccaatctgggcccatgcctcagggacatgacccagccctgcccttccatttctttgcaccctctttttcttggcactctcccttgtttttctgaagcgaagccttcctaaatccccagggaaacagcagcctgccagcctcctcagagcctcacctgttcctgcactggctgctgggcagctgctcgcaggcctgcgggcacagtctgtcctctgagagcctagctggggctccgtaagattatttattcacgacttccacctgctcttctcctctgtggcctgtaggttcttggtctttctttccaccgactgctactttctcagtcaaaggccctctctctcctggtttggggacatgaccatctgtaattaatgcctgtagtttcgtctttcctggggctgtttcttctctcttatgagactcttgtgagatgctttgcctggaactctgtagaccaggctgaccttagactcacagagatccacctggttctggcttctgagtgctggggttaaaggaatgcaccatcgttgcctgacatagaaacttctttattggggcagatgggaaatgagaaacagatgtcttcagggtctgagggaaagcctagagcagagaagccttcatcctgggagaaagaacagatcagcagcttatgaagttgttagggagtgcccaggggaatggtgaaggagaggtagtccccaagagcgccccagcggggccggtagatctggaagatggtgatccaggtggtgagaatgaatgatcaccaagaagaggaagcccacagctgagcgccaaacatagcctttgatttggctctgctctgtgtaggctggggcgaggaaagcctctctaaagaaccagaaaatgttgaccaaccaaagaaaaggcaggaacgcaaatccaccaagatagtacttccggcacaggttcaacttctcctcattggatacccgctccaagttcatagttgcgctggagcccggtggtcctaagggtctgcagagcaagacccaaatgacaggcgcaagatcacgagcaaccacgccccagtgcggtttatcccttctgcctgggattcctttgtgggagatttgcgggagatgccgcagttttcagtggagtttgttgtcgctttatttttttcttctcctgcccagcccccctcgcaccccaccctctccgccagttttcagtggagtttgttgtcgctttatttttttcttctcctgcccagcccccctcgcactccaccccctccgccgaaacttctctttcttacatatttcagccctttggctttttgcctccaatctgaagtcttctcagctttaccctccagttctacccttcggggttttaacattagcagcatgagtgagcatgctggtcctttcttctggttcctttacttgagcattctcagcttggtttgtgggtgttgggagaagtgtattgggggtgtgtctgtggaagtgggtgtgtctgtatgttgaggggtatttttacttttatttatgtctgtgtgcactgattggttgattgattttgtgtgtgtgtgtgtgtgtgtgcgcgcgcacgcacacacacacaaactgtttgagcagacaggctagccttgacttctagatcctcctgcttcaccctcttagcactaggataatagatatgcgcctctgtgcttaggtgagtgtcctgccgtcctgtctttctttcctggctatagcagggccacagcctatgtgtggaggtcagaggacaactctcagaaggagttggccctctccttccattttctgggtcctaaagatcagactcaagttgtgagacagggcagaaaacgtcattaccctgtgaaccatgtctgcctcctgtgtaagtacttctgagacagaattctagttctgtagcccaagctggcctagaacttactgtgtagaccagactgaccttcaactcaagtcggacctctcccctgcatgcagggtgtagggcttgattcacccctcccccatcccactcaaggtcttgtattaggggcatcagcctagctcccaggcagggagtagtgctgagtcagcctgcttctggaggaagaagggctcccttgtgagggtgggggtgggggggtgcttcctccttgtccttcctggaggattagacacaaggccaagtttgactgtagtaccagtgctttgtggtgtctggctccctcccctctggctgaggctatgggatctcatagccagatcagtagttcacacttgcagccagctcagctgcttagggggaagcccaggaggtctagaccagcctgtggagagagactccgtcctccctgctccccgtcactgtgctcccttagcatgggagcacctctggataagaggtgcagcaagctaggcttagcttgtcctcaccccaccccacgtcctcgaatttgtatcttagcacattgcacaggcccaacctttagtttctctatccaaaccctggggcagctttactgatccagtgttccccttcaacagcttccccaccgacgtcatgataaatcgaattaagtcgaagttgtaaaaagtcaggtttattggggcagctctgggtgggctcaccaatgtcacaggaaggggtcagcaaagttgtagaagttgccgggctgagctccagtagaggctgctgggggagtgggggaggggagtcgtgaagtgatggttattcatttggtaagtttagggccctgtaggtgggtctttaggtccagcggttactttggaatctggaactgggagctgactttgtccagtgttttagatgggcttttgcaagcatgggcgcctggtcagaagagaaagggggtagggtctcttggcccataccatcttaccggtgttccaaacttccagtctgccacccctcaagggaagttcttaccagcaacttacctcttgccagagtctacagagggttttggccccggtggctccagttggattcctagtggccctgcaccgctggcttctatggagcctaggcctcggccagcacttgccctcaacccttgagaaattaggtagacccaccccacgctgctgcctttcccgttcattcccaggagctttctggaaccttatatggcctgtgtgctttaagagatattctcagaggttcacctttgtgctgatatcccacatatgccttagggaaggtatcagattgcggttagaggcgatacaggcagctcactaggattaaggtcgcggttgtctagttggttgtgagccaccacttggttgctgggatttgaacttagaacctctgaaagagcggcccgtgctcttagccactgagccatctctccagcaatgggactgctttttattgcagtgttggctgctttttattgacctctttttttttttttttctggtttaatctttacagtgaacccttgtgtccatgtgatatttgtgcacatgtacatatatctgcatctgtgtttctatgtgaagtatacatgcatggtgtctgttatgaaaaataacacaggattaaagggccacctggagggcccatgccaaggtgtctccctgagaaatcccaccatgtgacagacctggctggtataagggaggtctattgtggggcaagggaggggagacagaaagggggagacagagacagggaggctggcagggaacatgtggggacagagaaagagagagagaataagaaaggggagatagagaaggggaccggggtaacatggtcctcttacaagctcccaggccacccacacctggtggcagctcaggtagcaatggaggcaggtgatgacctaagctgttgctaggtctctgttgttaggtccctgggaggaaaccagaatcacctgtaagccaatagtgtccttggcttccttcccacaggctggtgacatggctccctgatatccaggatgatatccagtggctacaatgggcgacatcccaggtttgtgcccgaatgacgatgtgcctgctcccctctaccaggtaaatacttgggcccagggtgtgtgggccagataggcatccctcccggttgttcccagagctcttagggtcagagcttgggtggtgacagccttaacaagccaggctcagccgcctgtccccagcatgccattaaagaaaccggtagcagagaaagcaggtttattcgaatataaaaaggttcaagcccccacccggttaatctttaagataccaacaggaggcttaagtttaaacagagttacacataaacagtctgaatcagggcgtggtcctgcccaccattgtctgggcttcaaggttccttctttctctccctagcatgagattcctgggacaatcccaattccttggcctccattgtatcaaagggctgaaaaccaaagggaaggcacagctgtctcttcagcatgcctcttctgccagaaccactgcaaggtttggtgctcaggctgtgcaaacattctagcaatgtttgactcagtgtcaagcaggtgacaaggaacatggtgctgtgtggggggaacccatggcccaggtgagggcttattggtgggtgaagctgtgggtgttcaggtggtggagaaggccttaagggatgggactgacacctcagcactgaaggcaggaggaagctgtggctctgggttgcacccctgcctggctccaccctctctggcatctgtagaagttacagctggttcttcctctcagccccatgctcccagaaataagactcagacccaaattatagttacaaataccttggccatatagctaggctcttctcagactagctcataacttaactcattaattttaacctccatcctgccacatggctggtggcctgtgctcaggtaccatgagtccagctcttcacatctttccggatgaatcttccataattctttctgcctcctggatgttccaccttctattccaccttttcctataggccatggttttgtttttgttttttttttccaaatttaatttaattaattaatttatttatttttggtttttcgagacagggtttctctgtatcgccctggctgtcctggaactcactatgtaagccaggctggcctcaaactcagaaatccgcctgcctctgcctcctgagtgctgggattaaaggcgtgcgcaaccatgcccggtgtggtttttttttttttttaattgacaggtggatgcatctatataatccataacatattctctctacaggtatctattaggttttgggtgaggtgtggagttctagggaactctgagagaaattcctggggagtaagtggtttatcaagttgattggaggagtttttaatgctatggacagacagacagaaggacaacagcatagtcggggctaccagggagttcaggccccggcatcggagatagaagcaggatggggtctttgaagagattctgagcccacacagcagaggagggactctctctttagagcttttgaggatgagggaggttgactgcaagagcctacagccaggctcgaggcaggcagggggtggggagcaggatgtaaaccccttcgatgctgacagactcacttctggggtaaaatattatgagatgcctgtcagtgtctgtgaagagacctgagcagagtctggattctgacatcaatcatgttcttacaatactgaagacctgagagcctgcaatcttggtttgtaaattgctggtctccgtgcttccagtgaacttggacattcttctcatggttggtccaggagaggccaaagctgagggcaccctgccttccacccccagtccagcttgaccttttatctggagcaacagtgtctagatgatgggtgggtgaggggtgctatactgtctgtccctctgggaagggttctgttacttttggaggcagctaggaagtttctctgtgcagctgccccctggtgctgtgtggtgacctcattgcctgtgaccccaggatcacaggatctgggctaaagtggtagtccatagaaaccaaagacaatgatttggtgtttagaaagctactcttggtctgggtgaagtctggtgcttaagggctatcacaaagagcgtgtcaaaccatctctcagcctgtgagtcagtggggagcccaagggcatcagtgtttggaaactggaatccaaaccgggcaatctcggaaggaaactgtttaggaattgtgatgggacgggccgtggctgtctctgaaaagggcctgccagataacttattacttttaaggacacctttggctcttactaatttataaagcattttatataaacacaccagggagtgcatggtgaactacacgtatgatcagttaagtggggctagaattaggtagggagagcatcggacctctgcctcctcaacctcaacttgcttgctttctccactggctccaaatctttgtatagtcatcagccatgaccacctctctccctccccatctactaccagcagcgttaatgggaataagtacccacttctctcaggtgtactatacagctgtgggtgtggtgtgtgtttcctgtaattcacactttagaaaggaaacaagcaaacaaaagaaaccaggtgctgcccatactcctaagtgtagacagtgaaggtgtgtgtctcccatgcctgagtctcctggaggcctagtgagctccaggttcatgcaagcacatcaggaggaatcatataatctcagcacggttgatccagatgggataagaaaggactctgggagagagaatgtggttctagagacaaagtgtctaggctacacagaagataagactgtcccaaggaaagaaaagaaaccaggaactagggtgcagctcagttgtcagaggacttctctaggcttgaagcccagagtccaatctcagcaccttataaactgtggagtgacaggcagtgacatcggcctgtaatcccaacactcaagcagtagaggcaagaggatcataagttcaaggtcttccttggctatttagggagttggaggttagctctggctacatgagaccctgtctcaaaaaaaaaaaaaaaaaaaagtagaaacttctgccttgctttgagctgcccctttctggacgtttctcatcagtagagaatattcctgccaccctatcagacaaaactcccactggtttggagtctctccattctcaggaacacctcaggagtcagacagtgagcagcagggagcaatgtcttgacttgtaagccccttagcaaggctggttcatttgtttattaaaagcaggtgtgggtgaatttatgcaaatgagtatgcaaactagtggaacagcagaaggattgaatggatacaccaaaaataaccacaactgtttaagggaaaagggtccataataaatgtggggaacaaaaaacaaataaatgtgattttttttagaaaaatgtattctctgtctcttctgcctctcctacaactaaaagggcctgtgggacagcttcctctataagtttgtgttcttccctgtaggcgtgcaggctgaatgtcctccacactgcaaaatgactgggttggtgctgtatagccccggctggtctggaactatgtagttcaggctcacctgctttttccattctcctgcctcagccttcccagtaccatgttcacacacaaccacatcagcttggaatgtgcatttctaggaagcgttgggaaaggacaatggcatagtctccagaaccactgagggaaaagtctggagcaaataactgtgacaaagggctgggtgcgcatgtgactctctctggccacaggtgaggccagccctgaagatgacaggagcactcaggcatccaacacacagggataacccgaacacaagcttggatcatgggcagtgttcaaggcttcaattttccccagtgtaacaccaacaagggggtagggcagggatcgtttactgatgaagaactacacttcccagcttcccctctgatggactacatttcccagtctcctcttgtgaactagtaagtcatttctacctctatagagggcttctcctgtggccattttggccacctgcatgtgtgggtgacaaaacgctccctcattgttaataaacatgatcctacctaaggtcagaccaaggcttgctctgtcttctctccctctacaactgctttttaaataatctaacagggagaacatatgagggctaaaggtatgttttaagtttaaattactgtgcttaagagacctatataaaaaaaaagcctctaacctgtaagtcccacttggcccaggacagataacttcctggattgctggaggctgttgtccacgtaagataacttctgcaaacaagcttggtggccccaactgcacaggatgtgatgaccacatataggtgagaggtatacttgcctccagtggatga

SEQ ID NO: 16 - DNA, 1961 bp, Mus musculus
acagcgtctccgcctccgccggcggagaccccaaggtatcgagactgcgggacccactgcccgcaggacatcgagtcacgatgttcccgagggagaccaagtggaacatctcattcgctggctgcggcttcctcggggtctaccacattggcgtggcctcctgcctccgtgagcacgcgcccttcctggtggccaacgccactcacatctacggagcctcggcaggggcgctcaccgccacagcgctggtcactggggcctgcctgggtgaagcaggtgccaacattattgaggtgtccaaggaggcccggaagcggttcctgggtcctctgcatccctccttcaacctggtgaagaccatccgtggctgtctactaaagaccctgcctgctgattgccatgagcgcgccaatggacgcctgggcatctccctgactcgtgtttcagacggagagaacgtcatcatatcccactttagctccaaggatgagctcatccaggccaatgtctgcagcacatttatcccggtgtactgtggcctcattcctcctaccctccaaggggtgcgctatgtggatggcggcatttcagacaacttgccactttatgagctgaagaataccatcacagtgtccccattctcaggcgagagtgacatctgccctcaggacagctccaccaacatccacgagcttcgcgtcaccaacaccagcatccagttcaaccttcgcaatctctaccgcctctcgaaggctctcttcccgccagagcccatggtcctccgagagatgtgcaaacagggctacagagatggacttcgattccttaggaggaatggcctactgaaccaacccaaccctttgctggcactgcccccagttgtcccccaggaagaggatgcagaggaagctgctgtggtggaggagagggctggagaggaggatcaattgcagccttatagaaaagatcgaattctagagcacctgcctgccagactcaatgaggccctgctggaggcctgtgtggaaccaaaggacctgatgaccaccctttccaacatgctaccagtgcgcctggcaacggccatgatggtgccctatactctgccgctggagagtgcagtgtccttcaccatccgcttgttggagtggctgcctgatgtccctgaagatatccggtggatgaaagagcagacgggtagcatctgccagtatctggtgatgagggccaagaggaaattgggtgaccatctgccttccagactgtctgagcaggtggaactgcgacgtgcccagtctctgccctctgtgccactgtcttgcgccacctacagtgaggccctacccaactgggtacgaaacaacctctcactgggggacgcgctggccaagtgggaagaatgccagcgtcagctactgctgggtctcttctgcaccaatgtggccttcccgccggatgccttgcgcatgcgcgcacctgccagccccactgccgcagatcctgccaccccacaggatccacctggcctcccgccttgctgagaatcaccattcccacatcgcccggctaccagccaagctccaagttgtcctgccccactaagaggagccccggggtggaacaagatcctgtctgccccggctctcccccttacatgctgtggaatgaggacataggaccctgcacagctgcaagtgggctttcgatgtgaaacctttcaccagccactcactatgctactcctggtggggagggatggggagtcgccctcccccggagcccacagagccctcccccgtcacgtcacctgtgccttactcctgcccaccaccttttcagtgcagggtcagtcttaagaactccacatctgctgctgctccctggtgtccaagtttccttgcagagtgtgtgaagaattatttatttttgccaaagcagatctaataaaagccacagctcag

SEQ ID NO: 17 - protein, 486 aa, Mus musculus
MFPRETKWNISFAGCGFLGVYHIGVASCLREHAPFLVANATHIYGASAGALTATALVTGACLGEAGANIIEVSKEARKRFLGPLHPSFNLVKTIRGCLLKTLPADCHERANGRLGISLTRVSDGENVIISHFSSKDELIQANVCSTFIPVYCGLIPPTLQGVRYVDGGISDNLPLYELKNTITVSPFSGESDICPQDSSTNIHELRVTNTSIQFNLRNLYRLSKALFPPEPMVLREMCKQGYRDGLRFLRRNGLLNQPNPLLALPPVVPQEEDAEEAAVVEERAGEEDQLQPYRKDRILEHLPARLNEALLEACVEPKDLMTTLSNMLPVRLATAMMVPYTLPLESAVSFTIRLLEWLPDVPEDIRWMKEQTGSICQYLVMRAKRKLGDHLPSRLSEQVELRRAQSLPSVPLSCATYSEALPNWVRNNLSLGDALAKWEECQRQLLLGLFCTNVAFPPDALRMRAPASPTAADPATPQDPPGLPPC

SEQ ID NO: 18 - DNA, 6884 bp, Mus musculus
taatttcattcttgctggttttgaagttctttgcggtgtatgtcaaggtcccgggtcacctgaataaaaggcactggctcagagatcttcaggttgaattgttgacaatacagttgtacttctgttgtgataggtctagagctaagctgggagtgagaatttctgggcaaaaagacactgtcacccataaacccaaccttgtttggggtggggtaaaacgagggctaggttttagcctgtccctcagctaagggaattctagtccggggaaaagggtgagagggtaacattacccaggcgcacaccacttcctggaacaatcagttgggttgaggcttttgttctgagaccaagtagaccccacaatcggaggagccacaggatcaggcagctaaaacccaagtgagcagctagggtatcgagggcccctatgatggtaagagcaggacttgagtctgtgacagtatttgctgggccagtgtcagtctgtttggcctgttggttttgggggctttagcctgagtggcccaggttttcaggcccatagggggtactatctgcgacttcgcccaggttctgtccagttcatctgtgtcctggtgggtatccctggagaaggaggagacacctgttcacaaactcctggtacccagatctgtacaaaacttgcacttctccatgtgctcggctctccctgagtccaggggctgcccaagctgtgggattgaagaagctgtgggattgaagaagctgtgggattgaagaagctgtggggagagagagaagctgaagcctgggaatcccctctccaggtctctcagatggctttgcccagcaggtgtcacgaaggggcggggcctcgggggcgcggccgttccctgcttgatccagttggatcgcgcgcccaggtcctgcgccctgccgaccaggccccgccctcaccccgcactaaaacacctcctcctctgcctcccggcacagcgtctccgcctccgccggcggagaccccaaggtatcgagactgcgggacccactgcccgcaggacatcgagtcacgatgttcccgagggagaccaagtggaacatctcattcgctggctgcggcttcctcggggtctaccacattggcgtggcctcctgcctccgtgagcacgcgcccttcctggtggccaacgccactcacatctacggagcctcggcaggggcgctcaccgccacagcgctggtcactggggcctgcctgggtgagccgagctgcgcggaacttgggcaggatggggtggggaccgcgcaggccggtggatgctctagcgtcccacgcctagccggcgtagggaagctgttccagtaactagttagtgcagtagaagcggtagagagggacatctctgcctgtattccggaactatgaccgaaagtccagttctgagctccggtccggggcgggttctggtttgtttttgttctggtttgtttttgttctaagagtgtctagggactggggtggggcctggcgcgctcttggctcatggcattctcttggtgcccatgtagcccgccttaaggggcacaaaggttggagaccagcaggtgccactcgactgttattggaaagagaaagtaaatcgtgggcggagctatgagcgagggagagaggaggggcattggtctccccgaactggctgccagcggtcactgtgtcctgaggctctgccgctacaaactgctgtgactctggggcctagtacaaggattcctcaaatgggcatctggacactgtgtcatggttcctaaaggggacctctgaacgcagctggagccagggctgggcataattgcttgctcggtccatggtgattgacagctgtagccagctgacccacttcaccgggtggccacacttctgtgctttgaagggtttcgggagtacagagatgggaaggggggctgggtctcacacttccggctaccacgtgggttgttgtttacagcacacaaagttcttcctccttaggagtcctgggaagtgctgcccatccccccgtcccataggtcaggcaaggtgacaagggcatcggcaggagattgtctagggttttctgcggtctctttcccatcacctttgccctgaggagggaagaggagaaggaatacagttctagttcttggtccctgaagttcaccctgtgatcttgcccgctggctctcatgggggctttcagaggagtgggtatagtgggtaggtggtctcaggacagacatgaggaccatacctgaattgagaatgagttagggatgccactcccacagaaccacttactaccaatactttgcacaggtgatcttgagcagctagaacaatgacaggttgcctactgtctgctgtaaacccctggtctttatgtcccatgcctgccaaggtgagcatcttggaggagggcatgggtcctcaggggggaaaacaaacaaaccctagattctcctctctctggccaccgtactattgttctgggtcatcatcaagggcctcctaagtgtcagagtgcttgcatttggcagaagtgagaccatgtcttcccaggaagtgggccctccgctgtaagctactgttcttgagggtgcacctcttgggctgagctgggcttcgtgggtctgcccacccacttagatctggccaagtaggtgatggttgaagtaggtcagagttgaccctccgtcttcccatgctaggccacgtgccataccatgttaggcacagtgactcatggggacaggcagttgggaaataccaggcacagggcaggttttaatcgcagctggccagctcctgctgtacgccatctccagggtggtggtctgccttcttctttaagtggcaaagtcccaattgtgagccagcccccagtgtgggcataggtgtcttcacagtcaatggaatgtcatccatctgagtatatcctgggtgtggagtcctggaggacagatggttactaagctgggaatgctgggaatgctgtccctgatcccggccctgctctcccaggtgaagcaggtgccaacattattgaggtgtccaaggaggcccggaagcggttcctgggtcctctgcatccctccttcaacctggtgaagaccatccgtggctgtctactaaagaccctgcctgctgattgccatgagcgcgccaatggacgcctgggcatctccctgactcgtgtttcagacggagagaacgtcatcatatcccactttagctccaaggatgagctcatccaggtaggcactgcagacaccgtgttggcggcggcagggagttaactcaaggcagagccttgtctactaccctgtgtcttgttcttcaggccaatgtctgcagcacatttatcccggtgtactgtggcctcattcctcctaccctccaaggggtggtaagtatctctgcttggccctgccctggaacgagaagcatcatgtactgtctctgggctgcaggaaaagggggtggaggagaaaggccactggggtgcctctctccatcttcagtccctcactgcctgttgcttgacttctcatgactcaagattcaggggagtcttggggaggctcagatttcctctggctggtgcccatctcaactccatctgtctggccatttgctgaggagtccccagtggccaatacgtagtgggccaaaggctcaagtggaaccgagtagggctgccaggacaccctgcagcccctcagtgtgtgactatgaatgtgtgttcttgcaaagcgctatgtggatggcggcatttcagacaacttgccactttatgagctgaagaataccatcacagtgtccccattctcaggcgagagtgacatctgccctcaggacagctccaccaacatccacgagcttcgcgtcaccaacaccagcatccagttcaaccttcgcaatctctaccgcctctcgaaggctctcttcccgccagagcccatggtgagcccgcacacacccagtcctgactaagccagcaccaggggagagaaccctggggacctctgagccctgggatgacagtggggcttttggatgctttccaaggttggcaggacttcaagccagggcaggttgttggtggaagtggcagttacagggactctacagcagtaattcaaccgcaccattcacactgatttatacctccgccacaggtcctccgagagatgtgcaaacagggctacagagatggacttcgattccttaggaggaatggtgtgtgggcagccctggggtggaagaagctggacctgcagtgccccctgctggccataacctctgatggcatctctctgaccccccccccccccccacaggcctactgaaccaacccaaccctttgctggcactgcccccagttgtcccccaggaagaggatgcagaggaagctgctgtggtggaggagagggctggagaggaggatcaattgcagccttatagaaaagatcgaattctagagcacctgcctgccagactcaatgagggtgcgccgccacggggagggaagctcagttaggaggcctaggaggcccccacttccctgattgactgccaccttcctctctgcagccctgctggaggcctgtgtggaaccaaaggacctgatgaccaccctttccaacatgctaccagtgcgcctggcaacggccatgatggtgccctatactctgccgctggagagtgcagtgtccttcaccatccggtgagggtggggaacggagtctgggggaggacacatcaaccggtacatcagggacagaggtcccggatcatccatgagcaatgggctccttgggagatttggggaggataggtcttcagaacctggcaagggagcaccagatcccagcctggtgtttctgagctcatgctctcggtgctcccttctcagcttgttggagtggctgcctgatgtccctgaagatatccggtggatgaaagagcagacgggtagcatctgccagtatctggtgatgagggccaagaggaaattgggtgaccatctgccttccaggtgagctgctctgaccgcctggccccctgggtcctgagtcttggaatggcagatctccactaagccttcctttcctccatgtctcctccccacctgcagactgtctgagcaggtggaactgcgacgtgcccagtctctgccctctgtgccactgtcttgcgccacctacagtgaggccctacccaactgggtacgaaacaacctctcactgggggacgcgctggccaagtgggaagaatgccagcgtcagctactgctgggtctcttctgcaccaatgtggccttcccgccggatgccttgcgcatgcgcgcacctgccagccccactgccgcagatcctgccaccccacaggatccacctggcctcccgccttgctgagaatcaccattcccacatcgcccggctaccagccaagctccaagttgtcctgccccactaagaggagccccggggtggaacaagatcctgtctgccccggctctcccccttacatgctgtggaatgaggacataggaccctgcacagctgcaagtgggctttcgatgtgaaacctttcaccagccactcactatgctactcctggtggggagggatggggagtcgccctcccccggagcccacagagccctcccccgtcacgtcacctgtgccttactcctgcccaccaccttttcagtgcagggtcagtcttaagaactccacatctgctgctgctccctggtgtccaagtttccttgcagagtgtgtgaagaattatttatttttgccaaagcagatctaataaaagccacagctcagcttctgccttcctcacttctgcatgttacccttgcctccgactgtggtcagcagacagagctttctcccagtgaactggtgtctcagtagctaacagttgggcttcacggggttctctgagtcctggcagctggggaccgtctcctaaggtcagtcctctggcaaggacacatgtaagctgctctgttctctctgaggcttgctgcctgtctcttgggccagcagatatgtttgggagtttgagccccttagtatcgataaaaaaactaaagtggtgggttagagttgctcagccgttgaacagctgtctaatatgcaccaggtccggattccatctcaagcactagggggaaaatgtttacaaaaggtgtcacccacacatccgctcctcatgtgacacagcgagtgagggcgtggatccctgtgaagggcgaagcttctggaactgcggtctggggacccgggaagattggttcctgacccatactggggcatcttgcccagaactttgtgcttggctcctctttcttggttgtcacatgctccactgccaggtgcatatgccctttcctgtactgtaaccatacctgcttcccgccagaaccttgggagagatcagaagtactgaggcaaagtagagttcaccatggtttcctgaagtatcctgtgctgtgtttgtgtgtgttgtaatagtcttggaggaaggggaactctgagtgggggtgagcttaactgtccagttggaggacttagcttagtcctccgagctcagcacccacttgctcctcccaaaggggtgggcttagctgtctatttgtagaacttagcttagtcctccgagctcagcacccacttgcccctcccaaaaggagcacagtggctgcggttgcagcccctagccaacaataacggttgttctggttatgggacctgggacagcaggactgggcgagccggccaggcaggtccctggcctaggtgccaggggccgccccttgtctccaggtcctgtcccatacagcaag

SEQ ID NO: 19 - DNA, 2172 bp, Mus musculus
acagttccttcctttgagctccagctgaggagtctgcagccccgcgtcccaagcaccctattatggacttcctggaggcagagggaggttggaacctgtccttctcaggctctggctacatgggcctctaccacgtgggtgtcacccagtgtctgcgacagcgagcgcctcgcctcatccagggtgcccgccgcttctatggctcctcctcaggggcactcaatgccatggccattgtctttggcaagtctgcagactttgcctgctccaatctcctggacttggtgaaacttgtggagcggctgagcctgggcatcttccatccggcctacggacccgctgaacacatcaggaaaaagctgtatgaaaatcttcctgacaactgccatattctagcctcccagaggctgggcatctccatgacccgatggcctgatggcaagaatttcatagtcaccgattttgccactcgggatgaattcatccaggctctgatctgcaccttgtatttacccttatattgtggggtgatccccccggcattcagaggccagcgcttcattgacggggctctgagcaacaacctgcccttctccgattgccccaccaccattaccgtctctcctttcaacgggacagtggacatctgtccccagaacatatcacatagtctttttgagctaactgccttcaatgccagtttccagatctctaccaggaacttctttagggggctcaaatctgtgttcccccccaagcctgaggtggtagctgaccattgccgacaaggttacctggacgccctgaggttcctggagagacggggacttaccaaggagccagtgctgtggtcgctggtatctaaggagcctccagccctcgtggaggggcccaggggcactggccatgaccagggccagaagactggcccaactgtcaggtgggacattcccaatgtgctagtcaaggatgtgcccaacttcgagctgctgtcaccggagctggaggcggcactgagaaaggcatgcaagagagacttctggacccgtgtccagtgttcagtgccagggaaggtgctggcctacctgctactgccctgtacactgcccttcgagtatgcctacttccggagcaggaggctgatggagtggctacccgaggctccagatgacttggattggatgaggagcatcctgaagagtaccaccctcgaggtctactccatggccaagtcatggcttctcaggttgggcagcccacctggcacccgagcagactccggtctcctccggcagcagcgggggacagctccttctgggaacaggcccttgaaccacaggttcgctgattgcgcactgggctaggccaacctcatatgtgaccctctgaactctgtccttgccctctggctgtagggactctggcaggtggcctctgtcaggttcagcccccaggatctctgagctcaactcctcactcaggaagcttgctgaattactggcaaaagacctcttcctgtccttgctaggccatgtgcttcttgtgtggacaggtggtttcctggcatggactgagagcccaccctctgcagctgttcctgggaccttgatctccaaggcctctctggatgcttccagcctactgtgtgctcaggccactgttctcttcttgtcagttttgtcagagaagaaaagacaacaagctgagacacacctcagttctggatcttcatgtgagagacacagccaggtgtctagcagaggtgtgttctgcactaggctggaactggagatggtgccccagggctctgatcatggtattgactgctctaactgggctcctttctctgacccacacttatctgagggattgatccctgtgatagtgaggatggaaggtggctggctggggaatccatgggggggggatcctgattatagatacttctgttagggataaagcccactgtcactgcaggctagggaagatttctgggtggcatacataccaaagcccctctggtctcttctggggtctcagttcttcctacccctgaagaaccaacacagtgcacctagccagactgtaggggacactcagaccaagtggagatgggactgcagagaaatggaggtgccagtgagagaagagattatgtaataaatgctgggc

SEQ ID NO: 20 - protein, 433 aa, Mus musculus
MDFLEAEGGWNLSFSGSGYMGLYHVGVTQCLRQRAPRLIQGARRFYGSSSGALNAMAIVFGKSADFACSNLLDLVKLVERLSLGIFHPAYGPAEHIRKKLYENLPDNCHILASQRLGISMTRWPDGKNFIVTDFATRDEFIQALICTLYLPLYCGVIPPAFRGQRFIDGALSNNLPFSDCPTTITVSPFNGTVDICPQNISHSLFELTAFNASFQISTRNFFRGLKSVFPPKPEVVADHCRQGYLDALRFLERRGLTKEPVLWSLVSKEPPALVEGPRGTGHDQGQKTGPTVRWDIPNVLVKDVPNFELLSPELEAALRKACKRDFWTRVQCSVPGKVLAYLLLPCTLPFEYAYFRSRRLMEWLPEAPDDLDWMRSILKSTTLEVYSMAKSWLLRLGSPPGTRADSGLLRQQRGTAPSGNRPLNHRFADCALG

SEQ ID NO: 21 - DNA, 12820 bp, Mus musculus
gtctccttccacctcccatcaccggggactccccagagtcacgccctctaggacacttcacctgcgacctttctaatagttacatttattatttttacaccacagagtgaggtttgcatttaagttgcaatgtgtttatcttagtgtgtgtgggcggtgtatgtgtggaggtcagagaacaacctacaggagccagtactttgctttggccatggatatgggggggggtagaactggggtggtcagggttggttgcgctgttatcggatgagccacgtggctggaaggcgagcctgtaggggggatgtttgcaccaggcagcatgcagtgtgatgctctctttccttattggttacttgcaagagtctcccatgctcagtgtgtgtcccgctgagttccactcaccccacttctttactggagtcctctttgctcactaccccacatactgctgctatcttcctcttcttggcatcctggggtgcacttcatttcccctgaccctcatggcatgtctggcttgacttccccatgatgacttggctgtgtagcacctatcatctcacctgctcagaaatatacatttgagagcctggtccatgccaggcgacttcccatcgtgactgcagagaggagagatcttgtgtgtttgaaggcactgttctatagcaacctttccttgcagagcctagatcccaggcctctcacctcccacttacggtgctggtccttgacctctggcctgaagattaggtgcagtccttcctgattgtgcctctttccctctttcaaaatgcagacacctggaggttaggtgagtgtaattgggaatcgtgtaggcttgtaatgtaatgcctggtgtacacgtggagagggtggctgcaccaacccctcacttgctagggccacagctgacagcctagggcggagccactctgctcagcctcccggagctccatccccatcccggcttagtcccatcacattggggccctagtgcacagttccttcctttgagctccagctgaggagtctgcagccccgcgtcccaagcaccctattatggacttcctggaggcagagggaggttggaacctgtccttctcaggctctggctacatgggcctctaccacgtgggtgtcacccagtgtctgcgacagcgagcgcctcgcctcatccagggtgcccgccgcttctatggctcctcctcaggggcactcaatgccatggccattgtctttggcaagtctgcaggtacttaggaagttctcagggtctaaggactcccgaagggcgctgagggatgtgtccctggggctttacagcctttgaatctgaggacccctgcagtcacttctatctctaaagatgggatcctggctttagatttggggtactacccctaaggtgttttggttttgtttttgttttgttttgtttttgagagttttggttaccaggactgggcagagatgttaaagaactgattcccaggcatccctgagagaagtggacttttggtgggtacgggtggccctcagtggtcccaggtcactgaggtcttacttagttcccactgccctgcagactttgcctgctccaatctcctggacttggtgaaacttgtggagcggctgagcctgggcatcttccatccggcctacggacccgctgaacacatcaggaaaaagctgtatgaaaatcttcctgacaactgccatattctagcctcccagaggctgggcatctccatgacccgatggcctgatggcaagaatttcatagtcaccgattttgccactcgggatgaattcatccaggtgagccagttgggacctggggcttggggaacacagtccacataataatagactgagtctaggggtgtgtgtgtgtgtgtttgtgtgtgtgtgtgtgtgtgtgtgtagagggggaggagcagtagctagactgtatccaagactggcctgagtccaaggctctgtagaggctgaggtggagaggaacccgaggtgctgtcagacctggggtagactgaggagctttcacagtcaggacctctaaggctgctcggtccacaggtgtctgccctgtcctgctcacccctctttcctaagcgttttctgattgccagagcaaactgtttcctgagagcatctaatatgtgtgggcactgctgtatgctgcggggttttgccaggcttgggcacatctgaaacaccttaccttctcagggcttatacctactgagcctacctcctgggaagggattgggaaagggcctatgtgaaattcaaccaagagaataaggcagggaaggaatgaagcctttctgacttgataggtttagggggtaaggaaagtggggccaaagttctgaaggaatatgggtctccatactggaaggcctcaactccttgtgtagagtcctggagtccctcagcgtagctctgcccacccttcctgtcatggcatctgtcccatctgctcctgatgctgaggtccagatagactcattctcatcttggtgccttgtccccgactctaggacagtctgactcaggagggcccctgtctactggaacataggtgtttggttgtcagtgtgtacctgagggggtgtcacttgctgtgggctgatgctcagggaggcacttcagagctgtcctggtgtgttcacctggacaaggtgctcagcgaatctcagctgtcacgaaccgcaggcattggtgggaaattcacatctcaaacatcggagccaggcctgtgtctgcttcccccagggacagggggagcaatactcttcagggttgtcaggtcaggaaggctctgtgtgccctgggagctacagtacttgcccagccctgcccctcccagcctagttggccacgctggaactctccttgcctgtcaccctctgcacgtgcctgtgcgtgttgctggcataagaccagggatgatgatgcagaccagagatgaggctaagcacgttgcaacacaggacaatgtgcacagccctttacagcacaaggccagcacaggataagtctgtaagcgaagcatctcccccaccccatcctccatcccccggccccacctttgcctaacatcagcaagacttgtcagggcttttattaggagatagtgtctgatgtggggaaagagagcctgacattcctcctcagaagtgcatttgtttggatcacctctgaaaacactctttgggaaaacacacagaggtttttggtggcacctccttgccagacctggggtatgtgtgtgtgaattggtactggtgtcttggttcatagattagacttgcagtctccaggcagctagggcgccctctgcagggagaggaaggtggcttcaaggatgcctgcgctcatgcgttcttttgagcacaagagagcacgtgagcactgagcgagggctatgctcttctcttccaggctctgatctgcaccttgtatttacccttatattgtggggtgatccccccggcattcagaggccaggtgagtttatccaggggggcccctgtctgcattccactctgggctccaggcagctaggacaagggcttgtccagaggtggagataggaatgaggaggccacccgatgttaaagcacctactgtgtgtcaagcctccccttcctagagagctagaaagctctagctagagagctcagaagctaggctagaggaaggaatggacttccattgatacggggagagtgaggggaagatggtgtgggctaatgcctctctgccttccagcgcttcattgacggggctctgagcaacaacctgcccttctccgattgccccaccaccattaccgtctctcctttcaacgggacagtggacatctgtccccagaacatatcacatagtctttttgagctaactgccttcaatgccagtttccagatctctaccaggaacttctttagggggctcaaatctgtgttcccccccaagcctgaggtaggtcaccctgcatggtccttgcctacgacccatcaacttcagttactgcccagcctcaagtcacacaaggcctggagcagaaacttgaccctccttctgtcccttcctgggagaggcctggagtccagctgtggtccacactcaactggcaggctccataagcagagacagttaattccattcctttttttgtttgttttttaaaaagatttattatatatatatatatatatatatatatatatatatatatatatataaatacactgtcgctgtcttcagacacaccagaagagggcatcaggtctcattacagatggttgtgagccaccatgtggttgccaggaattgaactcaggaccttcggaagagcagtcagtgctcttaaccactgagtcatctctccagccccctccattccttcttctagatttatcttttttatgcatctgtgtgtgtgcatgtagtgtgtgtgtatgtatttgtccatgtgaatgcagtgcttacagagaccagaagaggacattggacccccgcccccaggaactggaattattggcagttgtgagccccccaacatgactgcagggagttgagctgtgcgcttccgcaggggcagccagggcttcccgttgctgagtgatctcttcaatatctcaccagtttttgcctttttgagacagagtcttacaatgtagccctggctggtctggaactcaccatgtagatcaggttagatctgactgcctctgtaccccaagtactctgtgaacggtgtgtgccacaatggcccccaacctccagggagcttcacttctcagaggacagaggacataggctcaggcaggttaagtaactacttatccagtcacagagcccagaaatgggagagctaagatttactctgttagtccaatatggtcttgacagcaaacttccaagacttcagccagtatacatgtgctctcgcgcccgcacaaacacacacagagtgagatggctcaacaggtaaaacagatacctggtgtgcaagcctagtggctgagtttgattccaggaaccaacataaagagagggagggaagagatgactccacagagttgtcctctgagctccactttcacaccatgccacatccctgcgaagggtaaaggaagaggctgtcctgggcttgtggggatccaggtggagacccctcagaccctgacccaggcctggtcaccccgtctattggagggagatctcagatggaggtaggtgcctgagttctaggtaagctgagtgagctgcccaaggggagggcacgtcaggatcagatctgtgccctgtgcgctctgggtctccctgactagtatcgtgtctctggagttcccgagccctgtggagggtcactggggcagcaggctgtgcttgggctgtgtccatctgctgacatgtccccagtccatggcttgtagctagagagccccaagctctatccttcctctgccctgtgtcccaggtggtagctgaccattgccgacaaggttacctggacgccctgaggttcctggagagacggggtaggtggcagagggtggtaatgggatgatgtcaggtggactctgcctctctggcatttgaaccctgggatttcagtgcctgctctgcgtgtggctgaaccacagaggggagtctataagggagacatttgggtcctattctagagcttccaaagactggagcacggtgatagagatgggagggggtgatgggggggcacttccagagacaaactggagcagagaaagatcgtggtctgaggtagggagagtaagcagggagaacttcctggaggtggtggctgagctggatcaggaccaactatagagtgtctgattgggatgggaagacagagtcagacggctggaggggagctgtgctccaaggggtatgtcagctggcaggaggtgtgtggtctccaggggtggaaagcatggctgtcatcctctgaagcaaggttgagaggcgaacccaggctctaaaccttagacctatcctcaaacccagtgctccaccccaacctgcagagccttactctgggccagagcttgaaagtactgcatttaactctcatgacagtaggctttaagttctttttatagagggaaactgaggcagaagtgccaaggctcacatggctggcaggtgggagtaagacagaatctgagaacagggctctatctctaggcatgcgggacagcagggcagggcccactggggtccatcctgcagcccgagtcctggaggtttttttttgagcactgagacgtgtgggtagttaaaactctgttcctttggaggttgccactgtgccgactatcagatcttctccttctcaatccaggacttaccaaggagccagtgctgtggtcgctggtatctaaggagcctccagccctcgtggaggggcccaggggcactggccatgaccagggccagaagactggcccaactgtcaggtgggacattcccaatgtgctagtcaaggatgtgcccaacttcgagctgctgtcaccggagctggaggcgggtactgccccttctcattctcctcgctggcctgcgtggtcatatctgccaagcccttaccaaatgtcaagtgtggctggggttttttttttttctgtgtcatccactcttaactgcaacctatttcatcagtggggaaactgaggccctgagagaaggaacttaacagctattcctatctgccattagggtattgagcccaagcccgtgcctgggaaagacaggtctgtgtccaggccacattgaggtgcctagggatgaacacaggcacacatggcaggtggcatcccagagacacttcaatgggagctgctagccaaagaaggagggatgagagtttgggcagaggtcatcacatgggcaaaggccttggggctggagaggctgagctggtgggtctctgggatcatagcatagtgttagggtaagatggcagacatgaacttggggatgactgggtaggcaggaggagagagctggtgtcttggttacttttgtattgctgagataaaataccatgatcgaggtaacttataaaaggaagtgtttggctgggtgtgcctttaatcccacttgggaggcagaggcaggcagatttctgagttcgaggccagcctggcctacagagtgagttccaggacagaaagggctatgcagagaaaccctgtctcaaaacaaaacaaaacaaaacaaaacaaaacaaaacaaaagcaaaaaagggaagtgtttgatttagcttatagttccgtgatgctggtagaaacagctaagatcttctgtctcaaatttcaagagcctgttgggagtcttttgaaatctcaagcctgcccccagtgacacaattcctccaaggccacacccactaatccttcccaaacacttccaccaaccaaggagcaagtattcaaatatatgtgcctgtggaggccattctcattcaaaccacagctggggggaggcctggggtgcagtgaagctaatgagggagggcagtggccaaagaggttgaggtgatagtgaaggttcaagaagagacactgaggaagtaagagattaatttatatacagaaggcttttggtctgcactgagccctacatccaggcttctgaatttgggatcagtggggtgtggttactgcccctgtgcactgcagctggctggggcaggaaatgggagagtaagtctgatgcttagacaccataggttctcacccactgccttagaggagtgttctgcttgggacccacttcccatgactagggcatagatggagttgggtgttgtctccttgcctgcaatcagagctgcgataggagttaccagcagtctggagtggccatctggctcttgccctgatcagtcagggtgggagcactctgcttagcatggtaaataaaggaactgggcctggcaaacaaggatgctgtggtcacagctgctctgaggcgattgctgtgagaaaggcccggggatgcctgtttatccaaggcactgtgatggacttctcccggtaggaggccaggaaaatggagctcagaaccagagcctggcaggcctcaaggttgtcttggctctgcctcttcgtggaagagactctcactgggcacttctgaactaccactctgaaccttggtctcgtcttctgtagggggggcctcagtgggttaagcggagtgtgtatagggtgacccctgctggcagctatggctccatccatatggagatgctgaggttcacatactatcttcatcttctgggaccacaaggcttggtggctccagggtgacctgggctggctaggggagacaacctcacagcctgagggtacaaaggagccgtgggtgtcttcctgtcttgcagcactgagaaaggcatgcaagagagacttctggacccgtgtccagtgttcagtgccagggaaggtgctggcctacctgctactgccctgtacactgcccttcgagtatgcctacttccggagcaggaggtgagatagcctgggctggggaggggggaggttgtgaccagggagggtgggtaggtggctagaaatggcaggtacctgccttccccgcactagttgccacgggctggcccatggagtgcctgagtaggctctaccaaaagaactgataagggaggtgatgtggctggctacccagaattctggctagtacttagactgttctgtgaccttctgccctctgatccgtggtagagttgctgtgacaatgtgatgatgccattggcaactctctcagctgaactacctcagattggctgtgtggtgtgggcccgctctcctgacctctctaagtcatagcatgtttcctaaataacagggttccttcaggtcagactggctgcagctgtatccctgctgggttagtggcctacgctgcttcctttgctcactcactggctgctgaaggctgacctaggactaggtgtgagcttggggcaagagaggcaggctgaccttgacctggctcctggcctggtggagcacacagctcacagccggccacatcacctgggcattttagcttctaagaaagagccatactagcacagagctagcctggcacatactcagacctgtttgttcagggaaggaaggcaatgacatctgtattatagttttaaggatgagctacagtccgttaggcaaaggagaagaaagagccttccaagtggacagggtagtgcgtgcaaaggtcctggggcagaagggagtcaagcataccctaggaacccaaggggaggagggtatagggaaagctagagagtgactgtggccagacacacagacacacgaggtcagcaagacacagaggtgctatagggaggtctggtgttttcttggagaaaggatctttctaatcttgtaacccagagtggtattgtaatcctgaggattttgtaatccagagtggtcttgaactcacgatttcctctgcctggggttctagccatgtgccccaaccctagacccgagatctgtgcttctgaaaaacctcttccttagaagtctgtgatggtgagtcccagcactctgtgttgtactgcctggagctagtgatcaggtcagcagtcgcccaaagctgggggacaccttcctgctgacagactcaccttgcacagagctgtgggctaaagccacgagctgatgaaatgatgctgaaggaatgtaaaatctagccatctctatggtgtgagcctgccagagacagctcagaacacactgcgtctgcccttctgctttcttatgacccacaaggggcaggaagagggatggatagagataagtaggggaagggacttcggcagagggtcctcatttggcaggcgagcaaagctgggccccagagaggtcctttaaatctctaggcaacagagcagagtagcctccaacctaacatcacaccacacacttcccaccggaactctgtggaagttgaccccaagccccatccctccctaaagctcaaagtctacttcaggtcatcctaccatgcatattccccgtgaggctatcctgagatgatcttgctcaacctgggcactcagccctgctcccctctcacctctgatctggcaccatcccacggctccctgggaaattgacattgcctctcccacaggctgatggagtggctacccgaggctccagatgacttggattggatgaggagcatcctgaagagtaccaccctcgaggtctactccatggccaagtcatggcttctcaggttgggcaggtgaggtggcaggggagggggggtcatgtggagaggggctggagtatttgcatgatgcccacattcactacccagcactcagaacccagcaccgaaatcagaggactcagaacctagcacctactgcacagtatccatctagtgtcccgcacttaacacacagtacccagcacacatgcaatgtccttctgtctgtccacctgagggtgctgtgtgtgccatccggtatctgggatctagtcacgctagccagtctggccagtctgctggggatctttatcatggctctgtggttggaatacattccctcccccaccccctcgtgagcctcagcaaggctgatactatgtggctgagggatgctggcctcttttctgactgatatccctagtctcttaggaaagccaccctggtccagatgttctcttgtttggtcctgctgagggcttcagacttcctccagagggccagggaagaagtagcctgctccctgtggggctgccccctttgatgctctctaccttcaccaaaatccagtccttgaactcacagacgattcctgctccctctacagcccacctggcacccgagcagactccggtctcctccggcagcagcgggggacagctccttctgggaacaggcccttgaaccacaggttcgctgattgcgcactgggctaggccaacctcatatgtgaccctctgaactctgtccttgccctctggctgtagggactctggcaggtggcctctgtcaggttcagcccccaggatctctgagctcaactcctcactcaggaagcttgctgaattactggcaaaagacctcttcctgtccttgctaggccatgtgcttcttgtgtggacaggtggtttcctggcatggactgagagcccaccctctgcagctgttcctgggaccttgatctccaaggcctctctggatgcttccagcctactgtgtgctcaggccactgttctcttcttgtcagttttgtcagagaagaaaagacaacaagctgagacacacctcagttctggatcttcatgtgagagacacagccaggtgtctagcagaggtgtgttctgcactaggctggaactggagatggtgccccagggctctgatcatggtattgactgctctaactgggctcctttctctgacccacacttatctgagggattgatccctgtgatagtgaggatggaaggtggctggctggggaatccatgggggggggatcctgattatagatacttctgttagggataaagcccactgtcactgcaggctagggaagatttctgggtggcatacataccaaagcccctctggtctcttctggggtctcagttcttcctacccctgaagaaccaacacagtgcacctagccagactgtaggggacactcagaccaagtggagatgggactgcagagaaatggaggtgccagtgagagaagagattatgtaataaatgctgggcagaagaaatgatgtgtcattcattcattcatcgattgattatatgtacattggttattcttctatcattgattgattcatttatcattcatttatttaccatttatcattcattcattcatttggctcctcagatgggacataggtgcgaaattcttccatgagccctcccaggggtgcttagaaaggaacacagtgagtgaaaatatgaagccaagtggagagacagggaccagactgtcaggaggctgctccaagaaggctgctgttgggctgaggtctgagaagaggccagatgttatgcaaggccctggagggagaaccaggtacagaggtctggcaaactgagtgatgcccatgtcatagcatggtcacagctgtggcagagagattggacggcatgttgagagtctcaggcaggggtggtagcaaggctatgtcaccaagggctttggagatttctttatttttattgtgtgtgtgtattctgtgtgagtttatgtgcaccacttgtgtgctggcacccacagaagccagaagacggcatccaatcccctggaactagaattctaagcgtctgtgaggtgcctgaggaggatactgggaacttgggtcctccacaggagcagtgaaccttctcactagccccatcaacagggagtttggatcaggggaggggctggagtaatgaatgaagggtttggggtataggcctataatccagcagtgagggggcagaggcaggggaaaataccaagttccaggctatcctgggctacacagtgaaacacatctaagagaaaaattaatcccactcgccctgaaacctcagaggtgtgagccagtgacacacagacagggcagggctgaagggacgagagcagacagcgggagcaagagtctctcaacactctggcagtgccaggatcgagccagcttcacctcctcacggtctctcatgtctcccaggtgggccagacactgacttgatcccttacagg

SEQ ID NO: 22 - DNA, 2478 bp, Homo sapiens
atggaagaacaggtgttcaagggggacccggacacccctcactccatctccttctcgggcagtggattcctctccttctaccaggcgggggctgtggacgccctgcgggacctggccccccggatgctggaaacagcccaccgctttgcggggacatcggcaggtgctgtgatcgccgccctggccatctgcgggattgaaatggatgagtatctcagagtcctcaacgtgggtgtggccgaggtgaagaaatccttcctggggcccttgtccccgtcctgtaagatggtgcagatgatgaggcagtttctgtaccgggtcctgcccgaggactcctacaaggtcaccacggggaagctccatgtgagcctcacccgcttaacggacggggaaaatgtggtggtttcagagttcacgtccaaggaggagctcattgaggccctatactgcagctgcttcgtcccggtgtactgtggcctcatccccccgacttaccgcggtgtgaggtacatcgatgggggcttcacgggcatgcagccctgtgccttctggaccgacgccatcaccatctccaccttcagtgggcagcaggacatctgtccccgggactgcccggccatcttccacgacttccgcatgttcaactgctccttccagttctccctggagaacatcgccaggatgacccacgcattgttccccccggacctggtgatcctgcacgattactactaccgagggtacgaggatgcagttttgtacttgaggcggctgaatgctgtttatcttaattcttcctccaagagagtgattttcccccgggtggaagtgtactgccagatagaactcgcccttggcaatgagtgccctgaacgcagtcaaccaagccttcgagcacggcaggccagtctggaaggagccacacaacctcacaaggagtgggttcccaaaggggatggaaggggcagccatggtccgcctgtgtcccaacctgtgcagacacttgaattcacatgcgagtcacctgtttcagcaccagtctctccacttgagcagccacctgcacagccactggcctcttcaactccactttctctaagtggcatgccacctgtatcattcccagctgtgcacaagccacccagctccacacctggttcatcactgcccaccccaccacctggactgtcacctctgtcacctcagcagcaggtacaaccgtctggatcaccagccagatccctacactctcaggcacccacttcacacaggccatccctggggccttcaactgtgggggcacctcaaacactgccccgaagttctctttcagccttccctgctcagccacctgtggaggaactaggccaagaacagccccaagctgtagctcttcttgtctcttcaaaaccaaaaagcgccgtgcctctggttcatgtgaaggaaaccgtcagcaagccttatgtaacggagagccctgctgaagactcaaactgggtgaataaggtcttcaagaagaacaagcaaaagacaagtggcaccagaaaaggcttcccaagacattcgggatccaaaaaaccaagcagcaaagtgcagtgagcatgtctaatgttccttaaatcccacggagaggagcagctttgggaactgtgttcagagagattccgaggaatagaggagagtgtaagggagtagggggtgcagtgggagattgggctttggaacagacacatccgacataaaattcctgctctgccacagctccactcagggatcatggttgggacacttgctctccctgagcctccatttcctgtaaaatggggatgataccacttcataaagttgtgagagttaaatgtgatcgatgatgtaaattgcttcatagaatgcagaatgtgtaatagctcacaataagtaggtattatgtttacatattatgtttgtatttatgctacttaaatacaaaactggacaggccaggcatggtggctcatgcctgtaatcccagcactttgggaggctgaggtaggtggaaaacctgaggtcaggagttcaagaacagcctgaccaacatggtgaaactccatctctactaaaaatacaaaaattaaccaggcttgatggtgtgcacctgtaatctcagctactcgggaggctgaggcaagagaatcgcttgaacccaggaggcagaggttgcagtgcaccaagactgcgccattgcactccagcccgggcaacaagagcgaaaccccatctcgaaaaaaaacaaaacaaaactagacaagtgagtgcctacgtgacactcaaatgttgccagcatacagttaagggccctagtcaatgtaggcctgcttcttatagctttttgactatattatgctgtctttgacttagtcagtcaacacttattgagcacctactaagtgccaaacactctcctggactctggcaaaataaaaaatgaattaaaactct

SEQ ID NO: 23 - protein, 532 aa, Homo sapiens
MEEQVFKGDPDTPHSISFSGSGFLSFYQAGAVDALRDLAPRMLETAHRFAGTSAGAVIAALAICGIEMDEYLRVLNVGVAEVKKSFLGPLSPSCKMVQMMRQFLYRVLPEDSYKVTTGKLHVSLTRLTDGENVVVSEFTSKEELIEALYCSCFVPVYCGLIPPTYRGVRYIDGGFTGMQPCAFWTDAITISTFSGQQDICPRDCPAIFHDFRMFNCSFQFSLENIARMTHALFPPDLVILHDYYYRGYEDAVLYLRRLNAVYLNSSSKRVIFPRVEVYCQIELALGNECPERSQPSLRARQASLEGATQPHKEWVPKGDGRGSHGPPVSQPVQTLEFTCESPVSAPVSPLEQPPAQPLASSTPLSLSGMPPVSFPAVHKPPSSTPGSSLPTPPPGLSPLSPQQQVQPSGSPARSLHSQAPTSHRPSLGPSTVGAPQTLPRSSLSAFPAQPPVEELGQEQPQAVALLVSSKPKSAVPLVHVKETVSKPYVTESPAEDSNWVNKVFKKNKQKTSGTRKGFPRHSGSKKPSSKVQ

SEQ ID NO: 24 - DNA, 39757 bp, Homo sapiens
tgacctccccagctgctcctccttggggttaaacattgctatctcaaacttctcatttctgagcgggaagaagaacacgcaacacgaagcctcacacacttagaatgatctagatttcaggttgatttcttcctgcacacttagaagatagcatgagggcttggggcttgtcgagagggcactgaccaaggtcacagggtcagtgaagggcccaggagaagaaacagagagaggtgcagaggcggtggtgggtggggccgccaaagtcacacttctcttaaaattttacccagggccaatgctaacatgtaaataaacaagtgaataaacaaaggggtgtttatgtaagaaaaacaaataaaaatgcttgtgtaacacatcacacataccacatagcctgaaggagcccaggaggggcatctcttttgggggacccgtccaaaagagatggggtaggcaccgtcctcctggagggaggcgggcttggaagcccttcacggccgcagcagcatgttggggagggaggtgtacaccgataaccatcacggaggttttgcaactgaatgttgcagctgagggctgggcagagcccgcctccttgggcatggccctgtgctggggagcagtgaatgcgccctggtataccatggatgggcagagggctgaggatcccgtgcccgagatgaatctagccaagaaatgaagccagtggggggctcacaggaccaagaccctgctcacacaccgggggagccttctgcatctcattctggggcccctttccaacctttggagttgcctcctggcggagcttaacaggctgaggcagcttcctgagcagcctgtggttgctccagccgggtagagacagccacattccaagctccggggtggcagggaagctgggtagggagttcctacagggagcggcagcccaggctcgggcaggcaagtgctgaagggtggctccgccttccgcagaaagtcagaggccgaggagatggaagaacaggtgttcaagggggacccggacacccctcactccatctccttctcgggcagtggattcctctccttctaccaggcgggggctgtggacgccctgcgggacctggccccccggatgctggaaacagcccaccgctttgcggggacatcggcaggtgctgtgatcgccgccctggccatctgcgggattgaaatgggtgaggcctgtgttctgggtcccctgggaagtctcttgggggattccacagagacagaaggagcgtgagggaggctggggatgctttgggggacaaagccaggcctgggtggcaggaagggaagggaagagctggttggagtaggatagcggcagctgtgacgtttgaaggttggggtctgccaggccccagactctaacatgcagggtgagcctggacccctgtccctctccctcagtgcccttacctgtgagatgctctgtctcccagtgcctgggagagtaaaggacagtatctccctcgaacatcgaacatggtgctgtcagaagctacaccacggcacagtcatacattggggttcagtgttgctcctgcctggcctctttcaaaatgcaaatacccttgggagaggcaactcttcaaatctctatcaccctgatggggacgtattgtccaccatttccaggtggctggaaagatgaacgccctgaggctggaggggtagacaggagggagcccagagtgggagaagaataggaaggggtggggagagaatacagaagctctcacttcaccttctgtcctgctggtccaggagcctcagcgagaagaactgctacactgctaacccagagagagaaacactgtcccggacagcagatgcatctagcagccccaaggaacaagcccaacagcctagtgagctaccttggaggggcatttattcatttacagagctcctattctagacagagctctgttccaggcactgaggacaaggtccccgcccttgtggagcagaaattctagagaaaagaaacagataataaacattcaaacaaggcaccttctagtgtgaggtcgggaagtgatatgatctatgaataaaattaaagcaggaaagggactgagaatgaaatggagactatttcaggaaggtggtcagggaagcctttctgaggaggtgacattcaagcctagagctgggtatagtgatagaacaagctactgggagctcctgaaaaggagaattcctggtggaggagcagccacgaagcctgcggtgtggaaaaggtggtgggaggtcaacaggggccaggtcatgtggccttagccacaagctctcatctcacctaagggagggggagcacagacggctgtgacagcaagtggggagggggcagagggcgtgagctcctcaggtgggatcccagctctgccatttcatgatgcatgcctgaagtaagttattgaacgttctgcgcctctgtttctccgtccacaaatgggagctcctaatagcgcctagttcccggcattgttacagcagcggtctccagttgacaccagcgactggttttgtggaagacaatttttccacacaccagggttggtgggggatggtttcagaatgattcaagcgcattacatttgttgtgcactttacttctattatcattatgttttaatatataatgaaataattatacaactcaccataaggtagaatcagagccctgagcttgttttcctgcaagtaggcagtcccatctgagggtgatgggagacagtgacagatcattaggcattagattctcataaggagtgcacaacctagatccctcccatgcacagttcacaataaggttcacgctcctataagaatctagtgctgccactgatctgacaggacgcggagctcaggcggtaatgtgagtgatggggagccgctgtaaatacagaagaagcttcacctgctgcttacctcctgctgtgcggcctggttcctaacaggccacagacccggggccatggcctgggggttgggaacccctggttataggattggctgagctaatatgaatacctgcttagaacaatacctgacatatggcaagtgttgagaatgtggcaaccatctgattaggcttcttaagatctctgaacgatgggcagagtgtgtttgagggagcaaggtggaaactagataagggaagagattggattcaggacgtgctttgaaagtagagccagtgagactttctgaagggccggctgggctgtgtgagggagaggctgcagataatagccccctcccagcccaagcagggtgccttcctggctggggacctctatgctgggtggaaagtctttattttaaagaataatagtaatgcccattcactgagtacctctaatgtataatataattttttctttttaatgtctcacaagtaataaacacatttttctcactggacaaaattcaaacagtgcgaaagtgtatagagtaaaagtagaaaatagaggccagttgcagtggctcatgcctgtaatcccagcactttggaaggctgagacaggtggatcaggggttcaagaccagcctggccaaatggcgaaaccccttctctactaaaaaaatacaaaaattagccaggcgtggtggtgcacacctgtagtcccagctactcgggaggctgaggtgggagattgcttgaactccagaggcagaggttgcagtgagatcgcgctactacactccagcctgggtaacaaagccagactctgtctcaaataaataaataataaataaataaataaataaataaataaataaagagtagaaagaaagcctccatttgcccggctgcccctaatcccccactccctcctctagccagtgttaagggctaggcatggatctttccagatctttttaatgcacagtcttccttgaatgcagccattaatggctcttccccactgaactccaggccttcagaatgggggtagggtgacagcccccactctgtcctggagtcaccgaattcctgccttggaaacacctggtgcagggattctctctccttttccgggcgcctgcgttgctcatctgctccattaccctcactcttctgtctttatgtcatcttaaaatacagcatctgggtgcagtggctcatgcctgtgatcccagcactttgagaggccaaggcaggaagattgtgtgtgggtccaggagttcaagacaagcctggatagtacaggaagaccccatcgcaaaaaaaaaaaaaaaaaaaaaaaagatgaagaaagaaagaaaagatttttcagacctaatttaactcattcagtttgatgaatactgctgcaaacctacttggccgggctgtgacggtcatggacggcagtggtaggaactaaaacctgtaaaaaggacatcaagcagtggggtgtaatcaaggactagactgtgggctgctcgtaattatcgcagaagtccagggaagggtgagggaggtatgtgtgggactcacgctgtccgctcagcagcatgtttatgagacttgtcaatgtcactccagtctagttcattcactcattttaactgccatacaatttactcttaactagcacttccttttatatcaggcaccgttctacctctggcatattaacttgttcagtcctcacaacagccctcccaggtgaaaactgttattatccccattttacagaggaggaaaatgagtcacagaaaatttaaataacttgcctatagtcacacagatagcacttggtaaagctagaattcgaactcagtctggccaaagtctacattcttatcctttaaattgccctgcttaacaatgcgatgttaaggggtgaggaggcagggtcggggagtctctctgaacaggtggaatttccacacagacctgacgtgggtaagggaatgagcccaagaatatcttgggaagaacattctacgcagaagcaaaagtctcatcgttagttagaagccgaaaaggaaccagggctagatctttttagttagtcattttattgatggatctgaccagtttccttgtcgttacaaccatccttgtttgtgtctcccatgctcacatgacatgtatatcattctagggtatttgtgtcattaactttgttaatgactttgataagttactctccaaagcagttgtaccaatttaatcacaagcagcagatatgagttctaatttccccacaatgtcacacttgaaaatcttatgccaaccgaataaatgtgaaattgtgtctcatctactgtatttaatctacctatcctgactgctagtgatgttcagcatccccatgtacttattgaccactagacgtttcctcttctgtactgtgcctgctcaccacctttccctgttttttcctactgagttatctttttcctttaatgtgtaggtgttctttatatattctgtacacttatctttcaccagttatgcacaatgcacctctcttttcccactctatttcttgtatttttattttgcttcgatgtcttttgttattcagattttaattttaatgctgtcaaatgtatccatcttttcatcttacaacttgggttttgttttgttttgtttttgagactaggtctcatcctgtcacccacatcggagtgcagtggcacattcatggctcactgcagcctcgaactcctaggctcaagtgatcctcctgcatcagcctcctgagtagctgggaccacaggtgcatgccaccacgcccggctaattttaaaaatttttttgtagagtcagggtctcactatgttccccaggctggtcttgaactcctgggttcaagcaatcctccttcctcagcctcccaaaatgcaagagttacaggcctaaaccagtgcgcctggccatcctcttataatttgtgttttaaaatatctttattacaatgatttcttctacattgtctttcaaaagttttaaaattttcatatttagattaattcctcaggaatttattttgttgatggttgaggtagagatctaatttttaaattgttttgccatgtgatagccaattagaggctatctccacacttacttgaaatgctgcttctggaatattcaagtgtctcttctattctgttggctaatttgtctatttctgttttaatcattatagtcttataataaagggttcatcacatatgacaagcaatctcctcctcttcttcctccttttttcttcttcaaatattgcctccattatgtttggccctttctttgccccatgtgaaaagctctgctgggctttcgactgaaattgcatctttccatttgtaaatatagttgatctttctattctggtctttttgtgtccttcagtaaagattttgtgtgttttattagatttacttttcccttgtagttttattttttattgctacgggaaatgggaccttttttacctatggcattttctttctttctttctttctttcttttgagacagagtctcgctctgttgcccaggctgacatgcagtggtgtgatctcggcttactgcaacctccgcctcctgggttcaagagattctcctgcctcagcctcccgagtagctgggcttacaggcacccaccaccacgcccagctaatttttgtatttttagtagagacagggtttcaccacgttggtcaggctggtctcgaactcctcaccttaagcaatccacccacctcggcctcccaaagtgctgggattacaggcgtgagccaccgcacccagctagcattttctgaggctcttctcaaggccacttccccacttttgtagccagtttggggcttttctatcctgagatcctgtgtttttttcctgtgaaagctgaggtggcagaggtgcaacttctggcgtgtatgcccagttctgtgactaggccagttcaaggcacatgaaaatgttgtaaaagcacttaacatagtgcctggtttttactagagatgtggtaaccttcattaatccattcattcacctaggaggggtggtctccgtgattgccactgggggatccagaatttccaatcttgtcagaagtggggttggaaacgtgcctcaaagctgcatttgcataattttgaaaaacaaaatgtttcttatggaagaatagtgatttggcagacttaaaaccctactcccacttaagctacttcttggcacctttctccccataaaaaactggcaagccaatctgtccgtcccttcgttcctttcttcgttcgttcgttccttccttccttccttccttccttccttccttgcttccttccgtgttagttgagcacatactaggccctgcagaggatgcgaagatacaacacaaaatgaaactctgatcttgctgcaaggagcttacagtccactcaaacttaaaggcacctaccaaacagttataatacaagaaagttcctattgaatgttagagaattataaggcataggagtgcagagggatggagaaaacacttccaaacatggaaggcctcatagaagcggcatttgagcttgtccatgcttgaaatgtgagaagttggacaggtagagacaggttgggcaggaaccaaggcttccagcaaggacatagcatgacaacacgacaaagccacacagtggggaaacatgatgcctccagagcatggtaaacagtgcaagtcaactgacgtcaggagttgaaaggcgttgcctttcctgtggtccaagagccccctgccatatctgcactccagctagtcggaaagggaaaagggaagggcacagtcctttagagcaggacttggaggtaacacatgtcctgtcagctacatcccatggattggaatttagtcacatggtcacacctgactgcaagagaacccaggaaatgtagactttatttttgggaggccatgtggccagctaaaaattggaggttcgattacaaaggaagaaggggagaatgggtattggggacaaccagtagtctctgttgcaacaaccataatcaatggcatgaggatcctggagaaactctgcccctgcctgagaccaaaaccatgacttcctaatcacaaagttggccagatgcagtggctcgtgcctgtgagcccagaattatgggacgctgaggcgggagaattgcttaaggccaagagtttgaggccagactgggcaacaaagtgagaccctgtttctacaaaaaataaaataattagccaggtgtggtgacctgtccctatagtcccagctactcaggaggctggggtgggaggatcacttgagcccaggagtttgaggctgcagtgaactatgattctgccattgcactccagcctgggtgacagacagagaccatgtctctaaaaacaaacaaacaatcacaaagccaagtccagtttgttgtctgggaaagggctcagctctggcccaccagcagctgcccctgtggcagtgggagcatgagtgcctcagtcttgcagcagggggcctgggcaatgcaccatggcacctactgtgtctcctgcctagaatgaccctctgcccccctttcagagagaaaacctcctctccagacaccatcccattttaccccactccatctctcttttccatgatgtctagtttgcccataaaatctgcccacatattctagcatttgatgttgtacggtcttaactgtttcagatacagtgtctcatttcatcaactagattggttgagcaatctgagatcttgtgggtcctatggctcccacaaactgctcataaaacaagaactccttgataaactcctggtgtttgctggctattggccagtgtgatactgaatgtgtctttgctatctgtattttatagtttccctggctcttggataattttctgtagttatggatatgagatataacgctagaatgttacagtgtcaaggtcagtaagtccagctccctcattttgcagataattgtatttagaccctgggagagaaagtgactagttggaggcagacctcacttaagccaatagtttccaaactgtgttccagggatccttagggttctgcagatggggaggaaaagcaggggacagacacgtctgaagccccggctgcactagtacagccccgctgttgtccatcttatacatgggggatcttgctaaaattatatgtaaagaaaatctttcagtaatttcttttaaagtttgagaatcactgtttgagcccagttccctcaagatgcagacaaggagtttgaggatcagattttggctgaattacggtgatgaaatagaacattcattcgctcattcattctaggtcctggggatctggcagtgagcaaagcagtggatgtccacactcatgaagctgatattctcttggtgaaattaaaccatagaaaattaagcatataaaaaaggtcagcatgcgtgctcaaaggagaagataaagcaggggaaggatgggagaatgacacaaggggcacagtgttgggtgggctggccagaaggcccctctgaggtggatgaagctagggagagagccacgtgcactgagggaagagagttccaggctgacaggtctgcaggtgtagttgccccccggtgaaaacatgcttgtgttctcagaaagggagatggcttgtccctctggacgggaaacaggcttcaaagggatcttgcaaaacacatgagaggtccagaggagccatgtgttttgccccctgccccgcaaatgtgtgcacaacaggacccagcacaacttctcccatggagacagagcactggagaagtgttttggttggcctcccacactagggccagtgctacctttggcagctcctgcccacccagtaactgaaattctcctggggccctggagctgaggctggctggttattgtcattgataggtggctcaaggtgtattctggtccactggggctggcactggggagtcaagccttcctctttgagaacctcgctttagtaactcctttcctttgtcactcatgcacctatctttgttactcagttgaggaatgagacatagccaggattagtggagaatagagttcccaggaaggaggagggctagggctgaacttccaactctgcccccaagttctgtaggttcttggctggccagggacccatctcgcccttggttttgccatctgtaacttgggtgattggaaacacatcccttggacattatggtgaaagacaaaagaagtcatggatgcattcctggcccagtgcttggcaaatattagtcacaaaataagtctttgaagaatgaatgttgaatgaatggatgaatacgtgaaaagaaaattaccttgaaatagaaaagcatagataaagcagcactttccaaagtcaagaatgtcccagagaacaaattaaactttattaaaacaaacaggctgggcgtggtggctcacgcctgtaatcccagcactttgggaggccgaggcaggcagatcacgaggttaggagatcaagaccatcctggccaacacggtgaaaccctgtctctactaaaaatacaaaaattagctgggcgtggtggtgcgtgcctgtagtcgtagctactcgggaggctgaagcaggagaattgtgtgaacccgggaggcggcggttgcagtaaaccaatattgcaccactgcactccagcctggcaacacagcaagactccgtctcaaaaaacaaaaacaaaaacatagatgactggaatccagcctggcctgagcaatgtttttcatggtaattgtgagctcatctgcacaaggacttggtttttttgcaggaattctgtgtggccccggcagtggaaggacccatctcagctagtttgcattggctccggccaaacaccctaggattattgctgacttgggaccaattttcatgttaattactctgtaggttgtatcaattcaagctctaaaccatatgcaatacagatttgggtctttcattttttatgggaggctgtgttttttcacccagatccctgggcagacacaagccactggcaccttgtaatagcccagcagcctttggagggtcctagctttaagcagaggtctcatttcacccagcccaagaacgtagtccctgtccctgccagtttcttgtctgataacagtccccacagttaccacatcatcagctttccttctcatgccactggctttcagttccctcttcatttcttcctttcttgtgagttcaaccatgtcttgtaactgttttatattgtatttaatattgggcttttcgttgttattgttgttgttgtttgaggcagggtctttctctgtcacctaggctggagtgcagaggcgccatcatagctcactgtaaccttgaattcctgggctcaagtgatcctcctgcttcagcctcctgagtagttgggactataagcacgtgccaccatgtctggctaatttttaaaattttttttgtaaagacggggtctcactatgttacccacattagtctcgaactcctggcctcaatcaatccgcctgcctccggctcccacattgctgggattgcaggcatgagccatggcacctgatctcttgtttctgaagctggaaaggttcctctttagctcagcccaccatactactggagctggaagtctctccagtgttccttaatacatgaatgagaacctccattttttcctgttgcaggtgtgtgggtgagtgtgagtgggtgtgtacgtgtatatgttgcccagatgtaacttggatttcagtcctggctgctgggtacactttcagaatcaaaatgtcttcattaaaaatcactcccaaaattagttatctgagatggaagtgttttctgtaaaaaggtattctgctcaaattcccatcctagtgtcttttgaataacagctatttaccaagcacttagaatgtgccaggtcccgtgctaagtgcttcatatgcatcatttactctaatcccctcacaaccctatgaagcagacactatatttatactcactttacaaatgaggaaacagggatttagagagattagataattttcccaggtttatacaacagagccaagactgggatttctctaaatgtctacattctacatcctctctaatcatgatttcagaaattgctttgcaagcaacatgacagctttcttgttacactccatgtgtcaagcaattatttatacattggttaagaaagtgagatctggccgggtgcagtggctcacacctgtagtcccagcactttgggaggccaaggcaagtggatcacctgaggtcaggagtttgagactagcctggccaacatggtgaaaccttgtctctactaaaaatacaaaaaattagccgggcatggtggtaggcatctgtaatcccagctactcggggggctgaagcaggagaatcacttgaacccaggagatggggaggttgcagtgagccgagatcgcaccattgccctccagcctgggcaataaaagtgaaactctgtgaaagagagaaagaaagaaagaaggaaagaaagaaagaaagaaagaaagaaagaaagaaagaaagaaagaaggaaggaaggaaggaaggaaggaaggaaggaaggaaggaaggaaggaaagagagacagagagagagagaaagaaagaaagagacagagagaaagaaagaaagaaggaaggaaggaaggaagggaaggaaggaaggaaggaaggaaagaaagaaagaaggaaagaaagaaagaaagaaagaaagatcaaatggcatgacaaatacaaaaggactttttatacaatattatggaaatggtgtcattgtgcgatagttgttgtagctcccactaccatgttggaaccagtatcttccatctcactcaaatgaacttatgctttacatgtttaaacgtctctatccaaatggataaagattcattttccccaaacaatcccttggactttttagtagaagagcattgaatggaagatctggtaaagctttgaatgtgttggttacacagtagaaacatctatttggaaagcaactcatgtaggtgacatatgttaatccatccacctggtatcagcaggtgctaactgagcccctgtgagtgccaggcactgtatccctaacaaccttgcttgatctgtgtgtatgtgtgtgtttttaatttaatttaacttttttgagacagagtcttgctctgtcacccaggctggagtgcagtgacaagatctcagctcactgcaacctccacctcctgggttaaagcaattctcctgcctcagcctcccaagtagctggattacaggtgcatcccaccacgtccagctaatttttgtatttttagtagagatggggtttcaccatcttggccaggctggtcttgaacccctgacctcgtgatctgcccaccttggcctcccaaagtgctgggattacaggcatgagccaccgcacctgacctgatctgtgtttttttaaaaatttaataataaactttttattttggaataatgtcagcttcacagaaactttgctaaaatagtacagagctctgcgtatccttcacccagtctcccctaatgttaacatcttacataaccatggtccatttgccaacactaagacatcaatattggtacattcctattaactatactgcagacttattagaatctcaccaatttttctactaatgtctcttttctgtcccagaatccaatccaggattccacactgcatttggaggatccatttttaaaccaactcacttagacagtattaaaatagcattgttttctttgcttcactttgggctcaaattcaagaaagacagttgagctctttttccaattctagggggaagacagccaaacaaatggtaaataaacctccagactttttttggttcccctgagttaggagttcacccatgaatcctgtgggtaacccaactctgtccttacgaaatcaccacgtaaggaagaatctcctcagctgggcatggtggtgcacctgtagccccagctactcaggaggctgaggcaggagaatcacttaagcccgggagttcaagtccagcctgggcaatgtagtgagaccctatctctaaaaaaattaagaaagaaaaaacaaaaaaagaaagaatctcctcctatgccctagctcagagtctagtatgcatccatgtgggaacacaagttagaagcaggattgacagagcacactgcctgtggtttgcctcatcagacctcaagtccagctgagtcttaagactagggggtgacttagttcacagctgttgcatcatcccagtgtccccttggtagtgcatggagaatctgtacggcaggataggccacagcctagatgagaggcagaattaatttccaccttggagcagggccccaaaaggacttctgggtccaccaaagaaaacagagagaaaagagcaggggcaatggccctgaatacatgatcatttccagggaactttctagatgcattccctctctgcctgtttcacctcagggtgatcagtaagaacctgcagtgggatgttggaggttgtgcctggattctggggggaaaagctcatagtagggtgagggctgaggccccaggccctctacctgagggcaagcaaagagtcctgtggacaccttgctaggagcacaggtgatgaatcagcagaagacacttctgtttccatgacaatcggatataagccagtctgaacttttaaatccaattatttagtaaactcttagggttagaagaaggtctatgtggaggattgttattatccattaaagatgttgttttgtttggtttggtttgaagtgctgaataggtcagagttgttttgctgttggtgaaaccctgtaatgcatttagggcagagcagcaaaatgtgagttttcacatacattctagttctatttctgcaaacaaaaactgtgtgacctaggtcctagggtatgtccttcctttatccatccatccatccaaccaaccatccaaccatccatccactcatccatccatccatccatccatccatccatccatccatccatccatccggctgacatttactgaggctcatctttttcctgggcaagatttcttacctgtaaaatgtgagaattcattgggacttgtatttctgacctaggatcccagaatcttaggagtctcagggcagtgttttgggggctaccttcctaaggtgggtggggcgatgggacagtctcagaacagcctcaacccagaacagccacatttttagctgttttatatattgggtttctacttaagatttctttcgaacaaagcctcccttactgaaaactcactgccccagagggtctctgtaaaccttctagtagtgacactgtagcttcaagccaaattgtttttcaatcaatacccctgcagtgtagggcatagaaataaaatggtaaataaaccagggtcactgctcctcggaggctggctgtcactgtgaggataaactgagataataggtgggaaagcctgtttagggaaacgattgttgttaagatgataacaattattattacgttggggtagggtattcaagatatacccacaggaaaatgcagattacaattcagtctgcaattagggaccagatagcagccaactctcaggtgatccatgcagggattctgagcattcgtcggcgcctccccaggggctgctgcgccccctgcttcccgcgcgcccaccacgcacgctgctctgggagcagggccggcggcgccgccgcctcgcagcgattggttgaaccggaggttgttgctaggctaccagtgcgccctgagcctggggccccgcagtcccatcctctgtggcagatccatccctcactgcagacctaattccggtaccctgtgaacggcatcctcagcagcttaaattatcagccccaactgcccgcctttctatgatttttcatttcgcaagaggccatgtggagttggggaagagaagccttctgtttttatcctctgggtcatcttgaatagcgacttctctctctagacctcagtatcccgcttcagtttctggagttcattcattcatccattattcatgcctgcgtgcatgtttcttaagtgcccaccgtattccaggcactatattgagtgctagggacaaaagaatgaacaagacagagaccgtccttgccctcgtgaagaggggaacattatccacctaaacaaataaaataaacgcatgtacaattatcccttttcttccccctttgatgatttttctagcccctctttccccctactatatctgtagggtcccccaacttgacaggagagggagtgtctcccagtcaggcctacactactctgaaactggggtgcaccctcttactccaagctcgtattttgctcttttgattccacacatgagcctgaccacaccttgataaggatgtgggggcagggatcactgtctcctgttcagatgagcagtctgagagtctgaatcatcaaaatgccttgtcacataagaaattattgccatacccaatatttggtcatttactatgtgctggcactttaaaagctgtctgcctcatcataatccagtaaggtaggtattcttatactaattttacaggcaacaaagttgaggatgaaaggtgttatttaagtggccagtgatgatctagatagcatgtggtgaagctgggatttgaaccaaatctgcctggctccaaagcctaagctctttccaagaaatggatgtggtcatggaaagactctctgatcagctgggcacagtggctcatgcctgtaatccccacactttgggaggctgagatgggaggattgcttgagcccaggagtttgagaccagccttagcaacagagtgagaccctgtttctacaaaataaaaagaattatctggtatggtggcacatgctgtagttccagctactcaggaggctgaggtgggaggatcctttgaggagcctgggcaacagagggagaccctgtctctaaaaaaaaaaagtttaaattaaatttaaaaaagaaagactctctgagtggctgaaacttggaaacatagcttccaagtgccgttcaaaaatctcccagtctcaacatctttttacattttattttttagaggcagggtctcactctgtcatccaggctggagtgcagtggtgcaatcatggctcactgtggcctcgtccttccggactcaagtgatcctcccacctcaacctcctgagtagctgggactacagtggcatgccaccatgcctggctaatttaaattttttttttttttgtagagacaggggtcttgctatgttgccagagctgatctcaaactcccgggctcaagcgatcctcccatctcagcctcccaatgtgctgggattacaggcatgaaccaccacacccagcattgacccagcatcttatttacttatttttcttggcacattagactcataaaacctggtgagctgaccacagtaatcaatattgttaactctcgctgtctatccatctatctgggatgatctcgccttcaagcttttattgggtaattaagtcaagtagctcttccttgggggcatctccattgctctcaggtttttatattccgttgttcacacactgagagccttggtgggcatagaatatggcccatctctcccctggaggaatttccagccctcaggggaggtttctgaggagcttcctatgggcaaagctcagtgacaggctattgtgagaggaggaggagaaccagatttaccctcagagaatcccctcctctgagcagaagtggatgtgcctagggtgggctgaggggtgggagcagtcaaccttgagtaccctgatcaggacacattgggtccgcggtgctgcttctaataaccttgctgtggttagtgtgggactctgtgtctcagagcctggcagcctgcagctcggaaggaggcctttaaatgggagaaggtgctggaaaatatggtaatacataaataagctaaatttatcccctttctcagcacccagcaattgctgtgcttaacacttaactttctaccagcatagatttgtttaaccagattagttgctgggagagagagaaaggagagagatagacagagacagagacaaagagacacagagaaagacagtcagagactttgaagacagacagaaacacacacacacacacacacacacacacacacacacacacccctggagagaagtaagaagatgcagagataacagtatcctttggtgtgtggcttcagccagcagcaggagagctagactgagagaatcagacaacttcctgccctcttcctcccaggctacatgactggctagagcattgctaatttccagatagcaccattttcagatgtgagtactttggaatgtcgacactgtttgcattattttgtccactgttttgttcctgtcacccaaccctacccctgaataggtgagagttgactgtagatgaataatatactaaagccaccattaaagtaagcattataagactttaaatatgcgtgcctttgacctagcatcctcacttctaggaatttatcctaaggaaaataagtatatggataaagaagctagcatgagattgttcattgcatcgttatttagaaaagcaatgaaacaaactaaatagccaaccactcttggtttcctgatcaataaataatggtataaccttacaacagctttccataccgcttttaaaattagattataggctgggtgcagtggttcatgcctgtaatcccagcactttgggagaccaaagtgggaggattgcttgagaccaggggttcaagaccaacctgggcaacataaggagaccctattttttttttttttttttttgagacggagtctcgctctgtcgcccaggccggactgcggactgcagtggcgcaatctcggctcactgcaagctccgcttcccgggttcacgccattctcctgcctcagcctcctgagtagctgggactacaggcgcccgccaccgcgcccggctaattttttttttttgtatttttagtagagacggggtttcaccttgttagccaggatggtctcgatctcctgacctcatgatccacccgcctcggcctcccaaagtgctgggattacaggcgtgagccaccatgcccggctggagaccctatttttaaaaaaataaaaaaatcagctgggtgcggtggcacatgcctgtaatcccagctactcaagaggctgaggtgagaggattgcctgagccaggcattccagggtgcagtgagctatcatggtgtccagcttgggtgacagaacaaaactctatttgaaaaaacaaattaggttataaatggatatttgttgacctggaaaaacattcacaacatattaggtgaaagaagcaagttacaaatagtgtgtattatatgaccccttataagtatgaataaaaataaaatatttagcttaagaagacagaccaaatgttagtctctaggaggagggattccaagacattttaatttttttctttctgcttatctgcatttttaaaagtttttccacaataagcatgtctgatttttgtaataagaacaaatttccttgcttccctcccaggggtggcttctgctgggagatcaggcgttcctggctctcaggcctcatgggaaaagccattgacccttgccaggtgttaagctgctcactaagaattccctgccctggcttgactccccatcccagctggtgttatgggcgagacctcagtgatccaacaccttggtatccaaatgagcagttgccgcatcacctgtgagtgggttagctgctgccctcccttgaaggtaaacactgtttacctctgttggcacatggtaaggctaacctcactaaaaccgtagggcctttctgcctcccaggagcaccctcccctgtaggagggtgacaattacgatgctgcattgctcagggaggtccctgctacacatatgcaggtgtgattctgaaggttcgaaaaaaaaaaaggctgtcaccatggcaacccaggcttgctcagcctcttcattcctgagatcagtccaagccccagggaaagaaggtggggtatggagtcagaaaaatcactgcctggctgcctgacctcaggcaagacacttagcttttctctgtgtcagtttctgcgtctgtaaaatggtgctgaagcttttcttactcacctccaggactggggtggaataatcggtaaaccataaaacacaggaggagtagggcatccttagttttggcttgtccccagattgccatgtgactttgggtaaatcacttaacctctctgtgtctcaggtgctctatttataaataggcgagtaaggcacactgattacttcccggatatattgcaaggtgcctgtgagaatcccagaatctcaaagttggaaggaatcttagcccttagcactcatgcaggaatctttttgataaactctctgacagagatacttgcctagcctctgcctggttacctcctgtggcaaaaaactcatgaccgcacagcttcccatgtagccattgctacatgacactaaccataagaaagttctaccatatagcgtatcatagcatattttacaaataaatgtttgttttaaaaaagttagcaatcctttccagagctattttcactcctgattttcccaaaggcttgcacagggtagagatgagcaggggtcagcaaaaagcaggtgtgggctcggctgcagaagaagcttttggcttagtgtaatagcaggagcctgggctctggggtcacacatcctaggttgaaatgctactttacccacttacaaactggctgacgctgaccctgggaaagttatattacctatctgagcctcagtttcctgatcagtaaaaggaagatagaaatacctacctcccagtattgttgtgggaattaagcaagttagcacttcttattaagcttacaacccagacttccctggtggggttctggggacttggggtggggaagattacaccctcctctgccacccccgaatagcgcatctgcccactgagcaaatggcacaggcgagccttacatagtccatatctcagggtcatctccctctcagacctggaccagggccataaaggaaagatgatgcaatggtttcaagacaaagttcacctcctcctcatgattcaccggcccaaggccagcaccccttccctgcccacagactcagtgggcaccaagcaagtatcccagggcgtcacttaggaacaacttccaactgcaccagtgttttaataggaatctcatacaggaccccaaaaatgtcatctggcagtggacttccaggcagagtgacaagaagttagggaaagctattgggggttgggctaaagtgttcctttagctgttgatatgaaggtgtctgtcacgtaatgactcctggacaaaggtcaggatgcctgggtccagctccatcattgcctagctctgggctctccggagccagcgacttccttcctcaatatcttgcttccgtcatgtgcaagacagcaggccgattcctggggttttcccagcttccgaattctaggtttccccatttgttggattcagagatgagttggagaaaccaccctagacctcccttggcccctggccactgggtggcaccgaccaccccctcttctctgcttcctttgcagatgagtatctcagagtcctcaacgtgggtgtggccgaggtgaagaaatccttcctggggcccttgtccccgtcctgtaagatggtgcagatgatgaggcagtttctgtaccgggtcctgcccgaggactcctacaaggtcaccacggggaagctccatgtgagcctcacccgcttaacggacggggagaatgtggtggtttcagagttcacgtccaaggaggagctcattgaggcaagggggctgggctgggagggagggacacggagggggcgggggagggcggctcctgctctttctccacagctcaacccgatgccttatccacctgcctcacttccctagtccttcccttccttcatgcccccagagtctcgctgtgcaaggtgcagtgctagatgagatggacacagaagcccaggacgaaccaggcttgaccgccaccctcaatgcttcggccttcacatccatcctgcccggccccctgcgctaagcctggagcttcccagaggcccaggtgtgacttgcagagggcctgggtctccttcagggccttccaggctgggctgccatgcatccccccagaattactcccactttgtgggaacggtcaggggagaggggcccccatgtcccctttctgccccctctcttccagtggcccttgataagggattgaatggttaccaaatcagttaacacttagaaaagtgcacgctaatcctgagtgcgctgccctaagctcattaatattaattttagttatgaataatgattgctggaaagggccgcaggtaggtcccagtgtctttcgggacaaaggggttaagctgtgtggcctccttcccacatctccactctccttccttcccccacatacaggggcttcgccactccatcctcatcccatactggcctggcagtaatcctggggatgagggatgaataggcagtgggcacagacaccacctccgtccctcaggccccctgggtttatcagcagccagctcttccagatctgcctcctcacgcccacccccgttctgacttcactgcccactgctgcagttttctcttcccggccctctcccccaccttccactcctaccatgccaaccctagcaaaccacacacaagccaggcctctgggtctttgcacctgccgctccctctgcctgggatcccctccctccttcccacactgctttgcctgctgctgaaacccagtccctcctgccagggcacttcccttcatcccctcccaccctgcctgcacccctcttgtggagccacggtgcccatgctcacctccaccatgcccctgtcaccccactgcaccgtcacccacttgctcactccctctcatagggcactcccaaaggtgggcctctcagcctcctttgcttccagggcctggcccagggctgacacacattaatggttgttgaatcccactggattcatgaaagttcagcatcagaaggaaccccgaagctatggaggaccccctcctgctttaaagctgggacatcagggccagatggaccaagcaggttaccaaggacccaattagtgaaggccaggccaggaactgaactcggagctttgcttcccagtgtggcaccttcacctagaatttaaggaggtctgggctggtggtgggtggcccaggggctgacaggtgagctgtccccaccccacccccgggcctccagcatctcagccctgttctctccgcacaggccctatactgcagctgcttcgtcccggtgtactgtggcctcatccccccgacttaccgcggtgtggtgagtgcttcggcatggtgaggggtgagatgggatccaagggacctcgggtccctgtgactcacacctgggggagcaggggggtggtctcagaatgcagcgggaggacctagagttggagagtttctcatgttgttactcagaattcccaatgtaggtggctttctgggcctttaagatgaacttctccagctggggtgggttcctctctggacccacctacccactgccaagccgggcaaatcaaggctccataggcttggcaggttttcccagacttgacagagcagaggagatgaagcactgggacctggaaactcaaatgatgaggatgttttctgaggcctgtaggccagggccttacagactttaatgtgcccttgaatcactcggggtctcgtaaaattatgcaagttctgatccaggcggtctgaggcagtgcctgagggtctgcgcccctaaccagcctcaggtgctgcccgtgctgctggtgggtgggccacactctgagtggtgatggtctagctcatgccccctcctcattttatatgcagagaaagtcagggccagagactaggattcaggtagtagatggagcttgtattgcatgtcagccagccctgagagttagagcctcagtatttgttgtccttcgttcataggtgagaaaacagaagccccagaaaggaaggggcttgtgggtgaacaggtggtggggctgtagcagaggagcacgtctcctgctttacccagagtatgggtcccctgcacacagagccctgggagccccagtctcagcgccactaagatagtggagggaggatcagtgcaggacagagcaccgggctgggagtccagaggccaatgcagactcactaagtgaccaggggcacaccacgcacccaccaggacctccgtctccaggcttatcctgaggggtcttctggatcttccttgatgggcccttgaagctggtgccatatcccatgggccatttcgatgtaccccgagtggggctgagaactctggccccaagtgggcatttctcactctgcccccacagaggtacatcgatgggggcttcacgggcatgcagccctgtgccttctggaccgacgccatcaccatctccaccttcagtgggcagcaggacatctgtccccgggactgcccggccatcttccacgacttccgcatgttcaactgctccttccagttctccctggagaacatcgccaggatgacccacgcattgttccccccggacctggtggtgagaggcaggaggggtctggggagtagcagaaggtaccagggactaggggtggggttagagagccactggggcccactcaagttccatctgagtctcctcccctcaaatggtcctttaaacttcctcctagacctgcatcctggccttgctgctaactagctatgtgaccttgaacaagcgcctcaagctctcccagcttcaacattctccccggtaaagcgagatgatgccagaagtgctgatagaaatattaactgaacccatccattcagacagttcatgtcccaaatcaaaggtcggcaagctgcagcccacgggccaaatctagcctatggcttgtttttgtacagcctgtgagctaagaaagggttttccatttttagagagttatgggggaaagaaaaaaagaagaaaaagaaaaagaacattaggcagagactgcatgtggccagcaaagcccaaaataagtatttactatctggccctttacagaaaaggctttgtgggcagtagctcatgtaatcctctagcatcctctatgatcaatattataactgtccccattttatagatgaagaaattgaggctgtgagaggggacagcaacacagccaataaggggcagcccagagacctgaactcaggctccaaactttgctcttcatatggccccagtgcacctggttctgccatgagcaccccacactttcctcccaggggaggccgaggctccaccatgaatggtgtaactcatgaacggagcatcactggggcgtgtgatcaggaattgctcctcggttcttcttagcagacagtgagggatgggacactggatgggtacgttttgaaaagcccatcaaataaaattcaagagcaatgggagtagctgccctgggtcgggggaactgtggctcccttcctccccgcagccttggtaattctcctggtgcctccgcccacagatcctgcacgattactactaccgagggtacgaggatgcagttttgtacttgaggcggctgagtaagtaccggtggggccccaggtaagggcagtgttggagggtagggaaagttcaaacagtagaagggctgaatggaggggtattccccccagatttgtgacccggagacgcccttcacctgggagagctggaaatgggggtggggacctaacaggctgcatcgcccagcccagaccgctgggtccagaaagtaggatctccatgtaaagggggctttggggttaactaagggcacagtgtctaggacagttgtgcatgtatgcttatgaatgtaaaaactctatttaggaaatccagagttcagatcttcctggagaattccattttcatgtaatttcattctcttccactttattctctttctagtaaaatcacattattaatgtagaattggatgtcactagtttatatttgtgtaagactttgagtgacctattgatcagcaaatcacagagaagagcccttgccctatgtaggtttggaatggtggtccctccacctatatattctctgtgctttatcagggtggaagaaacaattccaatgacatgtcttttatgtgctaactccatggcaaacatctttgttaaaagaaatgaacaggactgggtgtggtggctcatgcctgtaatctcagcactgtaggagggcaaggcaggagaatcacttgagcccaggaatttgagaccagcctgggcgattgtgagattcccagctctacaagaaattaaacaattagctgagcatggtggcacacgcttatagccccagctacttgagatcaaggctgtagcgagccatgactgcaccactgtactctactctgggcaacagagtgagaccctgtctctaaaaaaagaaagaaatcaacaaaagctttgccatatttagaatcaattattttcagaaaaagggatcaccaggtcaaagggtttacctaatgttacatcagaagcattttccttgttgccacatgtctttgtgactgtcttttttggcagttgtataatattccatcatagctgtgcagaaaaatttgcttgatcattcctttgagtttccaaatgttcgtgattatgagtaattctgtggtaaactttttgtgtatgtagctttaaaaaatactctgtgggtgagattaaggggttggatgagattttcttcctaggaggtgctcacttatttttcccttcttcaaaattccttcctgcttcccaagctccccttatcctgtctctactttctcctgggtcctttctcccaacaaggggggtggagaggctggacggacaggagagcaggatggagaaaacctgggccctggaggcaagtagctctaaggcccccatcccacccacaagctgtgtgacctccggataattgcttcccctctctaaggcttggtttcctaatctgtaaaaaaaagggggtttagatcctagctcctgacacctactaactgtgtgatattgagcaggtaattaatctcctggggcctcagtttcctcatctgtaaaaggggccttatggttttattttgaagattaaaggagcaaagccacaagcccttatcacaatgctcaaataaatggtagctctcatgagaacgtcaaagtgctgagtcctagccacagtcagtcccgcctactgctgggtgaaatctgatggtggcacttcaggaatctgtccccaaaattgagccagtatatggatgggggcaggcttcttcaatggccctggaatcactccaagtaggaacagatacaactcctaaaggagaaatggtctcgtgctgactaccagctatttttgcttcctcttccgatttgccagtgtatggcacgggtttctcagcgcagatttcacctccaccacctcagtcctcttggatttggtgggactggatttttctctctgcatcagatgtgaacaggaactactgacacatactgagaaactgcagttggtctccagagccacactcttatccccacctgccatctgggaacagaaaatgggagtccgcccagcctcccctgggctgctttctgggtaggggtcacctccagcaggaaactacaccccaaaaggtgtcaccctccattcctcagctcacattctcacagcctctttctcctccctcccaagggaggagaattaattattcttgctgccaggcaagccactctgtctacactcggcccatccttgcagcggccttctgccaaagggaatggcagcgcaggctggcgtgcttgaaaatccgcattccccacacagatgtgtgaatgtgggtgctgagtgaggttggagctcagaggatgccatgtctaaaatatccatccggcattcaggcatgcctggccaaacctctcttccgccctcctcccccactcccttcttccctctcacagtttggtaagtgactctgtctctctgtacacacacacacacacacacacaccctgtgaaaccatcaccacaattaagttagtgaataattcgtcatccccaaaatttcctcatgccctttcgtgatccctcccaccttccctcaccccccgccattcccaagcaacccctgatctgctttctgccattatagattaattcactacaatgtgaactcttttttaaaaatctgaattctttcactctgcataattattctgagattcatccatgttgttgaatgtataaatagtttgttcctttttacgttccgtgccactgtatgaatgtaccacagctcatctaccaaaagagttgaatataccactgtatgcatataccacagcccatttacccagtcccctgttgatggacatttaggttattttcagatttgggctattatggataaagctgctatgaacactcacgcatgagtcttggtgtggacatatgctttcatttctcttgggtaaatacctagaaatggaatgacgggattgcatggtacattcatgtttaacatacatacacaaatattttaattgtggtaaaaaaacacgtaacaaattttaagcacacagttcagtagtgttaactatttcatattgttgtgcagcagatctctagaactttttcatcttgcaaaactgagcctttacacacattgatcaacaactcccccttttctcctcctccagcccccagcaaccaccatcccactttctgtgctgatgattttgactacactaggtacttcatataaatggaatcatgcggtatttgtctttttgtgactggcttatttaacttaacataatggcctcaaggttcatccatgttgtagctgtagcatgtgacaggattttcttccttttttgagacggagtctcgctctgttgtccaggctagagtgtggtggtgtgatctcagctcactgcaacctctgccacctgggttcaagcgattctcccgcctcagcctcctgagtacctgagactacaggcacacgccaccacgtccagctaatttttgtatttttagtagagacagggtttcaccatgttggccaggatggtctcgatctcctcacctcgtgatctgcctgcctcggcctcccaaagtgctgggattactggggtgagccaccgcgcctggccgattttcttcctttttagagctgaatatattccacatgttctttgtccatccatctgacaaaggacactggagttgcttccatctttcggctattgtggataatgctgcagtgcccatggatgtctcttccagatcctattcaattattttggatatataaccagaatatatgtttgactttttaataaactgccaaattgttttccaaagcagttgtaccatgtttcattcccaccagcagtggatgagcgttcccattcctccaaaacttttggggttttttttgtttttttgtctgtttttttttttttttttgagatggagtctcgctctgtcacccaggctggagtgcagtggcatgatctcggctcactgcaagctccacctcctgggttcacaccattctcctgcctcagtctcccgagtagctgggactacaggcgcccgctaccacgcccagctaattttttgcatttttagtagagacggggtttcgccatgttggccaggatggtctcgatctcttgacctcgtgatccacccgcctcggcctcccaaagtgctgggattacaggcgtgagccactgcgcccggccaacttttggtatttttaattaatgctcagaatttttcattttagcccttctaacaggtgcatgtcggtaacttattgtggttttaatcagtattcctctaatgactaatgactttgagcatcttttcatttgtctatttgcctcccatagatcttctttggtgaagcattcgttccactcttttgtacatttctcacctagactcttaataagctttgtttttttagggtagttttagatttatggataacttgtgaaaataatacagggtgtttgtctcacatctgttagacgttacagaatgatacatatataacacggtattcagtccagtacatttgttacaactcatgaaccaatcttgacatattattaactaagagccattgcttattcagattttctcagtttttacctaatttctttttctgttccaggaccccccacccagggatcacattacattttgttgtcacgtctcctcagtttcctcttgcctataacagtttctcagacttttcttattcttgtgtgtgtgtgtgtgtgagagagagagagagagagagagagagagagagagagacagagtcttgctgtgtcacccaggctggagtgcagtggtgcaatcttggctcactgcaagctccacctcccgggttcatgccattctcctgcctcagcctcccgagtagctggggctacaggtgcatgccactacccccggctaattttttgtatttttagtagagatggggtttcaccgtgttagccaggatggtctcaatctcctgacctcataatccgcccgcctcggcctcccaaagtgctgggattacagagacttttcttattcttgatgaccttgacagagttgactatcggtcaggtattttgtagaacgtccctcagttggggtttgtctgatgtttttctcaagattaagctggggttttgagttctggggaggagaccacagaggtaaggtgctattcacatcacgttacctcatgggcacgtgctgccaacatgacgtatccctgctgatgttgaccatgaccgcctggccgaggtagtgtccatcgggtttctccattgtgaagtactctttctccctctatccttactgcagtttttggaagaaagtcactgtgcacagctcatatttaaggagtgagaagttatgctccacttcctaaagggtcaagcatctacataaattactaagaattcttctgcaggggagctttctctcttctccctgatttacttacctacgtaatatatgtttatatcagtatggattcatggttatgttatactttgggttattattcaatactactttattgtgttcttcaaattgttccagctttggccattgggagccctttcagttggctctttcacacattttaaaaactgaattgtttgttttcttatttaagggctgttatgtggcctagataaacgccatccccccgcccccccacccacccactatttttgagacaggttctcactctgtcacccaagctggagcgtagtggcacgatcatggctcactgaagcttcagcttcccaggctcaggcaaccctttcacctttctcaatgagtcttacagccacagtgaccttctcagaaccagcctcagctgattacctcccctgcctaaaagtcatgctacaataatgcttgccatgaaaactaaggatcctagctgctgcctacagccctgacatggtccggcccctgccgcctcctcagcctcctcccccagcacagtacccccgccccacactgacctttcaggcccccgaccacctggtgtgtcctcaaactttgtggcctttgtgtgtgctgttccctctgctgaaacaccctcaccaccccgccctgaccatcctgggccacgtacccttcagatctctactcactgccaatccctcagatgacctgccctctctcacagcaccacattcctctcttcctgcacttgcctcagttgaaactctagataatttgggtgggcaacgcctgcttccccacactacaccgtaagtgccagaaaccatcacgtttgttttcactcactgtaaattcaagtaagtacagaaagacctttgaaaagcactgttcctgtttaggaaaataactcaagaaaccatttttgcaaaggccatgctgctgccagggctgagtaacaccccatgctattgtttatcctagatgctgtttatcttaattcttcctccaagagagtgattttcccccgggtggaagtgtactgccagatagaactcgcccttggcaatgagtgccctgaacgcagtcaaccaagccttcgagcacggcaggccagtctggaaggagccacacaacctcacaaggagtgggttcccaaaggggatggaaggggcagccatggtccgcctgtgtcccaacctgtgcagacacttgaattcacatgcgagtcacctgtttcagcaccagtctctccacttgagcagccacctgcacagccactggcctcttcaactccactttctctaagtggcatgccacctgtatcattcccagctgtgcacaagccacccagctccacacctggttcatcactgcccaccccaccacctggactgtcacctctgtcacctcagcagcaggtacaaccgtctggatcaccagccagatccctacactctcaggcacccacttcacccaggccatccctggggccttcaactgtgggggcacctcaaacactgccccgaagttctctttcagccttccctgctcagccacctgtggaggaactaggccaagaacagccccaaggtatggacccttctggcttgttatgactttctcatgcctggagccccttggcaagcgagcaaggacaccagggctgataaccgcttctttaggggtgcgtggctgaagttagcagtgagctttagcatctctgtccattatgaggacagtccgccacaagggggcagtgtaatagacattggttcatttcccttggtgcagtgcttctcaaagtgcgatccccctaccggcagcaaaagcatcacctgggaacttgctagaaattacagttctcaggtcctactgcagacctactaaattaccaactttggaggtgcggcccagctgccttttttacaagccctccaggtgattttttgcctgctcacatttgagaccgctgggccagtagatttttactgtgaaacctgctgtgctgggcgtgctactcaaagagtggtccgcagaccaacccagccctcaactgtgtgttactagcccaccacggagggtgtgcagaaattaagagtaatcatttagaaacgtttatagcactaaggtatttccacaacatcccggagcttgaccattttttactaattcatttgcattgtatttcacaaaattactagtttgttatagactacaaagaaatttttttttttagatggaatctcgctctgtcgcccaggctagagtgcagtggcgtgatcttggctcactgccagttctgcctcccgggttcacgccattcttctgcctcagcctcccgagtagctgggactacagatgcccgccaccacgcctggctaattttttgtatttttagtaaagacaggtttcactgtgttagccaggatggtctcaatctcctgactttgtgatctgcccgccttggcctcccaaagtgctgggactacaggcgtgagccaccgcgcccagccagaaatttttaaaaaactgtttccttactactacagacggtttcagaagcaccactatagggcctgcaaagataaaacgttctcaattccttactcaggaaatctccctttcagcagaggagataacagggatactaacctcaaaaatacaatcagggactgggtgtggtcactcacacctgtaattccagcactttgggaggccaaagcgggtggatcacctgaggtcaggagttcaagaccagcctgaccaacatggtgaaaccccatctctactaaatacaaaaaaatcagccaaacttggtggtgcatgcctgtaatcccagctacttgggaggctgaggcaggataatcgcttgaacccaggaggtggaggttgcagggagccaagattgtgccattgcactccagcctgggcaacaagagtgaaactccatttaaaaaatatatatatatcagaagtattaagtactatataactaataaaaacacagtgttggagggtattcgtagagaataagaccacttatctctggggcagtatgaaaagcttcttggaggtgacaggtgagcttaaatgggcaggattttgaacatcgtggtttgtgggattggatgggttggagtagaaagagcatttccgattttgtttatttaaaatgctcaggtgcagtgcaggaatgtgcagggtactttcaaggtactttaggaaggagaggacttgtgtgtggccaccatggatggtctgggtggagacagagggacagagacccaaatgaaggactgaagccagattgtagggtgctcatggtcctcagccaaacttccagagctattatggtaaacgtcacagatcggcaatgtctcctcatctgggaattagccccaacttggtagagatgccctttctttggaacaggtttactttgactcttttaatgagacaaaaacaaaacaccattaagaaaattaggctgtgactcatgcctttgagaggcagaggcaggcaagtcactttcagtcaggagttcgagaccagcctagccaacgtggtgaaaccccatctctactaaaaatacaaaaaattagccgggcatggtggcaagtacctgcagtcccagctactcaagaggctgaggcaggaggatcacttgaacctgggaggcggaggttgcagtgagtagagatcgtgccactgcactccagcctgggcgacagagtgagactccgtctcaggaaaaaaaaaaaaaaaaaaaaaagtcagaaaattgggcgcagtgttttacctgctctcttatttcctgttaaatacaaaacacttgttttacattaaccacacctgtgcatgaaggcggccatcttcaggtacctgagcaatcctgcaccccaagtcggcattcctcactcatatcgggggtggatagagggcatatatagtctagtgattctctactgggagtgattttgtctccaggagacatttagcaatgtctgcagacattttggttgtcacctagtggttacaggctggggacactgaaaaatatcctgtagtgcacagaagtctctgcacaaagtgctttctgaccgttagtgtcaatagcatgtactgagattgagaagccatgttatagcttacagctgagcctcaatggtgacatcctaggccatctatgtgagttaggcagagctgagaccccctagagcaagtttatccaacccgcggcccagatggttctgaatgcaccagtctataaaaaattcattaactttcttaaaacattgtgagattttttttggattttttttcagctcatcagctatcattactgttagtgtattttatgtgtggcccaagacaattcttcttccagtgtggcccaaggaagccaaaagattggacacccctgccctagagtttagtggtatcagtcatttaatgatggccattggtggcattggggaacaggaaaattgttaaaagccctaggaatatccaaaaacaaaaggacaagcctcattgtgaatagatttgggagagttagggacatacaacaagtctctaacaaaataacttcagaagaacccacatatactttatattacctttatttatatataatacataattacatattaaccttcatgtattatgtatccccatactcattaaaaacatgtaaagagcttacttaagagtaatggccatgaaactattaatactctgttctaagaaacaaacaaaaagcattacatgaagcatgtgatggaaagtccaggccccttcaggggattttcctgaaaaccttccctaacatgctgctttctgcgagggagctatttcttttcttttccttatttttctttacttttctcttttttttttttttttttttttggtggtggaggaggagacagtatctcactcccggctcactgcactctcaacttttgaggctcaggtgattctcttacctcagcctcccgagtagctgagatcacaggcatgcactacaacacccagctaaatttttgtatttttagtagagacagggtttcaccatgttggccagactggtctcaaattcctggcctcaagtgatctgcccgccttggcctcccaaagcgccgggattacaggcatgagccaccgggcctgcccctgggagttgtttctaatgttccaccaagggtccgagtcagccgctccacagatgccgcgggcctgcgtctcagtcaagtcccacgacatgactctggttgtaaagatgagaatttgaggacaagagagttctttgctgtttttaaacattctcatttactgactattgctatttcaaaaatgactccatatcccccctccccatctcactcccgtttcctatatctttacttttagctgtagctcttcttgtctcttcaaaaccaaaaagcgccgtgcctctggttcatgtgaaggaaaccgtcagcaagccttatgtaacgtaagtttccccttcgtggagcacgctctttcctttggacggaagaagcaagcccggctaagcgatatcttccaccaccttagctgccccaggaactctgggatccttttctggtgtgtgtgattgtgtgattccaggtccacccctttacatctcacaaagctgccttctcttgttcagttagggcagtgtgtggggaaggagcctgtccgtggtgatgatgtcattctcttctcctgcacactggggatgctccttctctgagtgcagtgcaagaatgtgcatcccctgccctcatattttactctctggtgatgccacagttccaagagggcctgcaggcatcctacctcccagccttaggtagctcctgcatgaaatgaattaagggtctctatcaaaggccttccttactccgaactattttctattagcatgataatgacctgatagtactagactgtagactgtccccagaaaatcttttttattatgcacatcaagtgtctgggttgcataacatacactccattatatctttgcaataactatgagaggacctggggatctgcttttttaaaaacttccctgaggaccacattacccaggctcccttgctggctgaatgccgtttgtactggtggtgggggaggcacagcaggagatcagaagttgggtcaagggtatttctccccacaaccccagcccccctctcatccctccctgccttgggaccactctggcagtagctgggtttctccatgaccacagctgcaggtattgagagagcagggcctgaggcattcctgcttttaatgtttgcactctctctcacatcaagcatattaaaatttacctgcttcccacattaagtataattttaaccataaaactttttgaaagagtttgtataaatttaacaattttgagcttaggcataagttatgcattttgttcacataatttaaacttttttgcatttacatataactctataaaagtcggagtttcatttgactcatgggcatgatgttacttagataaatattcattgattatattttaaaaaaagtttgctgggaaatccaatcctcagccagggttgagaaccacagcgcgggtgtgtccacctgcggagctgagcaggccaccatgttggaggaccgaggtcaggcccataatgaaccatctacttaatctctttgcctagggagagccctgctgaagactcaaactgggtgaataaggtcttcaagaagaacaagcaaaagacaagtggcaccagaaaaggcttcccaagacattcgggatccaaaaaaccaagcagcaaagtgcagtgagcatgtctaatgttccttaaatcccacggagaggagcagctttgggaactgtgttcagagagattccgaggaatagaggagagtgtaagggagtagggggtgcagtgggagattgggctttggaacagacacatccgacataaaattcctgctctgccacagctccactcagggatcatggttgggacacttgctctccctgagcctccatttcctgtaaaatggggatgataccacttcataaagttgtgagagttaaatgtgatcgatgatgtaaattgcttcatagaatgcagaatgtgtaatagctcacaataagtaggtattatgtttacatattatgtttgtatttatgctacttaaatacaaaactggacaggccaggcatggtggctcatgcctgtaatcccagcactttgggaggctgaggtaggtggaaaacctgaggtcaggagttcaagaacagcctgaccaacatggtgaaactccatctctactaaaaatacaaaaattaaccaggcttgatggtgtgcacctgtaatctcagctactcgggaggctgaggcaagagaatcgcttgaacccaggaggcagaggttgcagtgcaccaagactgcgccattgcactccagcccgggcaacaagagcgaaaccccatctcgaaaaaaaacaaaacaaaactagacaagtgagtgcctacgtgacactcaaatgttgccagcatacagttaagggccctagtcaatgtaggcctgcttcttatagctttttgactatattatgctgtctttgacttagtcagtcaacacttattgagcacctactaagtgccaaacactctcctggactctggcaaaataaaaaatgaattaaaactctatccccaagtttcaacagtttactcctagtctaactttagatcaaagataagactaaaacctaagaatctgatgaactttgagtcccacctaaaccaagttttaatttttttaccgagtcatcatattttaagtaaagccccatgcatgtatacaaacagactcgcaaattcaaaatgagagcctcagattcaaaccaagtcgggtattatttacagttatttcattgggttatcaaagtattttctgaattggccaggcatgttgacatgtgccggtagtcccagctattcgggaggctgaggcaggaggattgcttgagcccaggagttggagtctgggctgggcaacatagtattctctgaatggaacagacctgcactgctatataatttgcttcaagtctttgttggtcagaaaaacctctaatcattctgagcaacatccttcctggttgttcttgtcccaagtggggtccctggatgggcacagggatttatcagacagacaagggtcagaggctcacccggcggccaaaggctcatctcgaagtctaagcaatgcacttgatgagtggtgggaagcagtatggtagcggagattctgagagctgctgcaggaa

SEQ ID NO: 25 - DNA, 2152 bp, Mus musculus
cccgtccagagcagggtgcaggaatgcccccgctccaaggtgcgctccgaatgggtgaagagcgctgaagagccggtacctgacagaagtctagcaagggcatgaagtgagcaaggggtctgcaggaccacgcccctgcccatgactctaaggactgtctacatttcatccagtacctggggatccctatcagcccttccggggtacctctcagcagagctaaacaggcccaggcagtggctggatgaaccacccgtggatgcctgactgggctggtagagttgccacattccctgctcttggctgggggagaaggagatactgagccaaggcagacaggtgtgggttcttcctccgctgggaggccatagaccccgaacatggacgaacaggtgttcaaaggagacccagatacccctcattccatctccttctccgggagcgggttcctttcctactaccaggctggggccgtggatgctctgcgagatctggccccgaggatgctggacacagcccatcgctttgcagggacgtcagcgggagcggtgattgcggccttggtcgtctgcgggattgagatggagaaatacctcagagtgctgaatatgggcctggcggaggtgaagaagttcttcctgggacctctgtctccgtcctgcaagatggtacagatgatgcgacagtttttgtacgatgtgctaccggaggactcctacaagttcgccaccgggaagctgcatgtgagcctcacccgagtcacggacggagagaacgtcgtggtttccgagtaccgatccaaggaagagctcatcgaggcaaggccctgctgctttgttcctgtttactgtggcttcatccccccaacgtatcggggagagagatacatcgacggtggcttcacaagcatgcagccctgttccttctggacagactccatcaccatctccaccttcagcagccagcaggacatctgtccgagagactgccccaccatcttccatgacttccgaatgttcaacttctccttccagttctccctggagaatatcacccgcatgacacatgcgctgtttcccccggacctggtgattctgcaggaatattactatcggggatacaatgatgctgtctcatacctgcggagactgaatgcagcgtaccttgactctcccagcaagagagtgattttcccgagggttgaagtatactgccagatagaggtcgcccttggccatgagcccccacctccgagtctgcagaacctgccagccctgaggagaagcccagcagactcctcacaaacccatgcacaggggtctcccaaaaaggacagaaaggacagccattcctcagccgccccctcagtgcagacacctgaatctgggtgcaaggagtctgtggaatcacccgtgtcactacgggtctctatatccaagcaaccatctgtatcgccattatccccagcccagccggtcccagtaatgaggcccactggccccagggacagttgcccaataaatgttcaaactccaaacccggagcgaggagtgaagggtgccctggactctgccacagaacgaggaatgaaggatgctctggcctctgccactgacgagcagagcacaactaccttgccacctgtgttgctcccagctgcagactcacgaggctcaaagactggatcctctgtgcctattgggtcacccgagtcccctagactgctgctcagatcttcccagggagccacagcatccagggcaacacttggacttccgcccctaagtccttcaacaccacctgctgggccacctgtggaggatctaggcccagaacgacccacagctacagggtctcccgccttatcacagctgacaggctcggcagcaccgggtaccgggaagaaagccccccacaagcctctgctggtggagggtcctggtgaggattcaaatacggcaaaaacgatgttcaagaggaagcagaagaccaatgccaccagagagtgcttccaccgaaatgctcagtccaagaagccggctagcaaactgaagtaagcacctttgtcacttaactttcctgtactcccccaaaaagtttgggtcaccttataaacttcatccccagccgggattccagaacttacagctac

SEQ ID NO: 26 - protein, 558 aa, Mus musculus
MDEQVFKGDPDTPHSISFSGSGFLSYYQAGAVDALRDLAPRMLDTAHRFAGTSAGAVIAALVVCGIEMEKYLRVLNMGLAEVKKFFLGPLSPSCKMVQMMRQFLYDVLPEDSYKFATGKLHVSLTRVTDGENVVVSEYRSKEELIEARPCCFVPVYCGFIPPTYRGERYIDGGFTSMQPCSFWTDSITISTFSSQQDICPRDCPTIFHDFRMFNFSFQFSLENITRMTHALFPPDLVILQEYYYRGYNDAVSYLRRLNAAYLDSPSKRVIFPRVEVYCQIEVALGHEPPPPSLQNLPALRRSPADSSQTHAQGSPKKDRKDSHSSAAPSVQTPESGCKESVESPVSLRVSISKQPSVSPLSPAQPVPVMRPTGPRDSCPINVQTPNPERGVKGALDSATERGMKDALASATDEQSTTTLPPVLLPAADSRGSKTGSSVPIGSPESPRLLLRSSQGATASRATLGLPPLSPSTPPAGPPVEDLGPERPTATGSPALSQLTGSAAPGTGKKAPHKPLLVEGPGEDSNTAKTMFKRKQKTNATRECFHRNAQSKKPASKLK

SEQ ID NO: 27 - DNA, 34285 bp, Mus musculus
gctctctccctgaggaccggtgttcagttcccagcacctatatcaggcagctcacaactgcctgtgattccggctgcagggcatcggataccctcttcaggcctctggagctggcacaggactgagaggcaagagagaagacctgcacggaccaggccagggacagggagatgacagaataagaagaggtaggaaggaggctggcgacttctggctggttactgatggacgctgcgaagagggactctcattggaggagttgccgccatcagatcggccgccatcagatcggcaagtctatagggtattttcttgattaatgattgatggaagaaggctcagcccactgtgggcggtgccattcctaggcaggtggtctgagtctgtgtaagaaagcaggttgggggctggagagaaggctcagcggttaagagtagtgactgctcttccaaaggtcctgagttcaaatcccagcaaccgcatggtggctcacaaccatcctcgtaacaaatctgacgccctcttctggagtgtctgaagatgcagtgtactaaataaatctttacaagaaagaaagaaagaaagaaaggaaggaaggaaggaaggaaggaaggaaggagacagcgtgtagcgatgggggagcaaacctgcattggttcctctcgccctgccttgactttcatgatgaaccttaaactgtgagctgaaataacccttttctccccaagttgcttttggtcacggtgttttatcacagcaatggaaagcaaaccagggcaaacacccagagccatacttctctgttacagtttttgcccagagccaatcctaaggtaagaataaatagagaggtgttttgtgtaagaactttccagagtctgaatggggtgggggaggggcatggggtggtatgagttgctcctccccattgaaagccaaagccatcctgcagcccacaatggaagtgaggggaacacaggctggggagggggcgtggcggatagggggcgtggcgtgtaactctgcgtcctgtcagtgaagagcagcctgtcagtgagggaggcacagggcgaggcagagattttgcaactggatgctgagaggcaccgcctctgtggacaccgtccagagcagggtgcagggaatgccccgctcctaggtgcgctccggatgggtggagagcgctgaagagcctgtacctgacagaagtctagcaagggcatgaagtgagcaaggggtctgcaggaccacgcccctgcccatgactctaaggactgtctacatttcatccagtacctggggatccctatcagcccttccggggtacctctcagcagagctaaacaggcccaggcagtggctggatgaaccacccgtggatgcctgactgggctggtagagttgccacattccctgctcttggctgggggagaaggagatactgagccaaggcagacaggtgtgggttcttcctccgctgggaggccatagaccccgaacatggacgaacaggtgttcaaaggagacccagatacccctcattccatctccttctccgggagcgggttcctttcctactaccaggctggggccgtggatgctctgcgagatctggccccgaggatgctggacacagcccatcgctttgcagggacgtcagcgggagcggtgattgcggccttggtcgtctgcgggattgagatgggtaagacttttattctggtgtctttgaagggggctgctggagggggcttctcagaacagaccctggacaagcttggaagtgcacggggagagaggcgggaaagcctggcagccagggtagtcccccttcacacacacacacacacacacacacacacacacacacacacacacgtcctccgtcctcctcctcctccatgggcgaggcttcttttaaattgtaaacactgcctcaacttcccagtgaggaggaggggttcccaacatgcaaaccctgagttgaggagaagctggtgacagatgtgggacacagtgtgggagcttcagctgtgccccggtgggccagaggccacagtgctgacccagggctatgagcaacagccttcttggcccggccaacccccacctgctcgatgccaggctgtaatgtagagcccctgtgaagttctagaatgttctagagagaaaagacagcaaaattccgaatggagacgtctctcgtgtcatgatggaagggatgggggtggggggtgtctgtgaacaaattgcacccggctggggctaaagggggaaaggggaccgtgttccaaagatgtttgggggggtgggccatgttgaggaggaggcatctaggtggagactgcacaaagtaagacatttagagaaaggacatttctgggccccgggggacagacctgggggaggaggactggggctgaggttggaggaagcaacggagggatcagggatctggtgacttgagactgtatcagccagctccaacccagtgctgatgcacctgtggggtgccaagtctgcccccaaactgagtcctagccctggtattcaattctgtgcacacaaaagccagttgctgagctccctgtcttccggtttctgccccgtggtggcctgacctgggtttgtccaaggaggaacttgatggataggagtgaactgtttcatgactggggcacggagtgctggaaactgtcaactgtctaatttgggggttttaaggacgcctgactgctgggttggaggggccagagtagaaaccaactaggggatgagtgagctctgaggtgtcagttattggatctagccttgaactctctatgcagttgaggataacctgatcctcctgcctccgctcttccgggctaacctacacttcatgcccatgtttgtggtactgggaattgaacctaaggcttcgcacaggctaggccaacacccttcccagtgaaccacacccctagccttggcctctgctgccttatagtaagttcttaaaaatataccgtttcagactttaatcccagcactcaggaggcagaggcagttgcagctcttgagttcaaagccagcctggtctacagagtgagttccaggacagtcaaggctacacagaaaaaccttgtctcagaaaacagtatctatattcccagtaagtttatagcaagaccttttaggatcgattcagtttgttcaataggagaaataagaaacctgcttagccagcctgaggccaccctggactacagggcacgggaggaagtcatgaagaggactttaggagggatcaagccctagacccagcatgggtcatgaggatagcagtatgtaaggaggggtgaggtgtacggaccaatcttgttcagtcatttactgccccatgatttcctgataaccggaataccctttgtatcacatcctgtttaaagttcttagtatattaactcatccagactttacagccccattccgagggggaaactgttattatctccctttatataaagaaggaaacagagtcacaggacactaatacagcccgaggtggtatagacagtaggtggggcactcaaccggaacctagtctgtccagagtggacgcttgaatgacttaaaccatatcgctgcttggcagagtggactcccgtgggatgctggctttgtggctgaggggttagcatgggggccggagtttttaagacagacctgatacactgaaggatgggctggaaaatattttgggctgaggcatggtggggcgggcctttaatcctggtgcatgagaggcagaggcaggcagatctctgtgaatttaaggtcaacctggactacagagtgaattccaggttagccagggctatatagagagacctgtctcaaaacaaaaccaaacaaaaccaaaaataaagaaaattatatatatacatatatataatacatatatatatatatatatatatatatatatatatatatatataattttgggttttttttaaagaaagaaagcactttggggagaacatagaggatggattaactgggctgttgatctgttatcagcagctcagtgggggcctgaactcaagtttccatgtgccactactattaacctcctctcagttcattgctttgttgatggataccccaagtcctgtgattgtaaacaatgttggagtgagcaagctggctggagtcaggcatccccaggatgcgtgtaagcccttagactcaagagtagcctttggtgggctcttctatgccatgctggagaattctctgaagtgactatacagtttaatgcccagacacacacaggagtggtgatattgtgtgtatgtgtgacgtgtgtgtgaggtgtatgtttgatgtgatatgagatatgcatgtgtgtgagtgtatgtgcgtctgtgagagatgtgtgtatgtgtgtttatgtgcataagatatatgtgtgtatgtgtgtggtatgcatgtgtgtgtctcatgtgtgtaatgtgtgtgtacatgagatgtgtatgtgtatgagtgtgtgagatgtatgtatgtgtgtatgtgaggtgtgtgtgtgtatgtgtgtatgtgtcaaacatgtggaggccagaggagaaccttggtgttaagctcagaaacatcgtacacctccattgagttggtccccgtggcttgaaactcactcacaaggctagactgactggctaggaagccccaggagtcctcctacctctgcctctccagtgccagaatatcaagcataggcaagcacacccaaggatgtatttatttatttatttatttatttatttatttatttggtttttcgagacagggtttctctgtatagccctggctgtcctggaactcactttgtagaccaggctggcctcaaactcagaaatcagcctgcctctgcctcccgagtgctgggattaaaggcatgcgccaccacgcccggcacatttatttttttaatgtgtatgggagttttgcttgcatctatgtctgtgtgcttcatgcttccccctcctcctcttcttcctccccctcctcttccctctctctgttccttcaaaccttccctctattattctccgcctctttgccctctccaccatgaaaggctcagttgcagcgattttatctggaactaagcctcccatggtaaatataacccccatgccctttaataaagacactgggtgtcacttattatatgtttatcagtctaaggatctttgttttgtttgtgtctcactctgtagaccaggcttgcctggaactcactgtgattctcctgcctcagcccccgggactaggtttctctctaaggctcttatggtttttactgccgggagagggcttccctttccacggcacttcctgtggctcagcactgatatagaagggtccgaggggtttctctgcttgtcttattctcggggatttggcacgcatccgtagtagctgagaaagtgtgttcattgcctcatactttatgcaggaccacataactggagatctgttgctttttgaaattgtctttattatacttactgtgtgcatacacaagcaagtgtctgcgcgtgtgtgggtgcacaagtgccatgctgaacatgtgctcagaggacaactttcaggagctgcttctctctcctgcccccaggtaggtcctcggctgtgtccttgcatgacttgggccttctaaccttgactaggggcgtcaggggataaagaaatgacagataaacagaaaaactaggatggtgtgggcggagcttcctgatggagaaactgtagcacgtggaagctccggtgtgttcatcataaacagcaggacacggaggcagggtaattacatgtggataaaccaggagatacgatttatatacagctggacaaggggacagggttattatatgcagatataccaggagataaggtatatacatagctggacaagaaggcatggttagtatatgcaaccgggttaagaggtcagggatattatatactgctggaggcaggtttcgctaatcttggtaggagcagtctctccctgcaggggagcaacctgcaggctgtaaatatcagggaggtgggcattttctgcacacactatcaacacccacacagagactagaggatggctttgccatttccacgaggccgaggcattgggtctttgacatggtggtgcccaggtcaagagcacagtgacttagtccttcttttgcttagggtttcctctgcaccccacagtcctgggtatcaaactcagctcatcagactaggcaacaagcaactttacctaccgatccattccgatggtgtcctgccttctttttttctttaaaaattctttaagctcagtcacacatcttccctcaaagttgtttccactcagggcttctctgaccagagacttctctgtctcttccttggggaaactgaggctgcagagggcccatcttctaatgggtctacttcagtcggtacgacgacatcacaaaagcacctagcactctgcctggtcctcatcagagatgacaccaccatttatatgaggcgaggcagtctccatgacaaccaacagggatccaaagtttctgatgtggccagaagtgggacacagtctcacagagcttgatttcctgtattttgaaaataactgccttagagggtattttaagttacattcctttatcatgtttatgtgtgcgggcatggggggcacgtggcacacctggggggcggggcagaggacagctttcaggagtctgttctttctttccaccgtcccagggatcaaactcaaatcttcaggcttagcagcaagtgcctttgcacacggagccatcttgccagccccaaaagaatttctgtctcttatcaaaggataaagattccggtggtctggcagctccacccccttctgctgctactacagcaagaccccctcccccaaaagactttatggcaaaccttccccgcctctgtccatactttccatttctgtctgtctcagatagaccaggctggcccttcatgtagccaaggctgacctcaaactcatgttcctcctgtttgtgcctcccaagtacaggaagacagggatgtctcaccatgcctgcctctccatagtgtcgttctaacatgagaggaagtgtctgctgggttctagaaaggcacagagtgaggccactgcctgagggagccagcagtaggagtgggagttaggaagggagagacaggctgggcaggaccggaggcctccagcaagattgtgggaggacatggtgacagagtggagaaaccagtgtgtccagaagatggacggcacaggtcacctgatgtcacggtttcagagaagggaatttcagttcacaagggcttctcaagtgggaaggctgaagaaaggaagcaatgggtcctgcggaaaagcccaggcagaatcaccgaccatccagcaagtggccatgctggcatcccctgcattaccgaggtgcactacatggactgagatgacgtaagactgacatcatgggccaacacccacagtaagaaagacttgggctggaggcatgatgactcggtgggtacaggtcgtctccctccaggatgctgcagtaggagacacccttctcccgggagctgccttgtgacctccctccccttccccaccttacaatgtagcagaaagacttattggtgttctgacccagttttaccaaagaggaagaacatgaaagaaaagcatggcagagtgcaggctcacctgtaactccgaggttggagttggagggcccgggagttcgaggctagcctttgctacatagtgcatttgaaaccagtctgggctactggagactgtctcaaaccccaaaaaaatcgttttacggtggcagtgtaggccgtattcacccccacacttctgttgcagtaaataatatttgggggtttctaccctcccttgatcatataattcccaaataaaagacagagaatctttatataaagccttaaggcactagagcagggtgggtatccaccctctatgctagtttgtctacttctctgtccgtaatcccgagatatcgcttgccaagttctgcctggactgctcctactccagctggctggtcctcatggcaatgagttcaggatgtaccttccccatggggacttcttccttcctcctcctcctttctgcttggtgctctaccctcaggcccccaagcccagcctatctctcttctgcccagcttcaggctgcaggcatctttattcaaccaatagttttaaattgaggagcagggcttacccaacaaaagctggcctaggtgagcattcactggtcttgaggtaactaatccttgggatacaatgtttagcattacaatacttagcatcagactaaacctctacacacttcctctctctgagccacagtacagaaggctgccacggccagggcgctggtccttttagcccaagcccgggctcacctctctcgaaagctgtctgtatctcccaaagctcagaaaagaacaactcatttctcttcttaaaataacaacaaacaaacaaacaaacaaacaaacaaggcctttagccgtattgatacattgtttacctaccatgcagctcaccccacttaaagttagtttcaactttatccatggtttgttcaattgtacaatgaacgctacaatttcagaacactttgcccccaaaggaacctactccttagttgtcgcctgttccctagaagcctcagatccccatctgctctcagtctctccagatgaacacattttacacaattcctcacatttaataggtaggtgaggcgtatactagtttccatctggcttctttccctccctcgatgttaatcaaggttcaccaggatgtagcatgaaccaggtggtgttccgttccatggatggacttacaacatctattcattcattcttttgatgatggacatctgggttgtttttactttgggctatcatgaatagtgcttttgtgagcattcctgtccactttttaaaaaatgctttttaaaacaaacaaacaaacaaacaaacaaaaaacccaacatggatcaggatcaccgctttggccacgcttgagaacaggaactaagaaattgtgggcagcccgagtggctccttgctttcctgtggccttctgtatagggtttagcaaggaggggaaagacttcaggtcaggatccatcttcatgtcctctggccagagtgctacctttctggatcaatttacctgctttagagtggaagagacatcggcctccctgttagcacagagtcctcttggaatcagtagtgcccactctctggagacttcggcaaactcaggagactgttgggagagttgtattgattggggcagcagaagagggtcctgcctggtcagcacatgttctagttgccccaaagtcttcgtgtggattctctatagactccaagccccagtcccaagagtcctcaagtgccatcaaggagcacgcaggtgttgccctgggtcatgtccatgtccacattccatgtagtcagaaggggaagcagacacaccccttccctctgaggtgggacttggaggtgacacacactttgttagcacctcatctatacatcagaacttagtcacattgccttgcctgggagcaaaggatgctgggaattacaggcttgttctcacttccagtgggccccacatgcagccaaggaatggagagccaatggctaagttaagaggggatgctgggtaatgtagtctctgactatgactgttaatgggtggcataaaatgacagggtgcttgtctagagtgcatgaggccccaggtccaatcccagcaccatcaaaaagaaaaagaataaggaaactgttccctgcctcagaccaagttgaagactagagagccaccaggtcacaaggctgttttggctggggagggacgtctgtgatgcccttggcttgcggaggcctgggcaatgcttggtctgaacctcctgccttaggctaacctgctctgctcccagttgagccttggtctgttctgtagcggagggcagcctcattactctggagtgagagtggccaggaccaaaggagaggacttgacctcagcggcaggcaccaaggaggaactaggaaggctacctctgttcttcctagttcctccttggtgcctgccgctgaggtcaagtcctcacacacaggggtgtggcagggctctacagggctcttctctcctaccattagcagaaatgactaattatcgtagttgagagcaagagtggagccatggtgggcaggaggcctggccttgctctgccaaggtgtccagctggagagattcatggctgaaggggtcctgggaggtgacctgcaggtgcccgggcacacatacaacccaacaacatgtgagggacacccacagctttaccctaaggctgtggatggggccagggtagagttctaccttggaacacaatctccgatgacagcagagagagaagggggtgggagtggggtcgggggactgatggccctgaggtattgaaggagtctctagagaactttctagacatattcatttcttctgtgttcagcatgatccgtaagatccttccactctttcacagatgaatgctgggagtgcgtgcctggattcttggggagaggtgcacaggggcggggagacactcaaacttcatgcccaaagcaagcaaggggtctgtgacagatggctggcaatccagactaagagcccattcccacggcaactgacataagcctgctggagttccatttggttgttttgaggactggatctcactatgtagcctaggctggcccaaaaatcactaggtagctcaggctgctctctaactcacagcaatcttgctacatccgtttcttgagggggcgttacaggtttgaaccaccatagcatacttcagtatgtttttttttgttgttgttggtttttttttttttttttttttaattctaaggcttgctgggcagtggtggtacacgcctttaattccagcacttgggaggcagaggcaggcagatttctgagtttgaggccagcctggtctacaaagtgagttccaggacagccagggctacacagagaaaccctgtctcgaaaaggaagccaaaaaaaaaacctaagacttttaaaaaattgtgtgtgtacatgtgtgtatgtgcacatgcacatgtgtatgtacatgtgtgtatgtgcacatgcgcatgtgtgtgtgttgctaataatatatttctgctaacatagggtatatgacttaaagtgtatgtccattcacttatgcacctacccacccactccacccacccaaccacaactttatccatccatccacccatctacccacctacaatcttacccatccatctaccaccccacctatccacccacccatccatctaattattcatctacctattcatccatccatccactcacccacccacttaaccatccacacatctactcaccttcaactttaccctgggctggaatttctatttgcaaaagcagacaaacgggggctggtctggtatctgtgtgtgaggggctgctgtcagtgcgtacgtgtgcaggcacacgtgcatttgtgtgtacctggggtcaggaatcaatctgaggtgtctttcttcaagagccacccacctgtgttgtttgtttctattgtctcacagggaactgaggctttccagctagactagactggttagtgagcagcctggatctagctggctagggttttgagagcacatctgtaccttcagcttttgcataggttctggggaccagactcggatctcatgtcttcctggcaagcacttttatcccttgagcaatcctccctgtatctagcctggtattcttgacctagactcttagaactatccatttccaggaatcttgaggggtttcctgcttaaggtggggtcaggcagtctcacagggttcaacacccatccagcttcaatccttaacgacttttatataatcaattttcactttagatttgcctggcacaaagccttctctggctgaaaactggccataccagagggtccccacgggctttccatgagcgtcgggggagctttcaggcaagatgcttttatcaatgcctctgcagtgtgtggtacataagccgggatcccagcccttagagtctgactgtcactgtgaagataaacagactctaggtgggaaagcctgtttgggtgaactactttcattaagagagtaataacagttattattagaatgtggttaggtagtcaggatagacccacgggaaaacacagctgggatttcagcctgcaatttgtagccagacaggagcctaaggcttccagtttcctcacccagggattctgagccttgcctgggcttccctgggaatctgctcactcctgcctcccagaagccacctcagctcagcttcccccagcagtcaagggagaactggctggccaactgtgattggctaagctggaggctgttgctaggttaccagtctactgagactaggtgtgtgtgtatgtgtgtgtgtgtgcacacacgcagccccatcctccttcccagatgcattccacacagcaggcctaattcttgcacatccaagacaggatccagtcatttacagggtcggccaaccgtgccccttcacctctgtgtgggggaagggaagacgggcatctgctttaactcatcttgaataactatctgtctccttgggcctcagtgtccccctctgatatctggagctcactaatttattattcattatttgtgtatgcatccagttaattaagcatttgcctggactctagttgctatattgaatactaaggattagtaagacctctttgctcttgggaaaggggtaaatatttccaatttaatcaaataacacacacaggcatccaattatctcttcccctttggtgatcgttctagcccctgattcttttcaaacccagtttctagctgtctcccatagaagaggcatgttgccagattggccctgtaactcctgagaccgtgggcacaccctctcagccacgtttttcctatgtccatgtattttgtatcttattttaagtttatttattttcaggtgtatgggtgctttgtcagtgtgtatgtctgtgtgccacatgtgtgatatgctcaagaaggccagtagagggcgtcagatttccccagaactggcattgtagatggttgtgagccaccgtgtgtgtgccggctatcaaatctgggtcctctgaaagaacagccagtactcttaagcactgagccatctctccagccccctactttgacacatcctgaagaagatgcagggaaagatactgccttgttcagtggatgcttagttcacatggacagttacaggcgcgaccaagattgagtgcatactatatgcaggaagcgttcacactgtctcagaactcaataaggcagacatcctctttgggtggttggtttttgtgagacagggttttatgtagggcctctaacttgctttgtagcagaggatagccttgaactcctgatcttcctgcctccacctccacagttctggggttgcagatacacaccaagtccggtttattcattgctggagggggactaaggctttgtgcatgctgggaaagcatctaccaccaggacaaatcatcagtcccaaggaagatatctttgcccatgttaaaaatatgcagctgagttgggcatggtggcgcacacctttaatcccagcacttgggaggcagaggtaggcggatttctgagttctaggccagcctggtctacagagtgagttccaggacagccagggctacacagagaaaccctgtctcgtaaaaaaaacaaacaaacaaccccccccaaatgtaactgctgggcatggtggtgcttgcctttaatcccagcacttgaatggcagaagcaggaagatctctgagtttgagtctagcctggtctataaagcaagttccaggatagtcagggctacacaaagaaaccatctccatatgtccatgtatgtatatgtgtgtgtatatacatatgtgtacatgtgtgggtgtgtttggtcatgggaccacgcacaacacacatgtgctttgtggaggtcagagaacaacttttgcgaattagctctctccaccctgtggttcccagggataggacccaggctcactggtaagtgcctttacctgctaagctaccacactggactctcttttcctttaccttatagatggtgggattgaatgaacatatctaagtagccagtgctgtcccagacggttgtgtgaccccgggatgggaaacagttctgcttggctttgcagtacagatctgtggttacagacaatgaaactccccgagtacctgaaagtcagtgctgtggcttcatcctgcccttgacagactcctcatctctgccctcatttcctgctgaccttccttgtgctgtgccggaggcttgtgaagcctgagttggccacactggtcaatacaattgattcttgctggttacagacttcccgtttggcattctcatcactggggagctacagggaggaggttaaaggtcattcttggcaatatagtaagtgtgaggccagccgggggtgtaggaacccgtatgtcataactggacgtgttccctgtccccgtgtgacttcacggccaggatttcactgcagagttgagcagcatcttcgtttttctcagcggtcactgggcaagcacaagcagccatcgctgtccttgagactggctgggggtggaggatgttggctctgaaggattcccagtctatctggagggttcctgaagggcccttgaggatacagcatcctgatgggctactgagagagggaaggctacacacacacacacacacacacacacacacacacgaactgccacctgtagtagaagcagctggtggggactgggttgtgacagctagcctggagtttcctgaccaggacacattaagcccaagacactactttcaacccccacgttgctgcggttggtgtagtaaccatgacccggcagcccggaaattgtcactacctgtagtgacaaaattactggacagaagtaacgtaggaagagagctttatattggctcagtttgagggtgctgtccattgtgacagggacgtcacactggcaggagcttgagactcagagagagaccaagcctgatagcccagctcccttgctcccccttggcagtccaggaccctggcccaggcaagggtgctgcccacttgaagagagactgggtattctcttcagtaaactctatctagaatttccctgacacacatgccccaggtctgcctcccaggtgattccaggtagtttcctgttgacgatagtaaccaccacagaggtctttgaatgggagaaagtgctagaaaataacagacatgtaaagaaatgaaggctccaaacctctcccctccccctctccacctcttcctctccccttctccctctccccatttccctctccccatttccctctccccctctccctcttcccctctccctctctccctctccccctccccatttcccctccccctcctccctcctccctcccccctcacttctctctctgagacggggtcttccatgtgagcatggctggagaactctctatgtagacctgtaaagtaacttttgctaacctgatgtaattgtcttcttacgtcgatttgctaacccaggcctacctagtcctggaagcttctagcctctgtacaaactaatctaggcctagaatgttttcagcctctgagacttgccactgaataagctcaccatttctagctctttctgagctctggctggctggttcaactcagctgttcttgctcaaactcttctccaagctgactgatccaatctggcttctgactgaattgctctgctctgcctcatactaactctggcaatctcttctaatcttctggctccttctccttctctggctcatcctgtctccacctgtgtctagcttgttctctctctgcaatcatctgtgtaaaactctcccggtaaaactgcttcctctctttttctctctgtgttctctttcttaaacagactctctttccttctgttcacgtgagagttaagcatatactattctgtcaaatctttctctgatttgtcactttgtctaccactcaaatagacatcactttcaaacatggctgcttccttctacaaactcactttaccttcatcgtttgggagtaaaggtgcggattaaagctatgtctgtattgcacccagagggattaaaggtgtgtgctaagggcctacaccacaactagaagtaggaatctcagggttcacggtgtgatcaaatagccttcaacagtgaccagattcaatttgaacttgaagcaaccctcctgcctctatctccagtgctctgggatgaccagcatgagccatcagaactgctaatggttgactccacaaaaggaagtttgttagacctggttacttatccatttcccctccttactaccaaaaaagctgggggagagtgagagaggagggaaagacagtgagggaggagggagagacagtgagagaggagggagagacagtgagggaccccaagaacagacacacagggacccacaaggacagacacacagggaccccagggacagacacacagggacctcgaggttggacacacacacaaaccaggagggaagcaagaagatgcagagagacagacgacctttcggtttgtgcctgctttaagcatcaaggaagaattttaagccaaactcccccaggctgagagaaaccagttgcctccctgcctccctctggttttgtgtgtgtgtgtgtgtgtgtgtgtgtgtgtgtgtgtgtgtggtacacagctgatttccagactgaaccattttcagctgcatggatcctggaaatgtcagggctatttccattctcctcatccctgtcactcccctccctgttgactaggaatgaatcctgttctaaagctgaagttaaagcaggcacttgaagcctgactcgtgcagacctttgacctctcatgcccacttcactcatgcccacttctagtggatagtccggcacaggtagagaatatagcatgggttgttcttcttgtgtgtgtgtttaagtatttatatatatatcgttttttcgagacagggtttctctgtatagccctggctgtcctggaactcactttgtagaccaggctggcctcgaactcagaaatccacctgcctctgcctcccgagtgctgggattaaaggcatgtgccactacacccggcaacaaaggttttaaaatggttcatccgatggcatacatctgtcatcccagcactaggaaactggcagaagggagattaagagttcaaggttgttcttggctacatagtgagtctgaggtcagcctccgttacaggaaaccagttcccagaagcgcatacctccgtggtaatctgtcttctgggtgaccttggtggccacttgccaggttgttttctaacctgccagctggtcacctccctccctagagggaaggtgctgagtacacatgtctgtacatggcaaggctgacactgccgggcttgctactcccaggaaggagaaggagcagtccatgttcggagcaaagcctggggtccctatcgtgtaaggcaactgtgatctgggtgtgggtgaggggtggcttggcatcagaaaaaaaaacgagacaacagcaaaccaacattgcctgactgtgtgacctcaggcaaggcgcttttccctgcctcagtttctctacctgtccaacgccactctagcattagcagtctctaggattagaccagtaacgtataatccacgaactataaaatgcaataggaataagctgtccttttgtctctaaattgctgtgtgtgactctgggcaaatcagttgccttctctgtgtgtctgccgccaggcactagaatgaatgccagactctccaagctggaaagactccgcccttcccacccgcccacagagctgctggataagctctctgacaggggtgcttatgcaacctccgctctgctgcctcccacagcagagagctcatggctacacaagcccctggtacatatgcatgacgctaactttaaacaaatttttccaaatgagatagttctttaagggctggctgtgtagctcagtggtcaagcatcaaacgtatgcaaatatgtgcagagcccctagttcaatcgctggcaccaagagaaaacagtatttgtgggactggagaggtgacttcgtcattaagggcatctgttgctcttgtagagggcttaagttcagctcccactggccatgtcatatagctccaggtccaggggatcctacaaccctgtctagcatctgtgggcacacacacacacatgtgtacgtacatacacaggaataaaaataaaatgtaaaaatcaggttttgagggctggtgagatggctcagtgggtaagagcacccgactgctcttccgaaggtctggagttcaaatcccagcaaccacatggtggctcacaaccatccgtaacgagatatgacgccctcttctggagtgtctgaagacagccacagtgtacttacatataataaataaataaataggtaaatctttaaaaaaaaaatccggttttgagatcatagttaagctaagtggacagagttcttcccagcatgcatgcagccctgtgctcgctccttggcaccgcataaaccaagcataggtcatgcctggaaccctggctctattgaggaagagactgtagaatcggagttccaggtcatccttgcctacgtagagagggtgcagtctgccaagggtatgtggcctctaagcaagtgcccttgtcacttggtaaaagatttccaaccacatctgtgtttgtgtaggaccctcataatgacagcttggtggcagacctcaggtgcagggacaagaagctgccagagctgttttgcaggaagaaaatatctttctagtcgctgacaattcctgatgggtggccacgcatggtcaagaagcaggacacctgggtcccatgctggtgccagtgtctcctagctgtagggcatctaggacattctcctttatggcctggctctgttctgttcctgaggcccccccagcttctgggtctgaggcctcctcctcgctgtgaacggctgggtgaaatggttgacatcactgagccccgggggcgcctccccatgttcctgagcagttgctgagggtgcatccgccactctgctccctccacagagaaatacctcagagtgctgaatatgggcctggcggaggtgaagaagttcttcctgggacctctgtctccgtcctgcaagatggtacagatgatgcgacagtttttgtacgatgtgctaccggaggactcctacaagttcgccaccgggaagctgcatgtgagcctcacccgagtcacggacggagagaacgtcgtggtttccgagtaccgatccaaggaagagctcatcgaggcaaggccctggttggagcgcccccgcctgggagaacaggggcagctcagtccagcccactccccagcttcctcccttcttcccttttttgcctgcctggcccttcccttccttcgtgcccatggctctgccaggagcagttctacgagaggagactagattagtctactaggtggtccggtagattactaaggccccaactaggcatcaggatgggaagtgagtttcaggctgtttcccacgccagtacccatcctttaaaattagtggaaaattggtaggggtcaagggtcattggcacccaccccagggccttagcatctcagccccgtctctcctcaggccctgtattgcagctgctttgttcctgtttactgtggcttcatccccccaacgtatcggggagaggtgagtgactggggcaccttaaggtgaggatcgtttactactcagaattcctagctttttcagggctgctttctctaggtggtccaagacctctctagattcaccaaccctgtactgaacccagcaagtcaaagtcccaaggttgccgggttctcccagaagtcagcgtgcagaatacagaccccagaacacagggccctgagagcctgactgaggagaccccctgggggtatctgggtcagtggtcacaggctttcatgtgcccatgggtgactcagaggcatcctaagacaatgaggactcggctttaacagatctggacatgggggtgggtgggtatgggggacttttggtatagcattggaaatgtaaatgagctaaatacctaataaaaaaatggaaaaaaaaaaacagatctggacagaccaagattctgcatctcggactggctttagggtaccctgtgactggtctcctggctgcagtctgaatagtggggaggtaacattttatgggactcctgggataagagtactggtcatctcttgttctgttcataagtaaggaacccaatagaagccccaccccacccccaactccaaatgaaaggggttggagatggctgtactgtgaggttgtagaagggaccttggtctcttgagtcattcaaaggagggaccccatacagagaccattatcagcaagattctctaagggagaagagtccagggcccagagcattagacttgggagtccccaggcccttagagatgcagcaagtggccatgcccacaccaaccaccatttatcttggggacctcagcctcaaggctggttgcaaaaggtgtctcttccatggccccagctcccctgtgtcttctctggctgagcgtctctcttctcttccctgcagagatacatcgacggtggcttcacaagcatgcagccctgttccttctggacagactccatcaccatctccaccttcagcagccagcaggacatctgtccgagagactgccccaccatcttccatgacttccgaatgttcaacttctccttccagttctccctggagaatatcacccgcatgacacatgcgctgtttcccccggacctggtggtgagagacagggccgatggggtgggatccagaacagatggacccctcctcttgtgcctctcccctcagagggcccatggttttgccatgtttaaaggattgctaaaaacaatcagggctaaaaatgttgctcagttggtagaatgcttgcctagcacacatgaagccctgggttcgattccccagcaccacacaaactgaagacagtcgagtactgtaatcccagtactggggaggtagaggcaggaggttcaggagttcaaggtcatccttagctccacagggagtttgaggttagcctgggctacatgccatcctatgtccaaacaaaatcacacagcagaataagtggtcagcaaagacttaaatgtttccagtttgcccccccctcaaaaaaagctctgtggacatagctctcacacacccatccccatgacatctgcaaagacactgtcactgtccccatttgacagatgaagaaagtgaggctgtgggtagttacagctacacagccaatgaagagtagtgctggggtttgaactgaaacaccaagctctgtacttaggacccagatgtgtggtctctgattccccagaaactcccaccaactcccacagtggaggctggtgatactctgggaacaggtgactggcacgctgacacgcccacccgtactgtgttagcaattgcttcccagctctcctgggcagattgtgggggataggaggtatagcccagggcttgctgatatggttctgggaagagaggatatggggagtgtctatttatagtgtctatggtgggacgctggctctcttggtggctacagcatccatttatattcctctgttaccatttagattctgcaggaatattactatcggggatacaatgatgctgtctcatacctgcggagactgagtgagtacctggagcctaggggcggggcaaggagtgtggcaggggtgggtagaggacagtgtcccaaagctgtagccaagaaagctggacagaaggacagagacctagcagcccacactgcccaagaccgtgaaaaaaagtatatataaaatatgttttagtcttttgagacagggtttctctgtgtagccctggctgtcctagaactcactttgtagaccaggctggccttgaactcagaaatccacctgcctctgcctcccaagtgctgggattaaaggcgtgtgccaccacctcctggcttgtttttgtttttcgagacaaggtgaggaacttgaggttgggaagatgttataatttgatacgtaaattttggaggcgactctgtttccccagacaggcacatacacacacaccctctgcatctgggacaccgcttaactacaccctcgggttcctgtctcaatgcaaaggaagcccaggttcttcccgggctccttgtgatgctcgcgtcgtccgccaggtggcgccagctcgttggcgccagctcacgtgcaggagtccctccctcccgccaggcttcttggatttttctactggcacaggttcaggagctactggtacccaaagccatactatcggtcccacctgccacttgaaagcagtcagaatgctctctgcttggttgaggggggaggggaggcactcatcacacacaggatgaagccgccctggacccctcaatctcatcctcatcccgtctctcctccctctcttgcaaaagctctcatacacccagctactcctacagctaggaagccgttagccccacctgcctccctggcgtgggaaaggggaggctgaccgatgccagtctgctcccagctgtgcccagcaggcctcgcccgcactctttccgacaaggtcctcccatacatgcataatctatcatctgggtctttcttcagaggctgggaccgggactcgattagagtacttacctagcatgcatgaagacctgtgtttaatttccagcacttcataaacctagcagggtagtgcatgaggtaatcccagcactgggaatgtgggggaggggtgggatcaggaggaatagaagttcaaggtcatccttggctatgtagtaaatttaaggccaggctagcctgggctacataagatcttgtctgaaaaacaaacaaacaaacaaaaaaacggagctcctggagctcctttatggtcccccccaaccttcgccaacacccctcacccctacgatctgatctctgtccttacaatcttcttgcctagctcccaagttctggggccacgctgtgcccatcatgtatcatatttatgtagattttgcagacacacccatactacactatgtgcacatcagatctgatgcgctgctgtgtgtggcttgtttctcttagatgcagcgtaccttgactctcccagcaagagagtgattttcccgagggttgaagtatactgccagatagaggtcgcccttggccatgagcccccacctccgagtctgcagaacctgccagccctgaggagaagcccagcagactcctcacaaacccatgcacaggggtctcccaaaaaggacagaaaggacagccattcctcagccgccccctcagtgcagacacctgaatctgggtgcaaggagtctgtggaatcacccgtgtcactacgggtctctatatccaagcaaccatctgtatcgccattatccccagcccagccggtcccagtaatgaggcccactggccccagggacagttgcccaataaatgttcaaactccaaacccggagcgaggagtgaagggtgccctggactctgccacagaacgaggaatgaaggatgctctggcctctgccactgacgagcagagcacaactaccttgccacctgtgttgctcccagctgcagactcacgaggctcaaagactggatcctctgtgcctattgggtcacccgagtcccctagactgctgctcagatcttcccagggagccacagcatccagggcaacacttggacttccgcccctaagtccttcaacaccacctgctgggccacctgtggaggatctaggcccagaacgacccacaggtatgaccctccctgctctggcggaaccgagaaacaccagttctgagggtgacattagccatctacctgagcagcctccacaccttgcaccttgaagctcagcccctccccctggcagcaaaaaccaccaccaagaggctggtgagatggtgcagtggttaagagctattgctgttctcttagaggaccagagttcagtttggtggccaggacaggacactgggtggctcaaaagcatgtctgtcccctcttctggatcccatgggcacctactcttgcacacacctcccatttacacaactaaaaaaaaccaaacaaacccactaaatcttaaataaaataaaataaatttaatattgatattaaaggcttataatagagccatttatatttataactcatacaatttatacaaaattttaaccaataaaaataaactcatgagaaccagagttttatccccagaacccgcataaaagcttggtacagcggcccatgtctgtaactctagcgctgaggacaggaagacaggcagatctctggagcttgatgactgatgagccttaaaaaagtatttttaattaaatatataattccagcccttgtgaggcagaggcaagcagatctctttagagtaaggccaccctggtctatacagcaagttacagaacaatcggggtacacagagaaaccctgtcttgaaaaaaaccaaaaaaccacaaaaccaaacccaaatcagttgaggactacacccagtatccatctttgactccatttgaacactcactcgagatatacacactggtcctaataataatcttcattccaaggtggtggtttactccctcgcgctggtggatgttcaggccccgccctctgacgtcaccacggggcgggaacagtggcgtctgcccacagcagctgcagccttccgtctgcatcagatccactgatgtgataacttgactcttaaatacgaggagagcattctttgctggttttattgcttccttgcttgtttgcttattatttaaaaataatctcattctttgaaatattcgtacatgcctacatccagacgtgttttgaattttgatttgttggggattagtgagattcctcccacagctcatacaatttttaaaaaatcacaatggtttaaaaaaaattatgtttgctggatgcgctggcccctgcctttagtcttagcacttttgaggcaaaagcaagcagatctcgaagttcatggccagcctagtgttcacaaactagatcaggatacaggacagccagggctacgtagagagatcctgtctgaaaaaaaaaaaagtttctgtttactaagttcgtgtgcatatttctgtgccttgttcacatccatacctggtaccaaagatgtcagaagagggcgctggatcccctggagttacagttacaagcagctgtaagctctggggaggcagaggcaggtggatctcagagtttgaggtcagcctggactatagagtgcgttcgttccaggatagccaaagctacacagagaaacccagcctcgaagaactggaaaaagaaaaagaaaccaaataaaataaaataaaataaaattcctttaacaagagcatctcactattgggttcctggatagtctgcctctcactatgtaaaccaggcaagcctcaaactctgaaatccacctgcttctgtctcccgagtactgggactaaaggggtgctgagccactacaactggcatcctcgcccttcgctctcagacacactcaccattgttatttgcaaaatgacccagtcagtacccactacctgctcttttaactccggtgttccctccagctacagggtctcccgccttatcacagctgacaggctcggcagcaccgggtaccgggaagaaagccccccacaagcctctgctggtgtaagtttctctttcctcaggactgttctctcctttgctcaggggaggggccccagctaagtgaggtttctgcgctttccgctgcctgggtatcacctagtctgtggggcagccgctcacgctcggtgccctcttctctctcccgcagtgggacgctatgtgggtatacctgtccatggggcgataccacccctctccctcactccagcacacactgggtacaacctgttctaggacattgggggcgtaggcactccaggtcccgggtcttattttggatgaagccacaattgggatggtctaacaggtgcaccaacctagccttgggtagcctctaacggtagaaaaggactctcaaaagactctgttggagctgccatggaggtacgtggctttagcccctagcactataaccttgaagtgaatgaaaaggagagagtgggctgagcaggggcgagcatggttcgatgctctctgctcctgattgtgggtgtaacgagaccagctgttgcaagttcctgcagccttcacttcctatcagcaactgacagttgcctggatttgtgaaccaaatacaccctttctgccttaaatgactttgtcagggtgtttgaccacaacaacagaaaccaaaccaaaagagtatgcctcagttgtctgggttgtggaatgtacacgctccatcattatctctacagccgtgacactggcctgcagatctgtagtgcttcgaggtacctgagaactacatttcccaggctcccttgttggttggataacagttggattggccaatgaaagatcagtaagggggtcatgcatctgtcaaccccgacccccaactttgtcttttagaaaagcagggttgaaacattgtgttctttaattttattgttggatgtgggtctccttgcacagcccgggctggcactgagcgagcaatcctgtctctgccttccgagttcagagattataggtgtgtgccatcatgttctcaggcatttttccatagacacatgtatttgcttcacaccaatcttattaggatggatctgatctccccccaccccgttatgaaattttatctactactgagggtgtgtacagagatggctcagcggttagggacacacactgttcttgcagaagcactcgatccaagttcggttcccagcacacacgttgcgcagcccaccacggccctgcagctccaactccagaggctctgacatccgcttttggtctctgtgaatgtgtcctacatgtagacacacttgcacataaataaaaaatcttagggatgaagagatggctcagaggttaagagcactgactgatcttccagaggtcctgagttcaattcccagcaaccacgtggtagctcacaaccacctgtaatgggatctgatgccctcttctggggtgtctgaagacagctacagtgtacttacatataataataaacaaataaattttaaaaaatacaaataagccgggcgtggtggcgcatgcctttaatcccagcactcgggaggcagaggcaggcagatttctgagttcgaggccagcctggtctacagagtgagttccaggacagccagggctacacagagaaaccctgtctcgaaaataaataaataaataaataaataaataaaaagttaaaaaacttttaaaaaacattgacagtttaagttaaaattcgtaaagcatatatttgcataaagtttgtgtgatttttaacctctttgcaactacgtttagtaggcgtaagtttgggctttgtttgtctccttggtctggttactgcccagataaacagtgaatagtaatcagatgtgtttctggggacatcgaccttggccagggccactgggtctgctgagctggaacaggacttggacggtggcagacactgttcaggctgctgtaacaagcacctcctttgtctctttgcccagggagggtcctggtgaggattcaaatacggcaaaaacgatgttcaagaggaagcagaagaccaatgccaccagagagtgcttccaccgaaatgctcagtccaagaagccggctagcaaactgaagtaagggaaagacggctctgccaggagagctcagcgctcacagttagaacatctgggctctgagtctccattttctcacctgcaaaagcaggggcgacccggctcataagcctgtgagtgtgcagtgcaggccaccctatgaatgggatccttcagaagacatcagatgtcggggtagatggatcctggaggttttctagctacttctcaactgtgtgttgtgttgtgttgttttgagctgagtagccctgactgtcccagaactcatcatgtagaccaggctagcctcaaagtcactgggcagctaaagctaacttggagcttttgatctcctcctgctcccaactctcctgtcctggaattataaacaggtgacaccctatctagcttatgccattctttggccagcagacaccttgctcggacaaacaggaaagttgcttttgaccgtcctttagtccatttggtagaaatctctttaaacattattttatttatttatttatttatttatttatttatttatttatttatttatttatatgtaagtacactgtagtgtgtcttcagacactccagaagagagcatcagatttcgttatggatggttgtgagccaccatgtgattgctgggatttgaactcaggaccttcggaagagcagtcggcgctcttaaccactgagccagctcaccagccccgtcttgaaacatttttgacatttatttacttgctgattgtgcctgtgtgtgcgtgagtgtgtgctggagaggtcaaaggacagattttctggagttggctctgtctcgtgggtcccaaggcctgacctcagacgggcagattggaggcatgcactgaacggtcctcttgggttcttgaacttgtattttgttcagctgtcaattaacccctcattgtgagtagatgctgggaccctcctgctggctcacaattgcctgtgtatagagtctcatgtcacagataagtctaggaatctgcctgtgtttggagggtcaaggttcaaggctccctctctccaaatgccaagagtacttctatggtgggctctgcaggaccttgtaatttctcacagagggcaaggggctaccctggggtcacacagcaggtgaaggttaggcttgggactctaatggattttcttatttgccctcacgaatgtgtccttggcttctccaatccaggtctgcaccttgtccacttaactttcctgtactccccaagagagtttgggtcacctataaacctcatcccagccggatccaagactacagctaccccgaggtatggaagtcccctggatgtcacccaagagtgactctgcctctcctgtctcaggaggatgtagcaactctgagcatgtagcttcatttctgcccttccctccctttctggcacagccatggtcagctccagctttgtgtggacagctaagagctgcatgtggtggtgcacacctttattcccagcatttgggggaggagaagcaggtggatctctgtgagttcaaagctcacactttgactacattatgagaacttatgtcaaaaactccaaaatggggggttggtgagtggttaagagtgcagactgctcttctgaaggtcctgagttcaaatcccagcaaccacatggtggctcacaaccatctgtaacaaaatctgatgccctcttctggtgtgtctgaagacacactacagtgtacttacatataatatataaataaatcttttaaaaataataattgtatggttgggagtcacaacacgaggaactgtattaaagagtcgcagggttcggaaggctgagaactgctagttaagatattacccagctgggcggtggtggcgcacaccttcgtttccatcatcattcagggacagaagcaggcatgtctttgaatttaaggccagcctggtgtacagaatgagttccaggatggaccaggccacacagagaaatcctgccttaaaaataaaaaacaacagcacttgggaggcagaggcaggcagatttctgagttcaaggccagcttggtctacagagtgagttccaggacagccagggctacacagggaaaccctgtctcgaaaaacaaaaaacaaaagagagagagagagagagagagagagagagagagagagagagaaagagagaaagagagagagagggagaaaggaaggaagaaaggaagaaagaaaggtcactcccaatggggaaccccctttgcagagcaggtaagggccttggggtaataaagtgaacccctaaactgccttttgtaacaatcccagatcccttgttttgatcgagtacaacagagagaatgctgggaagaaggtcttgtcctaagtaagcgtcactcaaaatgacgactctttgtcctttcttagggagtgagtggccagaactcctaaaccggagcaggagagaccccagtgacctgggtccagccttccacccgaggcctgttgggaccaggggtgccctcagccatccagtgctacagaaagtctactcccccaagtttgggtgtcctggtctgaagagtctcctgcttccagtcttgctatcctcccaccattctgctctggatggacaggcagacatcaacacataccactcatagtccatccatctgggaggagggacacaatcctgcttttgtccaatcagggggcaagcatacatcctgactgtacttcctgtttatgtacctcctgcctacatgtgatcaagcacatctgtgctgttggagcaaacaagcttgtttacagaagcagaaaccagcagcttgctatcttacataactagcaccctccagtatccaggaagttatctgtccttgggctagtggggcttataggttcatctgtccctgagcaagtgggactttcaggttgaaaacatttttgtttcatagatttcttaggcacagtaattaaaacttaagatacaactttagcctttgcactttaagtctttggcagacctgttgcaggctctatgaaagcactgaatcaaataaaaaataaaaaagacatgactgctcccaggtatgggggaactagtttattgaagacacatgggagaaaatagccagagccatctgtgagcccagagtgaatgtgaccctgaggtgggccatgtgagaagggtcaggagccaagagaaccagatgtaaagggtaaaataaacatgtagccaaatggctggattatatagggaagagcagctgggggaagggcagcccagcacctatgctggagagttcatggtagaggcccagggaaggccagcctggagaaccctgtaacagctaggactgagggatgctgagtgcctggcctggtcctctttgctacattaataggcatctcagctgagaacaaaggagcactcaagacctggtgttaaccagtcctggcgactggaccacaggctcagcagatacaggcacaaatagaccagattctaggcaggtggcacacttgcctgggcttcacacttacccttctgagtctcttggtcgctgacgttgggaaggagggcatgttttgccaaaggcggctgtgaagaaagtgtaccctgcagcttgctgcaaggcggagtcactctggctaggatttggaagatgggtctcactctaagcctcaatctccagcctgtcttgcagaaggtggtccccattgctccagcccagccaggcccataccacaggccacaccagcatgtgcttacctcaaatgctggctttgaaactgagcacctccactgtccaaagagacagaaccccccttgttggttctctagggttctgttagtttcccctgttttgaggtaatatagatgtttattgcagttccagggttggcagataaaacccaagacgccttgtgactgaaacctcaggcaaacaccaaacaattgttttcctggtagctgttcctgacatctcaatcatttacctggattcagatttgctgggtgtagccgcctgcagccactgggaaggtggggttttatatgggaaaggctgggagagaaattaacgagccaagacaaggttttctgattaaggccaatgtttatttatgagtctgagcttataaagtccaccaccaggatctccttgctttgtcaggatagtactgaagaaagataaaatattgtaacttgtgagtttaagagcccacgcccctgcctgggactgagaacaaagcagaacagcccccaggcatgccagggcagggctgttgttaaggcattcctaagaacatcttgactgtaaagataaaaaggtatgcaaggaccagcagggctatattatttaagtcaacaccatctggccagaacctcccctctgtctattgccccttgacgccaggtaaagtcatacatcaactggttgctatgtgaacaaagataagcccccagcccacaggaacaaagtcctgatgccctgtgttctttctgttaatgtttgaataagccaatagtgtgtcgctaggctgaattccacacccctgagccccttaccccataaaaacccctagctttcaagcctcgtgtccgacatccgttatctcctgtgtgggatacatgtcagtccagagctccgtaattaaacgtcctcatgtaattacatcaagagatggtccttcgtgattttttgggtgcacgccaaatcggaaattgggtgggggtttccccactagatctaacagtaccaggagaacaggttcagcctctcagcaggtagtggcatcctggaggaaagctgcagttgaggcagggcaatagactttaccaccctaggtgggttagctggctgtggcaagtgaggtctgagccagcctgctcaaggctgggggaaggtacagctgggcatcctgtgttctcatttgctgactctgttaacttttattagtccctttaattccctctttttttttttttttttttccgagacagggtttctctgtatagccctggctgtcctggaactcactttgtagaccaggctggcctcgaactcagaaatctgcctgcctctgtctcccaagtgctgggattaaaggcgtgtgccaccacgcccagctattccctccttttttatttgtcttcaagcataaggtcttggtaggtagtgctagcaggcctggaactcactctgtagaccaggctgaccttgaactcactgagatccacctgcctctgcctcctcagtgcagggatcaaaggcctgtgctactgtacctggctctttatttatcaacatgcccatgcacggatctttgctagtggactggatcttacatagcttgagaaggctaggttcactctgtgacagacttcagattggcagttaaagagactaggcctaagaactgggcgagtccatcttgcgtgcgctcgactggccaggaagaacgacgctgcaacaggatccttctgcacacatttattgggagagcttgattgtagaggcgaaaagacctcgagcccagaactggtgctgcttttataggcctaggaggggcgtgtctcacacccggattggttatgcactaagcctcatttgcatgttcctcatctgattggttatactctcagtaccttacagaacctcattatcatacctcatttgcatgtctcacatctgattggttatactctcagtaccttacagaacctcattatcatgcctgggccaggcagtgtctttgcaaaaaactttactgcatatgtacacattggttgtttgtccaatcttatgcgtggtggccagcagtagtcagtgccactcagcaacggcacatgtggcttcccacaagtccaggcaagcccaagacagtcaattgctaatagaaaaaaatcccccaggctccagccttcatgaccaggtaatatcccagctacctggcctgaagcaaggacagtgggtcagctgcaatttccagacttcctcagctactttttcagta

SEQ ID NO: 28 - DNA, 17 bp, Artificial
  Oligonucleotide forward primer for Q-PCR
cgaggcgagcggtacgt

SEQ ID NO: 29 - DNA, 21 bp, Artificial
  Oligonucleotide reverse primer for Q-PCR
gtgacaccgtgatggtggttt

SEQ ID NO: 30 - DNA, 24 bp, Artificial
  Oligonucleotide probe tagged with FAM and TAMRA fluorescent dyes for Q-PCR
acggaggagtgagcgacaacgtcc

SEQ ID NO: 31 - DNA, 22 bp, Artificial
  Oligonucleotide forward primer for Q-PCR
cagcacatttatcccggtgtac

SEQ ID NO: 32 - DNA, 20 bp, Artificial
  Oligonucleotide reverse primer for Q-PCR
aaatgccgccatccacatag

SEQ ID NO: 33 - DNA, 25 bp, Artificial
  Oligonucleotide probe tagged with FAM and TAMRA fluorescent dyes for Q-PCR
tggcctcattcctcctaccctccaa

SEQ ID NO: 34 - DNA, 27 bp, Artificial
  Oligonucleotide forward primer for Q-PCR
ctctgatcatggtattgactgctctaa

SEQ ID NO: 35 - DNA, 24 bp, Artificial
  Oligonucleotide reverse primer for Q-PCR
tcctcactatcacagggatcaatc

SEQ ID NO: 36 - DNA, 30 bp, Artificial
  Oligonucleotide probe tagged with FAM and TAMRA fluorescent dyes for Q-PCR
tgggctcctttctctgacccacacttatct

SEQ ID NO: 37 - DNA, 23 bp, Artificial
  Oligonucleotide forward primer for Q-PCR
actgaatgcagcgtaccttgact

SEQ ID NO: 38 - DNA, 24 bp, Artificial
  Oligonucleotide reverse primer for Q-PCR
ggcgacctctatctggcagtatac

SEQ ID NO: 39 - DNA, 26 bp, Artificial
  Oligonucleotide probe tagged with FAM and TAMRA fluorescent dyes for Q-PCR
tcccagcaagagagtgattttcccga

SEQ ID NO: 40 - DNA, 50 bp, Artificial
  Oligonucleotide forward primer for PCR amplification
tatatactagtactagtcggaccatgtacgacgcagagcgcggctggagc

SEQ ID NO: 41 - DNA, 47 bp, Artificial
  Oligonucleotide reverse primer with no tag for PCR amplification
atataaagcttaagctttcatcacagactcttctctagtgaaaaact

SEQ ID NO: 42 - DNA, 80 bp, Artificial
  Oligonucleotide reverse primer with carboxy-V5 tag for PCR amplification
atataaagctttcacgtagaatcgagaccgaggagagggttagggataggcttacccagactcttctctagtgaaaaact

SEQ ID NO: 43 - DNA, 67 bp, Artificial
  Oligonucleotide forward primer for PCR amplifification
ggggacaagtttgtacaaaaaagcaggcttcgaaggagatagaaccatgtttccccgcgagaagacg

SEQ ID NO: 44 - DNA, 51 bp, Artificial
  Oligonucleotide reverse primer with no tag for PCR amplification
ggggaccactttgtacaagaaagctgggtcctacagccccagggccccgat

SEQ ID NO: 45 - DNA, 48 bp, Artificial
  Oligonucleotide reverse primer with carboxy-V5-6His tag for PCR amplification
ggggaccactttgtacaagaaagctgggtccagccccagggccccgat

SEQ ID NO: 46 - DNA, 44 bp, Artificial
  Oligonucleotide forward primer for PCR amplification
atatagaattccggaccatgaagcacatcaacctatcatttgca

SEQ ID NO: 47 - DNA, 38 bp, Artificial
  Oligonucleotide reverse primer with no tag for PCR amplification
atataaagctttcattcaaaccaattttctttaagtaa

SEQ ID NO: 48 - DNA, 80 bp, Artificial
  Oligonucleotide reverse primer with carboxy-V5 tag for PCR amplification
atataaagctttcacgtagaatcgagaccgaggagagggttagggataggcttaccttcaaaccaattttctttaagtaa

SEQ ID NO: 49 - DNA, 22 bp, Artificial
  Oligonucleotide forward primer for PCR amplification
atgggcttcttagaggaggagg

SEQ ID NO: 50 - DNA, 91 bp, Artificial
  Oligonucleotide reverse primer with no tag for PCR amplification
ggactagttcaatggtgatggtgatgatgggtacgcgtagagtcgagaccgaggagagggttagggataggcttaccggcctggtgggtgg

SEQ ID NO: 51 - DNA, 95 bp, Artificial
  Oligonucleotide reverse primer with carboxy-V5-6His tag for PCR amplification
ggactagttcaatggtgatggtgatgatgggtacgcgtagagtcgagaccgaggagagggttagggataggcttaccggcctggtgggtgggccc

SEQ ID NO: 52 - DNA, 1394 bp, Rattus sp.
atgtacgacccagagcgccgctggagcctgtcgttcgcaggctgcggcttcctaggcttctaccacatcggggctacgctatgtctgagcgagcgcgctccgcacatcctccgcgaagcgcgcactttcttcggctgctcggccggtgcactgcacgcggtcaccttcgtgtgcagtctccctctcgatcacatcatggagatcctcatggacctcgtgcggaaagccaggagccgcaacatcggcaccctccacccgttcttcaacattaacaagtgcgtcagagacggccttcaggagaccctcccagacaacgtccaccagatcatttctggcaaggtttacatctcactcaccagagtgtccgatggggagaacgtgctggtgtctgagttccattccaaagacgaagtggtggatgccctggtgtgctcctgcttcattcctctcttctctggcctaatccctccttccttccgaggtgagcggtacgtggatggaggagtgagtgacaacgtccctgtgctggacgccaaaaccaccatcacggtgtcccctttctatggtgagcatgacatctgtcccaaagtgaagtccaccaacttcctccaggtgaatatcaccaacctcagtcttcgtctctgcactgggaaccttcatcttctgaccagagcactcttcccatctgatgtgaaggtgatgggagagctgtgctttcaagggtacctggacgccttccggttcctggaagagaacggcatctgtaatgggccacagcgcagcctgagtctgtccttggagaaggaaatggcgccagaaaccatgataccctgcttggaaaatggccaccttgtagcagggaacaaggtgccagtaagctgtgtatgccttacagctgtgccgtcggatgagagcatctgggagatgctgtcccccaagctcagcacagctctgactgaagcgattaaagacagggggggctacctgaacaaagtctgcaacctcctgcccattaggatcctgtcctacatcttgctgccctgcactctgcccgtggagtcggccatcgctgcagtccacaggctggtgatgtggctccctgatatccatgaagatatccagtggctacagtgggcaacatcccaggtgtgtgcccgaatgaccatgtgcctgctcccctctaccagatccagagcatccaaggataaccatcaaacactcaagcatggatatcacccatctctccacaaaccccaaggcagctctgccggtttgtaaattgctggtctccgtgcttccgatgaacttgggcattctccctgtggatggttccaggagaggccatagctgaaggcactctgccttccaccccaagtccagtttgacctttatct

SEQ ID NO: 53 - protein, 425 aa, Rattus sp.
MYDPERRWSLSFAGCGFLGFYHIGATLCLSERAPHILREARTFFGCSAGALHAVTFVCSLPLDHIMEILMDLVRKARSRNIGTLHPFFNINKCVRDGLQETLPDNVHQIISGKVYISLTRVSDGENVLVSEFHSKDEVVDALVCSCFIPLFSGLIPPSFRGERYVDGGVSDNVPVLDAKTTITVSPFYGEHDICPKVKSTNFLQVNITNLSLRLCTGNLHLLTRALFPSDVKVMGELCFQGYLDAFRFLEENGICNGPQRSLSLSLEKEMAPETMIPCLENGHLVAGNKVPVSCVCLTAVPSDESIWEMLSPKLSTALTEAIKDRGGYLNKVCNLLPIRILSYILLPCTLPVESAIAAVHRLVMWLPDIHEDIQWLQWATSQVCARMTMCLLPSTRSRASKDNHQTLKHGYHPSLHKPQGSSAGL

SEQ ID NO: 54 - DNA, 21780 bp, Rattus sp.
  misc_feature (15383)..(16358): n is a, c, g, or t
actgtccaatcacgtatcgccttgacatcaacctatagaacagtcacagacaggaccagaaaccatctaactcctagctttcccaactcctgccttcattggcaccagtcaatcgctttttttatttctaagtgacacttcggcatggaaacttcaggtccttcctgcctggtgttgtcttcctgtaatcctagttacttatgaggctcaggcaaaggggtcagaggctgaagtccaagctgagtaactgacagagacggcttgttaaaagaagaaaggggctgggtgcagcttagtggtaaagcctttgcctagcacgcaggagggtcgcggttctagtactgtaaaattaaacaggagctgaaaaggtggttcggcggttgagagcacagctgtttttacagtggaccagaattggactccaagcacccacgcgatggctcacaatcgtctgtaactccagtactaggggatttcacgtcctcttatggcctccttggacactgcacacccgtggtgcgcagacatatatgcagacaaaatactcatacacatggggtggagtgggggagcctaagtatctttttaaaaggaagaaaatctttctccatgaattgaaaacgtacagaaagcaaaagaaataacgacaaaaacagaaaccctacaataaaataaaggaaagaccatatgtggatggtggcatctgtcgtgtaataaagaaaggagagagtgccaccggcatgccatccaggatctgaaaaaaaagagccgctttgcccagcgagcatccaagcctacgattagggaaagcccacgagggcggggcaggagtccagtacgcccattgggcctcccactggcttatttgcatggtccgagcggggcggggcattggctgacgtcacccgaggactgcttaggcggctgcggctccttaagctcagagcagcaacgcccggagcagaattgagctgcatcgccttccggagcctccagcgccatgtacgacccagagcgccgctggagcctgtcgttcgcaggctgcggcttcctaggcttctaccacatcggggctacgctatgtctgagcgagcgcgctccgcacatcctccgcgaagcgcgcactttcttcggctgctcggccggtgcactgcacgcggtcaccttcgtgtgcagtctccctctcggtgcgtccacggccactacccaaacaccgcgtgcggggaggggcgctccacttggggaatcggtaacactttccggggtgcccaaagaaacctgtccagagagctctcgcccttcccagtgtcgttgggcacccatctggagacccggggcctgtcacctggcgtggaatttccctgggcccgcggcagcagacgcattctgcacaggagactcttgagcatctttttcccggcccactccagcgctctggtttgaaatgggtccccaactcgggtcctgccagctgggtcgtcccagaacagcaaagaacgactcaggtttgttgggttctctggtccctgagccgcaaaaggtttgtcctgtgtggctctggctagagacccatagtgggtgccgccagaggggcagaaggcacccagactaggacatccttggtggcagtccaaccctccggtcgcctttagaggtggctgctgggggcggggtgtatgggttagagaagtattcggaacactccctagaagctggaatgggttcccagacaggttcagttccaccctctcctgtcttcactagcctgggcaagcggcccttcccagatcgatgtcactggcagtgacattctgggcatttgaatcttggcctaattaaacatttgcccatgcacccatgagcgataggagacgaatttacgctcctctccctctaaactactaggatgtgatgccctcgccagagtcatagctggatgcttttgaggttgtgggcagggcttggctacgtgcccacgggagaggtctcgaggctgcatgataatgtcccgcatcaaccctcattgatctgggcctctttaacactttactctttgctctgctggccagagccagcaggcagagactttggcaggcagggaaagcccctgctgttgggcggagctctgccattggacaggatcagtgagcagaccctgagctgatgcgactctgggattagaggacaagcaaatctggctgcgtgcctgcttgtctgtcttccccagtgagggagggagacggggagtaaagggcagtaagagtggcctctgggaagactacatcagcccagtggccatgacacaccctgggcttcagcactaaaggcagttcctttatgggggctctgagtcccaacagggatggattattgggacaaggtttaaatgcatcgggtcccaggaatcttaaacacacccacttttgtatagagctgtggttctcaacctgtgggtcacgacctctctggggggtctaatgaccctttcacaggggtcgcctaagaccattggaaaaacagatatttacatcatgattcataacagcaaaatcacattatgaagtattgaggaaaataattttatgtttggggtggggggagaccacaacatagggggggaccacaacatgagggactgtattaaagagttgcagcactaggaagattgaaaaccactgataaagagggtgtttgtaactgcagcctggggctgtcctgtcagatcacagagagggcagaatgacagattatctctggtgggagctgtgtaaggtgcactgcccagggcttggagcaggcacaggtgtttggggcatcggggtggctggactcggcagagcaggcagatgcaatcggccgccatgagttcatcctcaggcaaggctcagcaatgggtagggtttgctgggagtcagagttggatcaggacagggtgggatacgcctgtccttctggaactctgattcatgttgtttaggccagggcagagtgggtgggccagagggacgtggtgtgaagaatgagggcagatgggctgtgggtatgccgtgtacctacctactgggcaatctgggaagtctgggctcagaggcttacacggagactagtcactcagcagccttttctgggttgagcattttatagatgttattttactcaatccttcctgtctccaaaagtgaatacatttctgctctccttgaaagaggcagtctagtggctggagagatggctcagtgattaagatcgcttactgctcttgcagaggacccaagtacagttcccagtacctgctcggcagcttgctgaccacctatattctaggtcctgggaatgtgatgctctcttctgacctccatgcacacatgtggtgcacataagcttatacacacatatgcacttaaataatctttttttaaaaaaaaattaaagtcattctgaggtataggcagatggctgcgtgctttcctctgtaaggggcagaggtcagattaacatttaggtccaggcctgcgttattttttcttctgtaccctctgatgttgaatccatttgtgccaaggctggagtgctgacgtgttggtggcttgtggctttatggacatgcaagtggagttctcatgggcaagggccactggaaggcctaccccagcaccatctaaggaatatacaccacataccatacaggcttttatattttttttagttgctccattgtcagacaagggtgaaattgatttttttgagtcatgcccagaatatctttgtaatgactttgtaatgactgtaattactgttaatgagataatgtcacctaggctgtcacctatagccacctcaaatgtacccaatcatgacgggaacacaggatgtgctttatggtgtcttaattgggaccagccacagttcaagagttccgtgtggtttctgttctccattgtggagaaccctgttctacgtgttcgtcctctgtccccagatcacatcatggagatcctcatggacctcgtgcggaaagccaggagccgcaacatcggcaccctccacccgttcttcaacattaacaagtgcgtcagagacggccttcaggagaccctcccagacaacgtccaccagatcatttctggcaaggtttacatctcactcaccagagtgtccgatggggagaacgtgctggtgtctgagttccattccaaagacgaagtggtggatgtaagcgttttgcctctctgggtgtggtcgatggagcagcagcacaggctaccattgtctctgtggactggggccatgggaatactatgctttcagggactttgtgcgttcacctagaagccttactatgtcaggactcttaattctcttactttttccagtgctgggatgggagatggagccttggtcatatcaggcaggtgcttgactgtgcggctgcaccctaccacaagtttttttttttctttctttaagactcagagaactggttctgggtgtgccccacacctcagcatatgcatggtatgcagtaaggttggctttttgaataaagcttctctagggtggaaccattcttacttgcttctattgccagatctagcacagagaagatcatttttgttatatcaaaatgccacatttggccctttttattatatatatgtgtgtatatatatgtgtgtgtgtgtatgtgtgtgtgtgtgtatatatatatatatatatatatatatatatatatatatatgctcaagtgtccagcagaatgcctggtcctccattcgaaattctaggagcttctgaattaccaacaccgatctgttctcagtagacagtgtgctctccagaacagtgattctcaaaaagtgtggcctagggaaccacggttctatatgacctctaactggcttcaggagagctctgagggagagcccgggttcgtgcttcctgtagcatctgggtgagatgtatgattccagccccctatgccaccaagtcaacagtgtggtcctccttgggggtcaaggacagaagattgaactgtgtgaatggtatagttaagaagctggcattgcggctaagacctaagaacctcaaagacaagcatgaatgtcattcattcacccaggtattaaaacacaaacaaacaaataaacaaacccgctattacccgggaggcagaggatggttggtctctgtgagttcgatgccagcctggtctacagagggagttccaggataaggagaaatcctatctgggaaaacccaaaacaaccaaacaacagcaaagaccctactgtgtgcagagagcattttgcttgcactgctcacttggggaagtatagaagggcacagagaggcgctgtgtcctgagagtgtctactctttttctcacaggccctggtgtgctcctgcttcattcctctcttctctggcctaatccctccttccttccgaggtgaggtaagcgttgggggatggtcggacagctgagcgtttgaggccgccatttgtagtgagctagactctggctcccatgactgtaccgaaccggagagatgggatcttcagtcttgaggacagaagccatacctaggcattcccggcagactgatccacagacttacactcccaggagttcctccacctcctgtccactgtccgtcggatacactggcctggctcccttaatctgctcttgagtgccctagggaagctcctggtggcctagttaaagtagaagaggcttaggaattaaactactcttgttaggtagttgctttccatggaaatatgggctaactgtctatatcttttttaatcttttagacagggccttactttgtaactttgtaacaatgtaacgttggctagaattctctaggtagaccaggctggccttaaactcacaagagtttgcctctgcttgactctgtcgcctaagtactaagactaaaagacaagtgtcatcacacccaacagtgaaacaatctacgttttcttttgttgttgttgttgttgttttaagtttcttgaggaaacttatatacctgagtatatatacaatgtgtatgtatagcccagagctgggctgctagggaccctggcaattaagcaaatctttgatcttgatacttgatccccagtcttcttgtgacataatgtaggcatgtgatctttccagaattgagtggaaggggcccaggagcattatggtgatgtccctacagtccctaaaatgcactcctggatgatcctgtaaacaagagagtcaagagttcaaggccagccttagctacataagactctgtcatgacacagtgacttccataggtaatcccagcaattagggactgagactggagggtcatgagttcaaggccagcctgtaaccccttctccccaaaagaaaagtattacaggaaaacagactcaattaaataccttgtcctagatctccaaagggagtttggttttgaacctatggtcatgtgtctccttggtgcagggttcatatcacatactgtccccagtacatataacacccttagtttatatggtccttttatgcacatgatagggactcacctatgggtgtccaccccacattaggccttgagaagcacaccatggggtggactctcacctccatgtgtctctttgccaggcacttttacatggtgggcatcccttgcaagagtacagagcaaccttgaaagtcagaaagacgctcctgcaagagtggttcccagaccaccatactggctgcagtgtcttgtgaggcacttaagcccagtaccctccctttccccttccttacagcggtacgtggatggaggagtgagtgacaacgtccctgtgctggacgccaaaaccaccatcacggtgtcccctttctatggtgagcatgacatctgtcccaaagtgaagtccaccaacttcctccaggtgaatatcaccaacctcagtcttcgtctctgcactgggaaccttcatcttctgaccagagcactcttcccatctgatgtgaaggtgagccgggtgctggggtgccactctctgcccccccaaatagctggggacaccagttagtccctccatgagcagacagaaatagattatgccatgatggtgactcaaaaaggttgctgtcttaaagctgggggatcatatatcccccacccccacccctcacccccggtcataggctctgccagggatcacatgtacagaccctgagttcagactcttcttccaggaattcagggctaagaagtcaggggtagtggatggtgtctaaggttcttttcctgttgttattttttttttaaatcctgatgaggggaagttttctggaaaaaaaatttattcaggctcacagttcaaggtgcagtccacgatggcagggacggcagggaagtcagccgtctggggcttgtagcagttggtcgcatggaatccacaatcaggaagcagggtgtaatgaatgtgtgatgcagcccagcttcctttctcaagcagcaaaatcccagatcccagccagggagtggagccactcccagtgggtgggtcttcctgccccaattaacacaatcacagtaaactcccacagggatgcttagaggccatctcctaagggagtctagactgtgcctagtcaacagttgaccctacccatagaagggtacgtagggcggtacatacctgtcttccagcacctgggagactgaggcagggggatggagagctcgagttgagtctgggtgtcctagaaacaaaaaaattgaggtctggagagatggctcagcggttaagagcaccggctagtctctgaggggtccagacttcaattcccagcaaccacatggcagttcacaataataccaggctctgaggccctcttctggcatgccagcgtacaagtaggtagagcactcatatataaaataaggtttaaaaagaggttttaaaaaaatgtactagcaagatggcttggaggtgaagatccttctctgttctcatagagggcctgaatttgattcctaatacccacaacgggcacacaactgcctgtaacacaatgcctgtggcttctgtgagcacctgtatacacatgaacatacctatatgtgctgagtagcggtggcacaggtttttaatcccagcattagggaggcagagacaggcggatctctgtgagtttgaggccagcctggcctacagagctagttccaaggacagccagggctacacagagaaaccctgtcccaaccaaccccccccaaaagaaaatatttgtatcaaaaaataaataaaaatcttaaaaacacacccacaaacaggtgttgtgctgcattcctttaatcccagctctcaggagacagaagtgaatagatgtttgtgagtttaaactggggctacagaaagagaccctgcctcaaaaataaaaacaaacagagaaaaaaaacaacaacaacaacaaaaaaaacccaaacccatggacccaataaaatggcagtgtttaacggtgctgactaccaagcatgaggaccaagttcattcttgggaatccacagatcaagagaaagtaacaatttcctgtaaattgtcctctgatttccatgtgcacatgtggatgggcgcacgcgcgcccgtgcacacattaaatattttgaaatgaaacaggagcaaggtgcaagatgtttggattttcttttttgtggtattgaggatggaggccagggcctcacatatgccaggcaaaggctgagcactgagccacgcccccaaccctcactggtggattatgggtaaggcctcacagctgagccacacctccatgtcttcactggtgggttctgggcaagcgttacactgatgagccctgctcctggttcagaactcttttcactttggttcctatcatgattctgatttgcactgtggcaaggagtcaccacagtcccttttctgatgggcgttttagtgcagcaaccgtgtctccactagtcttttgagagaacctggctgcctgtacccaatttgctcaccaatcataatgcccaatggtgatcgtccaaggacatgttggctacagtgataaggcttagccccaggccgaggccacatggagcactgctcctgctgacagaatagagagaagacattggagctgagacctggggaagcacagagttaggtgaaccccctcacagcgcctccaagccttggatgtcctaggttctcagaggatgtggcagtcacagcccagtggttcacctgcctgatttgctcgcaggtgatgggagagctgtgctttcaagggtacctggacgccttccggttcctggaagagaacggtaggtcctggctggggattactgcagaatcgctctgttctgtcatccaggctcatggccttcccagagccttactttaccagcttcttaggccctgtgggatggggaaactttgcccatgttacagatggctggggaggagaggtcagcaggacaggggcagccatcgtgatacagagctgggatttgaacctgggaagcctgatggcactgtgtggcccctcctctgcaggcagcttgtttccttttctttgagctgttttgtgagcccttagcatataatggactgtttgacagagttcagggacagtttccagatgagggtgttaaaatctgctccctagccaaatgtaggttctgtgcactggacgaatgaggcttcttcctagcaggcccttccccctgagtcatccaagcttacctgggcaaccaggtcgggctgtgtatatgtagtcacacagtgggatggcattttgacattatgactgtctcagtttacacaggcatgtcctataatcacacagtgaggaaagccaccccttcttaaagcatgcccatgccataaaccaggcctggtagcatgggcctgtcatctcagctgcttgggaggctgaggcaggaggattgcagttcaaggtctaactgaaaaccagccagcctgggctccttagtggtgtgtatgtgtctgggctacttagtgagactctgtgtgtgtgtgtgtatgaatgtatgtatgtatgtatgtatgtatgtatgtatgtatgtatgtatgtgtgtctatgtctatatatgtatgtatgtgtgttctatgtatatgtgtatgtgtattaaatatgtgtgtatgtgtgtattacatgtgtgtatatgaatgtgtgtatatatattatatatatatgtatatatatagctataatacatgactaggactatagctcagtgctagtgcgcatgcctagtatatagaaggtgctgggttcagttgtcagacccattaggaaaagaaaggaagagaaagaaaagaagcaaaaaggaagaaaaaaagggattggtagaacatttgcccagcatgtgtggcctagaagcacattcatggagacagaggagaaaatacttttgtaaggacagatgtgtattcattacagagagcattcccgtatgctgaaccgggtcaccacagggccatgacaagagcctctgcagccatattgccagcctgagctgtatagtgaccgtggctcaaaggaaagaaacagcacaggagaggaagaaagacagagtcaaagagagaaggacagacggggactctgcgatagccatgtcacagagagttttatcttggacaccagagctagctctgccaagtaatgactgtgtgactttgaacaagcgtctgtgcttgtgattcctcagctgacagttgtctagtgctgcgaagagataccatgaccgtcgcaagtctcataaaggaaaacatttaatttccttcagaaaacattccatttttctgaactggcttacagttcacaggtttaatccattatatcgcggcaggaagcatggccattgcccaggcagacatggtgcaggagatggagctgagagttctacatcttgcaaacagcaaggagagactgtgttgcattgggtgttgcacgttgcttgagcatataagacctcaaagccctccgctacttcacataacaaggccacacccactccaaccaggccacacccactccaaccaggccacacccactccaacgaggccacacctaggaatcaaatacctgagcctatggggccattcctagaactgatggttgtagtagttgttgaatggcttctgctgttgttcagggcctggaatttgcgctcatgctgtgagagaagaattgtttgctcactgtagtattttactttaaagcttatgaacagttgcagagagaaacccacacagctagagagtgagctagcgaacccgccaggagccgcaaaccacactcccatctgccggactgcaaacaccgcactgttctgctgaggaatcacaaccgcagatgcttgttcaaagtcacgcggccattacttgacagagccagctctggtgttcaagatggaccccttaacccacagcctgcatggggttgcagcaagtgtgaagttgctggattgtttcgccttccctgctgtcaggggcagaatggggccggtagatgacgactatctccccctctatgtttccttccatgagtatctccgtgttctctctctaggcatctgtaatgggccacagcgcagcctgagtctgtccttggagaaggaaatggcgccagaaaccatgataccctgcttggaaaatggccaccttgtagcagggaacaaggtgccagtaagctgtgtatgccttacagctgtgccgtcggatgagagcatctgggagatgctgtcccccaagctcagcacaggtactgctattctggggcaggatgagacgagggcatgccagccggtgcaagggagtgggctgcctgctgtttgtgaggaggaaagataccacttgtcccacaagaattctctgggttaggaggacaggctgtggggatatctccagctactgcacaagatggccactgctaccttctgagaccctcccccccctttcctgtccttagagagcataatcaggagaaagttgatgtaggtgactccagtgctacccacacaggccccagcccagcagtcagacccgggcctatcctgccaaacttctccctattgcctccagctcagttaagagccggggatatgagggaggacctggggtccagcctcagtctcccaagtcttccttcagaatgcctttggggtcactgttgtcttcttgtccatctgttgctccttgtccgtctgtctctccttagctccctcgtccttcagccgtagttttaaacctccatgtgatgcattttatctcattggctgtccagtcgatcctgaatgttctaaatgttcgtgtccctcacaattcctgcgctgacagcagaacgtaaggctgtccttaaggggtgaagccagggatccaaggcaagccttagctacagagtgaattctgagtcagcctgggctacatggcatcctatctcaaacaaaaataattctcatactgagatctgagcccctcaaggggatggtgttggaaggcaagcattggaacaactcctgagggcaaggccttccaagtgagcttggaaattgccataaagaaatccaaccccgccaggtggtggtggcatacacctttagtcccagcgcctgggaggtagaggcaggcggatctctgagttccaaggccagcctgccctacaaaatgagttccaggacagctagagctacatagagaaaccctgtcttgaaaaagaaaaaaaacaaaacaaaacaaaaaaaaaaaaaaacaacaggtcagtgatagcctccagtcctttagcaatccgtgtcctggacctccaccagacaccagtgactgttggcaccttaacacatggctttccagcctccaaatgtgtgaggaggccgtgtttgttgtttacagaagtggcctggtaatggtgtgacagtggtgtgatccaccaagtcagacagtcttgtccgaaaaccagcttcctgcttggcccaggactggttcagccatgaatgaaacccagtgtctgccctccaggcagtagctgttcaaaggaaagtattaggaggtcatcagtggaagagggagtcagctgaggggtggggtcagggggagccgtgtgctccctcaaacttatctggaaacactgaagagtcatagacctaaatatgtgccagtagtggggaggggaatggggtagaaagagggagtctcggggctggggatttagctcagtggtagagcgcttacctaggaagcgcaaggccctgggttcggtccccagctccggaaaaaaagaaccaaaaaaaaaaaaaaaagaaaaaagaaagaaagagggagtctcactgtgtagccctcatcggccacaaacttgaaatcctcctgcttcagcttctcaagtgctgggattagaaccaccacgcctggcttgagtccgacctaatcaccctcgctgtgagagaaacagctcacaagctccagcttgttccatcagagcagctgctggccctttggcttgcttcaggcttcctatatttcaaagcccatccgagccccatcagaggggcttgaatcctggcgttctcaggagccattcagacttatagttccaaattgcctttttcttttagctctgactgaagcgattaaagacagggggggctacctgaacaaagtctgcaacctcctgcccattaggatcctgtcctacatcttgctgccctgcactctgcccgtggagtcggccatcgctgcagtccacaggtgagcgtcgaggcacgtgtgaaagtcagtgagctgatagtggcttctgcccaggctggctgtatgtcacatgactggaccccagcgagaggccgtccttccttgagccatggctgtccacaacatggtatacgctccgaagtcttctggagatccgtagagatagttatggctttaaactagcatctggtcccagccactcatgatcttctggggaaactaagaggtagaagccaccactctgtagctaccaagctggtgaattagaaggccaggcctagagacaagtccttgctatctatccattgggcctgtgtttagaatcacagtacttagctgagaggcccattctcctctggccatggttgcatgatttgagttagagtcagtagtgaggtggtcctgggacttggtaacaggctccctctcaaacagcacagcagagcagcagcttggtttagtgtgaaattaaaaacaatccatgctatcgccagctactctctagttgccctttcccctccctcactgggagctgcaatcccaggtcttccctgtttccccagaggttcccagccatccgtccctgtaacccagtaaaatcaaaactccagaggcttgtaaattatcagtcagatttataacagcaaattctcatcccacaaaaggcccacacaaagaactcaaaactcaattgttactgatgcaagctgccctatattattctattctcttctatgatagtcatagctacctgtggctatttcaagccacgacgatccggttcatcttcttcctccatcttcctctgtctttctccctgtctgtcctctgttctctctctgaaactcttagccccatcttcctttttcattgcccaatcacaggaccctagccttttatttcacttgacctcacctgcatacagacagcaatctacagtttagtcatcagaacagcaggttcaacatctggctgcatcccttccactgctgtgtggccctggactaatgaaggtgcctctctgggcttcagctttcctttgaagaaacagaagactgcctgggaggtcctgggtgtggagtactgtgatcacgtgtccagttagcaggggcgctgggaatttgttttcaggttgatttcccttgttggaaacttctcagggtttggttttgtggttgctactttggaattctgggtatttgcacgggcttaccagttaagcaaggtcaaacaggtaaaaatctcagaaatgttttgagcatcatgcggtcgctcagaatgcttcagatctttaacactgggtgtggtggctcccaccttcggccccagcacaggaggctgaggcagaaggatcaggaattcaaaactatcctgggccacagagttagtcaaggccagctcgggctttgtatagagaccctgtctcataaccaaaatgtgtctggggatgtaactcaatggttgagtggatagttagcatggttcaatccccatgctgccattaggaaggaggaggagggagggaaggaaagaggaagggagaggggaaaagtggggagaggagggaggggaagagagagtggacagagggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggtatgatatctagaggttctggatgtgtcatttgctctggacctctcctgatgtccttaccctgtctcccctgggctggtgcctcagggacatgactcagccctgcccttccatttctttggatcctgtctctttttcttggcattcctccttgtttttctgaatcggagcctacctaaatccccacggtaacagcgatctgccagcctcctcagagcctcacctgttttctacacctgagctgcagcccggcagctgctcccagaccctcgggcacggtctgccctctgagggcctggctggggccccctgtgctattcatgacttccacctgttctcctccacggcctgaaggtcctaatctttctgcccctcctcagccagtcttttgttctgcagactgctactttcccagtcaaaggccctctctctctcctggcttggggacacgacctcctgtaattgtgtctgtcgttccttctttcccgtggctgttcctcctctcttcctgagactcttgtgagatgttttgccaccgggttgaattctggcctcaggaaccttttcttttcaaattctttgtgtttttctttcgagacagggtatctctgtgtagtcctggctatcctggaactgtagaccaggctggcctgagactcacagagatccccccccgccccccccccccgctccccgttctgttctggtttctgagtgctgggactaaaggcgtgtcccatcgttgtttggcatccgaccttctcttttctacatatttcagtcctttggccttttgcctccagtctgaagtcttctcagatttaccttctagctccaccctcggagttttaacattaacagcatgagtgaacgtctatgctctgacttgtccttctgtttcctttacttgagccttctgggcttggtttgtgtgtgctgggagaggggtactgggggtgtgactgtggaagtgggtgtgtctgtatgttgagggctagtttgacgtttattttagtctgtatggattgatttttgtgtctgtgtgtgcccatgtgtgtgcatgtgtgtgtgtgtgtgtgtgtgtgtgtgtgtgttaacacagggtctcactgtttgaacagacaggctggccttgaattctcggtcctcctgcttcaacctcctagtgctaggacgctagatatgcaccactgtgcttaggtgcgtgtcctgccatcccagctatcccagctgtggctgggccacagcctatgtgtggaggtcagaggacaacccttagaagttggttctctccttccatagtccacactcaaggtgtcagccagggcagaaaacatcattaccctgtgaaccatctctttgccccatgtgtctgtacttctgagacagaattttaactctgtagcccaagctggcttagaacttactgtgtagaccacactgaccttaaactcaagtcagacctctcccaggcagtcggggtgtaaggcctgattctccgctcccccaccccacttacgttcctatgtcagcaacatcagcatgactcccaggataatgctggtgcagcctgcttctgcgggaagcaggtctcccttgggggtgcttcctccttggccttcctggagacttggatacaaggccaagtttgactgtattaccagtgcttcgaggtgtccgactccctccccttctggctgaggctactggaatcttttagccaggtcagcagttcgtacttgcaaccagcccaactgctgaggcaaaagaaaaaaaaaaaaaaaagcccagttggtctaggccagcctgtcgagatgtcaagagactcagagaagaaataaatagcccaggcctgcctccccgcttctcactttgctctcttggcaagcatactgcttgtgtgggtctgctcacataggataggatgtgcagtgaactagtcgtgtcttgtcccccacatttttatcttagtgcattgcacaggcccaacctttatccatcaaaatccttgggcagctttactgaccccatgtcccccttgaacagcttctccaccaatctaataaatcccataaagttgaagtcttgtaaaaaggcagctctgggtgggttcaccaatgtcacaggagggggtcatgggtgaagttgctaggctgagctccagtagagactgctaagggagcagggaggggagtttgtgaagtgatggttatttggtaagtgtagggccctgtaggtgggtcttctgtccagtggttactttggagtctgggactgggagctggctttgtccagtgctttacacaggcttttgcaagcatgggctcctggtcagaagagaagggtgttgcggggtggtctcctgacccataccatctcatctttattcccaaacacccctctggggtcagactcctgctttgggggaggctcttaccagcaacctacctcttgccagagtctgcagatggttcggcccctgtggtcctacagttggattcagagtggccatgcacagctggcctctctggagagcctaggctagccctcagccagcacttgccatcaacccttgagaacttaggtagactgaccccatgttgcggcctttcccactcattcccaggagctttctggccaccttaaatggcctgtgtgctttagagacttttgaaggttttctgttgtgatgacatcccaaatatgccttagggagggtatcacattggggttagaggtgacacaggcagctcacttggataaaagtcactgttgggactgcttttttcttttaaagatttatttctttattttatatatgagtacactgtagctgtcttcagacacaccagaagagggcatcggatcccattacagatggttgtgagctaccacgtggttgctgggatttgaactcaggacctctggaagagcagtcaggtgctcttaaccgctgagccatctctccactcctaggcctgctttttactgaccgtttttttctggtttaatcttttttttttttaattatttattatatatgagtacactgtagctgtcttcagatacaccagaagagggcatcagatctcgttacagatggttgtgagccaccatgttgttgctgggaattgaactcatgacctctggaagagcagtcaggtgctcttaaccgctgagccatctctccagccctctggtttaatctttaggctgatacagtgagcccatgtgtgcatgtgatatttgtgcacatgtacatatatgtgcatgtttattctatgcagagtactcatatagggtctctgttaggaaatataacacagggttaaggggtcacccagagagcccattgccaaggtgtctccctgagaaatcccatcatgcaacagacctggtataagggaaatctattgtgggggaagggaggggagccagacagagtcatgaggccggggttgggtggatagagaaagttagagagaaagggaggctggctgggaacatgtgggactagagagagaaagaaaatgagagagaataagaaggggagaggggctggtaacgagcagtccctttccaagctaccaggctgcctacacctggtggcagctcaggtagtaatgggggcaggtgatgacctaagctgttgctaggtgtctgttgctaggtccctgggaagagcctgccagaatcacctgtaagccaatagtgtccttggcttccttcccacaggctggtgatgtggctccctgatatccatgaagatatccagtggctacagtgggcaacatcccaggtgtgtgcccgaatgaccatgtgcctgctcccctctaccaggtaaagactttggtgcagggtatgtgggttagatgggcctccctgttgttcccagagctcttagggtcagagcttgggacatgacggtcctaacaagccaggctcagctacctgtccccaacatgctgattaaagaaaccagtagcagagaaagcaggtttattcgaaaataaaaaggttcaagccaccctgcccccacccattaatctttaggatactaacaggaagcttaagtttaaacaatcctgatcagggtgtggtcctgcccacctttgtctgggcttcggtttcttttgcaaggttcctgctttctctcccaacataaggttcctgggaaaattccaattccttggcctccactgtatcgaagggctgtaaccccgttgatgcggactcacttctctggtgaaaatattgtgctcccagcaagccaggattcgcaatgtcccaggaggtgcctgtcagtatctgtgagaaggagacctgagcagagactggataatcatattcttatgatacttgggaagggcctggaaccttactttttttttcctttcagatccagagcatccaaggataaccatcaaacactcaagcatggatatcacccatctctccacaaaccccaaggcagctctgccggtttgtaaattgctggtctccgtgcttccgatgaacttgggcattctccctgtggatggttccaggagaggccatagctgaaggcactctgccttccaccccaagtccagtttgacctttatctagagcaacagtgtctagatgataggtgggtggggggtgctgtctctctgtttccctctgggaagggttctgttaacttttggaggcagctaggaaatttctctccaggagctgagcctgtgcagctgcccccttggtgctgtgtggtaacctcattgcctgtgaccctaggatcataggatctgggctaaataggtagttcatagaaaccaaagacaataatttggtgtttagaaaactacttttggtctgggtgaagtctggtgcttgagagttagtgcagagagaacggtcaaaccgtctctcagcctgtggatctatggggattccaagggcttcagtgtttggaaacggcaatccaaacgggcaatcttgtgcaatcttggaaggagaactgttcaggaagtgtgatgggatgagctgtggctgtctctgaaaagggcctaccatataacttattactttcaaggatacctttggctcttactaaaatagtttataaagcattttatagaaacacaccagggaatgcgtggtgaactacatgtatgatcagtgaactgtgactagaattaaccttaaaatctcttgtatgtggggccagagcaacacaggtgggaaacgcagcggacctctgcctcctcggcctcaacatgaacttggcttgctttctccaccgtctccaaatctttgtatagtcatcgaccattaccacctctcctttcccatctactacagcagccttaatggggataagtacccccttttctcaggtgtccgaataagctgtgggtgtggcctgtgtttcctgtaat

SEQ ID NO: 55 - DNA, 1650 bp, Rattus sp.
atggacgaacaggtgttcaaaggagacccagacacccctcattccatctccttctccgggagcgggttcctgtcctactaccaggctggggccgtggatgctctgcgagatctggcccctaggatgctggacacggctcatcgctttgcagggacgtcagctggcgccgtgattgcagccttggtcatctgcgggattgagatggatgagtacctcagagtgctgaatatgggtctggccgaggtgaagaagttcttcctaggacctctgtctccgtcctgcaagatggtacaaatgatgcgacagtttttgtacgacgtgctccccgaggactcctacaaggccgccaccgggaagctccacgtgagcctcacccgggtcacggacggagagaacgtcgtggtttcggagtaccggtccaaggaggagctcattgaggctctgtattgtagctgctttgttcctgtctactgtggcttcatacccccaacatatcggggcgagagatacatcgacggcggcttcacaagcatgcagccctgcgccttctggacagactccatcaccatctccaccttcagcagccagcaggacatctgtccgagggactgccccgccatcttccatgacttccgaatgtttaacttctccttccagttctccttggagaatatcacgcgcatgacgcatgcgctgttccccccggacctggtgatcctgcaggaatattactaccggggatacgatgatgccgtctcatacctgcggaggctgaatgccgcgtacctcgactctcccagcaagagagtgatttttccgagggtagaagtatactgccagatagaggtcgcccttggccatgagcccccacctacgagtctgcagagcctcccagcacagagagacagccctgaggactgctcacaaacccatgtacagggggctctcaaagaggacagaaaggacagccattccccagcaaccccatcagcgcagacatctgaatctgggtgcaaggggtttgaggagtcacctgtgtccccaacggtctcttcatccaagcaaccagctgtgtcaccgttggtccaagcccagccgggcgcagtagtgaggcccgctgaccccagtgacagttgcccagtgaatgttcaaactccaaacctggagcaaggagtgaaggatgactgtcagccagacttggccactgatgaccaaagcacaaacagcctgctccctgcatcactcccagctgcagactcacaggaatcaaaggctggatcctctgtgtctgttgcgtcactcgagtctgctggactgctgctcaaatcttcccagggagccacagtagccggggcaacagctggacttccgcccctaagtccttcaacgccacccgcccagccacctgtggaagacctaggcccagaacaacccccagctacagggtctcccgcctcatcacagctgacaggctcggcagcaccggatacggggaagggagcccccgacaagcctctactggtggccagtcctgctgcagattcaaacacagcaaaaaagatgttcaggaggaaacagaaggcaaatgccaccagagagtgcttccagaaaaaccctcagtccaagaagccagctagcaaactgcagtga

SEQ ID NO: 56 - protein, 549 aa, Rattus sp.
MDEQVFKGDPDTPHSISFSGSGFLSYYQAGAVDALRDLAPRMLDTAHRFAGTSAGAVIAALVICGIEMEEYLRVLNMGLAEVKKFFLGPLSPSCKMVQMMRQFLYDVLPEDSYKAATGKLHVSLTRVTDGENVVVSEYRSKEELIEALYCSCFVPVYCGFIPPTYRGERYIDGGFTSMQPCAFWTDSITISTFSSQQDICPRDCPAIFHDFRMFNFSFQFSLENITRMTHALFPPDLVILQEYYYRGYDDAVSYLRRLNAAYLDSPSKRVIFPRVEVYCQIEVALGHEPPPTSLQSLPAQRDSPEDCSQTHVQGALKEDRKDSHSPATPSAQTSESGCKGFEESPVSPTVSSSKQPAVSPLVQAQPGAVVRPADPSDSCPVNVQTPNLEQGVKDDCQPDLATDDQSTNSLLPASLPAADSQESKAGSSVSVASLESAGLLLKSSQGATVAGATAGLPPLSPSTPPAQPPVEDLGPEQPPATGSPASSQLTGSAAPDTGKGAPDKPLLVASPAADSNTAKKMFRRKQKANATRECFQKNPQSKKPASKLQ

SEQ ID NO: 57 - DNA, 38939 bp, Rattus sp.
  misc_feature (330)..(330): n is a, c, g, or t
  misc_feature (333)..(339): n is a, c, g, or t
  misc_feature (341)..(342): n is a, c, g, or t
  misc_feature (2549)..(2551): n is a, c, g, or t
  misc_feature (2558)..(3539): n is a, c, g, or t
  misc_feature (4908)..(4957): n is a, c, g, or t
  misc_feature (4959)..(4997): n is a, c, g, or t
  misc_feature (5000)..(5148): n is a, c, g, or t
  misc_feature (5151)..(5217): n is a, c, g, or t
  misc_feature (5219)..(5251): n is a, c, g, or t
  misc_feature (5253)..(5283): n is a, c, g, or t
  misc_feature (5285)..(5285): n is a, c, g, or t
  misc_feature (5290)..(5290): n is a, c, g, or t
  misc_feature (9467)..(9467): n is a, c, g, or t
  misc_feature (9562)..(9562): n is a, c, g, or t
  misc_feature (9591)..(9591): n is a, c, g, or t
  misc_feature (9602)..(9604): n is a, c, g, or t
  misc_feature (9608)..(9609): n is a, c, g, or t
  misc_feature (9613)..(9619): n is a, c, g, or t
  misc_feature (9623)..(9623): n is a, c, g, or t
  misc_feature (10113)..(10113): n is a, c, g, or t
  misc_feature (10125)..(10125): n is a, c, g, or t
  misc_feature (10127)..(10127): n is a, c, g, or t
  misc_feature (10136)..(10136): n is a, c, g, or t
  misc_feature (10144)..(10144): n is a, c, g, or t
  misc_feature (10146)..(10147): n is a, c, g, or t
  misc_feature (10180)..(10180): n is a, c, g, or t
  misc_feature (10223)..(10223): n is a, c, g, or t
  misc_feature (10239)..(10239): n is a, c, g, or t
  misc_feature (10250)..(10252): n is a, c, g, or t
  misc_feature (10255)..(10255): n is a, c, g, or t
  misc_feature (10261)..(10261): n is a, c, g, or t
  misc_feature (10281)..(10281): n is a, c, g, or t
  misc_feature (10298)..(10298): n is a, c, g, or t
  misc_feature (10302)..(10302): n is a, c, g, or t
  misc_feature (10396)..(10398): n is a, c, g, or t
  misc_feature (11374)..(11374): n is a, c, g, or t
  misc_feature (11379)..(11380): n is a, c, g, or t
  misc_feature (11393)..(11395): n is a, c, g, or t
  misc_feature (11397)..(11398): n is a, c, g, or t
  misc_feature (11400)..(11400): n is a, c, g, or t
  misc_feature (11402)..(11404): n is a, c, g, or t
  misc_feature (11422)..(11423): n is a, c, g, or t
  misc_feature (11431)..(11435): n is a, c, g, or t
  misc_feature (11438)..(11446): n is a, c, g, or t
  misc_feature (11448)..(11448): n is a, c, g, or t
  misc_feature (11450)..(11455): n is a, c, g, or t
  misc_feature (11457)..(11457): n is a, c, g, or t
  misc_feature (11462)..(11468): n is a, c, g, or t
  misc_feature (11470)..(11472): n is a, c, g, or t
  misc_feature (11474)..(11476): n is a, c, g, or t
  misc_feature (11478)..(11480): n is a, c, g, or t
  misc_feature (11482)..(11482): n is a, c, g, or t
  misc_feature (11486)..(11486): n is a, c, g, or t
  misc_feature (11489)..(11501): n is a, c, g, or t
  misc_feature (11503)..(11525): n is a, c, g, or t
  misc_feature (11529)..(11578): n is a, c, g, or t
  misc_feature (11580)..(11661): n is a, c, g, or t
  misc_feature (11665)..(11682): n is a, c, g, or t
  misc_feature (11684)..(11695): n is a, c, g, or t
  misc_feature (11697)..(11701): n is a, c, g, or t
  misc_feature (11705)..(11706): n is a, c, g, or t
  misc_feature (11710)..(11713): n is a, c, g, or t
  misc_feature (11715)..(11715): n is a, c, g, or t
  misc_feature (11717)..(11731): n is a, c, g, or t
  misc_feature (11733)..(11736): n is a, c, g, or t
  misc_feature (11738)..(11759): n is a, c, g, or t
  misc_feature (11761)..(11772): n is a, c, g, or t
  misc_feature (11774)..(11789): n is a, c, g, or t
  misc_feature (11792)..(11803): n is a, c, g, or t
  misc_feature (11805)..(11861): n is a, c, g, or t
  misc_feature (11868)..(11873): n is a, c, g, or t
  misc_feature (11875)..(11893): n is a, c, g, or t
  misc_feature (11897)..(11998): n is a, c, g, or t
  misc_feature (12001)..(12025): n is a, c, g, or t
  misc_feature (12027)..(12044): n is a, c, g, or t
  misc_feature (12046)..(12056): n is a, c, g, or t
  misc_feature (12061)..(12068): n is a, c, g, or t
  misc_feature (12070)..(12074): n is a, c, g, or t
  misc_feature (12077)..(12077): n is a, c, g, or t
  misc_feature (12079)..(12079): n is a, c, g, or t
  misc_feature (12082)..(12083): n is a, c, g, or t
  misc_feature (12085)..(12099): n is a, c, g, or t
  misc_feature (12101)..(12105): n is a, c, g, or t
  misc_feature (12107)..(12116): n is a, c, g, or t
  misc_feature (12119)..(12119): n is a, c, g, or t
  misc_feature (12121)..(12135): n is a, c, g, or t
  misc_feature (12138)..(12140): n is a, c, g, or t
  misc_feature (12173)..(12175): n is a, c, g, or t
  misc_feature (12181)..(12181): n is a, c, g, or t
  misc_feature (12184)..(12184): n is a, c, g, or t
  misc_feature (12186)..(12186): n is a, c, g, or t
  misc_feature (17496)..(17496): n is a, c, g, or t
  misc_feature (17607)..(17607): n is a, c, g, or t
  misc_feature (17767)..(17767): n is a, c, g, or t
  misc_feature (27668)..(27668): n is a, c, g, or t
  misc_feature (30839)..(30839): n is a, c, g, or t
  misc_feature (30853)..(30853): n is a, c, g, or t
  misc_feature (30897)..(30897): n is a, c, g, or t
  misc_feature (30902)..(30902): n is a, c, g, or t
  misc_feature (30912)..(30913): n is a, c, g, or t
  misc_feature (30923)..(30923): n is a, c, g, or t
  misc_feature (30928)..(30932): n is a, c, g, or t
  misc_feature (30935)..(30950): n is a, c, g, or t
  misc_feature (30952)..(30956): n is a, c, g, or t
  misc_feature (30962)..(30971): n is a, c, g, or t
  misc_feature (30976)..(30976): n is a, c, g, or t
  misc_feature (30978)..(30978): n is a, c, g, or t
  misc_feature (30980)..(30980): n is a, c, g, or t
  misc_feature (30986)..(30987): n is a, c, g, or t
  misc_feature (30989)..(30989): n is a, c, g, or t
  misc_feature (30991)..(31006): n is a, c, g, or t
  misc_feature (31008)..(31008): n is a, c, g, or t
  misc_feature (31010)..(31015): n is a, c, g, or t
  misc_feature (31019)..(31045): n is a, c, g, or t
  misc_feature (31053)..(31061): n is a, c, g, or t
  misc_feature (31076)..(31215): n is a, c, g, or t
  misc_feature (31394)..(31394): n is a, c, g, or t
ccggcaagcagtaagcagcgttctcatggcctctgcatcagttcctgcctctctgccctgactttccctcatgatgaaccttaaactgtgagctgagatacccctttccttccccaagttgcttgtggtcaccgtgttatcaatggaaagcaaaccaggcagtcacccagagccaaactcttgttagtttttgcccagttttgtgctaaggtaagaatagagaggagcggggttggggatttggcttagtggtagagcgcttgcctaggaagcgcaaggtcctgggttcggtccccagccccggggggtggggggggagaatagagaggngcnnnnnnncnnctttccagagtctgaggagggtgaggaaggagcatgggggcgatatgagttgctcctccccattgaaagccaaagccttgcttgggggcggggggggggtgggatgggacacacacacacacgcacacacacacacacacacacacacacacacacacacggactgggggcgtggcgactaggggcgtggcatgtaactcagtccccagtcagtgagggaggtacagggagaggcagagattttgcaactggatgctaagggctgaaaggcgccgcctctgtggacacggcccagagcagggagcagggaatgcctcgctccagagtgcgcccaggatgggtggagagcgctgaagagcctgcgccccgctgtgagtctagctaggtcatgaagtcagcatggggtctgcaggaccacgcccctgcccatgactctgagaactgtctacatttcatccaggacctggggtcccttatcaggccttcaggggtgtctctcagcagagctcaacaggcccaggcagtggctggatgaaccacccgtgggtgcctggctgggccggtacattcccggctcttgactgacagggaaggagagactgagccaaggcagacaggtgtaggtttttcctccgctgggaggccagaggccccaaagatggacgaacaggtgttcaaaggagacccagacacccctcattccatctccttctccgggagcgggttcctgtcctactaccaggctggggccgtggatgctctgcgagatctggcccctaggatgctggacacggctcatcgctttgcagggacgtcagctggcgccgtgattgcagccttggtcatctgcgggattgagatgggtaagacctttactctggtgtctctgaagggggctgctggagggactcctcagagcagaccgtgtccaacttggaagtgcactgggggggttaaagccaagcttgacagccagggtagctcccccttcgcacgcacacgcacacgcacgcacgtatgcacgcacgcacatccaagcatgcgcttttcctcttcctcctgtgctgggccagaccactatagtctggtcatgaggagtgaggctgggtgatgttttttcttgtttgtttttttttttttggttcttttttttcggagctggggaccgaacccagggccttgcgcttcctaggtaagcgctctaccactgagctaaatccccagccccttttttcttgttttttaaatgcaagcactgccccctcttcttagtgaggaaggggctctcaacatgcaaaccctgaggactgaggagaagctgggaacagacagtgaggcacagtgtgggagcttcagcttcagtggtgggccggagaccacagggctgacccagggctctgagcaacagcctccttgtcctaacagtgtagagcacgggcccagccaccccgacccgctcaaggccaggctgtaatgtagaacccctgtggagctgaagttctagaacattctagagagaaaagacagataacaaacttccaaatggagaggtctctcgtgtcatgagggatggggtctgcaaacaaactgcagctgtctggggctgaagcgggaagggggaccgtgttccagagatgttcaggggcggccatgttgagggggaggccatccaagtggcctgtggcattcaattctgaacacacagaagccagtccctgtgctccagcttctgttatcaggacaatccctatgacggcctgacctgggtttgtcaaagaaggaacttcactgataggagtgaactgctttgtgattgagcatggagtgctggaaaccgccaacagtctggtttgggggttttaaggaggcctgactgctgggttggaggggcctgagtagaaaccgactaggggatgagtgtgctttgtggaggaggatggggattaggtttggaatgtgctagaaggttgagttgagaccaactgaagggtcagctattggatctctgaggaggcggcagccgatgggggtggggctctgccctgaagggcaacagtagggccctctctagttgccgcacacaggcaaggaaccgtctcctttttaatagtttataagaaacgatgggacaagagagttaaaatttctgcccaccctgcagcccctagtccactcaccctccccaagcctgggcaaagagagggatnnngatccgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaggccaccacggaccccagggagtgggatgaagtcatgaagaggccctctaggagggatcaaggcctagacccggcatggctcatgaatagagcaggatgtaaggaggggtgaggtgtatggaccaatccagtccagtcattttactggcccgtgatttcctgataaccggatgtcttttgtatcagatcctgtttgaagttttaagggtattaactcacccagactttacagctccttctaagggggaaactattattacctccctttatacaaagaaggaaacagtcacaggacacttacgcagcctgaggtcacacaaactacgtggggagttcaccctgagcctcatctgtccagagtggatgctggaatgacttaaaccatgtcaccacttggcagagtggacttcggggttaacagtgggtctcggggacagagtttttacagacctgattcactgaggggatggactggaaaacatattgggcggaggcatggtggtacaggcctttaatcctggtgcaccagaggtagaggcaggcagatctctgtgagttcaaggctagcctggactgcagcgtgaggtccaggacagccagggctatattggtctcaaagcaaaaccaaaaataaagaaaaattcataaaaaatatttttttttaaaaaaagaaagtacttaggggagaacatttaccagacagaggatgggtaaccaaggtgttgacctgttatcagaaactcagtgggagtctagaccgaagtccccatgtaccactaacaccaagccccgctcagttcattgccgtgttgatggataccctggggttgtaagcagtgttacagggagcagggctgtctggtgtctgccatgcccaggatgggtgcaagcccttagagtcaagagtagcctgtggtgggctctcctgtgccatgctctaggagtctctgaagtgactacagtttaatccccagacacacacaggagtcgggtgagtgtgtatgatgtgtgtgaggtgtgtatgatgcatgcatgtgagattatgtgtgtgtgatatatgtgagatatatgcatgtgaggtgtacgtgatgtgtatgtgtgagattatgtgtgtgtgatatatgtatgtacatgcatctgagatatttgtgtgtgatgtgtgtgtgtaagattatgtgtgtgatgtgaatgtgtaaaattatgtgtatatggtgtgtgtgatatatgtgtgtgtcatatgtgtgtgtgatatatgtatgtccacgcatgtgagatatgtgtgtacgatgtgtgtgtgatgtgtatgtgagatgtgtgtatgtgtaagattatgtgtgtgtggtgtgtgtgtgtgagatatatgtgtnnnnnnnnnnnnnnnnnnnnnnnnnnnnnnnnnnnnnnnnnnnnnnnnnnannnnnnnnnnnnnnnnnnnnnnnnnnnnnnnnnnnnnnnaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtnnnnnnnnnnnnnnnnnnnnnnnnnnnnnnnnnnnnnnnnnnnnnnnnnnnnnnnnnnnnnnnnnnnannnnnnnnnnnnnnnnnnnnnnnnnnnnnnnnnannnnnnnnnnnnnnnnnnnnnnnnnnnnnnntnccccntcctcctctccccctccctcttccttgaaaccttccttccattattctcagcctctttgccctctccaacaggaaaggcccagcagtgattttatctagaattaagcgtcccgtggtaaatataacccctgactccgctcactgtgccctttaatgaagacgctgtgtgtcactgtatatcgatttaaggatctctgtcttgtttttgtctcactctatagacaaggcttgcctggaactcatggtgagtctcctgcctcagtctccgggaccaggtttatctctaaggctcttaaggtttttactcctgtgaaggagcttctctttccactgcatttcctgatgggtcagtgctgacttagaagggtcggagggtttttccctgctggtcttgttctcggggattcagcacacatccgttgtagatgaaaaactatgttcattttctcgtactttatacgggaccaactcgtgtatataagtggagatctgtcgctttttgaaattatttgtattatgttcactgtgtgtgtgtgtgtgctcgggtgcacaagtgccacgtcaaacatgggtcagaggacagcctttaggagttgcttctctcctgccaccaggtgggtcccaggccatgtccttgcagggcttgggcttctaaccctgactagaagcgtcaggagatgaggacatggcagatgcacagaaaagctgggatgttgtgggcggagcttctgatggagacactgtagcatcctgggagctcagcgtgtttactatagacagcagtacacagaggtagggcattacacacagataaaccagcagatacaatttatacccagctggacaagggggcagggttattatacaccactggggcaggttcagctaatcttggtaggagcagtctctctctgcaggggagcaacctgcaggctgcaaatatcttggggagaaagctatggtgggtcctttcgccacacactatcaacatccacacagagactaaaggaaggctctgccattctcacgaggccaaggaatagatagggtctttgacatggcagtggccatatcaagagcacggtgacttggtccttcattttcttagggtttcctatgctcccaaggtcctgggtatcatactccgttcattaggcaggaagcccctttacatgccgatccattctgctggtgtcctgtctttttttttttttttcctttaaaaattcttttagcccagccacacttcttccaaagctgtttcccttcggagcttctctgacgagagacttctctgcctcatccctggggaactggtgacccagtctccttcagtcagtgcaaagcccttagcaccctgcctggtcctcatcggagatgacatcaccatttatttgaggagaggcagtctccatgacaaccagtggggttccagcgtttctgatgtggccagaagtgggacgcagtttcgcagagctgaatttcctgtattttggaaataactctttataggatattttaagttccattcattcattcctttatcgtgtctatgtgtgtgggtgtgttttgggggcacatggcacatctgtggggggtcagaggacaactttcaggagtctgttcttccttcccatgtgttggtcccagggatcaaactcagatcttcaggcttaaaagcaagtgcttcttgccagtcccaaaaataatttctatctcttctcaaaggaaaaagatttggtggtcaggcagccccacctctttcagctgctactacagaaagacgcctcccccaaaagactttatggtagccctttccccctctgcctatccttgccgtccctatctttctcatgctttcttttcttgagatggggagtctcaggtaggccaggctgacccttcatgtagccaaggttgacctcaaactcatgttcctcctgtctgcacctcccaaggacagggaccacaggtatgtctccccatgtctgactctccatggtgtccttctaagagaggaagtgtctcctgggttctagaaaggcacagagtgaggccagcgcctccagtcagaacttcctaagggagccagcagtgtgagtgggagttaggcagggaaagacaggctgggcaggaccagaggcctccagcaaggatgtgggaggacaggatgacaaggtggacaaaccagtgtgtctggatgatggacaacacaggtcacctgatgtcacggtttcagagaggggaacttcagttcatgaaggtttctcaagtgggaaagatgaggaaagggagcaatggattctgggaaaaagcccaagtagaactgccggccatgctgctggtggccatgccagtgtcccctgcattactgaaatacattatgtggactgagttgacctgtgactgacatcatggatcaaaacccacagtaagaaagatttgggctggagggaggatgactcggtgggtaaaggtgagcctgaggtctacctccaggatgataggatagaccattctcacaggctctgccttgtgacctccttccctcccccaacatagcagaaagacattggcgttctgacccagttccaccaaagaggaagaacgagaaaggaaagtctgtcagggtgcaactcacctgtgactccgagattggagttaggaacctattggagatagggagtttgaggctagcctttgctacacagttcatttgaaacctgcttgggctactggagactgtctcaaaacaaaatcaaaagattgcttaactgtggcaatataggctatatccacccgcacacttctgttgtggtaaatattatttgggggtctctaccctgcctaggtcatttagtccccaaataaaggacacagaacttttttttttctttttctttttttttttcggagctgggactgaacccagggccttgcgcttgctaggcaagcgctctaccgctgagctaaatccccaaccccaggacacagaacttttatatctgtaataagccttaaggcaggagagctgggcagatatcaaccctctgtgccagtttgtttccctctctgtctgcagtcccgaggtatcacctgccatgttccacctgggctgctcctactccagctggtccgcacagccatgtgttcaggatccacctttcccatggtgacttcttccttccccctcctctgcttcatccgctctcctcagagcccaagccccgcccacctctcttccgcccagctgtaggcgtctttattcaaccaacagttttaaattaaggagtaaggtttgccccacaaaagctggcctatgtgagcattcactcctcttgaggtaactaaacctgcggatacaatattcagccttacaatacatagccacggaccaaacctcaacactcttcctctctctgagccacacagtacagaaggctgcccagggccaaggcgctggctcttttagcccaagtcccagcttatctcactcagagaagctgtctgtctgccccaaagtccagaagcagaaaaactcatttctcttcttaaaataacaaaagaaaccaccaacaagacctttggccttattgatacatagtttacttatcatgcagatcaccctacttgaagtttatttaaagtttacccagggtttgttcagttgtacaacaagcgctacagtttcagaacgccttacccacaaaggaaccttactccttagttgtggcctccgttccccagaagtcaccgatcagtctctccagatgaacacatttaacatgatacaatgtttcacatttaatacacaggcgatgcatacaccagtttgcacctggcttctttcccagcgccgtcttatcaaggctcaccaggatatagcatgaatagttggtgttctgttccatggcggaacagtattctactgggtggacttaccacatttattcatttattctctcgatgatgaatgggtggtttttactgtgggctatcgtgaatagtgcttttgtgagcattcctgttcctttaaaaaatgggtaggggagggggtagccatctctccagctctagatggctcagtggttaagagcactgactgctcttccacaggtcccgagttcaattcccagcaaccgcatgggggctcacaactcatctgtaatgggatctnatgccctcttctagtgtgtctgaagacagcgatagtgtactcatatacataaaataaatacatctttttttttccttttctttttttcggagctngggaccgaaccagggccttgcacttcctnggcaagcgctnnnccanngagnnnnnnncccnaccccaaataaatctttttttaaaatgcttttttttaacgtggaaaaattacaggacagtgtcctcatgtcctcatttagccgcagcagcattgacccagggtgtgtcaactcagctccccaattgctttgttactgtagaaggctttctttttttaaatttttaatgtattttttaaaagatttatttatttattaattataagtacatcatcaaaagagggcatcaggtctcattacatgatggttgtgagccaccatgtggttgctgggaattgaactcaggacctctggaagagcagttggtgctcttaaccactgagccatctctccagctctagaagcctttaaaaaaaattagggctggagatgtggctcagttggtaggctgcctgtctgaaggtgagtttgatgcctgacactaggtaaagcgggcactcggttcttgccgccccttagtctgctctccaaaaggtagcggcagaactgangatctcccaggnangaattcagntgagcggncnngctttgcccaccgagaacaggagctaagaaatngtgggcagtcagagtggctcactccgggagctccttcctttcnagtggcctttctgggnggggttcagcnnnganggaaangacttcagatgaggtccatntgcatccccttccactntggntctggatgtcatctggccagagtgctactttctggatcagtgacctgcgtagaatgggagagacatgagtctccccgttggcacagagccctcnnngagtcagcggtgcccaatctccagagccttccaaagcccaggagactgttaggagagctgtgttgactgggggcaacagaagagggctcgtgcctctacaactcgttttctagttcccctaaggtctgcatgtggcattctctctagaccccaagccccccagtcccaagagtcaacaaaccatcaaggtacacataggtgttgccctgtgtcgtgtccatgtctgaattccgtctagtcagaaggggaaacagacataccccttccctctgaggtaggaccctggggggggggggtgacacataccttgttagctctcatctatagatcagaacttagtcacattgcctcacctggtagcaaaagatgctgggaaacacaggcttgctctcgggcaggaagcctggttttgctccgacaaggtgtccaactggaaaggttcacggctgaaggggcaccgagaggcaactcacaggtgtcctgtcacacacacaacccaacaacatgtgagaggtatccacagcgctggcttcatcctgaggtgactcactcacacagaaggatgtgatttctacgaggaacacaatctccaaagacaacacagagagaagtgcgtgtgtgtgtgtgtgtgtgtgtgtgtgtgtgtgtgtgtgtgtgtaggttgatggccctgaggtatttattgagtgagtctctagagaactttctagacatatatattccctctgttagttcaacatgacctgtaagacttctccactcttttgcagatgaatgccgggagaacgtgcctggattcttggggagaggtgcacaggggtgcggagacacccagaatgagtgtccaaagcaagaaaggggtctgtgacagacgtctataaaaccgggctatgagctcattcccacagcccctaacctaagactatcagagtcccatttggttgttttggggaccggatctcactatgtagcctangctgnnctaaaaatcactnnntnncncnnnctgccctctaactcatgnncatcatgnnnnntcnnnnnnnnnantnnnnnncnatggnnnnnnncnnncnnntnnnantacntcnnnnnnnnnnnnntnnnnnnnnnnnnnnnnnnnnnnnaaannnnnnnnnnnnnnnnnnnnnnnnnnnnnnnnnnnnnnnnnnnnnnnnnngnnnnnnnnnnnnnnnnnnnnnnnnnnnnnnnnnnnnnnnnnnnnnnnnnnnnnnnnnnnnnnnnnnnnnnnnnnnnnnnnnngggnnnnnnnnnnnnnnnnnngnnnnnnnnnnnngnnnnngggnngggnnnngngnnnnnnnnnnnnnnngnnnntnnnnnnnnnnnnnnnnnnnnnngnnnnnnnnnnnngnnnnnnnnnnnnnnnnagnnnnnnnnnnnngnnnnnnnnnnnnnnnnnnnnnnnnnnnnnnnnnnnnnnnnnnnnnnnnnnnnnnnnnggggggnnnnnngnnnnnnnnnnnnnnnnnnngggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagnnnnnnnnnnnnnnnnnnnnnnnnngnnnnnnnnnnnnnnnnnngnnnnnnnnnnnaagannnnnnnngnnnnnggngngtnngnnnnnnnnnnnnnnngnnnnngnnnnnnnnnnggngnnnnnnnnnnnnnnngannngtatccactcatccaccccctaccccacaaacnnnggaccnatncngctcccacccccccacccaccacccccaaccttacacacccacaaccttacacacccacttttccactcatccacaaccttacccatccacccacaaccttacccacccatccacctaccaccccacttatccatctgcccagccgcccacttaatcgtccacacatctacccaccttcaactttatcctgggctggaacttgtatctgcacaagcagacaaatggcgcctggtctggtgtttgtgtgtgaggggctccccatcagtgcatacgtgtgcaggtgcacatgcatttgtgtgcgcctggggtcaggagtcaaccaccgatgtctttcttcaagagccacggcctgttttgtttgtgtgtttgttttgtatctcacagagagctggggcccgctctggttagtagaccacaagcatcaggatggctccccctcccccgcactggggttgcgagagctcacgggtacattcagcttttgcatgggttctgggggtctaaactcggatctcatgtcttcctggcaagcatttttaccccttgagcaatcctccctgcgtccggcctagtattcttgacctagactctcagaactatgcatttccaggaattttgaggggtttcctgcttaaggtgaggccaggcagtctcacggggttcaacccccatccggcttcaatccttcacagctttgcgttcagtcgttttatacaatcaattttcactttagatttgcctggcacaaagccttctctgactgaaaactggccgcacagagtgtccccacgggtttccgtgagcgacagggtagctttaaggcagtctctgcagtgcgcggtgcgtaaaccgggatctctgcccttagagcctgactgtcgctgtgacgataaacaagactcaaggtgggaaagcctgtttgggtgaactactttcattaagataagataatagcggttattattagaatgtggttaggtagtcaagctagacagacccaggggaaaacacgcctgggaattcagcctgcaattgggaagccagacgggagcctaaggcgtgcaggttcctcggtcagggattctgagccttgccggggcttccctgggaaatctgctcactcctgcctcccagaagccgcctcacctcagcttcccctgagcagtcatctcaatataggggagagcgggctggccaactgtgattggctaagctggaggctgttgctaggttaccagtccacagagactaggtgagtgtgtatgtgcgctcagccccatcctccttccctgattcgtcccacacagcaggcctaattcttgcacatccaagaaccggatccagtcatttacgggggatcggagtgggggtgggggttgggggtgtcggcctgcagtgccccttctccactgtgtgggggaagggaagacaggcatctcaaatttaactctttgagtcatcttgaataactatttgtctctttgggcctcagtgtccctttttgatttctggagatcactaatttatccatcatctgtggatgcgttcagttaattaagcgtctgccaggattccagacactgtgtcgaacactaagggttagtaaggcctctttgctcttggggggaaaggggtaaattatttccaatttaatcaaataacatacaccggcgtcaaattatctcttttcttccccttcggtgatcgctccagcccccgattttttttttttcaaatgcagccactagccatctcccatagaagaggcatgttaccagattgaccctgtcattcccgagaccacgggtacaccctctccgccatgtttctgctatgtccatgtattttgtatcttattttaaacttatgtattttcatgtgtgtgggtgttttgcctgcacgtccgtctgcgcgccacatacgggatgtgctcatgagggccagaagagggcgccagatcccccagaactggcgttgcagatggttgcgagccaccctgtgggtgctggatatcaaatcagggtggaggtcagcgaacaacttctgtggattagttctctccaccctgtggttcccagggatagaacccaggctcccgcgcaagcgcctttacctgctgagcctccacgttggactcggtttctttattttatagacggtgggtggagccagtgctgccccagacggctggtgtgaccctgggattggaagcaaatctgcctggctttgcagtacagatctgaggtcacagaggatgaaacaccccgagtagctgaaagttagagctgtggcttcaccctgcccttcacagacacctcatctcggccctcatttcccgctgaacttccttgtgctggaggcttttgaagcctgcctgagtcggccacaccagtcaatacaattgattcttgctggttacagacttcccctccagcaatctcatcactcgggagacacagggaggaggctcgggggttgaaggtcattatttggcaatatagtaagtatttggccagcctgggatatatacagggggccgtataccataattgaacgtgtttcctgtctgtgtgtgacctcatggacaggctttcactgcagagtcaagcagcaacttctgttctctcagtgttggcaggacaagcacgacccgccgtggtcctagagacaggctgggtggaaggatgtcagccctgaagaattcccagtctatctggaaggttcctttgcggatacagcatcataatgggctattgaggagagaggaggcttcatagacaagcatgggcacacacgcacacacacacacacacataaacacacacgcacacacatacacacacacacacacacacgaactgccgcctgtacaggaagcagctggtagagactgggtcggggcaactcacctggggttccctgaccaggatacagtgagcccaagatactactttcatttattatatctaagtacactgtcgctgtcttcagacacagcagaagagggcatcagatctcattacagatggtcgtgagccaccatgtggttgctgggaattgaactcaggacctctggaagagcagtcagtgctctaaccactgatccatctctccagcccaagatactactttcaaaccccacgttgctgcggttggaaccatgactgaaggaacctggcagcctggaaattgtcactacttgtcaggacaaaactacttgataaaaataatgtagaaagaaagccttattttagctcagtttgagggtgccatccattgtgacagggacgtcacactggcaggagcttgagacggctggtaaggactcagagagagagatcaaggctggtagccacctccctttctcccctgtaggcagtccaggactccagcccaggcaatggtgctgtccactttcagactgagtattctgagtattctcttccttaactctgtctagaatttccctgacagacatgcccagaggtttgtctcccaggtgactctaggttgtgttcagttgacaatagtcacagaggcctttgaatgggagaaagtgctagaaaatcagacatgtagagaagtgaaggttccaaacccagcatattctctctctctctctctctctctctctctctctctctctctctctctgaggcacggtcttccatgcctgaggaactctctgtgtagacctataaagtaacttttactcacctgaagtcgtcttattacatccatctgctaacctaggccgagtcctggaagcttccagcctctatacaatctaatgtaggcctagaatgttttcagcctctgatactcgctgacgaatgggttcaccctttctacctctttctgaactctggctggctgattcaactcagctgttccggctccagctcttcttcaagctgactgattcagtctggcttctctcgggttctcactgaattgctctgcttggcctcatactaactctggcagtctgttctaatcttctggcccccttctcattctctggcttgttctctttctgcaaccatctgtgtaagattcctctccttttctctctctgtgatctcttaaattgtctctcttttttttttttaagatttattcatttattatatataagtacactgtagctgtcctcagacacaccagaagagggcatcggatctctttacagatggttgtgagccaccatgtggttgctgggaattgaactcagaacctctggaagagtagtcgggtgctcttaaccgctgagccatctctccagccctctctttctgatcccgtgagagttgggcaggtcctattctgtcaactctttctctgattcggcactttgtctgccactcaattagacatcactttcaaacatggctgcttccttctgtaaactcactttaccttcattgtctggggttaaaagtgggtactaaggttatgtctgtattgtacccagagggattaaaggtgcgtgctaggggttggggatttagctcagtggtagagcgcttgcctagcaagcgcaaggccctgggttcggtccccagctccgaaaaaaagaaaagaaaaaaaaaaaaaggtgcgtgctaagggcttacaccacaactagaaacagtttttttttccagtaaacaatggaatcccagggttcacagtatgatcaaatatcctgcaaaattgaccggattcaatttgaattcgaaccaaccctcctacctttatctcctgagctctgggatgaccaatacgaaccattacacctgctaatggttgtctccactaaaggacatttctttgacttggttacttatccatctctcctccccatcaccaaaacactgggaggagagttagggaggagggagagacagtgaggaaccctgaggacagacacataggacctggggacagacacacaaaccccgaggatggacacacaaggaccccaaggacagacacacagaccacaaggatagacacacagagaccccgaaaacagacacacaaagaaaccaggagggaagcaaaaagacgcagagagacagacgacctttcggtttgtgcctgctttaagcatcagggaagaattttaagccaacacccccgggctgagagaaaccaatcggcctccctctggctgtgtgtgtctgggtggtacacagctgatttccagaccgaaccattttcagatgcacgggccctgcaaatatcagtactatttctactctctcccctccctgccaactgggaatagatcctgttctaaagctcaagttaaaacaagcacttgnaggctttactcacgccgacctttgacctcgaatgcccacttctagtggtttgccccacagaaaaaagtccgggacacgggcagagaatttagcatagatgtttcttctggngatatatatttttagttcatctatctgtgtgttatgtttgagtgtttgctcgcagtgtatgtaggcctgtgtaccaagtgcatgtggcacccacagaggccagaagagggcgttgggtgccctggaactggagttacgattgtttaatgttgggaattgnagctggatcctgtgtaagaagcagccagtgctcttaaccactgagccatctctccggccccttggagtgtgtgcatgcttgtatgtgcctgtgtgtgcttgcatgcatgtatgtatgtgtatgtctgtgcatgcatgtgcatgtggtggtttgaatgaaaatgtcccccaaaggctccagagattgaatacgtgtcccaggtggttgagctctgtgtaggttcaggaggcagagccttgctggaagagtcactcatacatcatactcgttggtgtgtgtacatgccccggtggaggtcagagggcagcctgtaggactcagttctcccctagctctcgggtcaggaggttggaactcaagccctcaggcttggcagcgagtgacttaacccactgagccacctcacaggccttcgaaggctgcccatctttgtctcctccccttcctcctccttgtttcaagacagggtttctctgtgtagccctagctgcattggaacttgctttgtagaccaggttggctgagtaggagggctgcagagatggctcagcagttaagagcaccggttgctcttccagaggtcccgagttcaattcccagcaaccacatggtggctcagaaccatctgtaatggaatccgacgccctcttctggtctgcaggtgtacatgcagatagagccttttaaaaagggcaggccagacgttaatttctggatagaggaattttgactttttttttgtttgtttctttgcatttaaggcaggctctatgtagcccaggctatcctcaaactcctgattcctcctgcatgtacctcccaagtcctggggccacagtagacttgtgcctccatgcctgtttgtattggatttgtttggggtttctcacatgagtaggtgtgcttcctgtgacggaaacacatttcctgaatcatccccaggcgccgccagtgactttccagacctggtagaacaggctgaagcaggctgagggcgggtcaatgacctcgggtagcctgctaagaacttaaaaaaaaaaaaaaaagtctggagagactgctcagcagttatgagtactggctgctcctccagttgacctagatggactcccagagtccactagggctcacaaccacccgtaactccagttccagggcgtccatcaccgctctgacctgggaatcagcaaaggcatggcggcgcgagtagcttgtgccgtggcatgtgtacatggacaatgtttttaaacagctcctctgagggcacacatctgtcatcccagcactaggaaactggctgaagggagatcaggagtccaaggtcgtccttggctacatagtgagtctgaggccagactacgttacaggaaaactattcccacccttgtaaatctgtcttccccgagaccgagaccgagaccggagaccttggtggcctcttgccaggttgttttataaagatttatttatttacatgagtacattgtcgctgtcttcagacacaccagaagggggcatcagatcccattacagatagtggtaagccaccgtgtggttgctgggatttgaactcgggacctctggaagagcagccagtgctctaaccactgagccatctctccagcctgccaggttgttttctaacctgacagctggttgccaccctccctagagggaagacacgactgttcatgcctacacacgggaaggctgacattgccaagtttcctactcccaagaaggggtacaatcaatccacgttcacagcaaagtctggggtccctaccgtgtcctgcaactatgatctgggcgggggtgagggaatggcttggagtcagaaagcgaaacaccagcaaaccaacactgcctgactgtgtgacctcaggcaaggcgcttttcgctgcctcagtttctctatctgcacaacggcactccagcgttcgcagtctctggggttagaccagtgacatataatctgtgaactataaaatgcaataggataagctgtcctttggtctctacattgatgagtttgggcaaatcggttaccttctctgtgtgtctgatggagatccattgccaggcactagaatgcaagctggtaaggctcagcccttcccacccacccacagatctggttgataagctttctgacagagatacttatgcaacctcctctccgctgcctcccacgacagggagctcagggctatacaagtccctgatgcatatgcatgactttaaacagacgtttccaaatgagatgttgtttaaggactggctccgcagttcagtgatcaagtgcgcaatttatgcaaacgtgtgcaaaagcccagagttcaatccccggcaccaagagaaaacagtatttgtgggactggagaggtgacttagtggttaagggcatttgctgctcttacagaggatttaagttcagttcctactggccatgtaatacagctcccagctgcctttaactccagctcccggggatcctacaacccagtttaacttctgtggtcacacatatacatgaataaaaatgttaaaatctggggctggagggtggttcagtggttagagcactgactgctctcccagaggtcctgagttcaaatccctgagttcaaatggtggctcacaaccatctgtaatggggtctgatgccctcttctggtgaagacagcaacagtgtactcacatacataaaataaataataaatatttttttaaaaatgtaaaaatctggttatgagagcacagttactgctaaattagcagattccttcccagcacgtgtgcagtcctgggttctccccttggcatagcataaaccaagcatggtgtcactcctgaggagactgtagaattgggagttcaagatcatccttggctacacaatgtgggtgaagtctgccaaaggtatgtggcctctaagcaggtgcccttgtcacttggtaaaaggcttccaatctcatctgcatttgtgtgggaatctcatatgggtctctcatgagcacagcctggtagaagacctcaggtaggggttggggatttagctcagtggtagagctactgagcgcaaggccctgggttcggtccccagctccgaaaaaaagaacaaaaaaaaaaaaaaaaaaaaaaaaaaagacctcaggtgtggggaagctggaggagccgttttgcagggagaaaatatcttttcagtcactgaaaatttctattgggtggcggtacatggtcaagaagcaggacacccgagttccatgcggatgccggcgtctccaagctatagggcatctgagacttcctcatttgtggtcttgctctgttctgttcctgaggccccaggcttctgggttggagacctcctcgctgagaactgttgggtgaaatggctgacgtcagtgagcccagggcccctcccgcgtggtcccgcactgtcgctgagggtgcgtctcccctctgctccctccacagaggagtacctcagagtgctgaatatgggtctggccgaggtgaagaagttcttcctaggacctctgtctccgtcctgcaagatggtacaaatgatgcgacagtttttgtacgacgtgctccccgaggactcctacaaggccgccaccgggaagctccacgtgagcctcacccgggtcacggacggagagaacgtcgtggtttcggagtaccggtccaaggaggagctcattgaggtaagacactggttggggcgccacctcctggggaaacaggggcagcacggtccgacctcctgtcttcgtcgctcctgtctggggaggaggtccccagcctcctgtcttcgtcacttttctgatttgcctggtccttcccttcccttcccttcctgcctgtgtctctgccaggaacagttctacaagaagagactcgattagtctactaagtggtccagtagattaccaaggacccaacaagggatcagggtgggaagcaagttccagacagttccccaggccagtgcccttactttaaatgtaaggaatggagaattggtaggggtcaggggtcattagcacccaccctagtacctgtagcatctcagccctgtgtctccccagggcctgtagcatcttgctctgtctctccccaggctctgtattgtagctgctttgttcctgtctactgtggcttcatacccccaacatatcggggcgaggtgagtggcttaggtgagctgcgaccctggggcaccttaaggcagggaacatcgaccactcagagttcctacctttttaaggggcacctcctccggttggtccaagtccctctctagacccaccaaccccgtactgaacccagcaagtcaccaaggctcgccaggtgctcccagaagtcagtgtgcagaatacagaccccagaacccagggccctgagaccctgactgaggagaccttcggggctatcagggccaggctttcgtgtgctcatgagtaaactcagataattgggactttgctttaacagatccgggctggccaagattctgcatctctggcttaaggtacccatgaccagtctcctgtgaccggtctggatagggggggaggagggatgtaaccaagtggtccagatattttatggcagccctaggatacagaatctgtgtactggctagctcttattcagttcataagtaaggaaaccaacagaagtcccagcccccactccaaagaaaagggggtggaggttggtgtaccgtgggtgaggttgcagaagcggggatggtttcttaagtcattcagtagaggtaccccataggtgatatcaacaaggttcgtgagggagaacagtccagggccagagccttagacttggagtcccgacgccattatagatgcaagtgaccatgcccacgccacctaccatctatctcgggggacctcagcctcgaggcttgtcggtcgcaaagggggggggctctgctctcccatggccccagctcctccgagccttttctgactgagcgtctctcgtccctgaagagatacatcgacggcggcttcacaagcatgcagccctgcgccttctggacagactccatcaccatctccaccttcagcagccagcaggacatctgtccgagggactgccccgccatcttccatgacttccgaatgtttaacttctccttccagttctccttggagaatatcacgcgcatgacgcatgcgctgttccccccggacctggtggtgagagacagggcccgcagggtgggatccagaacagatggggaccctccttttgtgcctctcccctcaaaatgtccagggttttgctgtgcttaaaggaccgctaaaaaaaaaaccccagcaggctacagatgtcattcaattggtagagtgcttgcctagcgtgcatgaagcccggggttctattccccagcaccgcacaaactgaggacagtggaatattgtagtcccagtactggggaggtggaggcaggaggatcaggagttcaaggtcacccttagctggatggggaagtttgaggttagctcgggttacatgctatccggtctccaaacaaaatcccacaggttgagaacatgaggctgtggtggttacagctacacaaagagtagggctggagtttgaatcaaaacaccaagctctgtccttcttaagacccagacgtgcggtctctggttcccccagagacatcccaccaactcccacagtagaggttggtgatgctctgggaacagacgaccgacacactgatacgcccacccctaccatgttagcagttgcttcccgctctcctgggcagacagtgagggggtggggtatagcccggggcttgctgacatggtcctgggtaggagggactagaggagtgtctacttatagagcggctatggtgggacactggccctcttggtggctgcagcctcgtttgttttcctgtgtcaccatctagatcctgcaggaatattactaccggggatacgatgatgccgtctcatacctgcggaggctgagtgagtaactggagcctggggaagggagggcaaggtgtgtggaaggggtgggaatagaagtctgggtagaggacagggtcccaaagctgtagccaagaaagctggacagaagaacagcccaagacagaccactgtggctgtgagttgaaaaaatatatgtatatatcatatatttagtatcatatattttagtcttttgagacagggtttctctgtgcagccatggccatcctagaactcaccttgtagagcaagctggcctggaactcaagagatccacctgcctccgcctcccaagtgctgggactaaaggcgtgtgccaccgctgaccagctgagaatatttctaaaagtgtatttataaaattaaaaacagagaggttggcgctggagttcagtaggtagagcgcttgcctggcgtgcataaagccctgggtcactcttcaacgccccatcagacaaggactgtgaacgagtgaatgccgccactcagaataaggctaaaggattggatattcaaggttaccctcagatatgatagctgcttctaagccagtctgtgctgctccagacccggtctcaagtagatgtgactacatttgagtgatttactgatcaacatcctatattggtctgggaaggggacccggaccagtttcctctcgccttcatcagacgaataaatcaatacagaggctaattcgatcaaactccgtgaaggtgacaagcatctttatggagagagaccagagacctgcccatatttagggttaactacttagaatgtggttccaggggctggagaggtggctcagtggttaggaacactgactgctctcccagaggtcctgagttcaattcccagcaaccacatggtggttcacaactatctgtaatgggatctgatgccctcttctagtgtgtctgaagacagtgacagtgtacttatatataataaataaatctttagaatgtggttccagacaaagggaatcctaggggccaagggcgcttaccgaatgctggcctggcctttctccacgtggtcacacaatcttcagagtagatgtgtaatatcttatcacagtcgtacaaagtacttggcttctggactctgagttccaagcattccttattctgatatggctatacagctctgtaagtttaaaaagtatacttggggaaagaaatattttggggtgaaatgagggttgaacgatattttcttactagaatatatatatatatatatatatatatatatatatatatatatatatatatattcttttaaaaatttacttcctgactttctcctgggtccttattccttaatccaggcattcagggacttaggaacagaatgagggagagaggggacagacagacagacagacagacagacagacagacagacagacagagaaccacttgggtcctggagacgagtggggttctcagccccacccctgagctgggtgtgcttggaaaaatcactcttgctctcagagtcttagttcctttgtaaagaagggtttgtacccgagctcctgacacctttggggtgtccagtaatgagtctcctggggtctcggcttctgtatctataaggtagcgactgtctcagagagttttttttttggttcttttttttccggagctggggaccgaacccagggccttgcgcttcctaggtaagcgctctaccactgagctaaatccccagccctgtctcagagagttttactgtgagctcctggaggatggggcgggggaggtggttggccactactctttggccaccagctgggaagcccacaatcttcctgggtgatggtgtttgtgaggaactggaggttaggaagatcctacaatttgatacttacattttttttttttttgatacttacattttggaggagactccgtccccgtcccgcccccgtcccccccctccccccggccaggccacaaccttctgcatctgggtcacagcttaactacactctctggttcctgtccccgctgcaagggtagcctaggttctgcccaggctccttgtgatgcgcgggccttccgccagatggcgccagcgcacacgcacgagtccttccctcttgcccatccctccctccaggcttcttggatttttctactggcagaagatgtgatcagtagctcctgggaaaaccctgctggtacccagagccacactatgggttcccacctgccgctaggaagcagtcagaatgctctctgcttgattgaggggagaggggggagggactcatcacacacagaataaagctgccctggacccctcaatctcatcgtcaccccctctctcctccctcccttgcaaaagctctcatccacccagttactcctgcagttggaagccgctagccccgcctgcctccctggtgtgggaagggagaggcggagctgaaggatgccagtctgcacccagctgtgcccagcaagcttcgccctcactccttccgactaggtcctcccacacatgcacattctatcatccccgtctttctggagaggccgggactgggacttgagtagagtacttacctagcgtgtgtgaaaacccacgtttaatttccagtacttgtaaacctggcagggtggtgcgtgcggtaatcccagcactggggatgtgcgggaggggggggtagaatagacgttcaaggtcatccttggctatatagtaaatttgaggctaacctgggctacatgaaatcttgtctcaaaaaaaaagttttctggagctcctttatgatcccccaccttcgccaaaaacaccccacccccaccccacaatctgttctctgttcttacgatcttctagccttagccaagtgctgggacaaggctgtgctcatcagatatatttatgtagattttgcacacacacccatactagattatgagcatatcaaacctgatgtggtttctcctagatgccgcgtacctcgactctcccagcaagagagtgatttttccgagggtagaagtatactgccagatagaggtcgcccttggccatgagcccccacctacgagtctgcagagcctcccagcacagagagacagccctgaggactgctcacaaacccatgtacagggggctctcaaagaggacagaaaggacagccattccccagcaaccccatcagcgcagacatctgaatctgggtgcaaggggtttgaggagtcacctgtgtccccaacggtctcttcatccaagcaaccagctgtgtcaccgttggtccaagcccagccgggcgcagtagtgaggcccgctgaccccagtgacagttgcccagtgaatgttcaaactccaaacctggagcaaggagtgaaggatgactgtcagccagacttggccactgatgaccaaagcacaaacagcctgctccctgcatcactcccagctgcagactcacaggaatcaaaggctggatcctctgtgtctgttgcgtcactcgagtctgctggactgctgctcaaatcttcccagggagccacagtagccggggcaacagctggacttccgcccctaagtccttcaacgccacccgcccagccacctgtggaagacctaggcccagaacaaccctcaggtaatgccctccctgctctggtccacaggcaagctatcaaccatgtggagctgagaaacatcaattccaagggtgacattggccatctattttagcagcttccacaggggatactggcccttgccttgtgtctttgtccccatgtatcttgaacctccttctggcaacaaaaaacaccacgaagaggttggagagatgggctaaggacaattgctgttctcttagaggacctgagtttagnttcagagtggccaggagactgggtggctcaaaaccatgtcggtcccctcttctgggtcctatgggcacctgcacacacacgtgcacacacctgccatctacacaactagaaataaaataaatctggggggggttgtttttttttaaagatttatttatttattatatataagtacatactgtagctgtcttcagatacaccagaagagggcatcggatctctttacagatggttgtgagccaccatgtggttgctgggaattgaactcagaacctctggaagagcaatcgggtgctcttaaccgctgagccatctctccagcccataaatctgggtttttaggatttatttttttccttttggttttccaagacagggtttctctgtgtagccttgactgtcctggaacccacctggctggcctcaaaatccgagatcctcctgtctctgccttctgagtgctaggattaaagttgagtgccactgccactcagctaaaaaaaaaaaaatctcaaaaataaaatgaatttaatattgatattaaatgcttataataaagccatttatatttagaactcatacagttcatacaaatgtttaaccaataaagataaagtcatgaggacagagttttatccccagtactcacataaaggctgggtacagcatcctgtgtctgtaattctggcgccgagggcgtgaagacaggcagatccctgcagctccgatggctgatgagcctagcgggatcagtgaaaaattaaagtatatataattaaatgtgtaattccagcactcgtaaggcagaggcaggcagatctctttagtttgaggccagcctggtctatatcgcaagttacaggacaatcatggcacacagagaaaccctgtcttgaaaaaaagcaaaaaccaatcaatcgaggacgacacccagtgtccatctctgacaccatttgaacactcactccaaacacacacacagtggtcctaataataatcttcattccaagatgaaggtttacttgtgctggtggctgttcaggccccgccctttgacgtcaccttgggggcggggaagagcggggtctgcccacctccctctgctgcagccgtaggtctccaccggatccactgatgtggttgataatttggctcgtaaatatgagaaggagagcattcttggccgatttgattgcttacttgtttgtttgcttattattttaaaataaccttattaatcctttgaaatattcatacctgcctacatccgtatgtgtgtttttgaattttgatttgttgggtacggtgggatgcctcgcacagcacatgcgtggaagccagaggacaactagcaggagttgactctctcctcttctaaccacatgggtcctgcatagtgtagctctggttctcaggctccgagggaagtgcttttatccactgagccaccatgacgactcacttttgctgttgtttttaagacatttacagagactagagacacagcatttaaagagcactggctgctctcccagcatccccatgacctcttgcgtctgaattcaccatacacatttgggcaaaacacacatacacataagactttgaaaaatcgtaatgtttaaaaataaaacattatgtttcctgaatgccctggcccctgcctttaatcttagcactttcgaggctaaaggaagcagacctctgagttcatgaccagcctagtgtttacaggctggaccaggatccagggcagccagggctacatagaaagatttgggggggggggaagattttctttgatgaacattcagggttcgtgcgtgtttctgtgcctcgctcacacccatacctggtaccaaagaggtcagaagagggcgctggaaaccctggagcagttgtgagctttggggagtcagaggcaggtggatctctgagtttgaggccagcctggactacagagagttccaggatagccaaagctacacagagaaaccctggctcaaagaattgggaaaaagaaaaaaggaaaaattttttaaaaaaattaaaaaaaaaattgtttccttaatggtagcatttcaggctggagagatggataagtggttaagaacaccaactgctcttccaaaggtcctgaattcaattcccagccaccacatggtagctcacgaccatctgtaatggggtctggtgtgtctgaagacagctacagtgttctcatataaaatcaatcaatcaatcaatctttaaacagtagcatatcgctattgggtccctggatagtctggctctcgttatgtaaaccagaccagcctcaaactctgaaatccccttgcctctgactcctggaactaaaggagtgctggaccacagtatctgccatccttgttgttctcagacacactcaccgttgtttgcaaaatgacccagtacccagtacctgcccttatacctcccgtgtttcttccagctacagggtctcccgcctcatcacagctgacaggctcggcagcaccggatacggggaagggagcccccgacaagcctctactggtgtaagtttctccttcctcaggactgattcctttgcggggcgggctaaatgaagtttctgcactttcggctgcctgggtattacttagctcttcgagtctgtggggttcactcttcctgccctcttctctatcttgtagtgaggagctatgtggaatatatctgttcatgagattacaccttccctcttccccattccagaacacacggggtacaccctgttccagaacacacggggtacaccctgttctagaacagtgggagcgtgtgcattccaggtcccgggtcttattttgggtgaagccaccattggaacggtgtaacaggtgcaccatcctagcgttaggtagctcctcgtggtagaaaaggcctctcaaaggtctccattggagctggcgcagtggtgcgtggctttagccccagcactgtaaccttgaagtgaatgaaaaggagagagtaggctgagcagaggcgagcatggttcaatgctctctgctcctgactgtgagagtgacgagatggggtgctgcaaggtcctgcgtacttcacttcgtgtcagtgacggatcatcgcctggatttgtgagccaaataaaccgtttctgccttaagtgantttgtcagggcttntgaccaccacaacagaaaccaaaccaagagagcatgcctacggncttcncggatggggnntgcagatctntagtnnnnnaannnnnnnnnnnnnnnnannnnncaggcnnnnnnnnnngattngnanacagtnngntnnnnnnnnnnnnnnnngngnnnnnngtcnnnnnnnnnnnnnnnnnnnnnnnnnnntccctccnnnnnnnnnctctctctctctccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctggttaaggtcccctcagggatgtgtctccttactgacactcctcacttaggacgttgggtctcttatatatactccaagtataagagactgcactctagagtaagccaattgacattccagttctggatctgatttagactggctgagcaactttgaagaaattagttaatgtccanaagtctccccctctccctctctctctctctctctcacacacacacacacaaacacacacacacacaccccacacacacactttcaactctgtcttttagaaaagcggatttgggggctggggatttagctcagtggtaaagcgcttacctaggaagcacaaggccctgggttcggtccccagctccggaaaaaaaaaagaaaagcggagttgaaacattctgttcttaattttatcgttgaatgaacgatacaattacaaagcccgggctggcaatgagtgggcaacctgcctctgctttccgagtcttgagattataggtgtgtgccaccatgttcttgggcattcttccatagaacagaagtgtgttaacttcacaacaagcttactaagatgtttcccccattatgtaattttatctctacagttctccaactccagaggatctgacaccctcttttggtctctgtgattatgtccttacacgtaaacacacttacgcataccagataaatagttaccggtaaccagacgtgttactggggaacgtcaaccttagccagggccactgggtctgctgagctgaggcaggactctgacggtatcagacactgttcaggctctcataccaagacatctcctgtgtctctttgcccagggccagtcctgctgcagattcaaacacagcaaaaaagatgttcaggaggaaacagaaggcaaatgccaccagagagtgcttccagaaaaaccctcagtccaagaagccagctagcaaactgcagtaagtgtgagtctctgctctgccaggagagctcggcgcttagaacatctaggctctccattttctgaccttcaaaaacggggtgacccgggggctggggatttagctcagtggtagagcgcttacctaggaagcgcaaggccctgggttcggtccccagctctgaaaaaaagaaccaaaaaaaaaaaaaaaaaaacggggtgacccggttcataatcctgggagggtgcagccctcctgtgaaagggaaagccttcggaaaacagcatgtgtcaggtgcatgggtcctggaggtttgctagctacttctcaactgtgtattgtgttgttttgatcagagtagccctggctatcccataactcaccatgtagaccaggctagcctcaaactcactcactgtgcagctagagctaaccttgtacttttgatcccctcctgctcccggctccccagtcctgggattataaatatgtgacatcactctttggccagcaaaccccttacccgtacaaacagggagttgcttttgaggattctttagtccatttggcagaaatttccctaaacatttttggcatttatttactgactgattgtgtaatgtgcgtgagtgtgtgccggagaggtcaaaggacaggtttttctggaggcagatttcctgaccttctgtctcatgggtcccaggaactaacctcagacgggcagggttggaggcacgtgcctctacccactgaacatccctcttgggttcccaaacttgtattttattcagctgtcaattagtcttcttggggagatgctgggaccctgttgcctgctcacaactgcctgtagagagagtctcatgtaacagaggagtctaaggatctgtctgtgtttgagggtcaagggtcaaagctccctctgtgatccctgtgtccaaagcagccataatgttacccaagagccagtgaagcctgccaatccttgggtccagttggaaaccccagaataaggaactgcagctacaagggaagtaaataaatacaactttaaaattaaaacaagacccatccatgagggggtgcaagtctaatgccaagattacttctacggtgggctctgcaggatcttgcaactttccagagagggcaaggggctaccctggggtcacacagcaggtgaaagtgaggcttgagactcttcttatttcccctcatgaatgtgtccttggcgtctcaaatccaggtctgcaccttgtccacttgacttttctgtacttcccgagacagtctgggtcacctacaaacctcatcccaaccggattcaagaatacagctacactgaggtatggaactcctctggatgtcacctaagagtgaccctgcctctcctgtctcagggggatgcagcaactctgagcatatagcttcctttctgcccttccttcccttccaaggacagccttggtcagctccagcccattgcctggacagctgggagtgtggtggtgcacacctttagtgccagcatttggggaagcagaagcaggtggatcgctggggagttcaaagctagccaacatagccaacattatattaggagtccttgtgtcaaaaactccaaaatgggaacagagaaacacacacacacacacacacacacaccacacacacacacacacacacacacacacacatacacacacggcaagagagagagagaagccaaagcaagaaagcaagctagactgtttgaggaggtggtggtgcacacctttaactccagcactctagaggcagagctctgcaggaggatctctgtgagttcgaagtcagcctggtctacatatcaagttccaggacagccagggctacataataagatcctatcttaaaacaaaacaaggggctggggatttagctcagtggtagagcgcttgcctagcaagcacaaggccctgggttcggtccccagctccgaaaaaaagaaaaaagaaaaacaaaacaaaacaaaacaaaaggagagccaaacagaagttacattgattttgttaagaccaatgcttctcaatccctctgggataccatagcagatatctggagtatcagattgttacattacaattcataacagaagcaaaattacagttacaaagtagcaacaaaaataattgtatggttgggggtcaggcaacaggaggaactacattaaagggtcacagcattaggaaggttgagaagcacaggttaagataccgacagctgggggtacacacctttaccctacctctcagggacagaagcgggcatatctttgaatttaaggccagactgacctacagaataagttccaggacggccctggctacttagagaaaccctgccttaaaataatcaaaaccaagtcaaaccaaatagcccaaaacacactgtactgataacttccagatagtactcatagtactcatatgtccatacctggcatagaacagtaaccacgcaactgaactgagcagtggagagaccacaccacccttctggaaaaaacaagaaccaactaatcttttataatatttcaaacatttaaaaaaaatcatgccggttgtggtggtgcacgccagtaggatttgctgaaggaggcagaggcaggaggatcacgagtttgaggccagcctgggctacacattttggggttggggatttagctcagtggtaaagcacttgcctagcaagtgcaaggccctggcaaggccctgggttcggtccccagctccgaaaaaaagaaaagaaaagaaagaaaaaaaaagtcggggtgaactaagacaggatgtcttagggaagggtcactcaaactgacgactctttgtcctttcctagggcgtgagtggacagaattcctaggctggagcaggagaccccagtgacatgaagcctgggtccagcctccacctgaggtcttttgggaccaggggagccctcagctaccaaatgctacagaaagtctactcccccaaggttgggtttcctggtctgaagagtctcctgcttccagtcttgctgcccttccaccgttctgctctggatggacagacagacatcaacgcataccactcaccatccttccaggaggagggacacaattctgcttttgtccaatctggggcaagcatacatcctgactgtacttcctgcctatgttcctcccacctacatgtgaccgggcacatcctgtacagttggggcaagcaagctgtttacagaagtgaaaacaggtggcttgttgtcttacatgaaagatatcccccagcattcctggaagttatctgaccttgggcaagtggggcttacaggttaaaaacatctttgttttgtaggtctcttaagcacagcaattaaaacttaaaacgtaactttagcccttgcactctaagtcctccagcagaatcgttgcagaccccaggagagctctgaaagaaacaaaacgaaaaggatgtgactgctcctaggtatggtggaggagaaggtttattgcagacctatgggagaaaacagccagaggcatctggaagagtccagagtgaacgtgatactgaggtgggccatgtgaggagaaagagaaaggggagaaccaggagacccaaatgtaaaagggtaaaataaacacatagccaaaatgactggattgtatatgaaaaggcagctgggggcagggcagcccagtggctatgctagagagttcagggtagagtccctgggacaatcctataatagcagctagggactgagggatgctgagagagcctggcaggtccattttgatatgttaataggcatttctgctgagtccaaaggatgaaaccagtccaagggaccacaggctcagcagacgtgggcacgaacagaccagattctaggcaggttgcacacttgttcatacctcatgcttacccttccgagagtctgtctctgcaaccacggaccttggagggttcgttccgccacagtctactgtgaagaaagtgtaccctgcagagtgccacaagccagagtcaccctggctaggatttggacaatgggtctctctctaaggctcaaaatttccagcctgcccagcagaaggcgtcccccactgctccccagaaaggaaaccagcccagccagacccagcctgcccacgggccctgccagtatttgcttaactcaaatgcaaaacctagagccaagggtgaagctcaaatgtaagctggacttggccactggggaatctacagaaatgttagcagagaaagggggccctgtgccaaggaggccttgggggaggctatactccctaccccactcccaccccaagtcatttcaggaaagagtgggctttgaaagattagcaaagaatcaaagatcccagagaggaaatgtggtagctggactctaaaggataagagttcaaggtcatcctacatagtaagttagagactagcttaggctacataagactgtctcaaaaacggggcagagggcgctcccccccccactggagagatgcccagtggttagagcactgactgcttgtccagaggtcctgagttcaattcccaggaaccacatggtggctcacaaccatctgtaatgagatctggtgccctcttatggcctgtaggcatatgtgcaggcagaacactgtatacataataaataaagcttaaaaaaaaaagggggggggctggggatttagctcagcggtagagcgcttacctaggaagcacaaggccctgggttcggtccccagctccgaaaaaaagaaccaaaaaaaaaaaaaaaaaaaaaaaaaaaaaaagactgtctcaggggctgtctttaaatccaagatctgcggttctcacacaaacatgtgcaggccaatcaccaatacacattaaaaaaaaaaaaaaaacctcccagccatccacaaaattagatcaaataacatggtcgatccagtggtgtagaccagtgagggcaggacctggaggatggcatgtggcagcatgtccctgaggcaggactttcacccaagtatgaggacctttgccttcaggagagtctctcaaccctggcgtaccaattccctggcccaggggtacgtggtacctgttaaaattgtcttccctcagagtgcgtatgtcaagcctagaagcctcctgagtgaaaaaacaaaaaacatggctgcttctaggtatggtgggggagagatgtatttattatagaggtgtgggagagcccagagcctgtgccacgtagcaggagggggagtgaaagggggaggggaaaggagagggggaggaagagaaggtaaccaagtaccacagccaggaggccaaaggtacaaaatggatgggtggcaaaatgtctgcattatataggtgaagggcaacccagtccctggactggagagatcagggtagagggcggggtctgccagccacaccctgtaacaggtagggaccgagggatgctgggagaacctgaccaggtaggtcccttttgatcttaactaggcacctcagccatttatgcctgcttcgaccctgaacacctccaatgtccaaggagccagaacccccttattggtcctctaggattctgttaatttccccggttttgaggtaacgcgggtgtttgtagcagttccaggattggcagatgaagacccaggacaccttgtgactgaagcatcaggtgaacacggaacaattgctctcctggcagctgttcctaacatcacagtcatttacctgaggttctgatgtactggtgtcctgtgacttcgtctgctaaccgttaacttctattggtcccttcagtctctccctttctacttgtttgtcttcaaacatgaggccttggtaggctggtttagccagcctggagcacaccctgtagaccaggctggcattgaactcacagagatccacctgcctctgcctccccagagccaaaggcctgcaccgccatacccggggctcttcctttaactgatttctgtttgtggactgggatgtcttacatagcctgagaaggctaggctgattccacaacagacttcaaattggcagttaaggacactaggcctaaaactgggtgagtcgggctggagagatggctcagcggttaagagcactgactgctcttccagaggtcatgagttcaattcccagcaaccacatggtggctcacaaccatctgtaatgagatctggtgccctcttctggcctgcagtcacatatgcaggcagaatgctgtacataaattaaattaaaaaaaaacaaaaaaacaaaaaaactggatgagtcggggcaagtcaattactaataggaaaaacaaacaaacaaacaaacaaacaaacaaacaaaaaaccacccaggctccaaccttcacgcccaggtaatggaatatcccaaccacctgacctgaggtgaggacaatataggtcagcagcagtttccagatttcctcagctacctccccagcaagtttccagcccccacaccttggtaattgaatgtccaacagattaacacaaaggtccctaattaacaggagttccctaatgtgctttaaatcaggcctgcaatgtctctagcttgataaggggagaccccaatatgctgcacttctgcagaataaaacattctttgcgtttacatagtatttgaatccggggtatcattcttcagcaaatcatggaccctaatagcccaggctgtactcatactggatactgttaggtcccaaagaaaccccggg